SEQUENCE LISTING (46 sequences)

SEQ ID NO: 1 - DNA, 24 bp, Artificial Sequence
  source: /note=“Description of Artificial Sequence Synthetic oligonucleotide”
ctctctacggctaacctgaatgga

SEQ ID NO: 2 - protein, 8 aa, Artificial Sequence
  source: /note=“Description of Artificial Sequence Synthetic consensus peptide”
  VARIANT (2)..(2): /replace=“Ile”
  misc_feature (2)..(2): /note=“Residue given in the sequence has no preference with respect to the annotation for said position”
  MOD_RES (4)..(4): Any amino acid
DVEXNPGP

SEQ ID NO: 3 - protein, 8 aa, Foot and mouth disease virus 2A
DVESNPGP

SEQ ID NO: 4 - protein, 100 aa, Artificial Sequence
  source: /note=“Description of Artificial Sequence Synthetic polypeptide”
  misc_feature (1)..(100): /note=“This sequence may encompass 3 to 100 repeating residues”
  VARIANT (4)..(100): /replace =“ ”
  misc_feature (4)..(100): /note=“Residues given in the sequence have no preference with respect to the annotation for said positions”
KKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKK

SEQ ID NO: 5 - protein, 3 aa, Unknown
  source: /note=“Description of Unknown Integrin receptor-binding moiety”
RGD

SEQ ID NO: 6 - DNA, 2727 bp, Human cytomegalovirus
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagcgcgtgaaatacgtggaggacaaggtggtggaccccctgcctccttacctgaagggcctggacgacctgatgagcggactgggcgctgccggaaaagccgtgggagtggccattggagctgtgggcggagctgtggcctctgtcgtggaaggcgtcgccacctttctgaagaaccccttcggcgccttcaccatcatcctggtggccattgccgtcgtgatcatcacctacctgatctacacccggcagcggagactgtgtacccagcccctgcagaacctgttcccctacctggtgtccgccgatggcaccacagtgaccagcggctccaccaaggataccagcctgcaggccccacccagctacgaagagagcgtgtacaacagcggcagaaagggccctggccctcccagctctgatgccagcacagccgcccctccctacaccaacgagcaggcctaccagatgctgctggccctggctagactggatgccgagcagagggcccagcagaacggcaccgacagcctggatggcagaaccggcacccaggacaagggccagaagcccaacctgctggaccggctgcggcaccggaagaacggctaccggcacctgaaggacagcgacgaggaagagaacgtctgataa

SEQ ID NO: 7 - protein, 907 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 8 - DNA, 2256 bp, Human cytomegalovirus
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagcgcgtgaaatacgtggaggacaaggtggtggaccccctgcctccttacctgaagggcctggacgacctgatgagcggactgggcgctgccggaaaagccgtgggagtggccattggagctgtgggcggagctgtggcctctgtcgtggaaggcgtcgccacctttctgaagaactgataa

SEQ ID NO: 9 - protein, 750 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKN

SEQ ID NO: 10 - DNA, 2082 bp, Human cytomegalovirus
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagtgataa

SEQ ID NO: 11 - protein, 692 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQ

SEQ ID NO: 12 - DNA, 2232 bp, Human cytomegalovirus
atgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacagcagactgctgatgatgagcgtgtacgccctgagcgccatcatcggcatctacctgctgtaccggatgctgaaaacctgctgataa

SEQ ID NO: 13 - protein, 742 aa, Human cytomegalovirus
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 14 - DNA, 2151 bp, Human cytomegalovirus
atgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgactgataa

SEQ ID NO: 15 - protein, 715 aa, Human cytomegalovirus
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATD

SEQ ID NO: 16 - DNA, 840 bp, Human cytomegalovirus
atgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataa

SEQ ID NO: 17 - protein, 278 aa, Human cytomegalovirus
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 18 - DNA, 1119 bp, Human cytomegalovirus
atggcccccagccacgtggacaaagtgaacacccggacttggagcgccagcatcgtgttcatggtgctgaccttcgtgaacgtgtccgtgcacctggtgctgtccaacttcccccacctgggctacccctgcgtgtactaccacgtggtggacttcgagcggctgaacatgagcgcctacaacgtgatgcacctgcacacccccatgctgtttctggacagcgtgcagctcgtgtgctacgccgtgttcatgcagctggtgtttctggccgtgaccatctactacctcgtgtgctggatcaagatcagcatgcggaaggacaagggcatgagcctgaaccagagcacccgggacatcagctacatgggcgacagcctgaccgccttcctgttcatcctgagcatggacaccttccagctgttcaccctgaccatgagcttccggctgcccagcatgatcgccttcatggccgccgtgcactttttctgtctgaccatcttcaacgtgtccatggtcacccagtaccggtcctacaagcggagcctgttcttcttctcccggctgcaccccaagctgaagggcaccgtgcagttccggaccctgatcgtgaacctggtggaggtggccctgggcttcaataccaccgtggtggctatggccctgtgctacggcttcggcaacaacttcttcgtgcggaccggccatatggtgctggccgtgttcgtggtgtacgccatcatcagcatcatctactttctgctgatcgaggccgtgttcttccagtacgtgaaggtgcagttcggctaccatctgggcgcctttttcggcctgtgcggcctgatctaccccatcgtgcagtacgacaccttcctgagcaacgagtaccggaccggcatcagctggtccttcggaatgctgttcttcatctgggccatgttcaccacctgcagagccgtgcggtacttcagaggcagaggcagcggctccgtgaagtaccaggccctggccacagcctctggcgaagaggtggccgccctgagccaccacgacagcctggaaagcagacggctgcgggaggaagaggacgacgacgacgaggacttcgaggacgcctgataa

SEQ ID NO: 19 - protein, 371 aa, Human cytomegalovirus
MAPSHVDKVNTRTWSASIVFMVLTFVNVSVHLVLSNFPHLGYPCVYYHVVDFERLNMSAYNVMHLHTPMLFLDSVQLVCYAVFMQLVFLAVTIYYLVCWIKISMRKDKGMSLNQSTRDISYMGDSLTAFLFILSMDTFQLFTLTMSFRLPSMIAFMAAVHFFCLTIFNVSMVTQYRSYKRSLFFFSRLHPKLKGTVQFRTLIVNLVEVALGFNTTVVAMALCYGFGNNFFVRTGHMVLAVFVVYAIISIIYFLLIEAVFFQYVKVQFGYHLGAFFGLCGLIYPIVQYDTFLSNEYRTGISWSFGMLFFIWAMFTTCRAVRYFRGRGSGSVKYQALATASGEEVAALSHHDSLESRRLREEEDDDDEDFEDA

SEQ ID NO: 20 - DNA, 411 bp, Human cytomegalovirus
atggaatggaacaccctggtcctgggcctgctggtgctgtctgtcgtggccagcagcaacaacacatccacagccagcacccctagacctagcagcagcacccacgccagcactaccgtgaaggctaccaccgtggccaccacaagcaccaccactgctaccagcaccagctccaccacctctgccaagcctggctctaccacacacgaccccaacgtgatgaggccccacgcccacaacgacttctacaacgctcactgcaccagccacatgtacgagctgtccctgagcagctttgccgcctggtggaccatgctgaacgccctgatcctgatgggcgccttctgcatcgtgctgcggcactgctgcttccagaacttcaccgccaccaccaccaagggctactgataa

SEQ ID NO: 21 - protein, 135 aa, Human cytomegalovirus
MEWNTLVLGLLVLSVVASSNNTSTASTPRPSSSTHASTTVKATTVATTSTTTATSTSSTTSAKPGSTTHDPNVMRPHAHNDFYNAHCTSHMYELSLSSFAAWWTMLNALILMGAFCIVLRHCCFQNFTATTTKGY

SEQ ID NO: 22 - DNA, 1422 bp, Human cytomegalovirus
atgggcaagaaagaaatgatcatggtcaagggcatccccaagatcatgctgctgattagcatcacctttctgctgctgtccctgatcaactgcaacgtgctggtcaacagccggggcaccagaagatcctggccctacaccgtgctgtcctaccggggcaaagagatcctgaagaagcagaaagaggacatcctgaagcggctgatgagcaccagcagcgacggctaccggttcctgatgtaccccagccagcagaaattccacgccatcgtgatcagcatggacaagttcccccaggactacatcctggccggacccatccggaacgacagcatcacccacatgtggttcgacttctacagcacccagctgcggaagcccgccaaatacgtgtacagcgagtacaaccacaccgcccacaagatcaccctgaggcctcccccttgtggcaccgtgcccagcatgaactgcctgagcgagatgctgaacgtgtccaagcggaacgacaccggcgagaagggctgcggcaacttcaccaccttcaaccccatgttcttcaacgtgccccggtggaacaccaagctgtacatcggcagcaacaaagtgaacgtggacagccagaccatctactttctgggcctgaccgccctgctgctgagatacgcccagcggaactgcacccggtccttctacctggtcaacgccatgagccggaacctgttccgggtgcccaagtacatcaacggcaccaagctgaagaacaccatgcggaagctgaagcggaagcaggccctggtcaaagagcagccccagaagaagaacaagaagtcccagagcaccaccaccccctacctgagctacaccacctccaccgccttcaacgtgaccaccaacgtgacctacagcgccacagccgccgtgaccagagtggccacaagcaccaccggctaccggcccgacagcaactttatgaagtccatcatggccacccagctgagagatctggccacctgggtgtacaccaccctgcggtacagaaacgagcccttctgcaagcccgaccggaacagaaccgccgtgagcgagttcatgaagaatacccacgtgctgatcagaaacgagacaccctacaccatctacggcaccctggacatgagcagcctgtactacaacgagacaatgagcgtggagaacgagacagccagcgacaacaacgaaaccacccccacctcccccagcacccggttccagcggaccttcatcgaccccctgtgggactacctggacagcctgctgttcctggacaagatccggaacttcagcctgcagctgcccgcctacggcaatctgaccccccctgagcacagaagggccgccaacctgagcaccctgaacagcctgtggtggtggagccagtgataa

SEQ ID NO: 23 - protein, 472 aa, Human cytomegalovirus
MGKKEMIMVKGIPKIMLLISITFLLLSLINCNVLVNSRGTRRSWPYTVLSYRGKEILKKQKEDILKRLMSTSSDGYRFLMYPSQQKFHAIVISMDKFPQDYILAGPIRNDSITHMWFDFYSTQLRKPAKYVYSEYNHTAHKITLRPPPCGTVPSMNCLSEMLNVSKRNDTGEKGCGNFTTFNPMFFNVPRWNTKLYIGSNKVNVDSQTIYFLGLTALLLRYAQRNCTRSFYLVNAMSRNLFRVPKYINGTKLKNTMRKLKRKQALVKEQPQKKNKKSQSTTTPYLSYTTSTAFNVTTNVTYSATAAVTRVATSTTGYRPDSNFMKSIMATQLRDLATWVYTTLRYRNEPFCKPDRNRTAVSEFMKNTHVLIRNETPYTIYGTLDMSSLYYNETMSVENETASDNNETTPTSPSTRFQRTFIDPLWDYLDSLLFLDKIRNFSLQLPAYGNLTPPEHRRAANLSTLNSLWWWSQ

SEQ ID NO: 24 - DNA, 519 bp, Human cytomegalovirus
atgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataa

SEQ ID NO: 25 - protein, 171 aa, Human cytomegalovirus
MSPKDLTPFLTTLWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 26 - DNA, 648 bp, Human cytomegalovirus
atgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggcgcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataa

SEQ ID NO: 27 - protein, 214 aa, Human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 28 - DNA, 393 bp, Human cytomegalovirus
atgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataa

SEQ ID NO: 29 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 30 - DNA, 550 bp, Artificial Sequence
  source: /note=“Description of Artificial Sequence Synthetic EMCV IRES polynucleotide”
aacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataat

SEQ ID NO: 31 - DNA, 678 bp, Artificial Sequence
  source: /note=“Description of Artificial Sequence Synthetic EV71 IRES polynucleotide”
gtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcg

SEQ ID NO: 32 - protein, 868 aa, Varicella zoster virus
MFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 33 - protein, 841 aa, Varicella zoster virus
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPKYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGLPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIFALSEMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSKDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 34 - protein, 160 aa, Varicella zoster virus
MASHKWLLQMIVFLKTITIAYCLHLQDDTPLFFGAKPLSDVSLIITEPCVSSVYEAWDYAAPPVSNLSEALSGIVVKTKCPVPEVILWFKDKQMAYWTNPYVTLKGLTQSVGEEHKSGDIRDALLDALSGVWVDSTPSSTNIPENGCVWGADRLFQRVCQ

SEQ ID NO: 35 - protein, 354 aa, Varicella zoster virus
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 36 - protein, 623 aa, Varicella zoster virus
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 37 - DNA, 15271 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacagcagactgctgatgatgagcgtgtacgccctgagcgccatcatcggcatctacctgctgtaccggatgctgaaaacctgctgataatctagaggcccctataactctctacggctaacctgaatggactacgacatagtctagtccgccaagatgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataacgccggcggcccctataactctctacggctaacctgaatggactacgacatagtctagtccgccaagatgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagctgttgaattttgaccttcttaagcttgcgggagacgtcgagtccaaccccgggcccatgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggcgcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgctgctgaacttcgacctgctgaagctggccggcgacgtggagagcaaccccggcccccatatgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataacgttgcatcctgcaggatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacgctagagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 38 - DNA, 16405 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacagcagactgctgatgatgagcgtgtacgccctgagcgccatcatcggcatctacctgctgtaccggatgctgaaaacctgctgataatctagaggcccctataactctctacggctaacctgaatggactacgacatagtctagtccgccaagatgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataacgccggcggcccctataactctctacggctaacctgaatggactacgacatagtctagtccgccaagatgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataaggcgcgccaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatatgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggagcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataagtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgcatatgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataacgttgcatcctgcaggatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacgctagagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 39 - DNA, 15300 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacagcagactgctgatgatgagcgtgtacgccctgagcgccatcatcggcatctacctgctgtaccggatgctgaaaacctgctgataatctagaggcccctataactctctacggctaacctgaatggactacgacatagtctagtccgccaagatgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataacgccggcggcccctataactctctacggctaacctgaatggactacgacatagtctagtccgccaagatgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataaggcgcgccgcccctataactctctacggctaacctgaatggactacgacatagtctagtccgccaagatgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggagcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataagcggccgcgcccctataactctctacggctaacctgaatggactacgacatagtctagtccgccaagatgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataacgttgcatcctgcaggatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacgctagagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 40 - DNA, 16324 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgactgataatctagaggcccctataactctctacggctaacctgaatggactacgacatagtctagtccgccaagatgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataacgccggcggcccctataactctctacggctaacctgaatggactacgacatagtctagtccgccaagatgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataaggcgcgccaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatatgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggagcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataagtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgcatatgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataacgttgcatcctgcaggatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacgctagagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 41 - DNA, 16360 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacggcagcggatctgggtcccaccatcaccatcaccattgataatctagaggcccctataactctctacggctaacctgaatggactacgacatagtctagtccgccaagatgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataacgccggcggcccctataactctctacggctaacctgaatggactacgacatagtctagtccgccaagatgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataaggcgcgccaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatatgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggagcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataagtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgcatatgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataacgttgcatcctgcaggatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacgctagagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 42 - DNA, 11459 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataatctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaaccagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggcccacgcgtaatacgactcactatag

SEQ ID NO: 43 - DNA, 3567 bp, Artificial Sequence
  source: /note=“Description of Artificial Sequence Synthetic polynucleotide”
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggacggaccgaccatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgggtgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtaatagtaagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 44 - DNA, 5685 bp, Artificial Sequence
  source: /note=“Description of Artificial Sequence Synthetic polynucleotide”
ataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggacggaccgaccatgtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattaatagtaagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 45 - protein, 6 aa, Artificial Sequence
  source: /note=“Description of Artificial Sequence Synthetic 6xHis tag”
HHHHHH

SEQ ID NO: 46 - DNA, 30 bp, Artificial Sequence
  source: /note=“Description of Artificial Sequence Synthetic oligonucleotide”
aaaaaaaaaaaaaaaaaaaaaaaaaaaaaa

(19) United States
(12) Patent Application Publication
(10) Pub. No.: US 2017/0342442 A1
(43) Pub. Date: Nov. 30, 2017

(54) RECOMBINANT SELF-REPLICATING POLYCISTRONIC RNA MOLECULES

(71) Applicant: GLAXOSMITHKLINE BIOLOGICALS, SA, Rixensart (BE)

(72) Inventors: Anders Lilja, Somerville, MA (US); Peter Mason, Somerville, MA (US)

(73) Assignee: GLAXOSMITHKLINE BIOLOGICALS, SA, Rixensart (BE)

(21) Appl. No.: 15/677,435

(22) Filed: Aug. 15, 2017

Related U.S. Application Data
(63) Continuation of application No. 14/350,575, filed on Apr. 9, 2014, now abandoned.
(60) Provisional application No. 61/546,002, filed on Oct. 11, 2011.

(51) Int. Cl. C12N 15/86; A61K 39/12; A61K 39/25; A61K 39/00

(52) U.S. Cl. CPC C12N 15/86; A61K 39/25; A61K 39/12; C12N 2710/16734; C12N 2710/16034; A61K 2039/53; C12N 2770/36143; C12N 2830/20; C12N 2710/16134

(57) ABSTRACT

This disclosure provides recombinant polycistronic nucleic acid molecules that contain at at least four nucleotide sequences that encode a protein of interest, particularly proteins that form complexes in vivo, each operably linked to a separate subgenomic promoter. In some embodiments these proteins and the complexes they form elicit potent neutralizing antibodies. Thus, presentation of herpes virus proteins using the disclosed platforms permits the generation of broad and potent immune responses useful for vaccine development.

### CROSS REFERENCE TO RELATED APPLICATIONS

This application is a Continuation of application Ser. No. 14/350,575, filed Apr. 9, 2014, which is the U.S. National Stage of International Application No. PCT/US2012/05973, filed Oct. 11, 2012 and published in English, which claims the benefit of U.S. Provisional Application No. 61/546,002, filed Oct. 11, 2011. Each of the foregoing applications is incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Sep. 28, 2012, is named PAT054830.txt and is 233,480 bytes in size.

### BACKGROUND

Pathogens can lead to substantial morbidity and mortality in individuals. For example, Herpes viruses are widespread and cause a wide range of diseases in humans that in the worst cases can lead to substantial morbidity and mortality, primarily in immunocompromised individuals (e.g., transplant recipients and HIV-infected individuals). Humans are susceptible to infection by at least eight herpes viruses. Herpes simplex virus-1 (HSV-1, HHV-1), Herpes simplex virus-2 (HSV-2, HHV-2) and Varicella zoster virus (VZV, HHV-3) are alpha-subfamily viruses, cytomegalovirus (CMV, HHV-5) and Roseoloviruses (HHV-6 and HHV-7) are beta-subfamily viruses, Epstein-Barr virus (EBV, HHV-4) and Kaposi's sarcoma-associated herpesvirus (KSHV, HHV-8) are gamma-subfamily viruses that infect humans.

CMV infection leads to substantial morbidity and mortality in immunocompromised individuals (e.g., transplant recipients and HIV-infected individuals) and congenital infection can result in devastating defects in neurological development in neonates. CMV envelope glycoproteins gB, gH, gL, gM and gN represent attractive vaccine candidates as they are expressed on the viral surface and can elicit protective virus-neutralizing humoral immune responses. Some CMV vaccine strategies have targeted the major surface glycoprotein B (gB), which can induce a dominant antibody response. (Go and Pollard, JID 197:1631-1633 (2008)). CMV glycoprotein gB can induce a neutralizing antibody response, and a large fraction of the antibodies that neutralize infection of fibroblasts in sera from CMV-positive patients is directed against gB (Britt 1990). Similarly, it has been reported that gH and gM/gN are targets of the immune response to natural infection (Urban et al (1996) J. Gen. Virol. 77(Pt. 7):1537-47; Mach et al (2000) J. Virol. 74(24):11881-92).

Complexes of CMV proteins are also attractive vaccine candidates because they appear to be involved in important processes in the viral life cycle. For example, the gH/gL/gO complex seems to have important roles in both fibroblast and epithelial/endothelial cell entry. The prevailing model suggests that the gH/gL/gO complex mediates infection of fibroblasts. hCMV gO-null mutants produce small plaques on fibroblasts and very low titer virus indicating a role in entry (Dunn (2003), Proc. Natl. Acad. Sci. USA 100:14223-28; Hobom (2000) J. Virol. 74:7720-29). Recent studies suggest that gO is not incorporated into virions with gH/gL, but may act as a molecular chaperone, increasing gH/gL export from the ER to the Golgi apparatus and incorporation into virions (Ryckman (2009) J. Virol 82:60-70). Through pulse-chase experiments, it was shown that small amounts of gO remain bound to gH/gL for long periods of time but most gO dissociates and or is degraded from the gH/gL/gO complex, as it is not found in extracellular virions or secreted from cells. When gO was deleted from a clinical strain of CMV (TR) those viral particles had significantly reduced amounts of gH/gL incorporated into the virion. Additionally, gO deleted from TR virus also inhibited entry into epithelial and endothelial cells, suggesting that gH/gL is also required for epithelial/endothelial cell entry (Wille (2010) J. Virol. 84(5):2585-96).

CMV gH/gL can also associate with UL128, UL130, and UL131A (referred to here as UL131) and form a pentameric complex that is required for entry into several cell types, including epithelial cells, endothelial cells, and dendritic cells (Hahn et al (2004) J. Virol. 78(18):10023-33; Wang and Shenk (2005) Proc. Natl. Acad. Sci USA 102(50):18153-8; Gerna et al (2005). J. Gen. Virol. 84(Pt 6):1431-6; Ryckman et al (2008) J. Virol. 82:60-70). In contrast, this complex is not required for infection of fibroblasts. Laboratory hCMV isolates carry mutations in the UL128-UL131 locus, and mutations arise in clinical isolates after only a few passages in cultured fibroblasts (Akter et al (2003) J. Gen. Virol. 84(Pt 5):1117-22). During natural infection, the pentameric complex elicits antibodies that neutralize infection of epithelial cells, endothelial cells (and likely any other cell type where the pentameric complex mediates viral entry) with very high potency (Macagno et al (2010) J. Virol. 84(2):1005-13). It also appears that antibodies to this complex contribute significantly to the ability of human sera to neutralize infection of epithelial cells (Genini et al (2011) J. Clin. Virol. 52(2):113-8).

U.S. Pat. No. 5,767,250 discloses methods for making certain CMV protein complexes that contain gH and gL. The complexes are produced by introducing a DNA construct that encodes gH and a DNA construct that encodes gL into a cell so that the gH and gL are co-expressed.

WO 2004/076645 describes recombinant DNA molecules that encode CMV proteins. According to this document, combinations of distinct DNA molecules that encode different CMV proteins, can be introduced into cells to cause co-expression of the encoded CMV proteins. When gM and gN were co-expressed in this way, they formed a disulfide-linked complex. Rabbits immunized with DNA constructs that produced the gM/gN complex or with a DNA construct encoding gB produced equivalent neutralizing antibody responses.

A need exists for polycistronic nucleic acids that encode four or more proteins, for methods of expressing four or more proteins in the same cell, and for immunization methods that produce better immune responses.

### SUMMARY OF THE INVENTION

The invention relates to recombinant ploycistronic nucleic acid molecules, such as polycistronic self replicating RNA molecules, for co-delivery of 4 or more proteins, e.g., pathogen proteins such as herpes virus (e.g., CMV) proteins, to cells, particularly proteins that form complexes in vivo.

In one aspect the recombinant ploycistronic nucleic acid molecules, such as a polycistronic self replicating RNA molecule, comprises: a) a first nucleotide sequence encoding a first protein or fragment thereof that is operably linked to a first subgenomic promoter (SGP); b) a second nucleotide sequence encoding a second protein or fragment thereof that is operably linked to a second SGP; c) a third nucleotide sequence encoding a third protein or fragment thereof that is operably linked to a third SGP; and d) a fourth nucleotide sequence encoding a fourth protein or fragment thereof that is operably linked to a fourth SGP; wherein when the self-replicating RNA molecule is introduced into a suitable cell, the first and second proteins or fragments thereof are produced. Optionally, the recombinant ploycistronic nucleic acid molecules, such as a polycistronic self replicating RNA molecule, further comprises a fifth nucleotide sequence encoding a fifth protein or fragment thereof that is operably linked to a fifth SGP. Preferably, the first protein or fragment thereof, the second protein or fragment thereof, the third protein or fragment thereof, and the fourth protein or fragment thereof, and when present, the fifth protein or fragment thereof, form a protein complex.

In some embodiments, the first protein or fragment thereof and the second protein or fragment thereof, the third protein or fragment thereof, the fourth protein or fragment thereof and, when present, the fifth protein or fragment thereof are each from a herpes virus, for example, HHV-1, HHV-2, HHV-3, HHV-4, HHV-5, HHV-6, HHV-7, HHV-8 or HHV-9.

In some embodiments, the first protein or fragment thereof and the second protein or fragment thereof, the third protein or fragment thereof, the fourth protein or fragment thereof and, when present, the fifth protein or fragment thereof are each from HHV-5 (CMV). In such embodiments, the first protein or fragment, the second protein or fragment, the third protein or fragment, the fourth protein or fragment, and the fifth protein or fragment are independently selected from the group consisting of gB, gH, gL, gO, gM, gN, UL128, UL130, UL131, and a fragment of any one of the foregoing. For example, the first protein or fragment can be gH or a fragment thereof, and the second protein or fragment can be gL or a fragment thereof, the third protein or fragment can be UL128 or a fragment thereof, the fourth protein or fragment can be UL130 or a fragment thereof, and the fifth protein or fragment can be UL131 or a fragment thereof.

In some embodiments, the first protein or fragment thereof and the second protein or fragment thereof, the third protein or fragment thereof, the fourth protein or fragment thereof and, when present, the fifth protein or fragment thereof are each from HHV-3 (VZV). In such embodiments, the first protein or fragment, the second protein or fragment, the third protein or fragment, the fourth protein or fragment, and the fifth protein or fragment are independently selected from the group consisting of gB, gE, gH, gI, gL, and a fragment of any one of the foregoing.

The recombinant ploycistronic nucleic acid molecule, can be a polycistronic self replicating RNA molecule. The self replicating RNA molecules can be an alphavirus replicon. In such instances, the alphavirus replicon can be delivered in the form of an alphavirus replicon particle (VRP). The self replicating RNA molecule can also be in the form of a “naked” RNA molecule.

The invention also relates to a recombinant DNA molecule that encodes a self replicating RNA molecule as described herein. In some embodiments, the recombinant DNA molecule is a plasmid. In some embodiments, the recombinant DNA molecule includes a mammalian promoter that drives transcription of the encoded self replicating RNA molecule.

The invention also relates to compositions that comprise a self-replicating RNA molecule as described herein and a pharmaceutically acceptable vehicle. In some embodiments, the composition comprises a self-replicating RNA molecule that encodes CMV proteins, such as the pentameric complex gH/gL/UL128/UL130/UL131. The composition can also contain an RNA delivery system such as a liposome, a polymeric nanoparticle, an oil-in-water cationic nanoemulsion or combinations thereof. For example, the self-replicating RNA molecule can be encapsulated in a liposome.

In certain embodiments, the composition comprises a VRP that contains an alphavirus replicon that encodes CMV proteins. In some embodiments, the VRP comprises a replicon that encodes the pentameric complex gH/gL/UL128/UL130/UL131. The composition can also comprise an adjuvant.

The invention also relates to methods of forming a CMV protein complex. In some embodiments a self-replicating RNA encoding four or more CMV proteins is delivered to a cell, the cell is maintained under conditions suitable for expression of the CMV proteins, wherein a CMV protein complex is formed. In other embodiments, a VRP that contains a self-replicating RNA encoding four or more CMV proteins is delivered to a cell, the cell is maintained under conditions suitable for expression of the CMV proteins, wherein a CMV protein complex is formed. The method can be used to form a CMV protein complex in a cell in vivo.

The invention also relates to a method for inducing an immune response in an individual by administering a recombinant polycistronic nucleic acid molecule, such as a self-replicating RNA molecule, to the individual. In some embodiments, a self-replicating RNA encoding four or more CMV proteins is administered to the individual. The self-replicating RNA molecule can be administered as a composition that contains an RNA delivery system, such as a liposome. In other embodiments, a VRP that contains a self-replicating RNA encoding four or more CMV proteins is administered to the individual. Preferably, the induced immune response comprises the production of neutralizing anti-CMV antibodies. More preferably, the neutralizing antibodies are complement-independent.

The invention also relates to a method of inhibiting CMV entry into a cell comprising contacting the cell with a self-replicating RNA molecule that encodes four or more CMV proteins. The cell can be selected from the group consisting of an epithelial cell, an endothelial cell, a fibroblast and combinations thereof. In some embodiments, the cell is contacted with a VRP that contains a self-replicating RNA encoding four or more CMV proteins.

The invention also relates to the use of a self-replicating RNA molecule that encodes four or more CMV proteins (e.g., a VRP, a composition comprising the self-replicating RNA molecule and a liposome) from a CMV protein complex in a cell, to induce an immune response or to inhibit CMV entry into a cell.

### BRIEF DESCRIPTION OF THE DRAWINGS

FIG. 1 is a schematic of pentacistronic RNA replicons, A526, A527, A554, A555 and A556, that encode five CMV proteins. Subgenomic promoters are shown by arrows, other control elements are labeled. “NSP1,” “NSP2,” “NSP3,” and “NSP4,” are alphavirus nonstructural proteins 1-4, respectively, required for replication of the virus. NSP4 is shown in the schematic, NSP1, NSP2 and NSP3 are upstream of NSP4.

FIG. 2 is a fluorescence histogram showing that BHKV cells transfected with the A527 RNA replicon express the gH/gL/UL128/UL130/UL131 pentameric complex. Cell stain was performed using an antibody that binds a conformational epitope present on the pentameric complex.

### DETAILED DESCRIPTION

The invention provides platforms for co-delivery of protein (e.g., protein antigens), such as herpes virus proteins (e.g., CMV proteins), to cells, particularly proteins that form complexes in vivo. The recombinant polycistronic nucleic acid molecules described herein provide the advantage of delivering sequences that encode four or more proteins to a cell, and driving the expression of the proteins. Using this approach, the four or more encoded proteins can be expressed at sufficient intracellular levels for the formation of protein complexes containing the four or more proteins in vivo. For example, the encoded proteins or fragments thereof can be expressed at substantially the same level, or if desired, at different levels by selecting appropriate expression control sequences. This is a significantly more efficient way to produce protein complexes in vivo than by co-delivering two or more individual DNA molecules that encode different proteins to the same cell, which can be inefficient and highly variable. See, e.g., WO 2004/076645.

Preferably, the recombinant polycistronic nucleic acid molecule is a self-replicating RNA molecule as described herein, in which each of the nucleotide sequences that encode a protein is operably linked to its own alphavirus subgenomic promoter (SGP). These self-replicating RNA molecules are smaller than corresponding molecules that use other expression control sequences (e.g., other promoters). Without wishing to be bound by any particular theory, it is believed that this type of self-replicating RNA molecule can be packaged into a VRP more efficiently and with higher yields than corresponding molecules that contain other expression control sequences, such as IRES. It is also believed, that the self-replicating RNA molecules described herein, and VRPs containing them, can produce a better immune response than corresponding molecules that contain other expression control sequences, such as IRES.

In some embodiments, the delivered proteins or the complexes they form elicit potent neutralizing antibodies. The immune response produced by co-delivery of proteins, particularly those that form complexes in vivo, can be superior to the immune response produced using other approaches. For example, an RNA molecule that encodes CMV gH, gL, UL128, UL130 and UL131 can be expressed to produce the gH/gL/UL128/UL130/UL131 pentameric complex, and can induce better neutralizing titers and/or protective immunity in comparison to an RNA molecule that encodes a single CMV protein (e.g., gB, gH, gL etc.), or even a mixture of RNA molecules that individually encode gH, gL, UL128, UL130 and UL131.

In a general aspect, the invention relates to recombinant polycistronic nucleic acid molecule e.g., self replicating RNA molecules, for delivery of four or more proteins to cells. The recombinant polycistronic nucleic acid molecules, such as, for example, self replicating RNA molecules comprising a first sequence encoding a first protein or fragment thereof operably linked to a first SGP, a second sequence encoding a second protein or fragment thereof operably linked to a second SGP, a third sequence encoding a third protein or fragment thereof operably linked to a third SGP and a fourth sequence encoding a fourth protein or fragment thereof operably linked to a fourth SGP. If desired, a fifth sequence encoding a fifth protein or fragment thereof operably linked to a fifth SGP, and optionally additional sequences encoding other proteins or fragments thereof, can be present in the self replicating RNA molecules. In some embodiments, the sequences encoding the first, second, third, fourth, and fifth proteins encode herpesvirus (e.g., CMV) proteins or fragments thereof.

In the polycistronic nucleic acids described herein, the encoded first, second, third and fourth proteins or fragments, and the encoded fifth protein or fragments, if present, generally and preferably are from the same organism, such as a pathogen (e.g., virus, bacteria, fungus, parasite, archaea). In certain examples, the proteins or fragments encoded by a polycistronic self replicating RNA molecule are all herpes virus proteins, such as CMV proteins or VZV proteins.

The recombinant polycistronic nucleic acid molecule can be based on any desired nucleic acid such as DNA (e.g., plasmid or viral DNA) or RNA. Any suitable DNA or RNA can be used as the nucleic acid vector that carries the open reading frames that encode herpesvirus (e.g., CMV) proteins or fragments thereof. Suitable nucleic acid vectors have the capacity to carry and drive expression of more than one protein gene. Such nucleic acid vectors are known in the art and include, for example, plasmids, DNA obtained from DNA viruses such as vaccinia virus vectors (e.g., NYVAC, see U.S. Pat. No. 5,494,807), and poxvirus vectors (e.g., ALVAC canarypox vector, Sanofi Pasteur), and RNA obtained from suitable RNA viruses such as alphavirus. If desired, the recombinant polycistronic nucleic acid molecule can be modified, e.g., contain modified nucleobases and or linkages as described further herein. Preferably, the polycistronic nucleic acid molecule is an RNA molecule.

In some aspects, the invention is a polycistronic nucleic acid molecule that contains a sequence encoding a herpesvirus gH or fragment thereof, and a herpesvirus gL or a fragment thereof. The gH and gL proteins, or fragments thereof, can be from any desired herpes virus such as HSV-1, HSV-2, VZV, EBV type 1, EBV type 2, CMV, HHV-6 type A, HHV-6 type B, HHV-7, KSHV, and the like. Preferably, the herpesvirus is VZV, HSV-2, HSV-1, EBV (type 1 or type 2) or CMV. More preferably, the herpesvirus is VZV, HSV-2 or CMV. Even more preferably, the herpesvirus is CMV. The sequences of gH and gL proteins and of nucleic acids that encode the proteins from these viruses are well known in the art. Exemplary sequences are identified in Table 1. The polycistronic nucleic acid molecule can contain a first sequence encoding a gH protein disclosed in Table 1, or a fragment thereof, or a sequence that is at least about 90%, 91%, 92%, 93%, 94%, 95%, 96%, 97%, 98%, or 99% identical thereto. The polycistronic nucleic acid molecule can also contain a second sequence encoding a gL protein disclosed in Table 1, or a fragment thereof, or a sequence that is at least about 90%, 91%, 92%, 93%, 94%, 95%, 96%, 97%, 98%, or 99% identical thereto.

TABLE 1

| Virus | gH accession number | gL accession number |
|---|---|---|
| HSV-1 (HHV-1) | NP_044623.1 | NP_044602.1 |
| HSV-2 (HHV-2) | NP_044491.1 | NP_044470.1 |
| VZV (HHV-3) | NP_040160.1 | NP_040182.1 |
| EBV type 1 (HHV-4) | YP_401700.1 | YP_401678.1 |
| EBV type 2 (HHV-4) | YP_001129496.1 | YP_001129472.1 |
| CMV (HHV-5) | YP_081523.1 | YP_081555.1 |
| HHV-6 type A | NP_042941.1 | NP_042975.1 |
| HHV-6 type B | NP_050229.1 | NP_050261.1 |
| HHV-7 | YP_073788.1 | YP_073820.1 |
| KSHV (HHV-8) | YP_001129375.1 | YP_001129399.1 |

In this description of the invention, to facilitate a clear description of the nucleic acids, particular sequence components are referred to as a “first sequence,” a “second sequence,” etc. It is to be understood that the first and second sequences can appear in any desired order or orientation, and that no particular order or orientation is intended by the words “first”, “second” etc. Similarly, protein complexes are referred to by listing the proteins that are present in the complex, e.g., gH/gL. This is intended to describe the complex by the proteins that are present in the complex and does not indicate relative amounts of the proteins or the order or orientation of sequences that encode the proteins on a recombinant nucleic acid.

Certain preferred embodiments, such as alphavirus VRP and self-replicating RNA that contain sequences encoding CMV proteins, are further described herein. It is intended that the sequences encoding CMV proteins in such preferred embodiments, can be replaced with sequences encoding proteins from other pathogens, such as gH and gL from other herpesviruses.

### Alphavirus VRP Platforms

In some embodiments, CMV proteins are delivered to a cell using alphavirus replicon particles (VRP) which employ polycistronic replicons (or vectors) as described below. As used herein, “polycistronic” includes vectors comprising four or more cistrons. Cistrons in a polycistronic vector can encode CMV proteins from the same CMV strains or from different CMV strains. The cistrons can be oriented in any 5′-3′ order. Any nucleotide sequence encoding a CMV protein can be used to produce the protein. Exemplary sequences useful for preparing the polycistronic nucleic acids that encode two or more CMV proteins or fragments thereof are described herein.

As used herein, the term “alphavirus” has its conventional meaning in the art and includes various species such as Venezuelan equine encephalitis virus (VEE; e.g., Trinidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus.

An “alphavirus replicon particle” (VRP) or “replicon particle” is an alphavirus replicon packaged with alphavirus structural proteins.

An “alphavirus replicon” (or “replicon”) is an RNA molecule which can direct its own amplification in vivo in a target cell. The replicon encodes the polymerase(s) which catalyze RNA amplification (nsP1, nsP2, nsP3, nsP4) and contains cis RNA sequences required for replication which are recognized and utilized by the encoded polymerase(s). An alphavirus replicon typically contains the following ordered elements: 5′ viral sequences required in cis for replication, sequences which encode biologically active alphavirus nonstructural proteins (nsP1, nsP2, nsP3, nsP4), 3′ viral sequences required in cis for replication, and a polyadenylate tract. An alphavirus replicon also may contain one or more viral subgenomic “junction region” promoters directing the expression of heterologous nucleotide sequences, which may, in certain embodiments, be modified in order to increase or reduce viral transcription of the subgenomic fragment and heterologous sequence(s) to be expressed. Other control elements can be used, as described below.

Alphavirus replicons encoding CMV proteins can be used to produce VRPs. Such alphavirus replicons comprise sequences encoding at least two CMV proteins or fragments thereof. These sequences are operably linked to one or more suitable control elements, such as a subgenomic promoter, an IRES (e.g., EMCV, EV71), and a viral 2A site, which can be the same or different. Delivery of components of these complexes using the polycistronic vectors disclosed herein is an efficient way of providing nucleic acid sequences that encode two or more CMV proteins in desired relative amounts; whereas if multiple alphavirus constructs were used to deliver individual CMV proteins for complex formation, efficient co-delivery of VRPs would be required.

Any combination of suitable control elements can be used in any order. Preferably, each sequences that encodes a CMV protein is operably linked to a separate promoter, such as a subgenomic promoter

Subgenomic Promoters

Subgenomic promoters, also known as junction region promoters can be used to regulate protein expression. Alphaviral subgenomic promoters regulate expression of alphaviral structural proteins. See Strauss and Strauss, “The alphaviruses: gene expression, replication, and evolution,” Microbiol Rev. 1994 September; 58(3):491-562. A polycistronic polynucleotide can comprise a subgenomic promoter from any alphavirus. When two or more subgenomic promoters are present in a polycistronic polynucleotide, the promoters can be the same or different. For example, the subgenomic promoter can have the sequence CTCTCTACGGCTAACCTGAATGGA (SEQ ID NO: 1). In certain embodiments, subgenomic promoters can be modified in order to increase or reduce viral transcription of the proteins. See U.S. Pat. No. 6,592,874.

Internal Ribosomal Entry Site (IRES)

In some embodiments, one or more control elements is an internal ribosomal entry site (IRES). An IRES allows multiple proteins to be made from a single mRNA transcript as ribosomes bind to each IRES and initiate translation in the absence of a 5′-cap, which is normally required to initiate translation. For example, the IRES can be EV71 or EMCV.

Viral 2A Site

The FMDV 2A protein is a short peptide that serves to separate the structural proteins of FMDV from a nonstructural protein (FMDV 2B). Early work on this peptide suggested that it acts as an autocatalytic protease, but other work (e.g., Donnelly et al., (2001), J. Gen. Virol. 82, 1013-1025) suggests that this short sequence and the following single amino acid of FMDV 2B (Gly) acts as a translational stop-start. Regardless of the precise mode of action, the sequence can be inserted between two polypeptides, and affect the production of multiple individual polypeptides from a single open reading frame. In the context of this invention, FMDV 2A sequences can be inserted between the sequences encoding at least two CMV proteins, allowing for their synthesis as part of a single open reading frame. For example, the open reading frame may encode a gH protein and a gL protein separated by a sequence encoding a viral 2A site. A single mRNA is transcribed then, during the translation step, the gH and gL peptides are produced separately due to the activity of the viral 2A site. Any suitable viral 2A sequence may be used. Often, a viral 2A site comprises the consensus sequence Asp-Val/Ile-Glu-X-Asn-Pro-Gly-Pro, where X is any amino acid (SEQ ID NO: 2). For example, the Foot and Mouth Disease Virus 2A peptide sequence is DVESNPGP (SEQ ID NO: 3). See Trichas et al., “Use of the viral 2A peptide for bicistronic expression in transgenic mice,” BMC Biol. 2008 Sep. 15; 6:40, and Halpin et al., “Self-processing 2A-polyproteins—a system for co-ordinate expression of multiple proteins in transgenic plants,” Plant J. 1999 February; 17(4):453-9.

In some embodiments an alphavirus replicon is a chimeric replicon, such as a VEE-Sindbis chimeric replicon (VCR) or a VEE strain TC83 replicon (TC83R) or a TC83-Sindbis chimeric replicon (TC83CR). In some embodiments a VCR contains the packaging signal and 3′ UTR from a Sindbis replicon in place of sequences in nsP3 and at the 3′ end of the VEE replicon; see Perri et al., J. Virol. 77, 10394-403, 2003. In some embodiments, a TC83CR contains the packaging signal and 3′ UTR from a Sindbis replicon in place of sequences in nsP3 and at the 3′ end of a VEE strain TC83replicon.

### Producing VRPs

Methods of preparing VRPs are well known in the art. In some embodiments an alphavirus is assembled into a VRP using a packaging cell. An “alphavirus packaging cell” (or “packaging cell”) is a cell that contains one or more alphavirus structural protein expression cassettes and that produces recombinant alphavirus particles after introduction of an alphavirus replicon, eukaryotic layered vector initiation system (e.g., U.S. Pat. No. 5,814,482), or recombinant alphavirus particle. The one or more different alphavirus structural protein cassettes serve as “helpers” by providing the alphavirus structural proteins. An “alphavirus structural protein cassette” is an expression cassette that encodes one or more alphavirus structural proteins and comprises at least one and up to five copies (i.e., 1, 2, 3, 4, or 5) of an alphavirus replicase recognition sequence. Structural protein expression cassettes typically comprise, from 5′ to 3′, a 5′ sequence which initiates transcription of alphavirus RNA, an optional alphavirus subgenomic region promoter, a nucleotide sequence encoding the alphavirus structural protein, a 3′ untranslated region (which also directs RNA transcription), and a polyA tract. See, e.g., WO 2010/019437.

In preferred embodiments two different alphavirus structural protein cassettes (“split” defective helpers) are used in a packaging cell to minimize recombination events which could produce a replication-competent virus. In some embodiments an alphavirus structural protein cassette encodes the capsid protein (C) but not either of the glycoproteins (E2 and E1). In some embodiments an alphavirus structural protein cassette encodes the capsid protein and either the E1 or E2 glycoproteins (but not both). In some embodiments an alphavirus structural protein cassette encodes the E2 and E1 glycoproteins but not the capsid protein. In some embodiments an alphavirus structural protein cassette encodes the E1 or E2 glycoprotein (but not both) and not the capsid protein.

In some embodiments, VRPs are produced by the simultaneous introduction of replicons and helper RNAs into cells of various sources. Under these conditions, for example, BHKV cells (1×10⁷) are electroporated at, for example, 220 volts, 1000 μF, 2 manual pulses with 10 μg replicon RNA:6 μg defective helper Cap RNA:10 μg defective helper Gly RNA, alphavirus containing supernatant is collected ˜24 hours later. Replicons and/or helpers can also be introduced in DNA forms which launch suitable RNAs within the transfected cells.

A packaging cell may be a mammalian cell or a non-mammalian cell, such as an insect (e.g., SF9) or avian cell (e.g., a primary chick or duck fibroblast or fibroblast cell line). See U.S. Pat. No. 7,445,924. Avian sources of cells include, but are not limited to, avian embryonic stem cells such as EB66® (VIVALIS); chicken cells, including chicken embryonic stem cells such as EBx® cells, chicken embryonic fibroblasts, and chicken embryonic germ cells; duck cells such as the AGE1.CR and AGE1.CR.pIX cell lines (ProBioGen) which are described, for example, in Vaccine 27:4975-4982 (2009) and WO2005/042728); and geese cells. In some embodiments, a packaging cell is a primary duck fibroblast or duck retinal cell line, such as AGE.CR (PROBIOGEN).

Mammalian sources of cells for simultaneous nucleic acid introduction and/or packaging cells include, but are not limited to, human or non-human primate cells, including PerC6 (PER.C6) cells (CRUCELL N.V.), which are described, for example, in WO 01/38362 and WO 02/40665, as well as deposited under ECACC deposit number 96022940); MRC-5 (ATCC CCL-171); WI-38 (ATCC CCL-75); fetal rhesus lung cells (ATCC CL-160); human embryonic kidney cells (e.g., 293 cells, typically transformed by sheared adenovirus type 5 DNA); VERO cells from monkey kidneys); cells of horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001); cat, and rodent (e.g., hamster cells such as BHK21-F, HKCC cells, or Chinese hamster ovary (CHO) cells), and may be obtained from a wide variety of developmental stages, including for example, adult, neonatal, fetal, and embryo.

In some embodiments a packaging cell is stably transformed with one or more structural protein expression cassette(s). Structural protein expression cassettes can be introduced into cells using standard recombinant DNA techniques, including transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection. Structural protein expression cassettes typically are introduced into a host cell as DNA molecules, but can also be introduced as in vitro-transcribed RNA. Each expression cassette can be introduced separately or substantially simultaneously.

In some embodiments, stable alphavirus packaging cell lines are used to produce recombinant alphavirus particles. These are alphavirus-permissive cells comprising DNA cassettes expressing the defective helper RNA stably integrated into their genomes. See Polo et al., Proc. Natl. Acad. Sci. USA 96, 4598-603, 1999. The helper RNAs are constitutively expressed but the alphavirus structural proteins are not, because the genes are under the control of an alphavirus subgenomic promoter (Polo et al., 1999). Upon introduction of an alphavirus replicon into the genome of a packaging cell by transfection or VRP infection, replicase enzymes are produced and trigger expression of the capsid and glycoprotein genes on the helper RNAs, and output VRPs are produced. Introduction of the replicon can be accomplished by a variety of methods, including both transfection and infection with a seed stock of alphavirus replicon particles. The packaging cell is then incubated under conditions and for a time sufficient to produce packaged alphavirus replicon particles in the culture supernatant.

Thus, packaging cells allow VRPs to act as self-propagating viruses. This technology allows VRPs to be produced in much the same manner, and using the same equipment, as that used for live attenuated vaccines or other viral vectors that have producer cell lines available, such as replication-incompetent adenovirus vectors grown in cells expressing the adenovirus E1A and E1B genes.

In some embodiments, a two-step process is used: the first step comprises producing a seed stock of alphavirus replicon particles by transfecting a packaging cell with a replicon RNA or plasmid DNA-based replicon. A much larger stock of replicon particles is then produced in a second step, by infecting a fresh culture of packaging cells with the seed stock. This infection can be performed using various multiplicities of infection (MOI), including a MOI=0.00001, 0.00005, 0.0001, 0.0005, 0.001, 0.005, 0.01, 0.05, 0.1, 0.5, 1.0, 3, 5, 10 or 20. In some embodiments infection is performed at a low MOI (e.g., less than 1). Over time, replicon particles can be harvested from packaging cells infected with the seed stock. In some embodiments, replicon particles can then be passaged in yet larger cultures of naive packaging cells by repeated low-multiplicity infection, resulting in commercial scale preparations with the same high titer.

### Self-Replicating RNA Platforms

Four or more CMV proteins can be produced by expression of recombinant nucleic acids that encode the proteins in the cells of a subject. Preferably, the recombinant nucleic acid molecules encode four or more CMV proteins, e.g., are polycistronic. Preferred nucleic acids that can be administered to a subject to cause the production of CMV proteins are self-replicating RNA molecules. The self-replicating RNA molecules of the invention are based on the genomic RNA of RNA viruses, but lack the genes encoding one or more structural proteins. The self-replicating RNA molecules are capable of being translated to produce non-structural proteins of the RNA virus and CMV proteins encoded by the self-replicating RNA.

The self-replicating RNA generally contains at least one or more genes selected from the group consisting of viral replicase, viral proteases, viral helicases and other nonstructural viral proteins, and also comprise 5′- and 3′-end cis-active replication sequences, and a heterologous sequences that encodes two or more desired CMV proteins. A subgenomic promoter that directs expression of the heterologous sequence(s) can be included in the self-replicating RNA. If desired, a heterologous sequence may be fused in frame to other coding regions in the self-replicating RNA and/or may be under the control of an internal ribosome entry site (IRES).

Self-replicating RNA molecules of the invention can be designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alpha virus, such as Sinbis virus (SIN), Semliki forest virus and Venezuelan equine encephalitis virus (VEE), one or more genes encoding viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to a vertebrate cell even without any proteins, lead to the production of multiple daughter RNAs by transcription from itself (or from an antisense copy of itself). The self-replicating RNA can be directly translated after delivery to a cell, and this translation provides a RNA-dependent RNA polymerase which then produces transcripts from the delivered RNA. Thus the delivered RNA leads to the production of multiple daughter RNAs. These transcripts are antisense relative to the delivered RNA and may be translated themselves to provide in situ expression of encoded CMV protein, or may be transcribed to provide further transcripts with the same sense as the delivered RNA which are translated to provide in situ expression of the encoded CMV protein(s).

One suitable system for achieving self-replication is to use an alphavirus-based RNA replicon, such as an alphavirus replicon as described herein. These + stranded replicons are translated after delivery to a cell to give off a replicase (or replicase-transcriptase). The replicase is translated as a polyprotein which auto cleaves to provide a replication complex which creates genomic − strand copies of the + strand delivered RNA. These − strand transcripts can themselves be transcribed to give further copies of the + stranded parent RNA and also to give a subgenomic transcript which encodes two or more CMV proteins. Translation of the subgenomic transcript thus leads to in situ expression of the CMV protein(s) by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) two or more CMV proteins or fragments thereof. The polymerase can be an alphavirus replicase e.g. comprising alphavirus protein nsP4.

Whereas natural alphavirus genomes encode structural virion proteins in addition to the non structural replicase polyprotein, it is preferred that an alphavirus based self-replicating RNA molecule of the invention does not encode all alphavirus structural proteins. Thus the self replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self replicating RNAs of the invention and their place is taken by gene(s) encoding the desired gene product (CMV protein or fragment thereof), such that the subgenomic transcript encodes the desired gene product rather than the structural alphavirus virion proteins.

Thus a self-replicating RNA molecule useful with the invention have four or more sequences that encode different CMV proteins or fragments thereof. The sequences encoding the CMV proteins or fragments can be in any desired orientation, and can be operably linked to the same or separate promoters. In some embodiments the RNA may have one or more additional (downstream) sequences or open reading frames e.g. that encode other additional CMV proteins or fragments thereof. A self-replicating RNA molecule can have a 5′ sequence which is compatible with the encoded replicase.

In one aspect, the self-replicating RNA molecule is derived from or based on an alphavirus, such as an alphavirus replicon as defined herein. In other aspects, the self-replicating RNA molecule is derived from or based on a virus other than an alphavirus, preferably, a positive-stranded RNA viruses, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro virus (ATCC VR-66; ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The self-replicating RNA molecules of the invention can contain one or more modified nucleotides and therefore have improved stability and be resistant to degradation and clearance in vivo, and other advantages. Without wishing to be bound by any particular theory, it is believed that self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduce activation of the innate immune system and subsequent undesired consequences (e.g., inflammation at injection site, irritation at injection site, pain, and the like). It is also believed that the RNA molecules produced as a result of self-replication are recognized as foreign nucleic acids by the cytoplasmic immune receptors. Thus, self-replicating RNA molecules that contain modified nucleotides provide for efficient amplification of the RNA in a host cell and expression of CMV proteins, as well as adjuvant effects.

The RNA sequence can be modified with respect to its codon usage, for example, to increase translation efficacy and half-life of the RNA. A poly A tail (e.g., of about 30 adenosine residues or more (SEQ ID NO: 46)) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure can provide stability and translational efficacy to the RNA molecule. The 5′ cap of the RNA molecule may be further modified by a 2′-O-Methyltransferase which results in the generation of a cap 1 structure (m7Gppp [m2′-O]N), which may further increases translation efficacy.

As used herein, “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). If desired, a self replicating RNA molecule can contain chemical modifications in or on the sugar moiety of the nucleoside (e.g., ribose, deoxyribose, modified ribose, modified deoxyribose, six-membered sugar analog, or open-chain sugar analog), or the phosphate.

The self-replicating RNA molecules can contain at least one modified nucleotide, that preferably is not part of the 5′ cap. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties.

In some examples, between 0.001% and 99% or 100% of the nucleotides in a self-replicating RNA molecule are modified nucleotides. For example, 0.001%-25%, 0.01%-25%, 0.1%-25%, or 1%-25% of the nucleotides in a self-replicating RNA molecule are modified nucleotides.

In other examples, between 0.001% and 99% or 100% of a particular unmodified nucleotide in a self-replicating RNA molecule is replaced with a modified nucleotide. For example, about 1% of the nucleotides in the self-replicating RNA molecule that contain uridine can be modified, such as by replacement of uridine with pseudouridine. In other examples, the desired amount (percentage) of two, three, or four particular nucleotides (nucleotides that contain uridine, cytidine, guanosine, or adenine) in a self-replicating RNA molecule are modified nucleotides. For example, 0.001%-25%, 0.01%-25%, 0.1%-25, or 1%-25% of a particular nucleotide in a self-replicating RNA molecule are modified nucleotides. In other examples, 0.001%-20%, 0.001%-15%, 0.001%-10%, 0.01%-20%, 0.01%-15%, 0.1%-25, 0.01%-10%, 1%-20%, 1%-15%, 1%-10%, or about 5%, about 10%, about 15%, about 20% of a particular nucleotide in a self-replicating RNA molecule are modified nucleotides.

It is preferred that less than 100% of the nucleotides in a self-replicating RNA molecule are modified nucleotides. It is also preferred that less than 100% of a particular nucleotide in a self-replicating RNA molecule are modified nucleotides. Thus, preferred self-replicating RNA molecules comprise at least some unmodified nucleotides.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g. from U.S. Pat. Nos. 4,373,071, 4,458,066, 4,500,707, 4,668,777, 4,973,679, 5,047,524, 5,132,418, 5,153,319, 5,262,530, 5,700,642 all of which are incorporated herein by reference in their entirety, and many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmoSU (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers.

If desired, the self-replicating RNA molecule can contain phosphoramidate, phosphorothioate, and/or methylphosphonate linkages.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses.

Suitable synthetic methods can be used alone, or in combination with one or more other methods (e.g., recombinant DNA or RNA technology), to produce a self-replicating RNA molecule that contain one or more modified nucleotides. Suitable methods for de novo synthesis are well-known in the art and can be adapted for particular applications. Exemplary methods include, for example, chemical synthesis using suitable protecting groups such as CEM (Masuda et al., (2007) Nucleic Acids Symposium Series 51:3-4), the β-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedron Lett 27:4055-8; Gaffney B L et al. (1988) Tetrahedron Lett 29:2619-22). These chemistries can be performed or adapted for use with automated nucleic acid synthesizers that are commercially available. Additional suitable synthetic methods are disclosed in Uhlmann et al. (1990) Chem Rev 90:544-84, and Goodchild J (1990) Bioconjugate Chem 1: 165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation and expression of nucleic acid sequences are known and conventional in the art. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

The presence and/or quantity of one or more modified nucleotides in a self-replicating RNA molecule can be determined using any suitable method. For example, a self-replicating RNA can be digested to monophosphates (e.g., using nuclease P1) and dephosphorylated (e.g., using a suitable phosphatase such as CIAP), and the resulting nucleosides analyzed by reversed phase HPLC (e.g., usings a YMC Pack ODS-AQ column (5 micron, 4.6×250 mm) and elute using a gradient, 30% B (0-5 min) to 100% B (5-13 min) and at 100% B (13-40) min, flow Rate (0.7 ml/min), UV detection (wavelength: 260 nm), column temperature (30° C.). Buffer A (20 mM acetic acid-ammonium acetate pH 3.5), buffer B (20 mM acetic acid-ammonium acetate pH 3.5/methanol [90/10])).

The self-replicating RNA may be associated with a delivery system. The self-replicating RNA may be administered with or without an adjuvant.

### RNA Delivery Systems

The self-replicating RNA described herein are suitable for delivery in a variety of modalities, such as naked RNA delivery or in combination with lipids, polymers or other compounds that facilitate entry into the cells. Self-replicating RNA molecules can be introduced into target cells or subjects using any suitable technique, e.g., by direct injection, microinjection, electroporation, lipofection, biolystics, and the like. The self-replicating RNA molecule may also be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues (SEQ ID NO: 4)), which is itself coupled to an integrin receptor-binding moiety (e.g., a cyclic peptide having the sequence Arg-Gly-Asp (SEQ ID NO: 5).

The self-replicating RNA molecules can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

The self-replicating RNA can be delivered as naked RNA (e.g. merely as an aqueous solution of RNA) but, to enhance entry into cells and also subsequent intercellular effects, the self-replicating RNA is preferably administered in combination with a delivery system, such as a particulate or emulsion delivery system. A large number of delivery systems are well known to those of skill in the art. Such delivery systems include, for example liposome-based delivery (Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Feigner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; Ali et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

Three particularly useful delivery systems are (i) liposomes, (ii) non-toxic and biodegradable polymer microparticles, and (iii) cationic submicron oil-in-water emulsions.

Liposomes

Various amphiphilic lipids can form bilayers in an aqueous environment to encapsulate a RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic. Suitable classes of phospholipid include, but are not limited to, phosphatidylethanolamines, phosphatidylcholines, phosphatidylserines, and phosphatidylglycerols, and some useful phospholipids are listed in Table 2. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all of the component lipids in the mixture need to be amphiphilic e.g. one or more amphiphilic lipids can be mixed with cholesterol.

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-87.

A mixture of DSPC, DlinDMA, PEG-DMPG and cholesterol can be used to form liposomes. A separate aspect of the invention is a liposome comprising DSPC, DlinDMA, PEG-DMG and cholesterol. This liposome preferably encapsulates RNA, such as a self-replicating RNA e.g. encoding an immunogen.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter ≦50 nm, and LUVs have a diameter >50 nm. Liposomes useful with of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2.

Techniques for preparing suitable liposomes are well known in the art e.g. see Liposomes: Methods and Protocols, Volume 1: Pharmaceutical Nanocarriers: Methods and Protocols. (ed. Weissig). Humana Press, 2009. ISBN 160327359X; Liposome Technology, volumes I, II & III. (ed. Gregoriadis). Informa Healthcare, 2006; and Functional Polymer Colloids and Microparticles volume 4 (Microspheres, microcapsules & liposomes). (eds. Arshady & Guyot). Citus Books, 2002. One useful method involves mixing (i) an ethanolic solution of the lipids (ii) an aqueous solution of the nucleic acid and (iii) buffer, followed by mixing, equilibration, dilution and purification (Heyes et al. (2005) J Controlled Release 107:276-87.).

RNA is preferably encapsulated within the liposomes, and so the liposome forms a outer layer around an aqueous RNA-containing core. This encapsulation has been found to protect RNA from RNase digestion. The liposomes can include some external RNA (e.g. on the surface of the liposomes), but preferably, at least half of the RNA (and ideally substantially all of it) is encapsulated.

Polymeric Microparticles

Various polymers can form microparticles to encapsulate or adsorb RNA. The use of a substantially non-toxic polymer means that a recipient can safely receive the particles, and the use of a biodegradable polymer means that the particles can be metabolised after delivery to avoid long-term persistence. Useful polymers are also sterilisable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 55:45, 60:40, 75:25. Useful PLG polymers include those having a molecular weight between, for example, 5,000-200,000 Da e.g. between 10,000-100,000, 20,000-70,000, 40,000-50,000 Da.

The microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g. see Functional Polymer Colloids and Microparticles volume 4 (Microspheres, microcapsules & liposomes). (eds. Arshady & Guyot). Citus Books, 2002; Polymers in Drug Delivery. (eds. Uchegbu & Schatzlein). CRC Press, 2006. (in particular chapter 7) and Microparticulate Systems for the Delivery of Proteins and Vaccines. (eds. Cohen & Bernstein). CRC Press, 1996. To facilitate adsorption of RNA, a microparticle may include a cationic surfactant and/or lipid e.g. as disclosed in O'Hagan et al. (2001) J Virology 75:9037-9043; and Singh et al. (2003) Pharmaceutical Research 20: 247-251. An alternative way of making polymeric microparticles is by molding and curing e.g. as disclosed in WO2009/132206.

Microparticles of the invention can have a zeta potential of between 40-100 mV. RNA can be adsorbed to the microparticles, and adsorption is facilitated by including cationic materials (e.g. cationic lipids) in the microparticle.

Oil-in-Water Cationic Emulsions

Oil-in-water emulsions are known for adjuvanting influenza vaccines e.g. the MF59™ adjuvant in the FLUAD™ product, and the AS03 adjuvant in the PREPANDRIX™ product. RNA delivery can be accomplished with the use of an oil-in-water emulsion, provided that the emulsion includes one or more cationic molecules. For instance, a cationic lipid can be included in the emulsion to provide a positively charged droplet surface to which negatively-charged RNA can attach.

The emulsion comprises one or more oils. Suitable oil(s) include those from, for example, an animal (such as fish) or a vegetable source. The oil is ideally biodegradable (metabolizable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

Preferred emulsions comprise squalene, a shark liver oil which is a branched, unsaturated terpenoid (C₃₀H₅₀; [(CH₃)₂C[═CHCH₂CH₂C(CH₃)]₂═CHCH₂—]₂; 2,6,10,15,19,23-hexamethyl-2,6,10,14,18,22-tetracosahexaene; CAS RN 7683-64-9).

The oil in the emulsion may comprise a combination of oils e.g. squalene and at least one further oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. Preferably this lipid is a surfactant so that it can facilitate formation and stabilization of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′-[N—(N′,N′-Dimethylaminoethane)-carbamoyl]Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxyl-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (ĈGluPhCnN), ditetradecyl glutamate ester with pendant amino group (C14GIuCnN+), cationic derivatives of cholesterol, including but not limited to cholesteryl-3 β-oxysuccinamidoethylenetrimethylammonium salt, cholesteryl-3 β-oxysuccinamidoethylene-dimethylamine, cholesteryl-3 β-carboxyamidoethylenetrimethylammonium salt, and cholesteryl-3 β-carboxyamidoethylenedimethylamine. Other useful cationic lipids are described in US 2008/0085870 and US 2008/0057080, which are incorporated herein by reference. The cationic lipid is preferably biodegradable (metabolizable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, preferably 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and preferably at least 90%. Apparatuses for determining the average droplet size in an emulsion, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidization. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidization can be performed until an emulsion with a desired droplet size average and distribution are achieved.

As an alternative to microfluidization, thermal methods can be used to cause phase inversion. These methods can also provide a submicron emulsion with a tight particle size distribution.

Preferred emulsions can be filter sterilized i.e. their droplets can pass through a 220 nm filter. As well as providing a sterilization, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml, from about 0.6 mg/ml to about 25 mg/ml, from about 0.7 mg/ml to about 25 mg/ml, from about 0.8 mg/ml to about 25 mg/ml, from about 0.9 mg/ml to about 25 mg/ml, from about 1.0 mg/ml to about 25 mg/ml, from about 1.1 mg/ml to about 25 mg/ml, from about 1.2 mg/ml to about 25 mg/ml, from about 1.3 mg/ml to about 25 mg/ml, from about 1.4 mg/ml to about 25 mg/ml, from about 1.5 mg/ml to about 25 mg/ml, from about 1.6 mg/ml to about 25 mg/ml, from about 1.7 mg/ml to about 25 mg/ml, from about 0.5 mg/ml to about 24 mg/ml, from about 0.5 mg/ml to about 22 mg/ml, from about 0.5 mg/ml to about 20 mg/ml, from about 0.5 mg/ml to about 18 mg/ml, from about 0.5 mg/ml to about 15 mg/ml, from about 0.5 mg/ml to about 12 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1.9 mg/ml, from about 0.5 mg/ml to about 1.8 mg/ml, from about 0.5 mg/ml to about 1.7 mg/ml, from about 0.5 mg/ml to about 1.6 mg/ml, from about 0.6 mg/ml to about 1.6 mg/ml, from about 0.7 mg/ml to about 1.6 mg/ml, from about 0.8 mg/ml to about 1.6 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 12 mg/ml, about 18 mg/ml, about 20 mg/ml, about 21.8 mg/ml, about 24 mg/ml, etc. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.62 mg/ml to about 5 mg/ml, from about 1 mg/ml to about 5 mg/ml, from about 1.5 mg/ml to about 5 mg/ml, from about 2 mg/ml to about 5 mg/ml, from about 2.46 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, from about 0.1 mg/ml to about 4.92 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.46 mg/ml, from about 0.1 mg/ml to about 2 mg/ml, from about 0.1 mg/ml to about 1.5 mg/ml, from about 0.1 mg/ml to about 1 mg/ml, from about 0.1 mg/ml to about 0.62 mg/ml, about 0.15 mg/ml, about 0.3 mg/ml, about 0.6 mg/ml, about 0.62 mg/ml, about 0.9 mg/ml, about 1.2 mg/ml, about 2.46 mg/ml, about 4.92 mg/ml, etc. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 5 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.5 mg/ml, from about 0.1 mg/ml to about 2 mg/ml, from about 0.1 mg/ml to about 1.5 mg/ml, from about 0.1 mg/ml to about 1.45 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.6 mg/ml to about 5 mg/ml, from about 0.73 mg/ml to about 5 mg/ml, from about 0.8 mg/ml to about 5 mg/ml, from about 0.9 mg/ml to about 5 mg/ml, from about 1.0 mg/ml to about 5 mg/ml, from about 1.2 mg/ml to about 5 mg/ml, from about 1.45 mg/ml to about 5 mg/ml, from about 2 mg/ml to about 5 mg/ml, from about 2.5 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, about 1.2 mg/ml, about 1.45 mg/ml, etc. Alternatively, the cationic oil-in-water emulsion may comprise DDA at about 20 mg/ml, about 21 mg/ml, about 21.5 mg/ml, about 21.6 mg/ml, about 25 mg/ml. In an exemplary embodiment, the cationic oil-in-water emulsion comprises from about 0.73 mg/ml to about 1.45 mg/ml DDA, such as 1.45 mg/ml.

Catheters or like devices may be used to deliver the self-replicating RNA molecules of the invention, as naked RNA or in combination with a delivery system, into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

The present invention includes the use of suitable delivery systems, such as liposomes, polymer microparticles or submicron emulsion microparticles with encapsulated or adsorbed self-replicating RNA, to deliver a self-replicating RNA molecule that encodes two or more CMV proteins, for example, to elicit an immune response alone, or in combination with another macromolecule. The invention includes liposomes, microparticles and submicron emulsions with adsorbed and/or encapsulated self-replicating RNA molecules, and combinations thereof.

The self-replicating RNA molecules associated with liposomes and submicron emulsion microparticles can be effectively delivered to a host cell, and can induce an immune response to the protein encoded by the self-replicating RNA.

Polycistronic self replicating RNA molecules that encode CMV proteins, and VRPs produced using polycistronic alphavirus replicons, can be used to form CMV protein complexes in a cell. Complexes include, but are not limited to, gB/gH/gL; gH/gL; gH/gL/gO; gM/gN; gH/gL/UL128/UL130/UL131; and UL128/UL130/UL131.

In some embodiments combinations of VRPs are delivered to a cell. Combinations include, but are not limited to:
1. a gH/gL VRP
2. a gH/gL VRP and a gB VRP;
3. a gH/gL/gO VRP and a gB VRP;
4. a gB VRP and a gH/gL/UL128/UL130/UL131 VRP;
5. a gB VRP and UL128/UL130/UL131 VRP;
6. a gB VRP and a gM/gN VRP;
7. a gB VRP, a gH/gL VRP, and a UL128/UL130/UL131 VRP;
8. a gB VRP, a gH/gLgO VRP, and a UL128/UL130/UL131 VRP;
9. a gB VRP, a gM/gN VRP, a gH/gL VRP, and a UL128/UL130/UL131 VRP;
10. a gB VRP, a gM/gN VRP, a gH/gL/O VRP, and a UL128/UL130/UL131 VRP;
11. a gH/gL VRP and a UL128/UL130/UL131 VRP; and

In some embodiments combinations of self-replicating RNA molecules are delivered to a cell. Combinations include, but are not limited to:
1. a self-replicating RNA molecule encoding gH and gL
2. a self-replicating RNA molecule encoding gH and gL and a self-replicating RNA molecule encoding gB;
3. a self-replicating RNA molecule encoding gH, gL and gO and a self-replicating RNA molecule encoding gB;
4. a self-replicating RNA molecule encoding gB and a self-replicating RNA molecule encoding gH, gL, UL128, UL130 and UL131;
5. a self-replicating RNA molecule encoding gB and a self-replicating RNA molecule encoding UL128, UL130 and UL131;
6. a self-replicating RNA molecule encoding gB and a self-replicating RNA molecule encoding gM and gN;
7. a self-replicating RNA molecule encoding gB, a self-replicating RNA molecule encoding gH and gL, and a self-replicating RNA molecule encoding UL128, UL130 and UL131;
8. a self-replicating RNA molecule encoding gB, a self-replicating RNA molecule encoding gH, gL, and gO, and a self-replicating RNA molecule encoding UL128, UL130 and UL131;
9. a self-replicating RNA molecule encoding gB, a self-replicating RNA molecule encoding gM and gN, a self-replicating RNA molecule encoding gH and gL, and a self-replicating RNA molecule encoding UL128, UL130 and UL131;
10. a self-replicating RNA molecule encoding gB, a self-replicating RNA molecule encoding gM and gN, a self-replicating RNA molecule encoding gH, gL and gO, and a self-replicating RNA molecule encoding UL128, UL130 and UL131;
11. a self-replicating RNA molecule encoding gH and gL, and a self-replicating RNA molecule encoding UL128, UL130 and UL131; and

### CMV Proteins

Suitable CMV proteins include gB, gH, gL, gO, UL128, UL130, UL131 and can be from any CMV strain. For example, CMV proteins can be from Merlin, AD169, VR1814, Towne, Toledo, TR, PH, TB40, or Fix strains of CMV. Exemplary CMV proteins and fragments are described herein. These proteins and fragments can be encoded by any suitable nucleotide sequence, including sequences that are codon optimized or deoptimized for expression in a desired host, such as a human cell. Exemplary sequences of CMV proteins and nucleic acids encoding the proteins are provided in Table 2

TABLE 2

| | |
|---|---|
| Full length gH polynucleotide | (CMV gH FL) SEQ ID NO: 12 |
| Full length gH polypeptide | (CMV gH FL) SEQ ID NO: 13 |
| Full length gL polynucleotide | (CMV gL FL) SEQ ID NO: 16 |
| Full length gL polypeptide | (CMV gL FL) SEQ ID NO: 17 |
| Full length gO polynucleotide | (CMV gO FL) SEQ ID NO: 22 |
| Full length gO polypeptide | (CMV gO FL) SEQ ID NO: 23 |
| gH sol polynucleotide | (CMV gH sol) SEQ ID NO: 14 |
| gH sol polypeptide | (CMV gH sol) SEQ ID NO: 15 |
| Full length UL128 polynucleotide | (CMV UL128 FL) SEQ ID NO: 24 |
| Full length UL128 polypeptide | (CMV UL128 FL) SEQ ID NO: 25 |
| Full length UL130 polynucleotide | (CMV UL130 FL) SEQ ID NO: 26 |
| Full length UL130 polypeptide | (CMV UL130 FL) SEQ ID NO: 27 |
| Full length UL131 polynucleotide | (CMV UL131 FL) SEQ ID NO: 28 |
| Full length UL131 polypeptide | (CMV UL131 FL) SEQ ID NO: 29 |
| Full length gB polynucleotide | (CMV gB FL) SEQ ID NO: 6 |
| Full length gB polypeptide | (CMV gB FL) SEQ ID NO: 7 |
| gB sol 750 polynucleotide | (CMV gB 750) SEQ ID NO: 8 |
| gB sol 750 polypeptide | (CMV gB 750) SEQ ID NO: 9 |
| gB sol 692 polynucleotide | (CMV gB 692) SEQ ID NO: 10 |
| gB sol 692 polypeptide | (CMV gB 692) SEQ ID NO: 11 |
| Full length gM polynucleotide | (CMV gM FL) SEQ ID NO: 18 |
| Full length gM polypeptide | (CMV gM FL) SEQ ID NO: 19 |
| Full length gN polynucleotide | (CMV gN FL) SEQ ID NO: 20 |
| Full length gN polypeptide | (CMV gN FL) SEQ ID NO: 21 |

CMV gB Proteins

A gB protein can be full length or can omit one or more regions of the protein. Alternatively, fragments of a gB protein can be used. gB amino acids are numbered according to the full-length gB amino acid sequence (CMV gB FL) shown in SEQ ID NO: 7, which is 907 amino acids long. Suitable regions of a gB protein, which can be excluded from the full-length protein or included as fragments include: the signal sequence (amino acids 1-24), a gB-DLD disintegrin-like domain (amino acids 57-146), a furin cleavage site (amino acids 459-460), a heptad repeat region (amino acids 679-693), a membrane spanning domain (amino acids 751-771), and a cytoplasmic domain from amino acids 771-906. In some embodiments a gB protein includes amino acids 67-86 (Neutralizing Epitope AD2) and/or amino acids 532-635 (Immunodominant Epitope AD1). Specific examples of gB fragments, include “gB sol 692,” which includes the first 692 amino acids of gB, and “gB sol 750,” which includes the first 750 amino acids of gB. The signal sequence, amino acids 1-24, can be present or absent from gB sol 692 and gB sol 750 as desired. Optionally, the gB protein can be a gB fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, or 875 amino acids. A gB fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, or 897.

Optionally, a gB fragment can extend further into the N-terminus by 5, 10, 20, or 30 amino acids from the starting residue of the fragment. Optionally, a gB fragment can extend further into the C-terminus by 5, 10, 20, or 30 amino acids from the last residue of the fragment.

CMV gH Proteins

In some embodiments, a gH protein is a full-length gH protein (CMV gH FL, SEQ ID NO: 13, for example, which is a 743 amino acid protein). gH has a membrane spanning domain and a cytoplasmic domain starting at position 716 to position 743. Removing amino acids from 717 to 743 provides a soluble gH (e.g., CMV gH sol, SEQ ID NO:15). In some embodiments the gH protein can be a gH fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, or 725 amino acids. Optionally, the gH protein can be a gH fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, or 725 amino acids. A gH fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 731, 732 or 733.

gH residues are numbered according to the full-length gH amino acid sequence (CMV gH FL) shown in SEQ ID NO: 13. Optionally, a gH fragment can extend further into the N-terminus by 5, 10, 20, or 30 amino acids from the starting residue of the fragment. Optionally, a gH fragment can extend further into the C-terminus by 5, 10, 20, or 30 amino acids from the last residue of the fragment.

CMV gL Proteins

In some embodiments a gL protein is a full-length gL protein (CMV gL FL, SEQ ID NO:17, for example, which is a 278 amino acid protein). In some embodiments a gL fragment can be used. For example, the number of amino acids in the fragment can comprise 10, 15, 20, 30, 40, 50, 60, 70, 80, 90, 100, 125, 150, 175, 200, 225, or 250 amino acids. A gL fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, or 268.

gL residues are numbered according to the full-length gL amino acid sequence (CMV gL FL) shown in SEQ ID NO: 17. Optionally, a gL fragment can extend further into the N-terminus by 5, 10, 20, or 30 amino acids from the starting residue of the fragment. Optionally, a gL fragment can extend further into the C-terminus by 5, 10, 20, or 30 amino acids from the last residue of the fragment.

CMV gO Proteins

In some embodiments, a gO protein is a full-length gO protein (CMV gO FL, SEQ ID NO:23, for example, which is a 472 amino acid protein). In some embodiments the gO protein can be a gO fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, or 450 amino acids. A gO fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, or 462.

gO residues are numbered according to the full-length gO amino acid sequence (CMV gO FL) shown in SEQ ID NO: 23. Optionally, a gO fragment can extend further into the N-terminus by 5, 10, 20, or 30 amino acids from the starting residue of the fragment. Optionally, a gO fragment can extend further into the C-terminus by 5, 10, 20, or 30 amino acids from the last residue of the fragment.

CMV gM Proteins

In some embodiments, a gM protein is a full-length gM protein (CMV gM FL, SEQ ID NO:19, for example, which is a 371 amino acid protein). In some embodiments the gM protein can be a gM fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, or 350 amino acids. A gM fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, or 361.

gM residues are numbered according to the full-length gM amino acid sequence (CMV gM FL) shown in SEQ ID NO: 19. Optionally, a gM fragment can extend further into the N-terminus by 5, 10, 20, or 30 amino acids from the starting residue of the fragment. Optionally, a gM fragment can extend further into the C-terminus by 5, 10, 20, or 30 amino acids from the last residue of the fragment.

CMV gN Proteins

In some embodiments, a gN protein is a full-length gN protein (CMV gN FL, SEQ ID NO:21, for example, which is a 135 amino acid protein). In some embodiments the gN protein can be a gN fragment of 10 amino acids or longer. For example, the number of amino acids in the fragment can comprise 10, 15, 20, 30, 40, 50, 60, 70, 80, 90, 100, or 125 amino acids. A gN fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

gN residues are numbered according to the full-length gN amino acid sequence (CMV gN FL) shown in SEQ ID NO: 21. Optionally, a gN fragment can extend further into the N-terminus by 5, 10, 20, or 30 amino acids from the starting residue of the fragment. Optionally, a gN fragment can extend further into the C-terminus by 5, 10, 20, or 30 amino acids from the last residue of the fragment.

CMV UL128 Proteins

In some embodiments, a UL128 protein is a full-length UL128 protein (CMV UL128 FL, SEQ ID NO:25, for example, which is a 171 amino acid protein). In some embodiments the UL128 protein can be a UL128 fragment of 10 amino acids or longer. For example, the number of amino acids in the fragment can comprise 10, 15, 20, 30, 40, 50, 60, 70, 80, 90, 100, 125, or 150 amino acids. A UL128 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or 161.

UL128 residues are numbered according to the full-length UL128 amino acid sequence (CMV UL128 FL) shown in SEQ ID NO: 25. Optionally, a UL128 fragment can extend further into the N-terminus by 5, 10, 20, or 30 amino acids from the starting residue of the fragment. Optionally, a UL128 fragment can extend further into the C-terminus by 5, 10, 20, or 30 amino acids from the last residue of the fragment.

CMV UL130 Proteins

In some embodiments, a UL130 protein is a full-length UL130 protein (CMV UL130 FL, SEQ ID NO:27, for example, which is a 214 amino acid protein). In some embodiments the UL130 protein can be a UL130 fragment of 10 amino acids or longer. For example, the number of amino acids in the fragment can comprise 10, 15, 20, 30, 40, 50, 60, 70, 80, 90, 100, 125, 150, 175, or 200 amino acids. A UL130 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204.

UL130 residues are numbered according to the full-length UL130 amino acid sequence (CMV UL130 FL) shown in SEQ ID NO: 27. Optionally, a UL130 fragment can extend further into the N-terminus by 5, 10, 20, or 30 amino acids from the starting residue of the fragment. Optionally, a UL130 fragment can extend further into the C-terminus by 5, 10, 20, or 30 amino acids from the last residue of the fragment.

CMV UL131 Proteins

In some embodiments, a UL131 protein is a full-length UL131 protein (CMV UL131, SEQ ID NO:29, for example, which is a 129 amino acid protein). In some embodiments the UL131 protein can be a UL131 fragment of 10 amino acids or longer. For example, the number of amino acids in the fragment can comprise 10, 15, 20, 30, 40, 50, 60, 70, 80, 90, 100, 125, 150, 175, or 200 amino acids. A UL131 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119.

UL131 residues are numbered according to the full-length UL131 amino acid sequence (CMV UL131 FL) shown in SEQ ID NO: 29. Optionally, a UL131 fragment can extend further into the N-terminus by 5, 10, 20, or 30 amino acids from the starting residue of the fragment. Optionally, a UL131 fragment can extend further into the C-terminus by 5, 10, 20, or 30 amino acids from the last residue of the fragment.

As stated above, the foregoing description of certain preferred embodiments, such as alphavirus VRPs and self-replicating RNAs that contain sequences encoding CMV proteins or fragments thereof, is illustrative of the invention but does not limit the scope of the invention. It will be appreciated that the sequences encoding CMV proteins in such preferred embodiments, can be replaced with sequences encoding proteins, such as gH and gL, or fragments thereof that are 10 amino acids long or longer, from other herpesviruses such as HHV-1, HHV-2, HHV-3, HHV-4, HHV-6, HHV-7 and HHV-8. For example, suitable VZV (HHV-3) proteins include gB, gE, gH, gI, and gL, and fragments thereof that are 10 amino acids long or longer, and can be from any VZV strain. For example, VZV proteins or fragments thereof can be from pOka, Dumas, HJO, CA123, or DR strains of VZV. These exemplary VZV proteins and fragments thereof can be encoded by any suitable nucleotide sequence, including sequences that are codon optimized or deoptimized for expression in a desired host, such as a human cell. Exemplary sequences of VZV proteins are provided herein.

For example, in one embodiment, the polycistronic nucleic acid molecule contains a first sequence encoding a VZV gH protein or fragment thereof, and a second sequence encoding a VZV gL protein or fragment thereof.

Suitable antigens include proteins and peptides from a pathogen such as a virus, bacteria, fungus, protozoan, plant or from a tumor. Viral antigens and immunogens that can be encoded by the self-replicating RNA molecule include, but are not limited to, proteins and peptides from a Orthomyxoviruses, such as Influenza A, B and C; Paramyxoviridae viruses, such as Pneumoviruses (RSV), Paramyxoviruses (PIV), Metapneumovirus and Morbilliviruses (e.g., measles); Pneumoviruses, such as Respiratory syncytial virus (RSV), Bovine respiratory syncytial virus, Pneumonia virus of mice, and Turkey rhinotracheitis virus; Paramyxoviruses, such as Parainfluenza virus types 1-4 (PIV), Mumps virus, Sendai viruses, Simian virus 5, Bovine parainfluenza virus, Nipahvirus, Henipavirus and Newcastle disease virus; Poxviridae, including a Orthopoxvirus such as Variola vera (including but not limited to, Variola major and Variola minor); Metapneumoviruses, such as human metapneumovirus (hMPV) and avian metapneumoviruses (aMPV); Morbilliviruses, such as Measles; Picornaviruses, such as Enteroviruses, Rhinoviruses, Heparnavirus, Parechovirus, Cardioviruses and Aphthoviruses; Enteroviruseses, such as Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71, Bunyaviruses, including a Orthobunyavirus such as California encephalitis virus; a Phlebovirus, such as Rift Valley Fever virus; a Nairovirus, such as Crimean-Congo hemorrhagic fever virus; Heparnaviruses, such as, Hepatitis A virus (HAV); Togaviruses (Rubella), such as a Rubivirus, an Alphavirus, or an Arterivirus; Flaviviruses, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus; Pestiviruses, such as Bovine viral diarrhea (BVDV), Classical swine fever (CSFV) or Border disease (BDV); Hepadnaviruses, such as Hepatitis B virus, Hepatitis C virus; Rhabdoviruses, such as a Lyssavirus (Rabies virus) and Vesiculovirus (VSV), Caliciviridae, such as Norwalk virus, and Norwalk-like Viruses, such as Hawaii Virus and Snow Mountain Virus; Coronaviruses, such as SARS, Human respiratory coronavirus, Avian infectious bronchitis (IBV), Mouse hepatitis virus (MHV), and Porcine transmissible gastroenteritis virus (TGEV); Retroviruses such as an Oncovirus, a Lentivirus or a Spumavirus; Reoviruses, as an Orthoreovirus, a Rotavirus, an Orbivirus, or a Coltivirus; Parvoviruses, such as Parvovirus B19; Delta hepatitis virus (HDV); Hepatitis E virus (HEV); Hepatitis G virus (HGV); Human Herpesviruses, such as, by way Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8); Papovaviruses, such as Papillomaviruses and Polyomaviruses, Adenoviruess and Arenaviruses.

In some embodiments, the antigen protein is from a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

In some embodiments the antigen protein is from a parasite from the Plasmodium genus, such as P.falciparum, P. vivax, P. malariae or P. ovale. Thus the invention may be used for immunizing against malaria. In some embodiments the antigen elicits an immune response against a parasite from the Caligidae family, particularly those from the Lepeophtheirus and Caligus genera e.g. sea lice such as Lepeophtheirus salmonis or Caligus rogercresseyi.

Bacterial antigens and immunogens that can be encoded by the self-replicating RNA molecule include, but are not limited to, proteins and peptides from Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Staphylococcus epidermis, Haemophilus influenzae, Clostridium tetani (Tetanus), Clostridium perfringens, Clostridium botulinums (Botulism), Cornynebacterium diphtherias (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae), Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli (such as enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC; such as uropathogenic E. coli (UPEC) and meningitis/sepsis-associated E. coli (MNEC)), and/or enterohemorrhagic E. coli (EHEC), Bacillus anthracia (anthrax), Yersinia pestis (plague), Mycobacterium tuberculosis, Rickettsia, Listeria monocytogenes, Chlamydia pneumoniae, Vibrio cholerae, Salmonella typhi (typhoid fever), Borrelia burgdorfer, Porphyromonas gingivalis, Klebsiella, Mycoplasma pneumoniae, etc.

Fungal antigens and immunogens that can be encoded by the self-replicating RNA molecule include, but are not limited to, proteins and peptides from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Protazoan antigens and immunogens that can be encoded by the self-replicating RNA molecule include, but are not limited to, proteins and peptides from Entamoeba histolytica, Giardia lambli, Cryptosporidium parvum, Cyclospora cayatanensis and Toxoplasma.

Plant antigens and immunogens that can be encoded by the self-replicating RNA molecule include, but are not limited to, proteins and peptides from Ricinus communis.

Suitable antigens include proteins and peptides from a virus such as, for example, human immunodeficiency virus (HIV), hepatitis A virus (HAV), hepatitis B virus (HBV), hepatitis C virus (HCV), herpes simplex virus (HSV), cytomegalovirus (CMV), influenza virus (flu), respiratory syncytial virus (RSV), parvovorus, norovirus, human papilloma virus (HPV), rhinovirus, yellow fever virus, rabies virus, Dengue fever virus, measles virus, mumps virus, rubella virus, varicella zoster virus, enterovirus (e.g., enterovirus 71), ebola virus, and bovine diarrhea virus. Preferably, the antigenic substance is selected from the group consisting of HSV glycoprotein gD, HIV glycoprotein gp120, HIV glycoprotein gp 40, HIV p55 gag, and polypeptides from the pol and tat regions. In other preferred embodiments of the invention, the antigen protein or peptides are derived from a bacterium such as, for example, Helicobacter pylori, Haemophilus influenza, Vibrio cholerae (cholera), C. diphtherias (diphtheria), C. tetani (tetanus), Neisseria meningitidis, B. pertussis, Mycobacterium tuberculosis, and the like.

HIV antigens that can be encoded by the self-replicating RNA molecules of the invention are described in U.S. application Ser. No. 490,858, filed Mar. 9, 1990, and published European application number 181150 (May 14, 1986), as well as U.S. application Ser. Nos. 60/168,471; 09/475,515; 09/475,504; and Ser. No. 09/610,313, the disclosures of which are incorporated herein by reference in their entirety.

Cytomegalovirus antigens that can be encoded by the self-replicating RNA molecules of the invention are described in U.S. Pat. No. 4,689,225, U.S. application Ser. No. 367,363, filed Jun. 16, 1989 and PCT Publication WO 89/07143, the disclosures of which are incorporated herein by reference in their entirety.

Hepatitis C antigens that can be encoded by the self-replicating RNA molecules of the invention are described in PCT/US88/04125, published European application number 318216 (May 31, 1989), published Japanese application number 1-500565 filed Nov. 18, 1988, Canadian application 583,561, and EPO 388,232, disclosures of which are incorporated herein by reference in their entirety. A different set of HCV antigens is described in European patent application 90/302866.0, filed Mar. 16, 1990, and U.S. application Ser. No. 456,637, filed Dec. 21, 1989, and PCT/US90/01348, the disclosures of which are incorporated herein by reference in their entirety.

In some embodiments, the antigen is derived from an allergen, such as pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In certain embodiments, a tumor immunogen or antigen, or cancer immunogen or antigen, can be encoded by the self-replicating RNA molecule. In certain embodiments, the tumor immunogens and antigens are peptide-containing tumor antigens, such as a polypeptide tumor antigen or glycoprotein tumor antigens.

Tumor immunogens and antigens appropriate for the use herein encompass a wide variety of molecules, such as (a) polypeptide-containing tumor antigens, including polypeptides (which can range, for example, from 8-20 amino acids in length, although lengths outside this range are also common), lipopolypeptides and glycoproteins.

In certain embodiments, tumor immunogens are, for example, (a) full length molecules associated with cancer cells, (b) homologs and modified forms of the same, including molecules with deleted, added and/or substituted portions, and (c) fragments of the same. Tumor immunogens include, for example, class I-restricted antigens recognized by CD8+ lymphocytes or class II-restricted antigens recognized by CD4+ lymphocytes.

In certain embodiments, tumor immunogens include, but are not limited to, (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g., prostate cancer, (f) immunoglobulin idiotypes (associated with myeloma and B cell lymphomas, for example).

In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

### Methods and Uses

In some embodiments, self-replicating RNA molecules or VRPs are administered to an individual to stimulate an immune response. In such embodiments, self-replicating RNA molecules or VRPs typically are present in a composition which may comprise a pharmaceutically acceptable carrier and, optionally, an adjuvant. See, e.g., U.S. Pat. No. 6,299,884; U.S. Pat. No. 7,641,911; U.S. Pat. No. 7,306,805; and US 2007/0207090.

The immune response can comprise a humoral immune response, a cell-mediated immune response, or both. In some embodiments an immune response is induced against each delivered CMV protein. A cell-mediated immune response can comprise a Helper T-cell (Tₕ) response, a CD8+ cytotoxic T-cell (CTL) response, or both. In some embodiments the immune response comprises a humoral immune response, and the antibodies are neutralizing antibodies. Neutralizing antibodies block viral infection of cells. CMV infects epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing; i.e., an antibody generated against an administered composition neutralizes a CMV virus of a strain other than the strain used in the composition.

A useful measure of antibody potency in the art is “50% neutralization titer.” To determine 50% neutralizing titer, serum from immunized animals is diluted to assess how dilute serum can be yet retain the ability to block entry of 50% of viruses into cells. For example, a titer of 700 means that serum retained the ability to neutralize 50% of virus after being diluted 700-fold. Thus, higher titers indicate more potent neutralizing antibody responses. In some embodiments, this titer is in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50% neutralization titer range can have an upper limit of about 400, about 600, about 800, about 1000, about 1500, about 200, about 2500, about 3000, about 3500, about 4000, about 4500, about 5000, about 5500, about 6000, about 6500, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 6500. “About” means plus or minus 10% of the recited value. Neutralization titer can be measured as described in the specific examples, below.

An immune response can be stimulated by administering VRPs or self-replicating RNA to an individual, typically a mammal, including a human. In some embodiments the immune response induced is a protective immune response, i.e., the response reduces the risk or severity of CMV infection. Stimulating a protective immune response is particularly desirable in some populations particularly at risk from CMV infection and disease. For example, at-risk populations include solid organ transplant (SOT) patients, bone marrow transplant patients, and hematopoietic stem cell transplant (HSCT) patients. VRPs can be administered to a transplant donor pre-transplant, or a transplant recipient pre- and/or post-transplant. Because vertical transmission from mother to child is a common source of infecting infants, administering VRPs or self-replicating RNA to a woman who can become pregnant is particularly useful.

Any suitable route of administration can be used. For example, a composition can be administered intra-muscularly, intra-peritoneally, sub-cutaneously, or trans-dermally. Some embodiments will be administered through an intra-mucosal route such as intra-orally, intra-nasally, intra-vaginally, and intra-rectally. Compositions can be administered according to any suitable schedule.

All patents, patent applications, and references cited in this disclosure, including nucleotide and amino acid sequences referred to by accession number, are expressly incorporated herein by reference. The above disclosure is a general description. A more complete understanding can be obtained by reference to the following specific examples, which are provided for purposes of illustration only.

### Example: Bicistronic and Pentacistronic Nucleic Acids Encoding CMV Proteins

RNA Synthesis

Plasmid DNA encoding alphavirus replicons served as a template for synthesis of RNA in vitro. Alphavirus replicons contain the genetic elements required for RNA replication but lack those encoding gene products necessary for particle assembly; the structural genes of the alphavirus genome are replaced by sequences encoding a heterologous protein. Upon delivery of the replicons to eukaryotic cells, the positive-stranded RNA is translated to produce four non-structural proteins, which together replicate the genomic RNA and transcribe abundant subgenomic mRNAs encoding the heterologous gene product or gene of interest (GOI). Due to the lack of expression of the alphavirus structural proteins, replicons are incapable of inducing the generation of infectious particles. A bacteriophage (T7 or SP6) promoter upstream of the alphavirus cDNA facilitates the synthesis of the replicon RNA in vitro and the hepatitis delta virus (HDV) ribozyme immediately downstream of the poly(A)-tail generates the correct 3′-end through its self-cleaving activity.

In order to allow the formation of an antigenic protein complex, the expression of the individual components of said complex in the same cell is of paramount importance. In theory, this can be accomplished by co-transfecting cells with the genes encoding the individual components. However, in case of non-virally or VRP delivered alphavirus replicon RNAs, this strategy is hampered by inefficient co-delivery of multiple RNAs to the same cell or, alternatively, by inefficient launch of multiple self-replicating RNAs in an individual cell. A potentially more efficient way to facilitate co-expression of components of a protein complex is to deliver the respective genes as part of the same self-replicating RNA molecule. To this end, we engineered alphavirus replicon constructs encoding multiple genes of interest. Every GOI is preceded by its own subgenomic promoter which is recognized by the alphavirus transcription machinery. Thereby, multiple subgenomic messenger RNA species are synthesized in an individual cell allowing the assembly of multi-component protein complexes.

Following linearization of the plasmid DNA downstream of the HDV ribozyme with a suitable restriction endonuclease, run-off transcripts were synthesized in vitro using T7 bacteriophage derived DNA-dependent RNA polymerase. Transcriptions were performed for 2 hours at 37° C. in the presence of 7.5 mM of each of the nucleoside triphosphates (ATP, CTP, GTP and UTP) following the instructions provided by the manufacturer (Ambion, Austin, Tex.). Following transcription, the template DNA was digested with TURBO DNase (Ambion, Austin, Tex.). The replicon RNA was precipitated with LiCl and reconstituted in nuclease-free water. Uncapped RNA was capped post-transcripionally with Vaccinia Capping Enzyme (VCE) using the ScriptCap m⁷G Capping System (Epicentre Biotechnologies, Madison, Wis.) as outlined in the user manual. Post-transcriptionally capped RNA was precipitated with LiCl and reconstituted in nuclease-free water. The concentration of the RNA samples was determined by measuring the optical density at 260 nm. Integrity of the in vitro transcripts was confirmed by denaturing agarose gel electrophoresis.

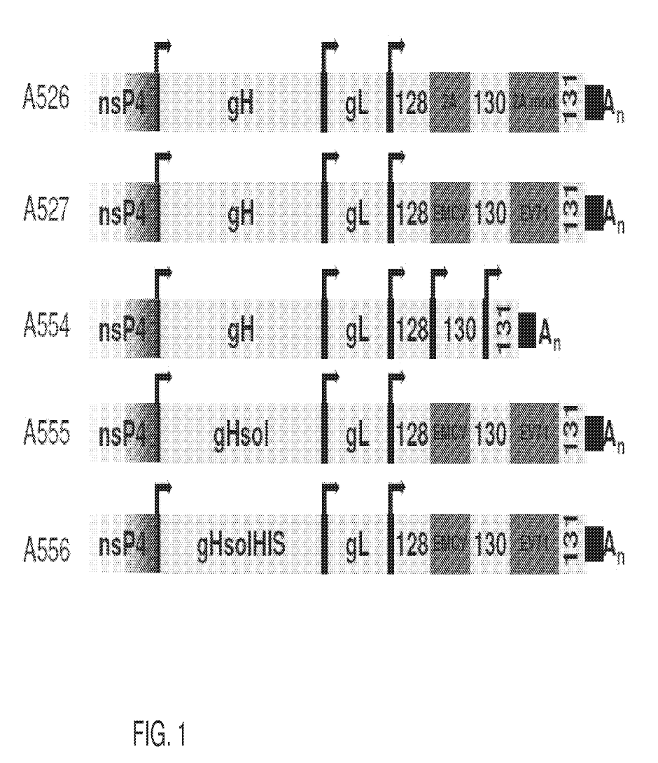

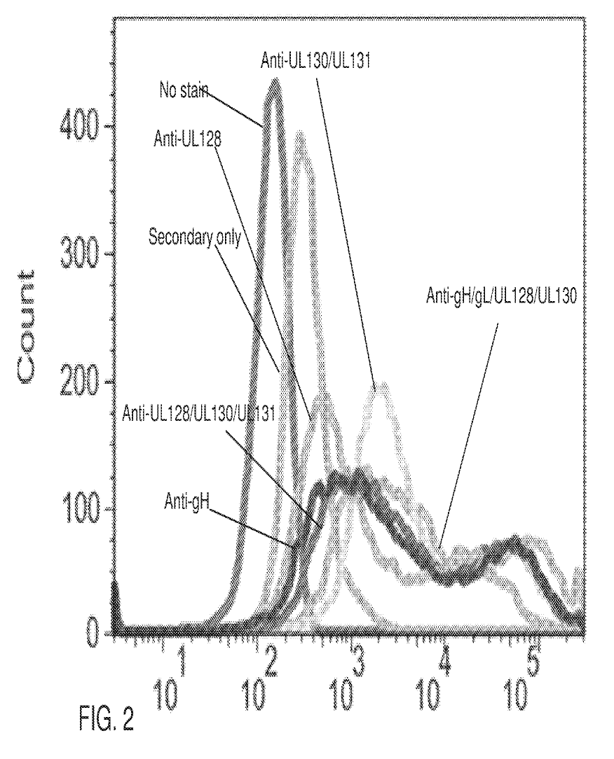

Bicistronic and pentacistronic alphavirus replicons that express glycoprotein complexes from human cytomegalovirus (HCMV) were prepared, and are shown schematically in FIG. 1. The alphavirus replicons were based on venezuelan equine encephalitis virus (VEE). The alphavirus replicons were based on venezuelan equine encephalitis virus (VEE). The replicons were packaged into viral replicon particles (VRPs), encapsulated in lipid nanoparticles (LNP), or formulated with a cationic nanoemulsion (CNE). Expression of the encoded HCMV proteins and protein complexes from each of the replicons was confirmed by immunoblot, co-immunoprecipitation, and flow cytometry. Flow cytometry was used to verify expression of the pentameric gH/gL/UL128/UL130/UL131 complex from pentameric replicons encoding the protein components of the complex, using human monoclonal antibodies specific to conformational epitopes present on the pentameric complex (Macagno et al (2010), J. Virol. 84(2):1005-13). FIG. 2 shows that these antibodies bind to BHKV cells transfected with replicon RNA expressing the HCMV gH/gL/UL128/UL130/UL131 pentameric complex (A527). Similar results were obtained when cells were infected with VRPs made from the same replicon construct. This shows that replicons designed to express the pentameric complex do indeed express the desired antigen and not the potential byproduct gH/gL.

The VRPs, RNA encaspulated in LNPs, and RNA formulated with a cationic oil-in-water nanoemulsion (CNE) were used to immunize Balb/c mice by intramuscular injections in the rear quadriceps. The mice were immunized three times, three weeks apart, and serum samples were collected prior to each immunization as well as three weeks after the third and final immunization. The sera were evaluated in microneutralization assays and to measure the potency of the neutralizing antibody response that was elicited by the vaccinations. The titers are expressed as 50% neutralizing titer.

The immunogenicity of LNP-encapsulated RNAs encoding the pentameric complex (A526 and A527) compared to LNP-encapsulated RNA and VRPs (A160) expressing gH/gL was assessed. Table 3 shows that replicons expressing the pentameric complex elicited more potently neutralizing antibodies than replicons expressing gH/gL.

TABLE 3
Neutralizing antibody titers.

| | Titer | Titer | Titer |
|---|---|---|---|
| Replicon | post 1^{st} | post 2^{nd} | post 3^{rd} |
| C313 VEE/SIN gH FL/gL VRP 10⁶ IU | 126 | 6,296 | 26,525 |
| A160 gH FL/gL 1 μg LNP | 347 | 9,848 | 42,319 |
| A526 Pentameric 2A 1 μg LNP | 179 | 12,210 | 80,000 |
| A527 Pentameric IRES 1 μg LNP | 1,510 | 51,200 | 130,000 |

The pentacistronic VEE-based RNA replicon that elicited the highest titers of neutralizing antibodies (A527) was packaged as VRPs and the immunogenicity of the VRPs were compared to gH/gL-expressing VRPs and LNP-encapsulated replicons expressing gH/gL and pentameric complex. Table 4 shows that VRPs expressing the pentameric complex elicited higher titers of neutralizing antibodies than VRPs expressing gH/gL. Moreover, 10⁶ infectious units of VRPs are at least as potent as 1 μg of LNP-encapsulated RNA when the VRPs and the RNA encoded the same protein complexes.

TABLE 4
Neutralizing antibody titers. Sera were collected
three weeks after the second immunization.

| Replicon | 50% Neutralizing Titer |
|---|---|
| A160 gH FL/gL VRP 10⁶ IU | 14,833 |
| A527 Pentameric IRES VRP 10⁶ IU | 51,200 |
| A160 gH FL/gL LNP 0.01 μg | 4,570 |
| A160 gH FL/gL LNP 0.1 μg | 9,415 |
| A160 gH FL/gL LNP 1 μg | 14,427 |
| A527 Pentameric IRES 0.01 μg LNP | 12,693 |
| A527 Pentameric IRES 0.1 μg LNP | 10,309 |
| A527 Pentameric IRES 1 μg LNP | 43,157 |

The breadth and potency of HCMV neutralizing activity in sera from mice immunized with VEE-based RNA encoding the pentameric complex (A527) was assessed by using the sera to block infection of fibroblasts and epithelial cells with different strains of HCMV. Table 5 shows that anti-gH/gL/UL128/UL130/UL131 immune sera broadly and potently neutralized infection of epithelial cells. This effect was complement independent. In contrast, the sera had a reduced or not detectable effect on infection of fibroblasts. These results are what is expected for immune sera that contains mostly antibodies specific for the gH/gL/UL128/UL130/UL131 pentameric complex, because the pentameric complex is not required for infection of fibroblasts and, consequently, antibodies to UL128, UL130, and UL131 do not block infection of fibroblasts (Adler et al (2006), J. Gen. Virol. 87(Pt.9):2451-60; Wang and Shenk (2005), Proc. Natl. Acad. Sci. USA 102(50):18153-8). Thus, these data demonstrate that the pentameric replicons encoding the gH/gL/UL128/UL130/UL131pentameric complex specifically elicit antibodies to the complex in vivo.

TABLE 5
Neutralizing antibody titers in sera from mice immunized
with the A527 RNA replicon encapsulated in LNPs.
The replicon expresses the HCMV pentameric complex
using subgenomic promoters and IRESes.

| | Serum from mice immunized with A527 | | |
|---|---|---|---|
| | pentameric IRES RNA in LNPs | | |
| | | Without | With |
| HCMV Strain | Cell | complement | complement |
| Towne | Fibroblasts | 3433 | 1574 |
| AD169 | (MRC-5) | 2292 | <1000 |
| TB40-UL32-EGFP | | <1000 | <1000 |
| VR1814 | | 4683 | 1324 |
| TB40-UL32-EGFP | Epithelial cells | 86991 | 59778 |
| VR1814 | (ARPE-19) | 82714 | 37293 |
| 8819 (clinical | | 94418 | 43269 |
| isolate) | | | |
| 8822 (clinical | | 85219 | 49742 |
| isolate) | | | |

To see if bicistronic and pentacistronic replicons expressing the gH/gL and pentameric complexes would elicit neutralizing antibodies in different formulations, cotton rats were immunized with bicistronic or pentacistronic replicons mixed with a cationic nanoemulsion (CNE). Table 6 shows that replicons in CNE elicited comparable neutralizing antibody titers to the same replicons encapsulated in LNPs.

TABLE 6
Neutralizing antibody titers. The sera were collected
three weeks after the second immunization.

| Replicon | 50% Neutralizing Titer |
|---|---|
| A160 gH FL/gL VRP 10⁶ IU | 594 |
| A160 gH FL/gL 1 μg LNP | 141 |
| A527 Pentameric IRES 1 μg LNP | 4,416 |
| A160 gH FL/gL 1 μg CNE | 413 |
| A527 Pentameric IRES 1 μg CNE | 4,411 |

SEQUENCES

CMV gB FL:
1 -
(SEQ ID NO: 6)
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagcgcgtgaaatacgtggaggacaaggtggtgga
ccccctgcctccttacctgaagggcctggacgacctgatgagcggactgggcgctgccggaa
aagccgtgggagtggccattggagctgtgggcggagctgtggcctctgtcgtggaaggcgtc
gccacctttctgaagaaccccttcggcgccttcaccatcatcctggtggccattgccgtcgt
gatcatcacctacctgatctacacccggcagcggagactgtgtacccagcccctgcagaacc
tgttcccctacctggtgtccgccgatggcaccacagtgaccagcggctccaccaaggatacc
agcctgcaggccccacccagctacgaagagagcgtgtacaacagcggcagaaagggccctgg
ccctcccagctctgatgccagcacagccgcccctccctacaccaacgagcaggcctaccaga
tgctgctggccctggctagactggatgccgagcagagggcccagcagaacggcaccgacagc
ctggatggcagaaccggcacccaggacaagggccagaagcccaacctgctggaccggctgcg
gcaccggaagaacggctaccggcacctgaaggacagcgacgaggaagagaacgtctgataa - 2727
CMV gB FL
(SEQ ID NO: 7)
MESRIWCLVVCVNLCIVCLGAAVSSSSTRGTSATHSHHSSHTTSAAHSRSGSVSQRVTSSQT
VSHGVNETIYNTTLKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPMWEIHHINSHSQCY
SSYSRVIAGTVFVAYHRDSYENKTMQLMPDDYSNTHSTRYVTVKDQWHSRGSTWLYRETCNL
NCMVTITTARSKYPYHFFATSTGDVVDISPFYNGTNRNASYFGENADKFFIFPNYTIVSDFG
RPNSALETHRLVAFLERADSVISWDIQDEKNVTCQLTFWEASERTIRSEAEDSYHFSSAKMT
ATFLSKKQEVNMSDSALDCVRDEAINKLQQIFNTSYNQTYEKYGNVSVFETTGGLVVFWQGI
KQKSLVELERLANRSSLNLTHNRTKRSTDGNNATHLSNMESVHNLVYAQLQFTYDTLRGYIN
RALAQIAEAWCVDQRRTLEVFKELSKINPSAILSAIYNKPIAARFMGDVLGLASCVTINQTS
VKVLRDMNVKESPGRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQKELRSSNVFDLEE
IMREFNSYKQRVKYVEDKVVDPLPPYLKGLDDLMSGLGAAGKAVGVAIGAVGGAVASVVEGV
ATFLKNPFGAFTIILVAIAVVIITYLIYTRQRRLCTQPLQNLFPYLVSADGTTVTSGSTKDT
SLQAPPSYEESVYNSGRKGPGPPSSDASTAAPPYTNEQAYQMLLALARLDAEQRAQQNGTDS
LDGRTGTQDKGQKPNLLDRLRHRKNGYRHLKDSDEEENV--
CMV gB sol 750:
1-
(SEQ ID NO: 8)
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagcgcgtgaaatacgtggaggacaaggtggtgga
ccccctgcctccttacctgaagggcctggacgacctgatgagcggactgggcgctgccggaa
aagccgtgggagtggccattggagctgtgggcggagctgtggcctctgtcgtggaaggcgtc
gccacctttctgaagaactgataa - 2256
Cmv gB sol 750
(SEQ ID NO: 9)
MESRIWCLVVCVNLCIVCLGAAVSSSSTRGTSATHSHHSSHTTSAAHSRSGSVSQRVTSSQT
VSHGVNETIYNTTLKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPMWEIHHINSHSQCY
SSYSRVIAGTVFVAYHRDSYENKTMQLMPDDYSNTHSTRYVTVKDQWHSRGSTWLYRETCNL
NCMVTITTARSKYPYHFFATSTGDVVDISPFYNGTNRNASYFGENADKFFIFPNYTIVSDFG
RPNSALETHRLVAFLERADSVISWDIQDEKNVTCQLTFWEASERTIRSEAEDSYHFSSAKMT
ATFLSKKQEVNMSDSALDCVRDEAINKLQQIFNTSYNQTYEKYGNVSVFETTGGLVVFWQGI
KQKSLVELERLANRSSLNLTHNRTKRSTDGNNATHLSNMESVHNLVYAQLQFTYDTLRGYIN
RALAQIAEAWCVDQRRTLEVFKELSKINPSAILSAIYNKPIAARFMGDVLGLASCVTINQTS
VKVLRDMNVKESPGRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQKELRSSNVFDLEE
IMREFNSYKQRVKYVEDKVVDPLPPYLKGLDDLMSGLGAAGKAVGVAIGAVGGAVASVVEGV
ATFLKN--
CMV gB sol 692:
1-
(SEQ ID NO: 10)
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagtgataa - 2082
Cmv gB sol 692;
(SEQ ID NO: 11)
MESRIWCLVVCVNLCIVCLGAAVSSSSTRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEGIMVVYKRNIVAHTFKVRVYQKVLTFR
RSYAYIHTTYLLGSNTEYVAPPMWEIHHINSHSQCYSSYSRVIAGTVFVAYHRDSYENKTMQLMPDDYSNTHSTR
YVTVKDQWHSRGSTWLYRETCNLNCMVTITTARSKYPYHFFATSTGDVVDISPFYNGTNRNASYFGENADKFFIF
PNYTIVSDFGRPNSALETHRLVAFLERADSVISWDIQDEKNVTCQLTFWEASERTIRSEAEDSYHFSSAKMTATF
LSKKQEVNMSDSALDCVRDEAINKLQQIFNTSYNQTYEKYGNVSVFETTGGLVVFWQGIKQKSLVELERLANRSS
LNLTHNRTKRSTDGNNATHLSNMESVHNLVYAQLQFTYDTLRGYINRALAQIAEAWCVDQRRTLEVFKELSKINP
SAILSAIYNKPIAARFMGDVLGLASCVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIALDIDPLENTDFRVLELYSQKELRSS
NVFDLEEIMREFNSYKQ-
CMV gH FL:
1 -
(SEQ ID NO: 12)
atgaggcctggcctgccctcctacctgatcatcctggccgtgtgcctgttcagccacctgctgtccagcagatac
ggcgccgaggccgtgagcgagcccctggacaaggctttccacctgctgctgaacacctacggcagacccatccgg
tttctgcgggagaacaccacccagtgcacctacaacagcagcctgcggaacagcaccgtcgtgagagagaacgcc
atcagcttcaactttttccagagctacaaccagtactacgtgttccacatgcccagatgcctgtttgccggccct
ctggccgagcagttcctgaaccaggtggacctgaccgagacactggaaagataccagcagcggctgaatacctac
gccctggtgtccaaggacctggccagctaccggtcctttagccagcagctcaaggctcaggatagcctcggcgag
cagcctaccaccgtgccccctcccatcgacctgagcatcccccacgtgtggatgcctccccagaccacccctcac
ggctggaccgagagccacaccacctccggcctgcacagaccccacttcaaccagacctgcatcctgttcgacggc
cacgacctgctgtttagcaccgtgaccccctgcctgcaccagggcttctacctgatcgacgagctgagatacgtg
aagatcaccctgaccgaggatttcttcgtggtcaccgtgtccatcgacgacgacacccccatgctgctgatcttc
ggccacctgcccagagtgctgttcaaggccccctaccagcgggacaacttcatcctgcggcagaccgagaagcac
gagctgctggtgctggtcaagaaggaccagctgaaccggcactcctacctgaaggaccccgacttcctggacgcc
gccctggacttcaactacctggacctgagcgccctgctgagaaacagcttccacagatacgccgtggacgtgctg
aagtccggacggtgccagatgctcgatcggcggaccgtggagatggccttcgcctatgccctcgccctgttcgcc
gctgccagacaggaagaggctggcgcccaggtgtcagtgcccagagccctggatagacaggccgccctgctgcag
atccaggaattcatgatcacctgcctgagccagaccccccctagaaccaccctgctgctgtaccccacagccgtg
gatctggccaagagggccctgtggacccccaaccagatcaccgacatcacaagcctcgtgcggctcgtgtacatc
ctgagcaagcagaaccagcagcacctgatcccccagtgggccctgagacagatcgccgacttcgccctgaagctg
cacaagacccatctggccagctttctgagcgccttcgccaggcaggaactgtacctgatgggcagcctggtccac
agcatgctggtgcataccaccgagcggcgggagatcttcatcgtggagacaggcctgtgtagcctggccgagctg
tcccactttacccagctgctggcccaccctcaccacgagtacctgagcgacctgtacaccccctgcagcagcagc
ggcagacgggaccacagcctggaacggctgaccagactgttccccgatgccaccgtgcctgctacagtgcctgcc
gccctgtccatcctgtccaccatgcagcccagcaccctggaaaccttccccgacctgttctgcctgcccctgggc
gagagctttagcgccctgaccgtgtccgagcacgtgtcctacatcgtgaccaatcagtacctgatcaagggcatc
agctaccccgtgtccaccacagtcgtgggccagagcctgatcatcacccagaccgacagccagaccaagtgcgag
ctgacccggaacatgcacaccacacacagcatcaccgtggccctgaacatcagcctggaaaactgcgctttctgt
cagtctgccctgctggaatacgacgatacccagggcgtgatcaacatcatgtacatgcacgacagcgacgacgtg
ctgttcgccctggacccctacaacgaggtggtggtgtccagcccccggacccactacctgatgctgctgaagaac
ggcaccgtgctggaagtgaccgacgtggtggtggacgccaccgacagcagactgctgatgatgagcgtgtacgcc
ctgagcgccatcatcggcatctacctgctgtaccggatgctgaaaacctgctgataa - 2232
Cmv gH FL;
(SEQ ID NO: 13)
MRPGLPSYLIILAVCLFSHLLSSRYGAEAVSEPLDKAFHLLLNTYGRPIRFLRENTTQCTYN
SSLRNSTVVRENAISFNFFQSYNQYYVFHMPRCLFAGPLAEQFLNQVDLTETLERYQQRLNT
YALVSKDLASYRSFSQQLKAQDSLGEQPTTVPPPIDLSIPHVWMPPQTTPHGWTESHTTSGL
HRPHFNQTCILFDGHDLLFSTVTPCLHQGFYLIDELRYVKITLTEDFFVVTVSIDDDTPMLL
IFGHLPRVLFKAPYQRDNFILRQTEKHELLVLVKKDQLNRHSYLKDPDFLDAALDFNYLDLS
ALLRNSFHRYAVDVLKSGRCQMLDRRTVEMAFAYALALFAAARQEEAGAQVSVPRALDRQAA
LLQIQEFMITCLSQTPPRTTLLLYPTAVDLAKRALWTPNQITDITSLVRLVYILSKQNQQHL
IPQWALRQIADFALKLHKTHLASFLSAFARQELYLMGSLVHSMLVHTTERREIFIVETGLCS
LAELSHFTQLLAHPHHEYLSDLYTPCSSSGRRDHSLERLTRLFPDATVPATVPAALSILSTM
QPSTLETFPDLFCLPLGESFSALTVSEHVSYIVTNQYLIKGISYPVSTTVVGQSLIITQTDS
QTKCELTRNMHTTHSITVALNISLENCAFCQSALLEYDDTQGVINIMYMHDSDDVLFALDPY
NEVVVSSPRTHYLMLLKNGTVLEVTDVVVDATDSRLLMMSVYALSAIIGIYLLYRMLKTC--
CMV gH sol:
1-
(SEQ ID NO: 14)
atgaggcctggcctgccctcctacctgatcatcctggccgtgtgcctgttcagccacctgct
gtccagcagatacggcgccgaggccgtgagcgagcccctggacaaggctttccacctgctgc
tgaacacctacggcagacccatccggtttctgcgggagaacaccacccagtgcacctacaac
agcagcctgcggaacagcaccgtcgtgagagagaacgccatcagcttcaactttttccagag
ctacaaccagtactacgtgttccacatgcccagatgcctgtttgccggccctctggccgagc
agttcctgaaccaggtggacctgaccgagacactggaaagataccagcagcggctgaatacc
tacgccctggtgtccaaggacctggccagctaccggtcctttagccagcagctcaaggctca
ggatagcctcggcgagcagcctaccaccgtgccccctcccatcgacctgagcatcccccacg
tgtggatgcctccccagaccacccctcacggctggaccgagagccacaccacctccggcctg
cacagaccccacttcaaccagacctgcatcctgttcgacggccacgacctgctgtttagcac
cgtgaccccctgcctgcaccagggcttctacctgatcgacgagctgagatacgtgaagatca
ccctgaccgaggatttcttcgtggtcaccgtgtccatcgacgacgacacccccatgctgctg
atcttcggccacctgcccagagtgctgttcaaggccccctaccagcgggacaacttcatcct
gcggcagaccgagaagcacgagctgctggtgctggtcaagaaggaccagctgaaccggcact
cctacctgaaggaccccgacttcctggacgccgccctggacttcaactacctggacctgagc
gccctgctgagaaacagcttccacagatacgccgtggacgtgctgaagtccggacggtgcca
gatgctcgatcggcggaccgtggagatggccttcgcctatgccctcgccctgttcgccgctg
ccagacaggaagaggctggcgcccaggtgtcagtgcccagagccctggatagacaggccgcc
ctgctgcagatccaggaattcatgatcacctgcctgagccagaccccccctagaaccaccct
gctgctgtaccccacagccgtggatctggccaagagggccctgtggacccccaaccagatca
ccgacatcacaagcctcgtgcggctcgtgtacatcctgagcaagcagaaccagcagcacctg
atcccccagtgggccctgagacagatcgccgacttcgccctgaagctgcacaagacccatct
ggccagctttctgagcgccttcgccaggcaggaactgtacctgatgggcagcctggtccaca
gcatgctggtgcataccaccgagcggcgggagatcttcatcgtggagacaggcctgtgtagc
ctggccgagctgtcccactttacccagctgctggcccaccctcaccacgagtacctgagcga
cctgtacaccccctgcagcagcagcggcagacgggaccacagcctggaacggctgaccagac
tgttccccgatgccaccgtgcctgctacagtgcctgccgccctgtccatcctgtccaccatg
cagcccagcaccctggaaaccttccccgacctgttctgcctgcccctgggcgagagctttag
cgccctgaccgtgtccgagcacgtgtcctacatcgtgaccaatcagtacctgatcaagggca
tcagctaccccgtgtccaccacagtcgtgggccagagcctgatcatcacccagaccgacagc
cagaccaagtgcgagctgacccggaacatgcacaccacacacagcatcaccgtggccctgaa
catcagcctggaaaactgcgctttctgtcagtctgccctgctggaatacgacgatacccagg
gcgtgatcaacatcatgtacatgcacgacagcgacgacgtgctgttcgccctggacccctac
aacgaggtggtggtgtccagcccccggacccactacctgatgctgctgaagaacggcaccgt
gctggaagtgaccgacgtggtggtggacgccaccgactgataa - 2151
CMV gH sol;
(SEQ ID NO: 15)
MRPGLPSYLIILAVCLFSHLLSSRYGAEAVSEPLDKAFHLLLNTYGRPIRFLRENTTQCTYN
SSLRNSTVVRENAISFNFFQSYNQYYVFHMPRCLFAGPLAEQFLNQVDLTETLERYQQRLNT
YALVSKDLASYRSFSQQLKAQDSLGEQPTTVPPPIDLSIPHVWMPPQTTPHGWTESHTTSGL
HRPHFNQTCILFDGHDLLFSTVTPCLHQGFYLIDELRYVKITLTEDFFVVTVSIDDDTPMLL
IFGHLPRVLFKAPYQRDNFILRQTEKHELLVLVKKDQLNRHSYLKDPDFLDAALDFNYLDLS
ALLRNSFHRYAVDVLKSGRCQMLDRRTVEMAFAYALALFAAARQEEAGAQVSVPRALDRQAA
LLQIQEFMITCLSQTPPRTTLLLYPTAVDLAKRALWTPNQITDITSLVRLVYILSKQNQQHL
IPQWALRQIADFALKLHKTHLASFLSAFARQELYLMGSLVHSMLVHTTERREIFIVETGLCS
LAELSHFTQLLAHPHHEYLSDLYTPCSSSGRRDHSLERLTRLFPDATVPATVPAALSILSTM
QPSTLETFPDLFCLPLGESFSALTVSEHVSYIVTNQYLIKGISYPVSTTVVGQSLIITQTDS
QTKCELTRNMHTTHSITVALNISLENCAFCQSALLEYDDTQGVINIMYMHDSDDVLFALDPY
NEVVVSSPRTHYLMLLKNGTVLEVTDVVVDATD--
CMV gL fl:
1-
(SEQ ID NO: 16)
atgtgcagaaggcccgactgcggcttcagcttcagccctggacccgtgatcctgctgtggtg
ctgcctgctgctgcctatcgtgtcctctgccgccgtgtctgtggcccctacagccgccgaga
aggtgccagccgagtgccccgagctgaccagaagatgcctgctgggcgaggtgttcgagggc
gacaagtacgagagctggctgcggcccctggtcaacgtgaccggcagagatggccccctgag
ccagctgatccggtacagacccgtgacccccgaggccgccaatagcgtgctgctggacgagg
ccttcctggataccctggccctgctgtacaacaaccccgaccagctgagagccctgctgacc
ctgctgtccagcgacaccgcccccagatggatgaccgtgatgcggggctacagcgagtgtgg
agatggcagccctgccgtgtacacctgcgtggacgacctgtgcagaggctacgacctgacca
gactgagctacggccggtccatcttcacagagcacgtgctgggcttcgagctggtgcccccc
agcctgttcaacgtggtggtggccatccggaacgaggccaccagaaccaacagagccgtgcg
gctgcctgtgtctacagccgctgcacctgagggcatcacactgttctacggcctgtacaacg
ccgtgaaagagttctgcctccggcaccagctggatccccccctgctgagacacctggacaag
tactacgccggcctgcccccagagctgaagcagaccagagtgaacctgcccgcccacagcag
atatggccctcaggccgtggacgccagatgataa - 840
CMV gL FL;
(SEQ ID NO: 17)
MCRRPDCGFSFSPGPVILLWCCLLLPIVSSAAVSVAPTAAEKVPAECPELTRRCLLGEVFEG
DKYESWLRPLVNVTGRDGPLSQLIRYRPVTPEAANSVLLDEAFLDTLALLYNNPDQLRALLT
LLSSDTAPRWMTVMRGYSECGDGSPAVYTCVDDLCRGYDLTRLSYGRSIFTEHVLGFELVPP
SLFNVVVAIRNEATRTNRAVRLPVSTAAAPEGITLFYGLYNAVKEFCLRHQLDPPLLRHLDK
YYAGLPPELKQTRVNLPAHSRYGPQAVDAR--
CMV gM FL:
1-
(SEQ ID NO: 18)
atggcccccagccacgtggacaaagtgaacacccggacttggagcgccagcatcgtgttcat
ggtgctgaccttcgtgaacgtgtccgtgcacctggtgctgtccaacttcccccacctgggct
acccctgcgtgtactaccacgtggtggacttcgagcggctgaacatgagcgcctacaacgtg
atgcacctgcacacccccatgctgtttctggacagcgtgcagctcgtgtgctacgccgtgtt
catgcagctggtgtttctggccgtgaccatctactacctcgtgtgctggatcaagatcagca
tgcggaaggacaagggcatgagcctgaaccagagcacccgggacatcagctacatgggcgac
agcctgaccgccttcctgttcatcctgagcatggacaccttccagctgttcaccctgaccat
gagcttccggctgcccagcatgatcgccttcatggccgccgtgcactttttctgtctgacca
tcttcaacgtgtccatggtcacccagtaccggtcctacaagcggagcctgttcttcttctcc
cggctgcaccccaagctgaagggcaccgtgcagttccggaccctgatcgtgaacctggtgga
ggtggccctgggcttcaataccaccgtggtggctatggccctgtgctacggcttcggcaaca
acttcttcgtgcggaccggccatatggtgctggccgtgttcgtggtgtacgccatcatcagc
atcatctactttctgctgatcgaggccgtgttcttccagtacgtgaaggtgcagttcggcta
ccatctgggcgcctttttcggcctgtgcggcctgatctaccccatcgtgcagtacgacacct
tcctgagcaacgagtaccggaccggcatcagctggtccttcggaatgctgttcttcatctgg
gccatgttcaccacctgcagagccgtgcggtacttcagaggcagaggcagcggctccgtgaa
gtaccaggccctggccacagcctctggcgaagaggtggccgccctgagccaccacgacagcc
tggaaagcagacggctgcgggaggaagaggacgacgacgacgaggacttcgaggacgcctga
taa - 1119
CMV gM FL;
(SEQ ID NO: 19)
MAPSHVDKVNTRTWSASIVFMVLTFVNVSVHLVLSNFPHLGYPCVYYHVVDFERLNMSAYNV
MHLHTPMLFLDSVQLVCYAVFMQLVFLAVTIYYLVCWIKISMRKDKGMSLNQSTRDISYMGD
SLTAFLFILSMDTFQLFTLTMSFRLPSMIAFMAAVHFFCLTIFNVSMVTQYRSYKRSLFFFS
RLHPKLKGTVQFRTLIVNLVEVALGFNITVVAMALCYGFGNNFFVRTGHMVLAVFVVYAIIS
IIYFLLIEAVFFQYVKVQFGYHLGAFFGLCGLIYPIVQYDTFLSNEYRTGISWSFGMLFFIW
AMFTTCRAVRYFRGRGSGSVKYQALATASGEEVAALSHHDSLESRRLREEEDDDDEDFEDA--
CMV gN FL:
1-
(SEQ ID NO: 20)
atggaatggaacaccctggtcctgggcctgctggtgctgtctgtcgtggccagcagcaacaa
cacatccacagccagcacccctagacctagcagcagcacccacgccagcactaccgtgaagg
ctaccaccgtggccaccacaagcaccaccactgctaccagcaccagctccaccacctctgcc
aagcctggctctaccacacacgaccccaacgtgatgaggccccacgcccacaacgacttcta
caacgctcactgcaccagccacatgtacgagctgtccctgagcagctttgccgcctggtgga
ccatgctgaacgccctgatcctgatgggcgccttctgcatcgtgctgcggcactgctgcttc
cagaacttcaccgccaccaccaccaagggctactgataa - 411
CMV gN FL;
(SEQ ID NO: 21)
MEWNTLVLGLLVLSVVASSNNTSTASTPRPSSSTHASTTVKATTVATTSTTTATSTSSTTSA
KPGSTTHDPNVMRPHAHNDFYNAHCTSHMYELSLSSFAAWWTMLNALILMGAFCIVLRHCCF
QNFTATTTKGY--
CMV gO FL:
1-
(SEQ ID NO: 22)
atgggcaagaaagaaatgatcatggtcaagggcatccccaagatcatgctgctgattagcat
cacctttctgctgctgtccctgatcaactgcaacgtgctggtcaacagccggggcaccagaa
gatcctggccctacaccgtgctgtcctaccggggcaaagagatcctgaagaagcagaaagag
gacatcctgaagcggctgatgagcaccagcagcgacggctaccggttcctgatgtaccccag
ccagcagaaattccacgccatcgtgatcagcatggacaagttcccccaggactacatcctgg
ccggacccatccggaacgacagcatcacccacatgtggttcgacttctacagcacccagctg
cggaagcccgccaaatacgtgtacagcgagtacaaccacaccgcccacaagatcaccctgag
gcctcccccttgtggcaccgtgcccagcatgaactgcctgagcgagatgctgaacgtgtcca
agcggaacgacaccggcgagaagggctgcggcaacttcaccaccttcaaccccatgttcttc
aacgtgccccggtggaacaccaagctgtacatcggcagcaacaaagtgaacgtggacagcca
gaccatctactttctgggcctgaccgccctgctgctgagatacgcccagcggaactgcaccc
ggtccttctacctggtcaacgccatgagccggaacctgttccgggtgcccaagtacatcaac
ggcaccaagctgaagaacaccatgcggaagctgaagcggaagcaggccctggtcaaagagca
gccccagaagaagaacaagaagtcccagagcaccaccaccccctacctgagctacaccacct
ccaccgccttcaacgtgaccaccaacgtgacctacagcgccacagccgccgtgaccagagtg
gccacaagcaccaccggctaccggcccgacagcaactttatgaagtccatcatggccaccca
gctgagagatctggccacctgggtgtacaccaccctgcggtacagaaacgagcccttctgca
agcccgaccggaacagaaccgccgtgagcgagttcatgaagaatacccacgtgctgatcaga
aacgagacaccctacaccatctacggcaccctggacatgagcagcctgtactacaacgagac
aatgagcgtggagaacgagacagccagcgacaacaacgaaaccacccccacctcccccagca
cccggttccagcggaccttcatcgaccccctgtgggactacctggacagcctgctgttcctg
gacaagatccggaacttcagcctgcagctgcccgcctacggcaatctgaccccccctgagca
cagaagggccgccaacctgagcaccctgaacagcctgtggtggtggagccagtgataa - 1422
CMV gO FL;
(SEQ ID NO: 23)
MGKKEMIMVKGIPKIMLLISITFLLLSLINCNVLVNSRGTRRSWPYTVLSYRGKEILKKQKE
DILKRLMSTSSDGYRFLMYPSQQKFHAIVISMDKFPQDYILAGPIRNDSITHMWFDFYSTQL
RKPAKYVYSEYNHTAHKITLRPPPCGTVPSMNCLSEMLNVSKRNDTGEKGCGNFTTFNPMFF
NVPRWNTKLYIGSNKVNVDSQTIYFLGLTALLLRYAQRNCTRSFYLVNAMSRNLFRVPKYIN
GTKLKNTMRKLKRKQALVKEQPQKKNKKSQSTTTPYLSYTTSTAFNVTTNVTYSATAAVTRV
ATSTTGYRPDSNFMKSIMATQLRDLATWVYTTLRYRNEPFCKPDRNRTAVSEFMKNTHVLIR
NETPYTIYGTLDMSSLYYNETMSVENETASDNNETTPTSPSTRFQRTFIDPLWDYLDSLLFL
DKIRNFSLQLPAYGNLTPPEHRRAANLSTLNSLWWWSQ--
CMV UL128 FL:
1-
(SEQ ID NO: 24)
atgagccccaaggacctgacccccttcctgacaaccctgtggctgctcctgggccatagcag
agtgcctagagtgcgggccgaggaatgctgcgagttcatcaacgtgaaccacccccccgagc
ggtgctacgacttcaagatgtgcaaccggttcaccgtggccctgagatgccccgacggcgaa
gtgtgctacagccccgagaaaaccgccgagatccggggcatcgtgaccaccatgacccacag
cctgacccggcaggtggtgcacaacaagctgaccagctgcaactacaaccccctgtacctgg
aagccgacggccggatcagatgcggcaaagtgaacgacaaggcccagtacctgctgggagcc
gccggaagcgtgccctaccggtggatcaacctggaatacgacaagatcacccggatcgtggg
cctggaccagtacctggaaagcgtgaagaagcacaagcggctggacgtgtgcagagccaaga
tgggctacatgctgcagtgataa - 519
CMV UL128 FL;
(SEQ ID NO: 25)
MSPKDLTPFLTTLWLLLGHSRVPRVRAEECCEFINVNHPPERCYDFKMCNRFTVALRCPDGE
VCYSPEKTAEIRGIVTTMTHSLTRQVVHNKLTSCNYNPLYLEADGRIRCGKVNDKAQYLLGA
AGSVPYRWINLEYDKITRIVGLDQYLESVKKHKRLDVCRAKMGYMLQ--
CMV UL130 FL:
1-
(SEQ ID NO: 26)
atgctgcggctgctgctgagacaccacttccactgcctgctgctgtgtgccgtgtgggccac
cccttgtctggccagcccttggagcaccctgaccgccaaccagaaccctagccccccttggt
ccaagctgacctacagcaagccccacgacgccgccaccttctactgcccctttctgtacccc
agccctcccagaagccccctgcagttcagcggcttccagagagtgtccaccggccctgagtg
ccggaacgagacactgtacctgctgtacaaccgggagggccagacactggtggagcggagca
gcacctgggtgaaaaaagtgatctggtatctgagcggccggaaccagaccatcctgcagcgg
atgcccagaaccgccagcaagcccagcgacggcaacgtgcagatcagcgtggaggacgccaa
aatcttcggcgcccacatggtgcccaagcagaccaagctgctgagattcgtggtcaacgacg
gcaccagatatcagatgtgcgtgatgaagctggaaagctgggcccacgtgttccgggactac
tccgtgagcttccaggtccggctgaccttcaccgaggccaacaaccagacctacaccttctg
cacccaccccaacctgatcgtgtgataa - 648
CMV UL130 FL;
(SEQ ID NO: 27)
MLRLLLRHHFHCLLLCAVWATPCLASPWSTLTANQNPSPPWSKLTYSKPHDAATFYCPFLYP
SPPRSPLQFSGFQRVSTGPECRNETLYLLYNREGQTLVERSSTWVKKVIWYLSGRNQTILQR
MPRTASKPSDGNVQISVEDAKIFGAHMVPKQTKLLRFVVNDGTRYQMCVMKLESWAHVFRDY
SVSFQVRLTFTEANNQTYTFCTHPNLIV--
CMV UL131 FL:
1-
(SEQ ID NO: 28)
atgcggctgtgcagagtgtggctgtccgtgtgcctgtgtgccgtggtgctgggccagtgcca
gagagagacagccgagaagaacgactactaccgggtgccccactactgggatgcctgcagca
gagccctgcccgaccagacccggtacaaatacgtggagcagctcgtggacctgaccctgaac
taccactacgacgccagccacggcctggacaacttcgacgtgctgaagcggatcaacgtgac
cgaggtgtccctgctgatcagcgacttccggcggcagaacagaagaggcggcaccaacaagc
ggaccaccttcaacgccgctggctctctggcccctcacgccagatccctggaattcagcgtg
cggctgttcgccaactgataa - 393
CMV UL131 FL;
(SEQ ID NO: 29)
MRLCRVWLSVCLCAVVLGQCQRETAEKNDYYRVPHYWDACSRALPDQTRYKYVEQLVDLTLN
YHYDASHGLDNFDVLKRINVTEVSLLISDFRRQNRRGGTNKRTTFNAAGSLAPHARSLEFSV
RLFAN--
EMCV IRES nucleotide sequence;
(SEQ ID NO: 30)
aacgttactggccgaagccgcttggaataaggccggtgtgcgtttgtctatatgttattttc
caccatattgccgtcttttggcaatgtgagggcccggaaacctggccctgtcttcttgacga
gcattcctaggggtctttcccctctcgccaaaggaatgcaaggtctgttgaatgtcgtgaag
gaagcagttcctctggaagcttcttgaagacaaacaacgtctgtagcgaccctttgcaggca
gcggaaccccccacctggcgacaggtgcctctgcggccaaaagccacgtgtataagatacac
ctgcaaaggcggcacaaccccagtgccacgttgtgagttggatagttgtggaaagagtcaaa
tggctctcctcaagcgtattcaacaaggggctgaaggatgcccagaaggtaccccattgtat
gggatctgatctggggcctcggtgcacatgctttacatgtgtttagtcgaggttaaaaaaac
gtctaggccccccgaaccacggggacgtggttttcctttgaaaaacacgataat
EV71 IRES nucleotide sequence;
(SEQ ID NO: 31)
gtacctttgtacgcctgttttataccccctccctgatttgcaacttagaagcaacgc
aaaccagatcaatagtaggtgtgacataccagtcgcatcttgatcaagcacttctgtatccc
cggaccgagtatcaatagactgtgcacacggttgaaggagaaaacgtccgttacccggctaa
ctacttcgagaagcctagtaacgccattgaagttgcagagtgtttcgctcagcactcccccc
gtgtagatcaggtcgatgagtcaccgcattccccacgggcgaccgtggcggtggctgcgttg
gcggcctgcctatggggtaacccataggacgctctaatacggacatggcgtgaagagtctat
tgagctagttagtagtcctccggcccctgaatgcggctaatcctaactgcggagcacatacc
cttaatccaaagggcagtgtgtcgtaacgggcaactctgcagcggaaccgactactttgggt
gtccgtgtttctttttattcttgtattggctgcttatggtgacaattaaagaattgttacca
tatagctattggattggccatccagtgtcaaacagagctattgtatatctctttgttggatt
cacacctctcactcttgaaacgttacacaccctcaattacattatactgctgaacacgaagc
g
VEE Subgenomic Promoter
(SEQ ID NO: 1)
5′-CTCTCTACGGCTAACCTGAATGGA-3′
VZV gB
(SEQ ID NO: 32)
MFVTAVVSVSPSSFYESLQVEPTQSEDITRSAHLGDGDEIREAIHKSQDAETKPTFYVCPPP
TGSTIVRLEPPRTCPDYHLGKNFTEGIAVVYKENIAAYKFKATVYYKDVIVSTAWAGSSYTQ
ITNRYADRVPIPVSEITDTIDKFGKCSSKATYVRNNHKVEAFNEDKNPQDMPLIASKYNSVG
SKAWHTTNDTYMVAGTPGTYRTGTSVNCIIEEVEARSIFPYDSFGLSTGDITYMSPFFGLRD
GAYREHSNYAMDRFHQFEGYRQRDLDTRALLEPAARNFLVTPHLTVGWNWKPKRTEVCSLVK
WREVEDVVRDEYAHNFRFTMKTLSTTFISETNEFNLNQIHLSQCVKEEARAIINRIYTTRYN
SSHVRTGDIQTYLARGGFVVVFQPLLSNSLARLYLQELVRENTNHSPQKHPTRNTRSRRSVP
VELRANRTITTTSSVEFAMLQFTYDHIQEHVNEMLARISSSWCQLQNRERALWSGLFPINPS
ALASTILDQRVKARILGDVISVSNCPELGSDTRIILQNSMRVSGSTTRCYSRPLISIVSLNG
SGTVEGQLGTDNELIMSRDLLEPCVANHKRYFLFGHHYVYYEDYRYVREIAVHDVGMISTYV
DLNLTLLKDREFMPLQVYTRDELRDTGLLDYSEIQRRNQMHSLRFYDIDKVVQYDSGTAIMQ
GMAQFFQGLGTAGQAVGHVVLGATGALLSTVHGFTTFLSNPFGALAVGLLVLAGLVAAFFAY
RYVLKLKTSPMKALYPLTTKGLKQLPEGMDPFAEKPNATDTPIEEIGDSQNTEPSVNSGFDP
DKFREAQEMIKYMTLVSAAERQESKARKKNKTSALLTSRLTGLALRNRRGYSRVRTENVTGV
VZV gH
(SEQ ID NO: 33)
MFALVLAVVILPLWTTANKSYVTPTPATRSIGHMSALLREYSDRNMSLKLEAFYPTGFDEEL
IKSLHWGNDRKHVFLVIVKVNPTTHEGDVGLVIFPKYLLSPYHFKAEHRAPFPAGRFGFLSH
PVTPDVSFFDSSFAPYLTTQHLVAFTTFPPNPLVWHLERAETAATAERPFGVSLLPARPTVP
KNTILEHKAHFATWDALARHTFFSAEATITNSTLRIHVPLFGSVWPIRYWATGSVLLTSDSG
RVEVNIGVGFMSSLISLSSGLPIELIVVPHTVKLNAVTSDTTWFQLNPPGPDPGPSYRVYLL
GRGLDMNFSKHATVDICAYPEESLDYRYHLSMAHTEALRMTTKADQHDINEESYYHIAARIA
TSIFALSEMGRTTEYFLLDEIVDVQYQLKFLNYILMRIGAGAHPNTISGTSDLIFADPSQLH
DELSLLFGQVKPANVDYFISYDEARDQLKTAYALSRGQDHVNALSLARRVIMSTYKGLLVKQ
NLNATERQALFFASMILLNFREGLENSSRVLDGRTTLLLMTSMCTAAHATQAALNIQEGLAY
LNPSKHMFTIPNVYSPCMGSLRTDLTEEIHVMNLLSAIPTRPGLNEVLHTQLDESEIFDAAF
KTMMIFTTWTAKDLHILHTHVPEVFTCQDAAARNGEYVLILPAVQGHSYVITRNKPQRGLVY
SLADVDVYNPISVVYLSKDTCVSEHGVIETVALPHPDNLKECLYCGSVFLRYLTTGAIMDII
IIDSKDTERQLAAMGNSTIPPFNPDMHGDDSKAVLLFPNGTVVTLLGFERRQAIRMSGQYLG
ASLGGAFLAVVGFGIIGWMLCGNSRLREYNKIPLT
VZV gL
(SEQ ID NO: 34)
MASHKWLLQMIVFLKTITIAYCLHLQDDTPLFFGAKPLSDVSLIITEPCVSSVYEAWDYAAP
PVSNLSEALSGIVVKTKCPVPEVILWFKDKQMAYWINPYVTLKGLTQSVGEEHKSGDIRDAL
LDALSGVWVDSTPSSTNIPENGCVWGADRLFQRVCQ
VZV gI
(SEQ ID NO: 35)
MFLIQCLISAVIFYIQVTNALIFKGDHVSLQVNSSLTSILIPMQNDNYTEIKGQLVFIGEQL
PTGTNYSGTLELLYADTVAFCFRSVQVIRYDGCPRIRTSAFISCRYKHSWHYGNSTDRISTE
PDAGVMLKITKPGINDAGVYVLLVRLDHSRSTDGFILGVNVYTAGSHHNIHGVIYTSPSLQN
GYSTRALFQQARLCDLPATPKGSGTSLFQHMLDLRAGKSLEDNPWLHEDVVTTETKSVVKEG
IENHVYPTDMSTLPEKSLNDPPENLLIIIPIVASVMILTAMVIVIVISVKRRRIKKHPIYRP
NTKTRRGIQNATPESDVMLEAAIAQLATIREESPPHSVVNPFVK
VZV gE
(SEQ ID NO: 36)
MGTVNKPVVGVLMGFGIITGTLRITNPVRASVLRYDDFHIDEDKLDTNSVYEPYYHSDHAES
SWVNRGESSRKAYDHNSPYIWPRNDYDGFLENAHEHHGVYNQGRGIDSGERLMQPTQMSAQE
DLGDDTGIHVIPTLNGDDRHKIVNVDQRQYGDVFKGDLNPKPQGQRLIEVSVEENHPFTLRA
PIQRIYGVRYTETWSFLPSLTCTGDAAPAIQHICLKHTTCFQDVVVDVDCAENTKEDQLAEI
SYRFQGKKEADQPWIVVNTSTLFDELELDPPEIEPGVLKVLRTEKQYLGVYIWNMRGSDGTS
TYATFLVTWKGDEKTRNPTPAVTPQPRGAEFHMWNYHSHVFSVGDTFSLAMHLQYKIHEAPF
DLLLEWLYVPIDPTCQPMRLYSTCLYHPNAPQCLSHMNSGCTFTSPHLAQRVASTVYQNCEH
ADNYTAYCLGISHMEPSFGLILHDGGTTLKFVDTPESLSGLYVFVVYFNGHVEAVAYTVVST
VDHFVNAIEERGFPPTAGQPPATTKPKEITPVNPGTSPLLRYAAWTGGLAAVVLLCLVIFLI
CTAKRMRVKAYRVDKSPYNQSMYYAGLPVDDFEDSESTDTEEEFGNAIGGSHGGSSYTVYID
KTR
A526 Vector: SGP-gH-SGP-gL-SGP-UL128-2A-UL130-2Amod-UL131
(SEQ ID NO: 37)
ATAGGCGGCGCATGAGAGAAGCCCAGACCAATTACCTACCCAAAATGGAGAAAGTTCACGTTGACATCGAGGAAG
ACAGCCCATTCCTCAGAGCTTTGCAGCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAAACGGAGGTGGACCCATCCGACACGA
TCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGG
AATTGGACAAGAAAATGAAGGAGCTCGCCGCCGTCATGAGCGACCCTGACCTGGAAACTGAGACTATGTGCCTCC
ACGACGACGAGTCGTGTCGCTACGAAGGGCAAGTCGCTGTTTACCAGGATGTATACGCGGTTGACGGACCGACAA
GTCTCTATCACCAAGCCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAACGGCTCGTAACATAG
GCCTATGCAGCTCTGACGTTATGGAGCGGTCACGTAGAGGGATGTCCATTCTTAGAAAGAAGTATTTGAAACCAT
CCAACAATGTTCTATTCTCTGTTGGCTCGACCATCTACCACGAGAAGAGGGACTTACTGAGGAGCTGGCACCTGC
CGTCTGTATTTCACTTACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTACGATGCACCGCGAGG
GATTCTTGTGCTGCAAAGTGACAGACACATTGAACGGGGAGAGGGTCTCTTTTCCCGTGTGCACGTATGTGCCAG
CTACATTGTGTGACCAAATGACTGGCATACTGGCAACAGATGTCAGTGCGGACGACGCGCAAAAACTGCTGGTTG
GGCTCAACCAGCGTATAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAAGGCCACTAGGACTAC
GAGATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACATTGG
AGATCGGGCTGAGAACAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGTTGCGCGCAGCTCTAC
CACCTTTGGCAGCTGATGTTGAGGAGCCCACTCTGGAAGCCGATGTAGACTTGATGTTACAAGAGGCTGGGGCCG
GCTCAGTGGAGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGACATG
CAATACCCGTCCAGGACTTTCAAGCTCTGAGTGAAAGTGCCACCATTGTGTACAACGAACGTGAGTTCGTAAACA
GGTACCTGCACCATATTGCCACACATGGAGGAGCGCTGAACACTGATGAAGAATATTACAAAACTGTCAAGCCCA
GCGAGCACGACGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGACCAGCCGCTC
CTTACCAAGTACCAACCATAGGGGTGTATGGCGTGCCAGGATCAGGCAAGTCTGGCATCATTAAAAGCGCAGTCA
CCAAAAAAGATCTAGTGGTGAGCGCCAAGAAAGAAAACTGTGCAGAAATTATAAGGGACGTCAAGAAAATGAAAG
GGCTGGACGTCAATGCCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTTAACCACGAGATTTGCA
CACAAGTCTTCCACAAAAGCATCTCTCGCCGTTGCACTAAATCTGTGACTTCGGTCGTCTCAACCTTGTTTTACG
ACAAAAAAATGAGAACGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACAAAGGCAACGAAATAA
TGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAG
CCGGCGACCCATGGATAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCCAGAATAAGGCAAACG
TGTGTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTGGAC
TCGATCTGGACTCCGGTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACCCACAACTGCCTCGGG
CAGTTGCCACTGGAAGAGTCTATGACATGAACACTGGTACACTGCGCAATTATGATCCGCGCATAAACCTAGTAC
CTGTAAACAGAAGACTGCCTCATGCTTTAGTCCTCCACCATAATGAACACCCACAGAGTGACTTTTCTTCATTCG
TCAGCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATGACA
TAATATTTGTTAATGTGAGGACCCCATATAAATACCATCACTATCAGCAGTGTGAAGACCATGCCATTAAGCTTA
GCATGTTGACCAAGAAAGCTTGTCTGCATCTGAATCCCGGCGGAACCTGTGTCAGCATAGGTTATGGTTACGCTG
ACAGGGCCAGCGAAAGCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGCACAATCCTTACAAGC
TTTCATCAACCTTGACCAACATTTATACAGGTTCCAGACTCCACGAAGCCGGATGTGCACCCTCATATCATGTGG
TGCGAGGGGATATTGCCACGGCCACCGAAGGAGTGATTATAAATGCTGCTAACAGCAAAGGACAACCTGGCGGAG
GGGTGTGCGGAGCGCTGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTTCGGAGGTTGAAGGTG
ACAAACAGTTGGCAGAGGCTTATGAGTCCATCGCTAAGATTGTCAACGATAACAATTACAAGTCAGTAGCGATTC
CACTGTTGTCCACCGGCATCTTTTCCGGGAACAAAGATCGACTAACCCAATCATTGAACCATTTGCTGACAGCTT
TAGACACCACTGATGCAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTGATGCAGAGCTGGTGA
GGGTGCATCCGAAGAGTTCTTTGGCTGGAAGGAAGGGCTACAGCACAAGCGATGGCAAAACTTTCTCATATTTGG
AAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAAGAGTACAGCGCCTAA
AAGCCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCGTGG
AAACACCACCGGTAGACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGGAAGAAGAGGATAGCA
TAAGTTTGCTGTCAGATGGCCCGACCCACCAGGTGCTGCAAGTCGAGGCAGACATTCACGGGCCGCCCTCTGTAT
CTAGCTCATCCTGGTCCATTCCTCATGCATCCGACTTTGATGTGGACAGTTTATCCATACTTGACACCCTGGAGG
GAGCTAGCGTGACCAGCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAAGAACACCGTCACTTG
CACCCAGCAGGGCCTGCTCGAGAACCAGCCTAGTTTCCACCCCGCCAGGCGTGAATAGGGTGATCACTAGAGAGG
AGCTCGAGGCGCTTACCCCGTCACGCACTCCTAGCAGGTCGGTCTCGAGAACCAGCCTGGTCTCCAACCCGCCAG
GCGTAAATAGGGTGATTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAACGGTGCTATCCGAAG
TGGTGTTGGAGAGGACCGAATTGGAGATTTCGTATGCCCCGCGCCTCGACCAAGAAAAAGAAGAATTACTACGCA
AGAAATTACAGTTAAATCCCACACCTGCTAACAGAAGCAGATACCAGTCCAGGAAGGTGGAGAACATGAAAGCCA
TAACAGCTAGACGTATTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCGCAGTGGAAGCCTGTA
ACGCCATGTTGAAAGAGAACTTTCCGACTGTGGCTTCTTACTGTATTATTCCAGAGTACGATGCCTATTTGGACA
TGGTTGACGGAGCTTCATGCTGCTTAGACACTGCCAGTTTTTGCCCTGCAAAGCTGCGCAGCTTTCCAAAGAAAC
ACTCCTATTTGGAACCCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGGCGGCCTTTAATGTGG
AATGCTTCAAGAAATATGCGTGTAATAATGAATATTGGGAAACGTTTAAAGAAAACCCCATCAGGCTTACTGAAG
AAAACGTGGTAAATTACATTACCAAATTAAAAGGACCAAAAGCTGCTGCTCTTTTTGCGAAGACACATAATTTGA
ATATGTTGCAGGACATACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAGCGTATCTGTGCGGAA
TCCACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGTTTG
ATAAAAGTGAGGACGACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTAAATTTAAATTCGGAG
CCATGATGAAATCTGGAATGTTCCTCACACTGTTTGTGAACACAGTCATTAACATTGTAATCGCAAGCAGAGTGT
TGAGAGAACGGCTAACCGGATCACCATGTGCAGCATTCATTGGAGATGACAATATCGTGAAAGGAGTCAAATCGG
ACAAATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACCCCC
TAAAAAGGCTGTTTAAGCTTGGCAAACCTCTGGCAGCAGACGATGAACATGATGATGACAGGAGAAGGGCATTGC
ATGAAGAGTCAACACGCTGGAACCGAGTGGGTATTCTTTCAGAGCTGTGCAAGGCAGTAGAATCAAGGTATGAAA
CCGTAGGAACTTCCATCATAGTTATGGCCATGACTACTCTAGCTAGCAGTGTTAAATCATTCAGCTACCTGAGAG

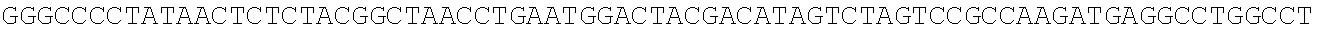

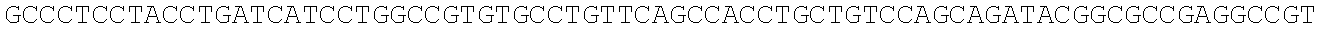

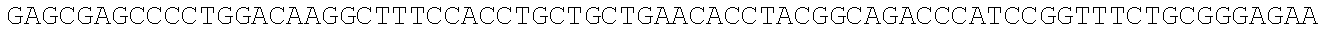

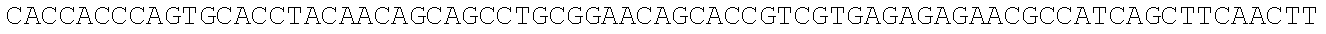

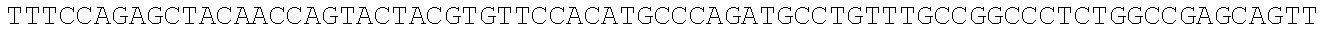

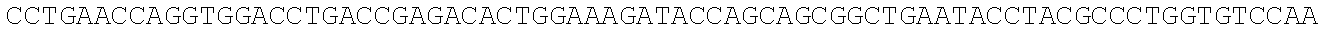

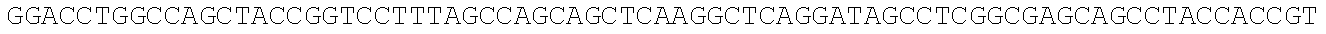

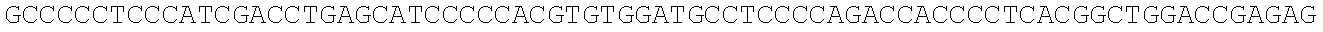

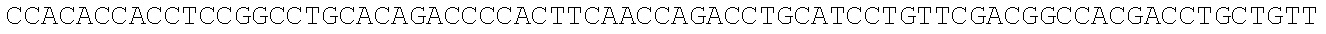

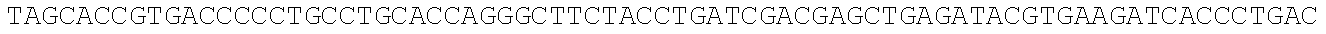

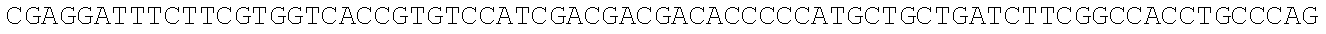

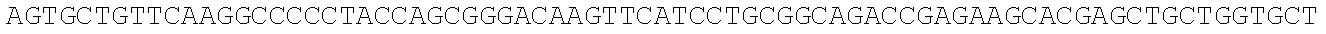

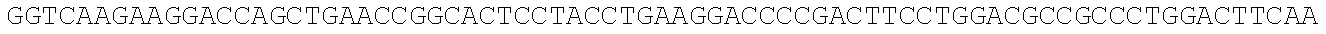

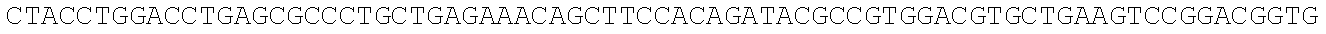

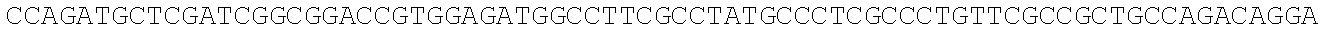

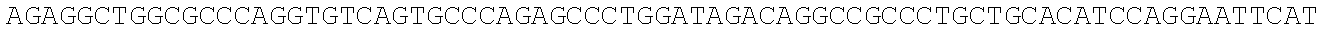

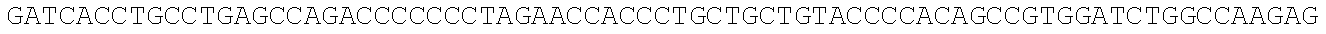

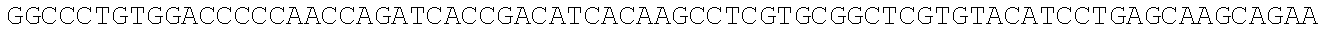

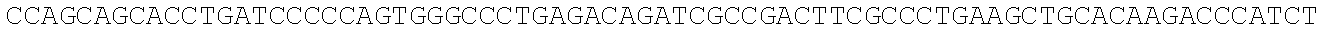

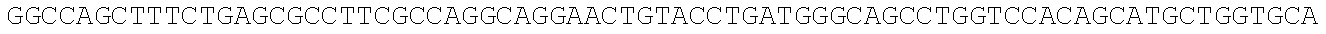

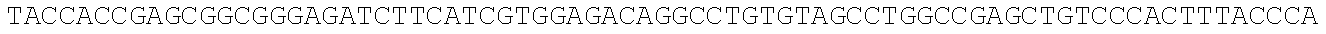

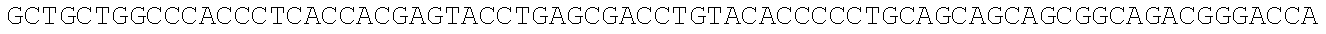

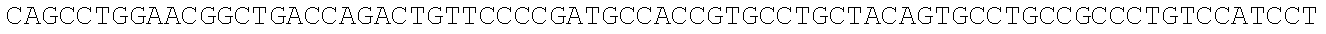

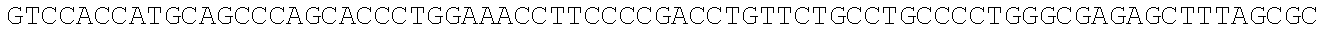

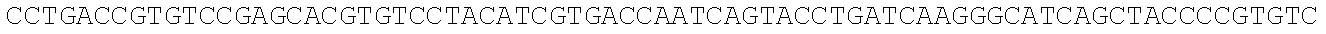

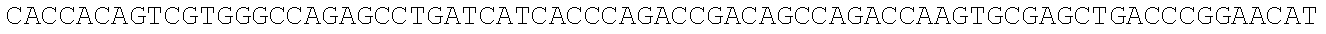

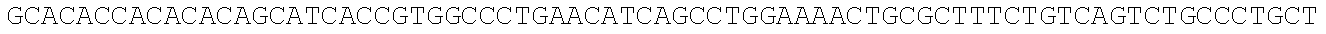

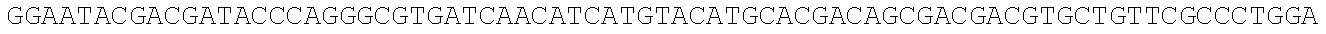

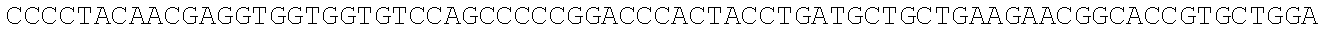

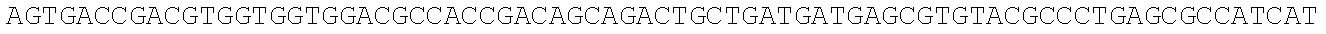

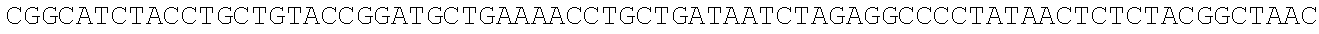

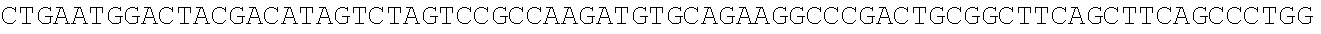

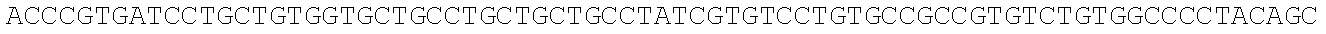

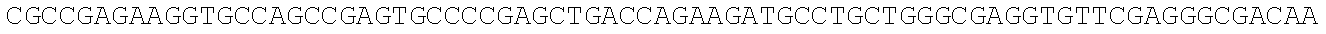

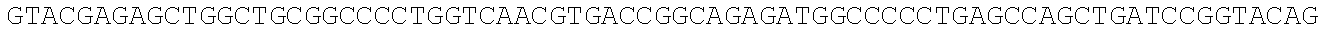

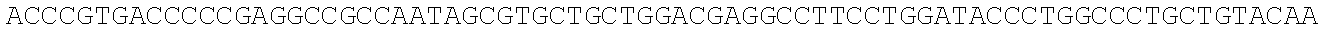

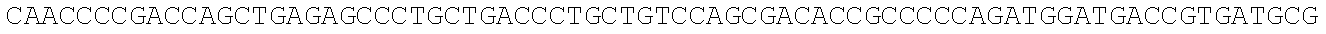

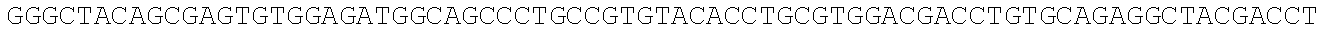

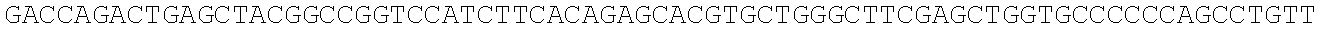

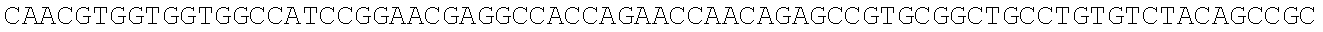

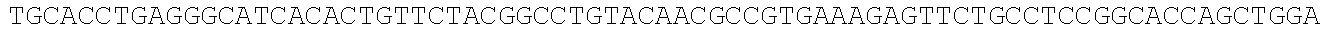

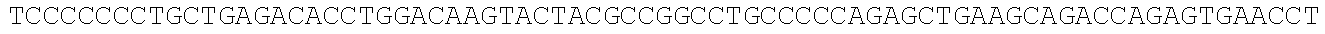

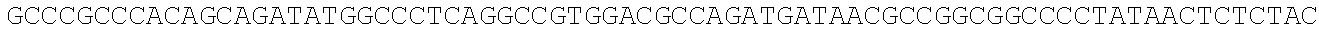

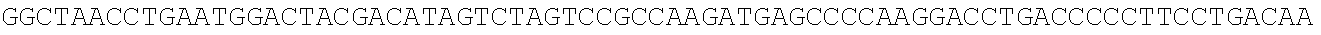

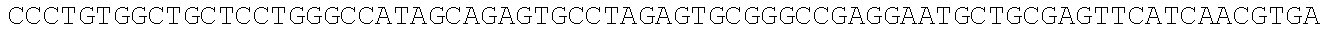

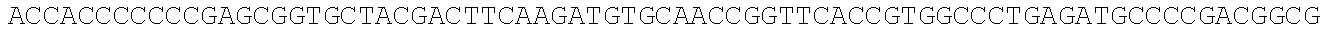

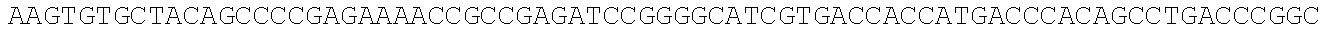

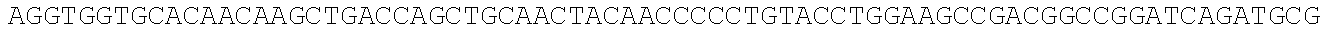

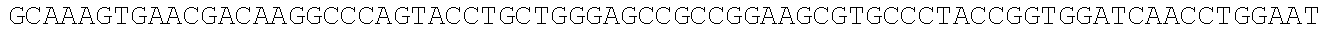

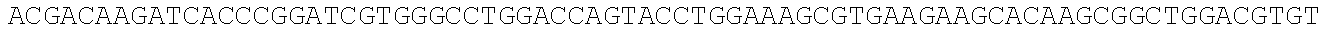

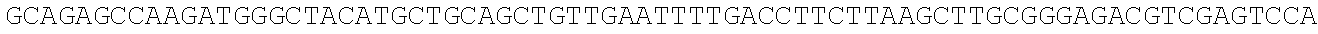

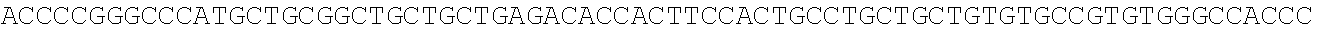

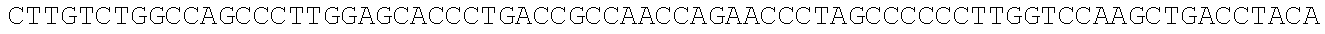

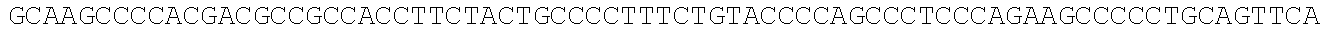

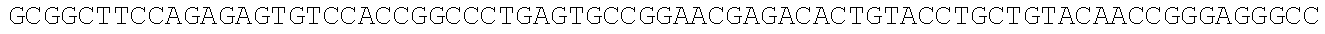

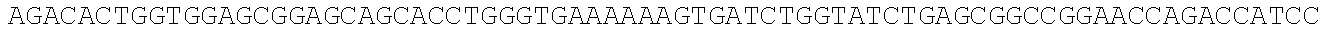

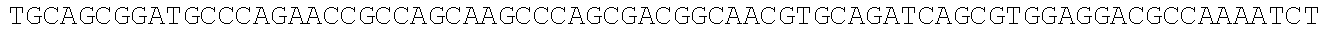

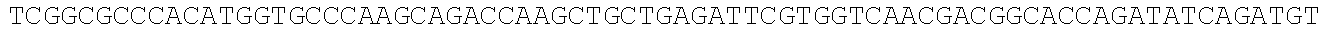

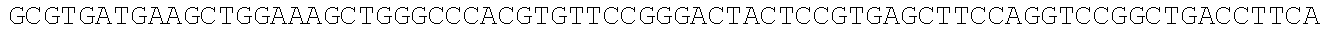

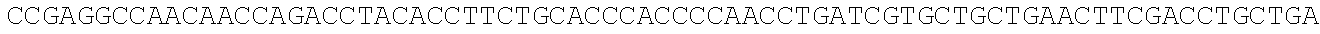

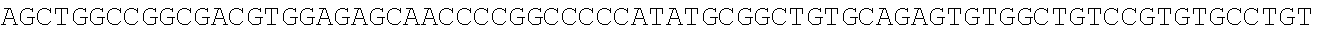

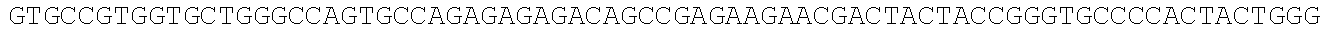

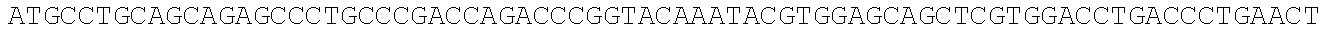

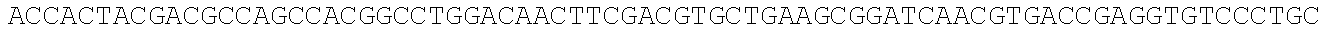

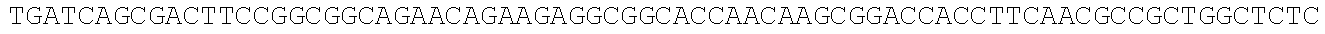

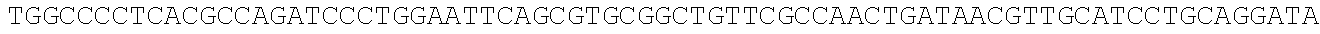

CAGCAGCAATTGGCAAGCTGCTTACATAGAACTCGCGGCGATTGGCATGCCGCCTTAAAATTTTTATTTTATTTT
TCTTTTCTTTTCCGAATCGGATTTTGTTTTTAATATTTCAAAAAAAAAAAAAAAAAAAAAAAAAAAAAAAAAAAG
GGTCGGCATGGCATCTCCACCTCCTCGCGGTCCGACCTGGGCATCCGAAGGAGGACGCACGTCCACTCGGATGGC
TAAGGGAGAGCCACGTTTAAACGCTAGAGCAAGACGTTTCCCGTTGAATATGGCTCATAACACCCCTTGTATTAC
TGTTTATGTAAGCAGACAGTTTTATTGTTCATGATGATATATTTTTATCTTGTGCAATGTAACATCAGAGATTTT
GAGACACAACGTGGCTTTGTTGAATAAATCGAACTTTTGCTGAGTTGAAGGATCAGATCACGCATCTTCCCGACA
ACGCAGACCGTTCCGTGGCAAAGCAAAAGTTCAAAATCACCAACTGGTCCACCTACAACAAAGCTCTCATCAACC
GTGGCTCCCTCACTTTCTGGCTGGATGATGGGGCGATTCAGGCCTGGTATGAGTCAGCAACACCTTCTTCACGAG
GCAGACCTCAGCGCTAGCGGAGTGTATACTGGCTTACTATGTTGGCACTGATGAGGGTGTCAGTGAAGTGCTTCA
TGTGGCAGGAGAAAAAAGGCTGCACCGGTGCGTCAGCAGAATATGTGATACAGGATATATTCCGCTTCCTCGCTC
ACTGACTCGCTACGCTCGGTCGTTCGACTGCGGCGAGCGGAAATGGCTTACGAACGGGGCGGAGATTTCCTGGAA
GATGCCAGGAAGATACTTAACAGGGAAGTGAGAGGGCCGCGGCAAAGCCGTTTTTCCATAGGCTCCGCCCCCCTG
ACAAGCATCACGAAATCTGACGCTCAAATCAGTGGTGGCGAAACCCGACAGGACTATAAAGATACCAGGCGTTTC
CCCTGGCGGCTCCCTCGTGCGCTCTCCTGTTCCTGCCTTTCGGTTTACCGGTGTCATTCCGCTGTTATGGCCGCG
TTTGTCTCATTCCACGCCTGACACTCAGTTCCGGGTAGGCAGTTCGCTCCAAGCTGGACTGTATGCACGAACCCC
CCGTTCAGTCCGACCGCTGCGCCTTATCCGGTAACTATCGTCTTGAGTCCAACCCGGAAAGACATGCAAAAGCAC
CACTGGCAGCAGCCACTGGTAATTGATTTAGAGGAGTTAGTCTTGAAGTCATGCGCCGGTTAAGGCTAAACTGAA
AGGACAAGTTTTGGTGACTGCGCTCCTCCAAGCCAGTTACCTCGGTTCAAAGAGTTGGTAGCTCAGAGAACCTTC
GAAAAACCGCCCTGCAAGGCGGTTTTTTCGTTTTCAGAGCAAGAGATTACGCGCAGACCAAAACGATCTCAAGAA
GATCATCTTATTAAGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGAGTAAACT
TGGTCTGACAGTTATTAGAAAAATTCATCCAGCAGACGATAAAACGCAATACGCTGGCTATCCGGTGCCGCAATG
CCATACAGCACCAGAAAACGATCCGCCCATTCGCCGCCCAGTTCTTCCGCAATATCACGGGTGGCCAGCGCAATA
TCCTGATAACGATCCGCCACGCCCAGACGGCCGCAATCAATAAAGCCGCTAAAACGGCCATTTTCCACCATAATG
TTCGGCAGGCACGCATCACCATGGGTCACCACCAGATCTTCGCCATCCGGCATGCTCGCTTTCAGACGCGCAAAC
AGCTCTGCCGGTGCCAGGCCCTGATGTTCTTCATCCAGATCATCCTGATCCACCAGGCCCGCTTCCATACGGGTA
CGCGCACGTTCAATACGATGTTTCGCCTGATGATCAAACGGACAGGTCGCCGGGTCCAGGGTATGCAGACGACGC
ATGGCATCCGCCATAATGCTCACTTTTTCTGCCGGCGCCAGATGGCTAGACAGCAGATCCTGACCCGGCACTTCG
CCCAGCAGCAGCCAATCACGGCCCGCTTCGGTCACCACATCCAGCACCGCCGCACACGGAACACCGGTGGTGGCC
AGCCAGCTCAGACGCGCCGCTTCATCCTGCAGCTCGTTCAGCGCACCGCTCAGATCGGTTTTCACAAACAGCACC
GGACGACCCTGCGCGCTCAGACGAAACACCGCCGCATCAGAGCAGCCAATGGTCTGCTGCGCCCAATCATAGCCA
AACAGACGTTCCACCCACGCTGCCGGGCTACCCGCATGCAGGCCATCCTGTTCAATCATACTCTTCCTTTTTCAA
TATTATTGAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCCTTATAAATCAAAAGAAT
AGACCGAGATAGGGTTGAGTGGCCGCTACAGGGCGCTCCCATTCGCCATTCAGGCTGCGCAACTGTTGGGAAGGG
CGTTTCGGTGCGGGCCTCTTCGCTATTACGCCAGCTGGCGAAAGGGGGATGTGCTGCAAGGCGATTAAGTTGGGT
AACGCCAGGGTTTTCCCAGTCACACGCGTAATACGACTCACTATAG
A527 Vector: SGP-gH-SGP-gL-SGP-UL128-EMCV-UL130-EV71-UL131
(SEQ ID NO: 38)
ATAGGCGGCGCATGAGAGAAGCCCAGACCAATTACCTACCCAAAATGGAGAAAGTTCACGTTGACATCGAGGAAG
ACAGCCCATTCCTCAGAGCTTTGCAGCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAAACGGAGGTGGACCCATCCGACACGA
TCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGG
AATTGGACAAGAAAATGAAGGAGCTCGCCGCCGTCATGAGCGACCCTGACCTGGAAACTGAGACTATGTGCCTCC
ACGACGACGAGTCGTGTCGCTACGAAGGGCAAGTCGCTGTTTACCAGGATGTATACGCGGTTGACGGACCGACAA
GTCTCTATCACCAAGCCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAACGGCTCGTAACATAG
GCCTATGCAGCTCTGACGTTATGGAGCGGTCACGTAGAGGGATGTCCATTCTTAGAAAGAAGTATTTGAAACCAT
CCAACAATGTTCTATTCTCTGTTGGCTCGACCATCTACCACGAGAAGAGGGACTTACTGAGGAGCTGGCACCTGC
CGTCTGTATTTCACTTACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTACGATGCACCGCGAGG
GATTCTTGTGCTGCAAAGTGACAGACACATTGAACGGGGAGAGGGTCTCTTTTCCCGTGTGCACGTATGTGCCAG
CTACATTGTGTGACCAAATGACTGGCATACTGGCAACAGATGTCAGTGCGGACGACGCGCAAAAACTGCTGGTTG
GGCTCAACCAGCGTATAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAAGGCCACTAGGACTAC
GAGATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACATTGG
AGATCGGGCTGAGAACAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGTTGCGCGCAGCTCTAC
CACCTTTGGCAGCTGATGTTGAGGAGCCCACTCTGGAAGCCGATGTAGACTTGATGTTACAAGAGGCTGGGGCCG
GCTCAGTGGAGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGACATG
CAATACCCGTCCAGGACTTTCAAGCTCTGAGTGAAAGTGCCACCATTGTGTACAACGAACGTGAGTTCGTAAACA
GGTACCTGCACCATATTGCCACACATGGAGGAGCGCTGAACACTGATGAAGAATATTACAAAACTGTCAAGCCCA
GCGAGCACGACGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGACCAGCCGCTC
CTTACCAAGTACCAACCATAGGGGTGTATGGCGTGCCAGGATCAGGCAAGTCTGGCATCATTAAAAGCGCAGTCA
CCAAAAAAGATCTAGTGGTGAGCGCCAAGAAAGAAAACTGTGCAGAAATTATAAGGGACGTCAAGAAAATGAAAG
GGCTGGACGTCAATGCCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTTAACCACGAGATTTGCA
CACAAGTCTTCCACAAAAGCATCTCTCGCCGTTGCACTAAATCTGTGACTTCGGTCGTCTCAACCTTGTTTTACG
ACAAAAAAATGAGAACGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACAAAGGCAACGAAATAA
TGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAG
CCGGCGACCCATGGATAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCCAGAATAAGGCAAACG
TGTGTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTGGAC
TCGATCTGGACTCCGGTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACCCACAACTGCCTCGGG
CAGTTGCCACTGGAAGAGTCTATGACATGAACACTGGTACACTGCGCAATTATGATCCGCGCATAAACCTAGTAC
CTGTAAACAGAAGACTGCCTCATGCTTTAGTCCTCCACCATAATGAACACCCACAGAGTGACTTTTCTTCATTCG
TCAGCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATGACA
TAATATTTGTTAATGTGAGGACCCCATATAAATACCATCACTATCAGCAGTGTGAAGACCATGCCATTAAGCTTA
GCATGTTGACCAAGAAAGCTTGTCTGCATCTGAATCCCGGCGGAACCTGTGTCAGCATAGGTTATGGTTACGCTG
ACAGGGCCAGCGAAAGCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGCACAATCCTTACAAGC
TTTCATCAACCTTGACCAACATTTATACAGGTTCCAGACTCCACGAAGCCGGATGTGCACCCTCATATCATGTGG
TGCGAGGGGATATTGCCACGGCCACCGAAGGAGTGATTATAAATGCTGCTAACAGCAAAGGACAACCTGGCGGAG
GGGTGTGCGGAGCGCTGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTTCGGAGGTTGAAGGTG
ACAAACAGTTGGCAGAGGCTTATGAGTCCATCGCTAAGATTGTCAACGATAACAATTACAAGTCAGTAGCGATTC
CACTGTTGTCCACCGGCATCTTTTCCGGGAACAAAGATCGACTAACCCAATCATTGAACCATTTGCTGACAGCTT
TAGACACCACTGATGCAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTGATGCAGAGCTGGTGA
GGGTGCATCCGAAGAGTTCTTTGGCTGGAAGGAAGGGCTACAGCACAAGCGATGGCAAAACTTTCTCATATTTGG
AAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAAGAGTACAGCGCCTAA
AAGCCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCGTGG
AAACACCACCGGTAGACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGGAAGAAGAGGATAGCA
TAAGTTTGCTGTCAGATGGCCCGACCCACCAGGTGCTGCAAGTCGAGGCAGACATTCACGGGCCGCCCTCTGTAT
CTAGCTCATCCTGGTCCATTCCTCATGCATCCGACTTTGATGTGGACAGTTTATCCATACTTGACACCCTGGAGG
GAGCTAGCGTGACCAGCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAAGAACACCGTCACTTG
CACCCAGCAGGGCCTGCTCGAGAACCAGCCTAGTTTCCACCCCGCCAGGCGTGAATAGGGTGATCACTAGAGAGG
AGCTCGAGGCGCTTACCCCGTCACGCACTCCTAGCAGGTCGGTCTCGAGAACCAGCCTGGTCTCCAACCCGCCAG
GCGTAAATAGGGTGATTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAACGGTGCTATCCGAAG
TGGTGTTGGAGAGGACCGAATTGGAGATTTCGTATGCCCCGCGCCTCGACCAAGAAAAAGAAGAATTACTACGCA
AGAAATTACAGTTAAATCCCACACCTGCTAACAGAAGCAGATACCAGTCCAGGAAGGTGGAGAACATGAAAGCCA
TAACAGCTAGACGTATTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCGCAGTGGAAGCCTGTA
ACGCCATGTTGAAAGAGAACTTTCCGACTGTGGCTTCTTACTGTATTATTCCAGAGTACGATGCCTATTTGGACA
TGGTTGACGGAGCTTCATGCTGCTTAGACACTGCCAGTTTTTGCCCTGCAAAGCTGCGCAGCTTTCCAAAGAAAC
ACTCCTATTTGGAACCCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGGCGGCCTTTAATGTGG
AATGCTTCAAGAAATATGCGTGTAATAATGAATATTGGGAAACGTTTAAAGAAAACCCCATCAGGCTTACTGAAG
AAAACGTGGTAAATTACATTACCAAATTAAAAGGACCAAAAGCTGCTGCTCTTTTTGCGAAGACACATAATTTGA
ATATGTTGCAGGACATACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAGCGTATCTGTGCGGAA
TCCACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGTTTG
ATAAAAGTGAGGACGACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTAAATTTAAATTCGGAG
CCATGATGAAATCTGGAATGTTCCTCACACTGTTTGTGAACACAGTCATTAACATTGTAATCGCAAGCAGAGTGT
TGAGAGAACGGCTAACCGGATCACCATGTGCAGCATTCATTGGAGATGACAATATCGTGAAAGGAGTCAAATCGG
ACAAATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACCCCC
TAAAAAGGCTGTTTAAGCTTGGCAAACCTCTGGCAGCAGACGATGAACATGATGATGACAGGAGAAGGGCATTGC
ATGAAGAGTCAACACGCTGGAACCGAGTGGGTATTCTTTCAGAGCTGTGCAAGGCAGTAGAATCAAGGTATGAAA
CCGTAGGAACTTCCATCATAGTTATGGCCATGACTACTCTAGCTAGCAGTGTTAAATCATTCAGCTACCTGAGAG

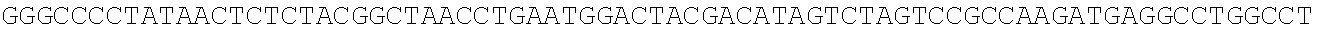

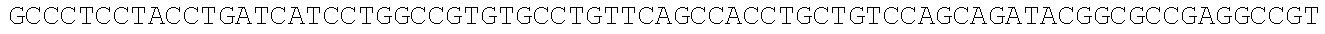

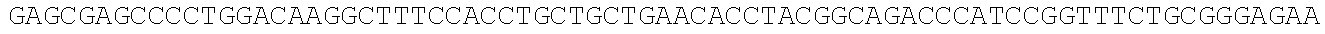

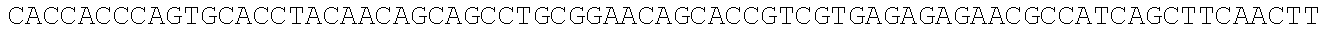

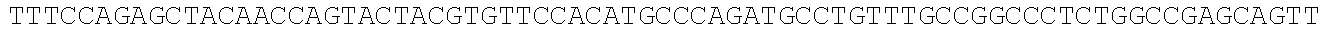

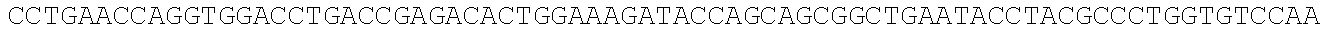

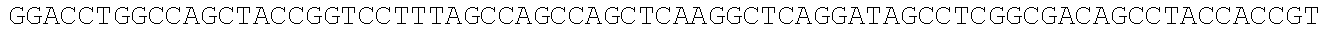

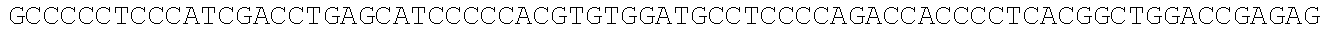

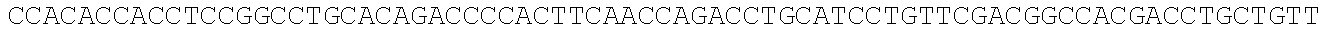

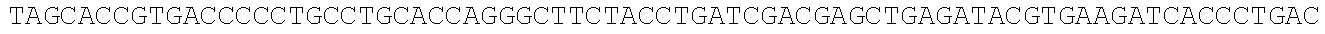

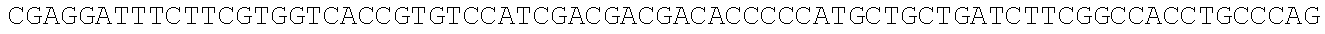

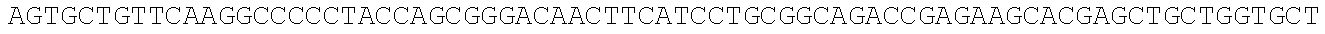

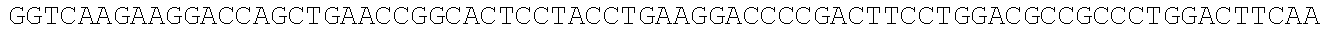

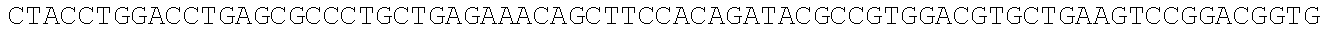

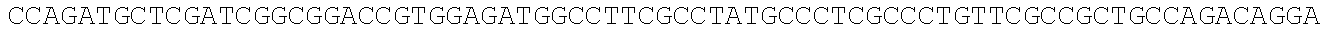

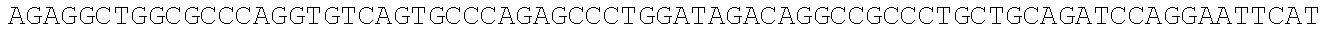

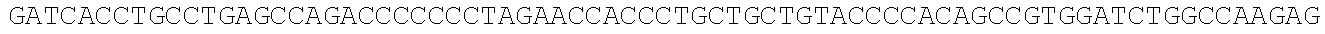

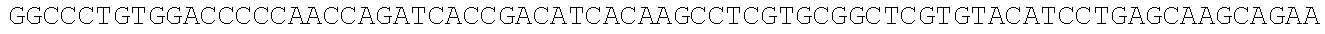

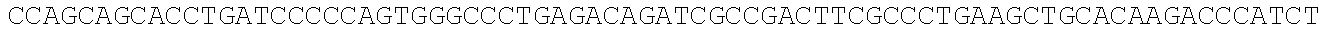

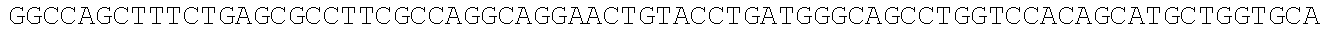

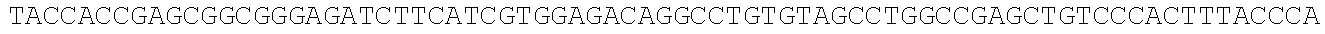

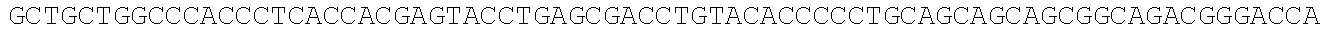

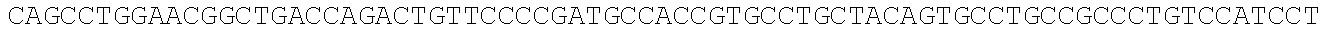

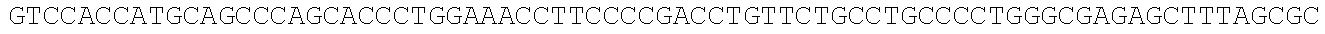

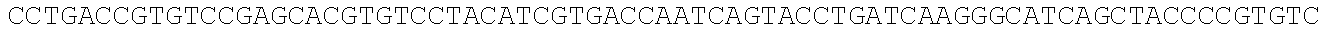

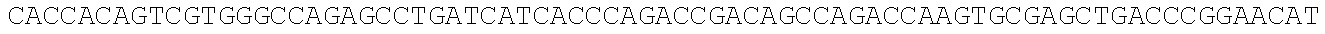

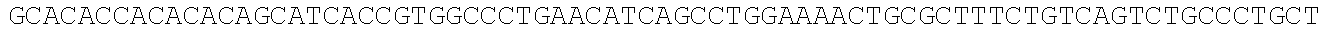

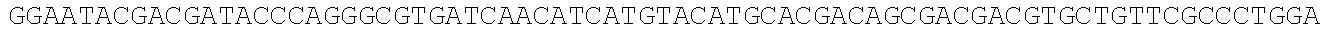

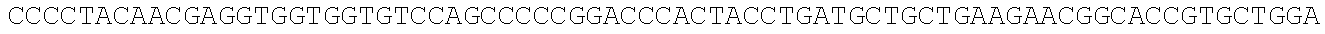

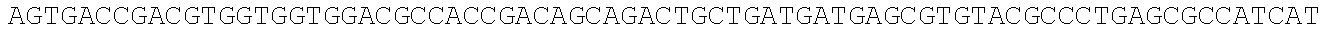

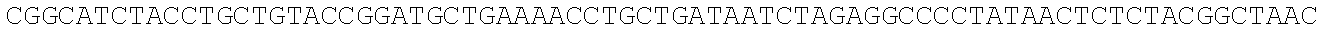

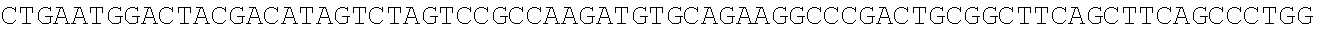

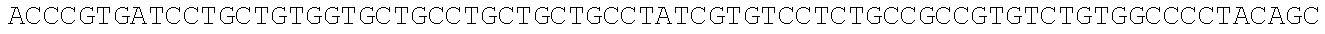

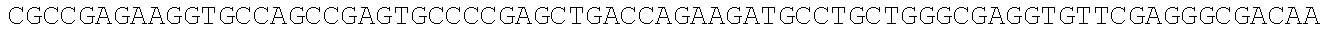

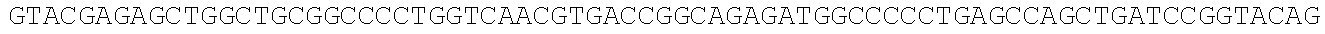

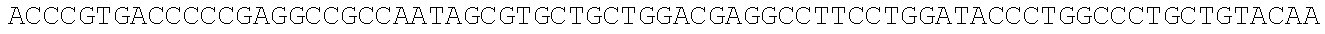

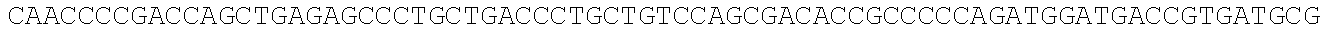

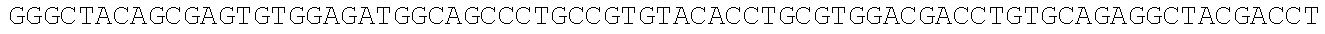

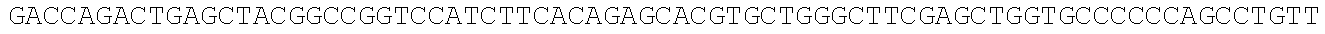

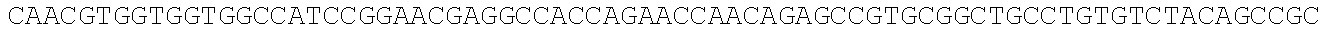

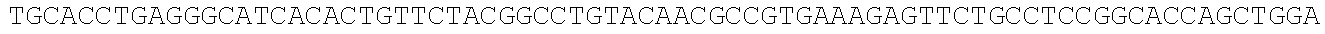

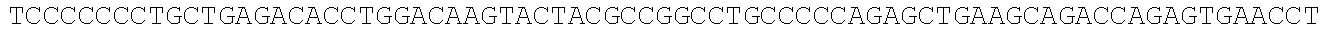

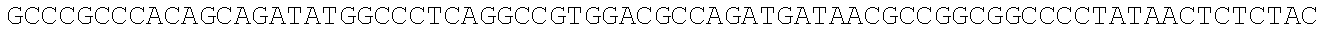

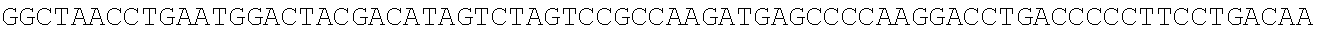

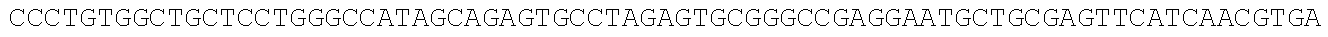

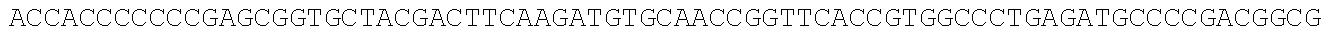

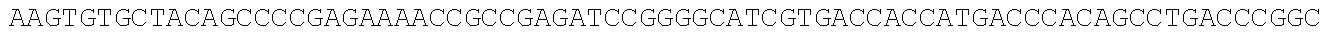

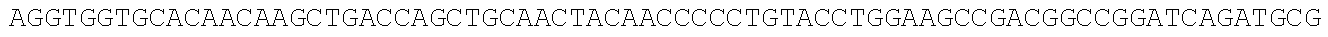

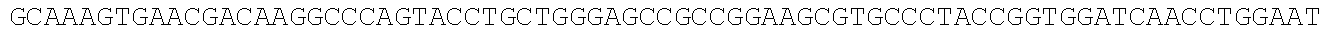

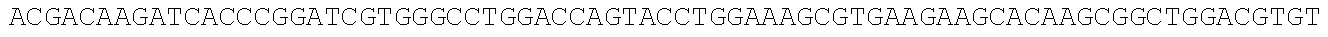

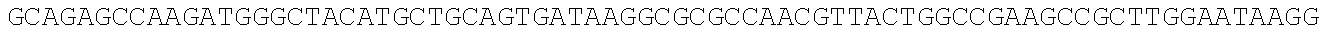

CCGGTGTGCGTTTGTCTATATGTTATTTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAAGGAATGCAAGGTCTGTTGAATGTCGT
GAAGGAAGCAGTTCCTCTGGAAGCTTCTTGAAGACAAACAACGTCTGTAGCGACCCTTTGCAGGCAGCGGAACCC
CCCACCTGGCGACAGGTGCCTCTGCGGCCAAAAGCCACGTGTATAAGATACACCTGCAAAGGCGGCACAACCCCA
GTGCCACGTTGTGAGTTGGATAGTTGTGGAAAGAGTCAAATGGCTCTCCTCAAGCGTATTCAACAAGGGGCTGAA
GGATGCCCAGAAGGTACCCCATTGTATGGGATCTGATCTGGGGCCTCGGTGCACATGCTTTACATGTGTTTAGTC

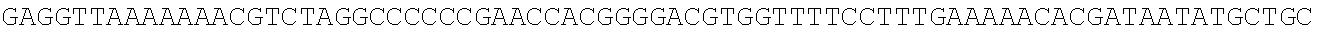

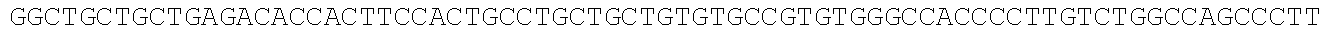

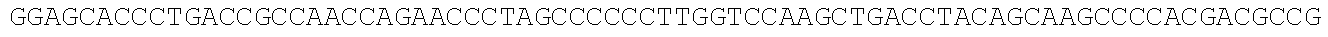

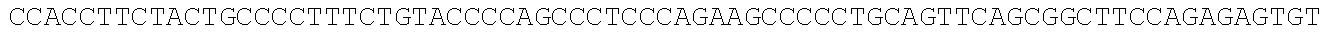

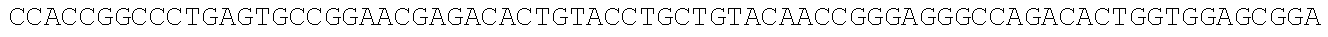

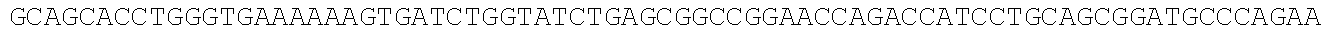

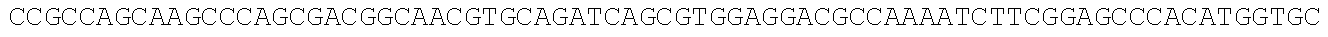

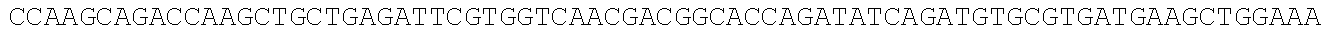

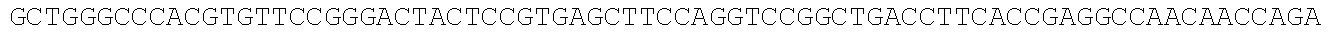

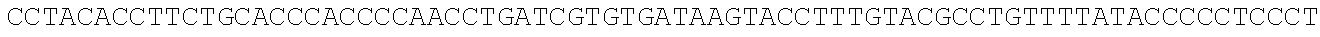

GATTTGCAACTTAGAAGCAACGCAAACCAGATCAATAGTAGGTGTGACATACCAGTCGCATCTTGATCAAGCACT
TCTGTATCCCCGGACCGAGTATCAATAGACTGTGCACACGGTTGAAGGAGAAAACGTCCGTTACCCGGCTAACTA
CTTCGAGAAGCCTAGTAACGCCATTGAAGTTGCAGAGTGTTTCGCTCAGCACTCCCCCCGTGTAGATCAGGTCGA
TGAGTCACCGCATTCCCCACGGGCGACCGTGGCGGTGGCTGCGTTGGCGGCCTGCCTATGGGGTAACCCATAGGA
CGCTCTAATACGGACATGGCGTGAAGAGTCTATTGAGCTAGTTAGTAGTCCTCCGGCCCCTGAATGCGGCTAATC
CTAACTGCGGAGCACATACCCTTAATCCAAAGGGCAGTGTGTCGTAACGGGCAACTCTGCAGCGGAACCGACTAC
TTTGGGTGTCCGTGTTTCTTTTTATTCTTGTATTGGCTGCTTATGGTGACAATTAAAGAATTGTTACCATATAGC
TATTGGATTGGCCATCCAGTGTCAAACAGAGCTATTGTATATCTCTTTGTTGGATTCACACCTCTCACTCTTGAA

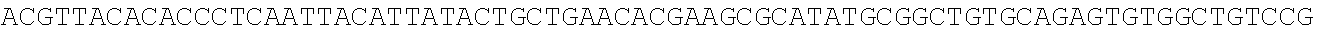

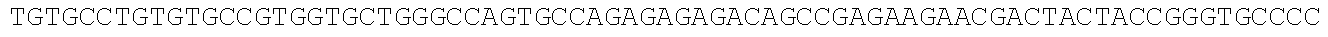

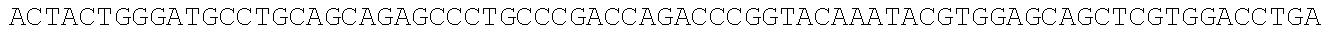

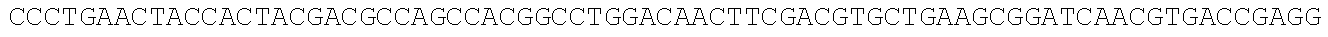

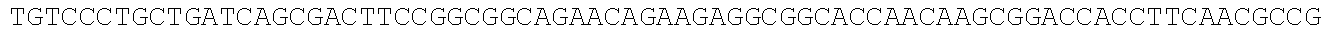

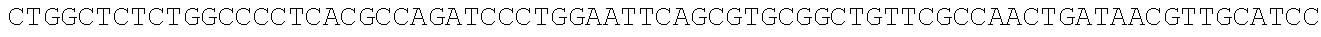

TGCAGGATACAGCAGCAATTGGCAAGCTGCTTACATAGAACTCGCGGCGATTGGCATGCCGCCTTAAAATTTTTA
TTTTATTTTTCTTTTCTTTTCCGAATCGGATTTTGTTTTTAATATTTCAAAAAAAAAAAAAAAAAAAAAAAAAAA
AAAAAAAAGGGTCGGCATGGCATCTCCACCTCCTCGCGGTCCGACCTGGGCATCCGAAGGAGGACGCACGTCCAC
TCGGATGGCTAAGGGAGAGCCACGTTTAAACGCTAGAGCAAGACGTTTCCCGTTGAATATGGCTCATAACACCCC
TTGTATTACTGTTTATGTAAGCAGACAGTTTTATTGTTCATGATGATATATTTTTATCTTGTGCAATGTAACATC
AGAGATTTTGAGACACAACGTGGCTTTGTTGAATAAATCGAACTTTTGCTGAGTTGAAGGATCAGATCACGCATC
TTCCCGACAACGCAGACCGTTCCGTGGCAAAGCAAAAGTTCAAAATCACCAACTGGTCCACCTACAACAAAGCTC
TCATCAACCGTGGCTCCCTCACTTTCTGGCTGGATGATGGGGCGATTCAGGCCTGGTATGAGTCAGCAACACCTT
CTTCACGAGGCAGACCTCAGCGCTAGCGGAGTGTATACTGGCTTACTATGTTGGCACTGATGAGGGTGTCAGTGA
AGTGCTTCATGTGGCAGGAGAAAAAAGGCTGCACCGGTGCGTCAGCAGAATATGTGATACAGGATATATTCCGCT
TCCTCGCTCACTGACTCGCTACGCTCGGTCGTTCGACTGCGGCGAGCGGAAATGGCTTACGAACGGGGCGGAGAT
TTCCTGGAAGATGCCAGGAAGATACTTAACAGGGAAGTGAGAGGGCCGCGGCAAAGCCGTTTTTCCATAGGCTCC
GCCCCCCTGACAAGCATCACGAAATCTGACGCTCAAATCAGTGGTGGCGAAACCCGACAGGACTATAAAGATACC
AGGCGTTTCCCCTGGCGGCTCCCTCGTGCGCTCTCCTGTTCCTGCCTTTCGGTTTACCGGTGTCATTCCGCTGTT
ATGGCCGCGTTTGTCTCATTCCACGCCTGACACTCAGTTCCGGGTAGGCAGTTCGCTCCAAGCTGGACTGTATGC
ACGAACCCCCCGTTCAGTCCGACCGCTGCGCCTTATCCGGTAACTATCGTCTTGAGTCCAACCCGGAAAGACATG
CAAAAGCACCACTGGCAGCAGCCACTGGTAATTGATTTAGAGGAGTTAGTCTTGAAGTCATGCGCCGGTTAAGGC
TAAACTGAAAGGACAAGTTTTGGTGACTGCGCTCCTCCAAGCCAGTTACCTCGGTTCAAAGAGTTGGTAGCTCAG
AGAACCTTCGAAAAACCGCCCTGCAAGGCGGTTTTTTCGTTTTCAGAGCAAGAGATTACGCGCAGACCAAAACGA
TCTCAAGAAGATCATCTTATTAAGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGTATATAT
GAGTAAACTTGGTCTGACAGTTATTAGAAAAATTCATCCAGCAGACGATAAAACGCAATACGCTGGCTATCCGGT
GCCGCAATGCCATACAGCACCAGAAAACGATCCGCCCATTCGCCGCCCAGTTCTTCCGCAATATCACGGGTGGCC
AGCGCAATATCCTGATAACGATCCGCCACGCCCAGACGGCCGCAATCAATAAAGCCGCTAAAACGGCCATTTTCC
ACCATAATGTTCGGCAGGCACGCATCACCATGGGTCACCACCAGATCTTCGCCATCCGGCATGCTCGCTTTCAGA
CGCGCAAACAGCTCTGCCGGTGCCAGGCCCTGATGTTCTTCATCCAGATCATCCTGATCCACCAGGCCCGCTTCC
ATACGGGTACGCGCACGTTCAATACGATGTTTCGCCTGATGATCAAACGGACAGGTCGCCGGGTCCAGGGTATGC
AGACGACGCATGGCATCCGCCATAATGCTCACTTTTTCTGCCGGCGCCAGATGGCTAGACAGCAGATCCTGACCC
GGCACTTCGCCCAGCAGCAGCCAATCACGGCCCGCTTCGGTCACCACATCCAGCACCGCCGCACACGGAACACCG
GTGGTGGCCAGCCAGCTCAGACGCGCCGCTTCATCCTGCAGCTCGTTCAGCGCACCGCTCAGATCGGTTTTCACA
AACAGCACCGGACGACCCTGCGCGCTCAGACGAAACACCGCCGCATCAGAGCAGCCAATGGTCTGCTGCGCCCAA
TCATAGCCAAACAGACGTTCCACCCACGCTGCCGGGCTACCCGCATGCAGGCCATCCTGTTCAATCATACTCTTC
CTTTTTCAATATTATTGAAGCATTTATCAGGGTTATTGTCTCATGAGCGGATACATATTTGAATGTATTTAGAAA
AATAAACAAATAGGGGTTCCGCGCACATTTCCCCGAAAAGTGCCACCTAAATTGTAAGCGTTAATATTTTGTTAA
AATTCGCGTTAAATTTTTGTTAAATCAGCTCATTTTTTAACCAATAGGCCGAAATCGGCAAAATCCCTTATAAAT
CAAAAGAATAGACCGAGATAGGGTTGAGTGGCCGCTACAGGGCGCTCCCATTCGCCATTCAGGCTGCGCAACTGT
TGGGAAGGGCGTTTCGGTGCGGGCCTCTTCGCTATTACGCCAGCTGGCGAAAGGGGGATGTGCTGCAAGGCGATT
AAGTTGGGTAACGCCAGGGTTTTCCCAGTCACACGCGTAATACGACTCACTATAG
A554 Vector: SGP-gH-SGP-gL-SGP-UL128-SGP-UL130-SGP-UL131
(SEQ ID NO: 39)
ATAGGCGGCGCATGAGAGAAGCCCAGACCAATTACCTACCCAAAATGGAGAAAGTTCACGTTGACATCGAGGAAG
ACAGCCCATTCCTCAGAGCTTTGCAGCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAAACGGAGGTGGACCCATCCGACACGA
TCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGG
AATTGGACAAGAAAATGAAGGAGCTCGCCGCCGTCATGAGCGACCCTGACCTGGAAACTGAGACTATGTGCCTCC
ACGACGACGAGTCGTGTCGCTACGAAGGGCAAGTCGCTGTTTACCAGGATGTATACGCGGTTGACGGACCGACAA
GTCTCTATCACCAAGCCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAACGGCTCGTAACATAG
GCCTATGCAGCTCTGACGTTATGGAGCGGTCACGTAGAGGGATGTCCATTCTTAGAAAGAAGTATTTGAAACCAT
CCAACAATGTTCTATTCTCTGTTGGCTCGACCATCTACCACGAGAAGAGGGACTTACTGAGGAGCTGGCACCTGC
CGTCTGTATTTCACTTACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTACGATGCACCGCGAGG
GATTCTTGTGCTGCAAAGTGACAGACACATTGAACGGGGAGAGGGTCTCTTTTCCCGTGTGCACGTATGTGCCAG
CTACATTGTGTGACCAAATGACTGGCATACTGGCAACAGATGTCAGTGCGGACGACGCGCAAAAACTGCTGGTTG
GGCTCAACCAGCGTATAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAAGGCCACTAGGACTAC
GAGATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACATTGG
AGATCGGGCTGAGAACAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGTTGCGCGCAGCTCTAC
CACCTTTGGCAGCTGATGTTGAGGAGCCCACTCTGGAAGCCGATGTAGACTTGATGTTACAAGAGGCTGGGGCCG
GCTCAGTGGAGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGACATG
CAATACCCGTCCAGGACTTTCAAGCTCTGAGTGAAAGTGCCACCATTGTGTACAACGAACGTGAGTTCGTAAACA
GGTACCTGCACCATATTGCCACACATGGAGGAGCGCTGAACACTGATGAAGAATATTACAAAACTGTCAAGCCCA
GCGAGCACGACGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGACCAGCCGCTC
CTTACCAAGTACCAACCATAGGGGTGTATGGCGTGCCAGGATCAGGCAAGTCTGGCATCATTAAAAGCGCAGTCA
CCAAAAAAGATCTAGTGGTGAGCGCCAAGAAAGAAAACTGTGCAGAAATTATAAGGGACGTCAAGAAAATGAAAG
GGCTGGACGTCAATGCCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTTAACCACGAGATTTGCA
CACAAGTCTTCCACAAAAGCATCTCTCGCCGTTGCACTAAATCTGTGACTTCGGTCGTCTCAACCTTGTTTTACG
ACAAAAAAATGAGAACGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACAAAGGCAACGAAATAA
TGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAG
CCGGCGACCCATGGATAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCCAGAATAAGGCAAACG
TGTGTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTGGAC
TCGATCTGGACTCCGGTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACCCACAACTGCCTCGGG
CAGTTGCCACTGGAAGAGTCTATGACATGAACACTGGTACACTGCGCAATTATGATCCGCGCATAAACCTAGTAC
CTGTAAACAGAAGACTGCCTCATGCTTTAGTCCTCCACCATAATGAACACCCACAGAGTGACTTTTCTTCATTCG
TCAGCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATGACA
TAATATTTGTTAATGTGAGGACCCCATATAAATACCATCACTATCAGCAGTGTGAAGACCATGCCATTAAGCTTA
GCATGTTGACCAAGAAAGCTTGTCTGCATCTGAATCCCGGCGGAACCTGTGTCAGCATAGGTTATGGTTACGCTG
ACAGGGCCAGCGAAAGCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGCACAATCCTTACAAGC
TTTCATCAACCTTGACCAACATTTATACAGGTTCCAGACTCCACGAAGCCGGATGTGCACCCTCATATCATGTGG
TGCGAGGGGATATTGCCACGGCCACCGAAGGAGTGATTATAAATGCTGCTAACAGCAAAGGACAACCTGGCGGAG
GGGTGTGCGGAGCGCTGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTTCGGAGGTTGAAGGTG
ACAAACAGTTGGCAGAGGCTTATGAGTCCATCGCTAAGATTGTCAACGATAACAATTACAAGTCAGTAGCGATTC
CACTGTTGTCCACCGGCATCTTTTCCGGGAACAAAGATCGACTAACCCAATCATTGAACCATTTGCTGACAGCTT
TAGACACCACTGATGCAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTGATGCAGAGCTGGTGA
GGGTGCATCCGAAGAGTTCTTTGGCTGGAAGGAAGGGCTACAGCACAAGCGATGGCAAAACTTTCTCATATTTGG
AAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAAGAGTACAGCGCCTAA
AAGCCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCGTGG
AAACACCACCGGTAGACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGGAAGAAGAGGATAGCA
TAAGTTTGCTGTCAGATGGCCCGACCCACCAGGTGCTGCAAGTCGAGGCAGACATTCACGGGCCGCCCTCTGTAT
CTAGCTCATCCTGGTCCATTCCTCATGCATCCGACTTTGATGTGGACAGTTTATCCATACTTGACACCCTGGAGG
GAGCTAGCGTGACCAGCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAAGAACACCGTCACTTG
CACCCAGCAGGGCCTGCTCGAGAACCAGCCTAGTTTCCACCCCGCCAGGCGTGAATAGGGTGATCACTAGAGAGG
AGCTCGAGGCGCTTACCCCGTCACGCACTCCTAGCAGGTCGGTCTCGAGAACCAGCCTGGTCTCCAACCCGCCAG
GCGTAAATAGGGTGATTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAACGGTGCTATCCGAAG
TGGTGTTGGAGAGGACCGAATTGGAGATTTCGTATGCCCCGCGCCTCGACCAAGAAAAAGAAGAATTACTACGCA
AGAAATTACAGTTAAATCCCACACCTGCTAACAGAAGCAGATACCAGTCCAGGAAGGTGGAGAACATGAAAGCCA
TAACAGCTAGACGTATTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCGCAGTGGAAGCCTGTA
ACGCCATGTTGAAAGAGAACTTTCCGACTGTGGCTTCTTACTGTATTATTCCAGAGTACGATGCCTATTTGGACA
TGGTTGACGGAGCTTCATGCTGCTTAGACACTGCCAGTTTTTGCCCTGCAAAGCTGCGCAGCTTTCCAAAGAAAC
ACTCCTATTTGGAACCCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGGCGGCCTTTAATGTGG
AATGCTTCAAGAAATATGCGTGTAATAATGAATATTGGGAAACGTTTAAAGAAAACCCCATCAGGCTTACTGAAG
AAAACGTGGTAAATTACATTACCAAATTAAAAGGACCAAAAGCTGCTGCTCTTTTTGCGAAGACACATAATTTGA
ATATGTTGCAGGACATACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAGCGTATCTGTGCGGAA
TCCACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGTTTG
ATAAAAGTGAGGACGACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTAAATTTAAATTCGGAG
CCATGATGAAATCTGGAATGTTCCTCACACTGTTTGTGAACACAGTCATTAACATTGTAATCGCAAGCAGAGTGT
TGAGAGAACGGCTAACCGGATCACCATGTGCAGCATTCATTGGAGATGACAATATCGTGAAAGGAGTCAAATCGG
ACAAATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACCCCC
TAAAAAGGCTGTTTAAGCTTGGCAAACCTCTGGCAGCAGACGATGAACATGATGATGACAGGAGAAGGGCATTGC
ATGAAGAGTCAACACGCTGGAACCGAGTGGGTATTCTTTCAGAGCTGTGCAAGGCAGTAGAATCAAGGTATGAAA
CCGTAGGAACTTCCATCATAGTTATGGCCATGACTACTCTAGCTAGCAGTGTTAAATCATTCAGCTACCTGAGAG

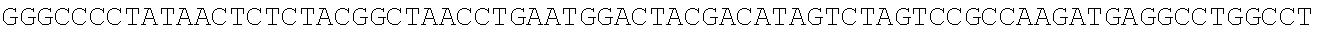

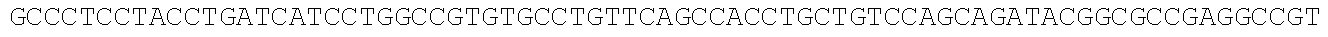

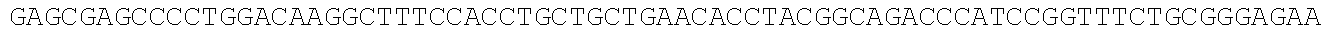

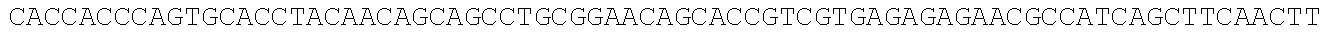

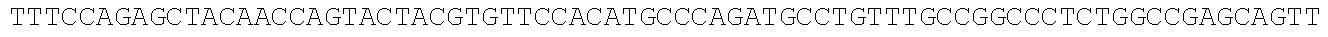

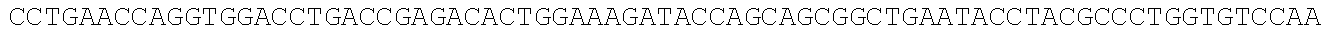

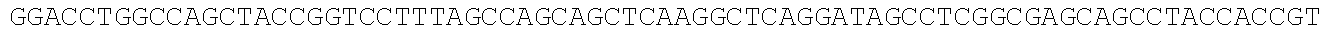

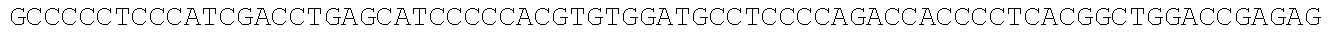

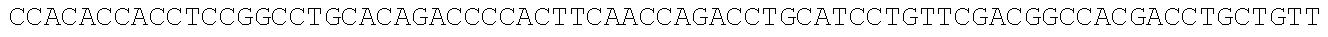

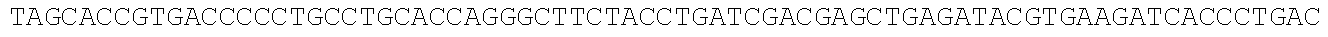

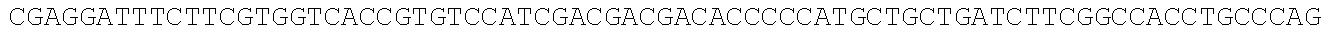

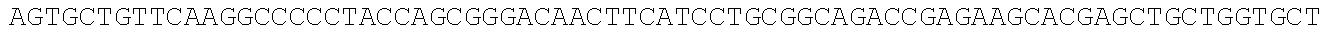

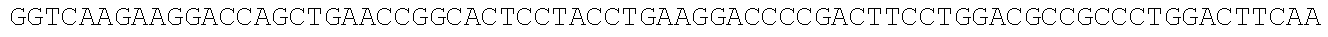

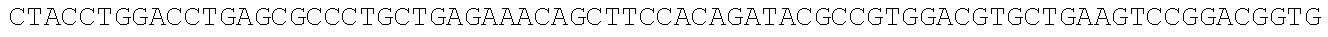

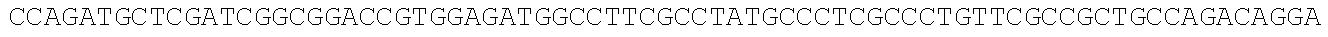

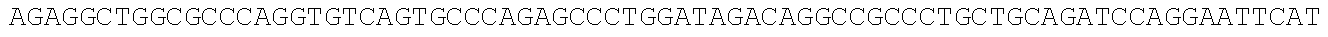

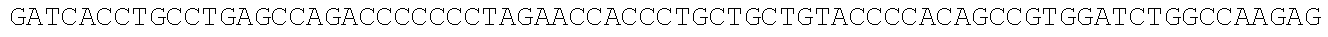

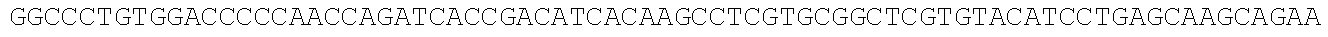

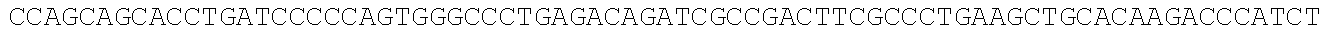

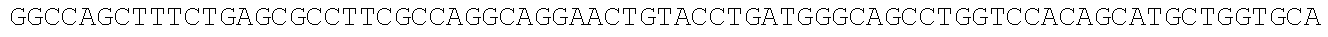

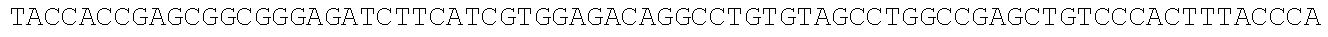

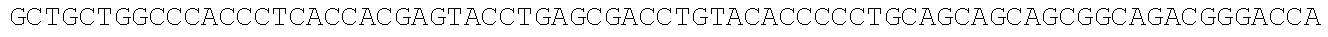

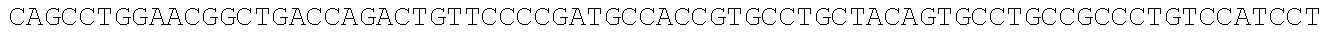

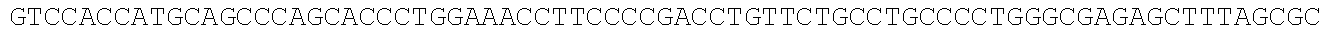

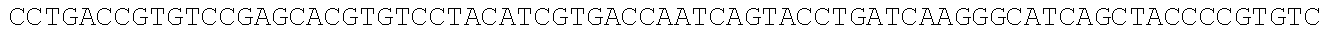

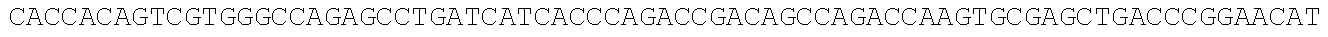

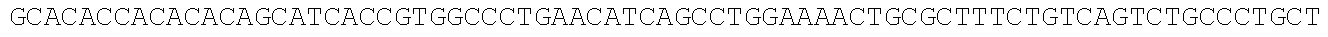

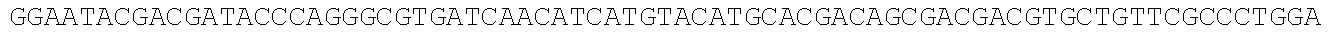

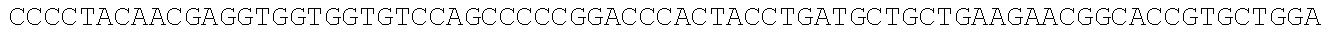

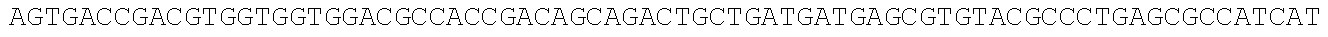

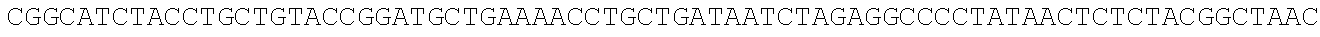

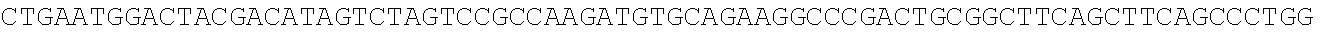

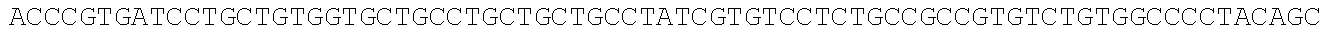

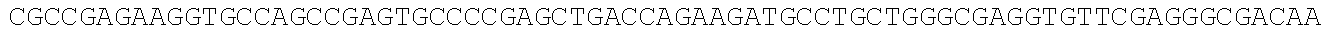

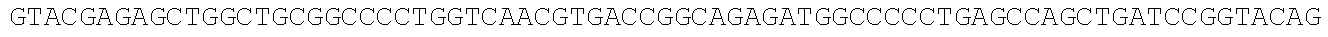

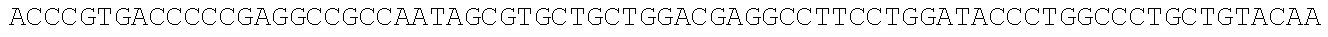

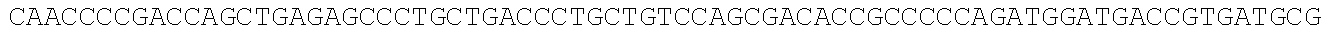

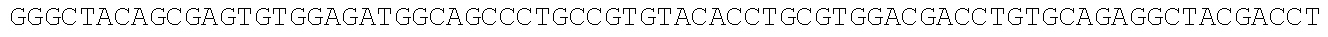

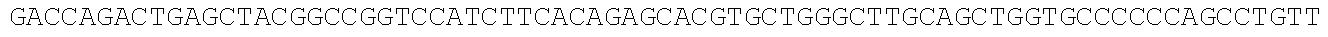

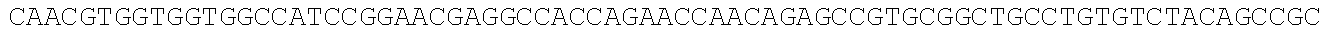

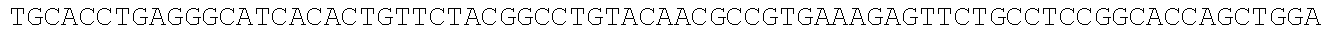

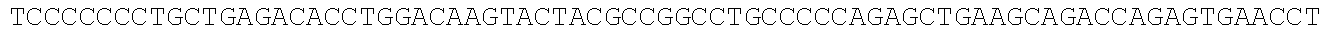

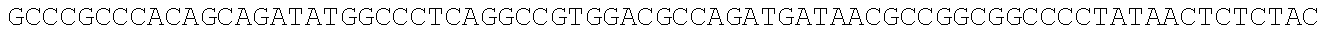

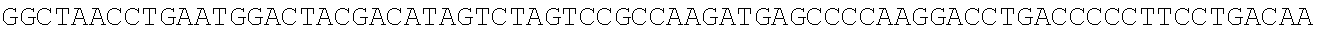

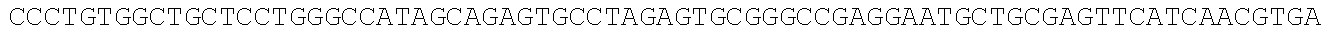

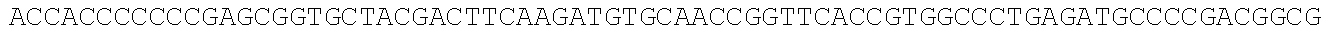

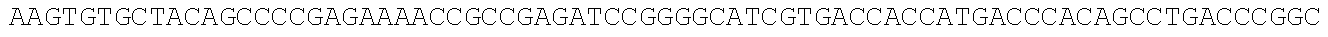

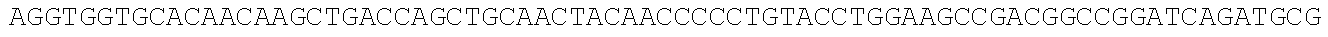

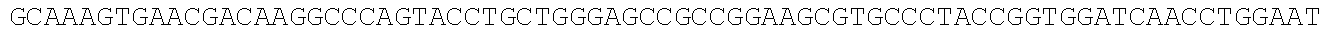

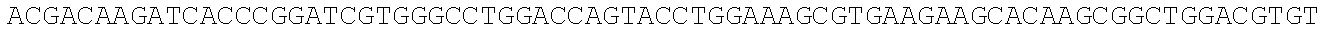

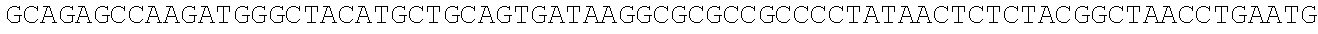

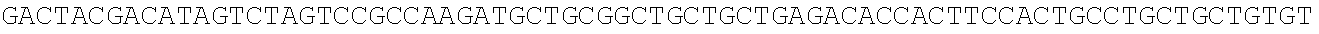

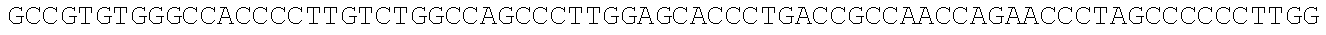

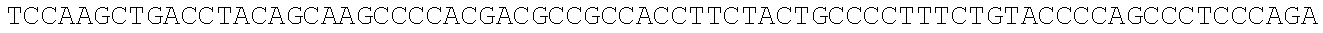

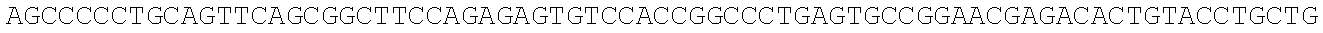

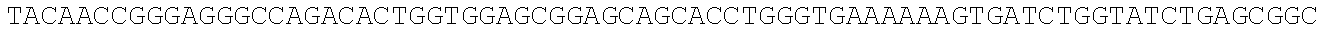

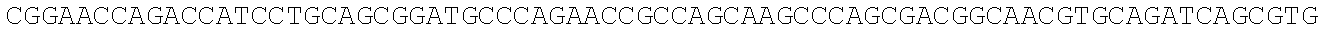

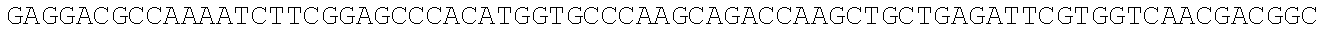

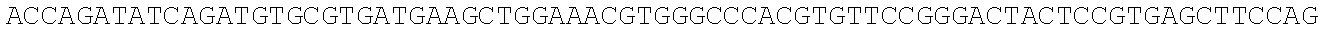

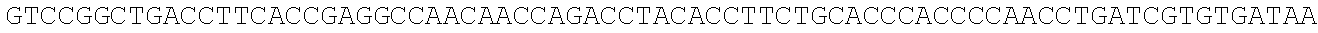

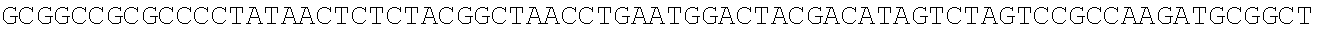

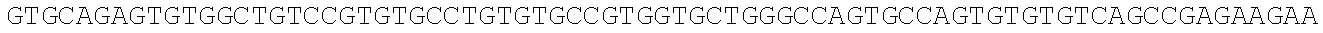

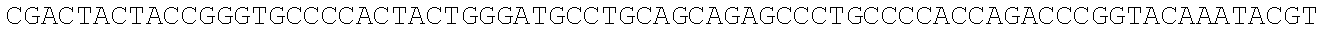

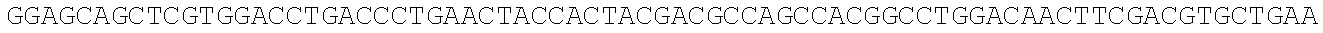

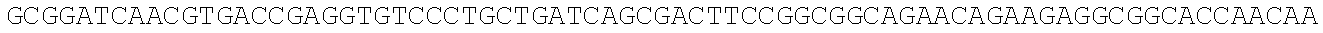

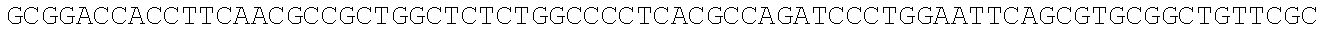

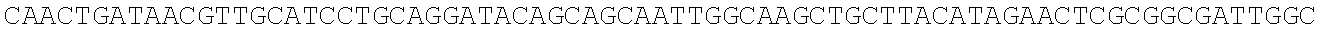

ATGCCGCCTTAAAATTTTTATTTTATTTTTCTTTTCTTTTCCGAATCGGATTTTGTTTTTAATATTTCAAAAAAA
AAAAAAAAAAAAAAAAAAAAAAAAAAAAGGGTCGGCATGGCATCTCCACCTCCTCGCGGTCCGACCTGGGCATCC
GAAGGAGGACGCACGTCCACTCGGATGGCTAAGGGAGAGCCACGTTTAAACGCTAGAGCAAGACGTTTCCCGTTG
AATATGGCTCATAACACCCCTTGTATTACTGTTTATGTAAGCAGACAGTTTTATTGTTCATGATGATATATTTTT
ATCTTGTGCAATGTAACATCAGAGATTTTGAGACACAACGTGGCTTTGTTGAATAAATCGAACTTTTGCTGAGTT
GAAGGATCAGATCACGCATCTTCCCGACAACGCAGACCGTTCCGTGGCAAAGCAAAAGTTCAAAATCACCAACTG
GTCCACCTACAACAAAGCTCTCATCAACCGTGGCTCCCTCACTTTCTGGCTGGATGATGGGGCGATTCAGGCCTG
GTATGAGTCAGCAACACCTTCTTCACGAGGCAGACCTCAGCGCTAGCGGAGTGTATACTGGCTTACTATGTTGGC
ACTGATGAGGGTGTCAGTGAAGTGCTTCATGTGGCAGGAGAAAAAAGGCTGCACCGGTGCGTCAGCAGAATATGT
GATACAGGATATATTCCGCTTCCTCGCTCACTGACTCGCTACGCTCGGTCGTTCGACTGCGGCGAGCGGAAATGG
CTTACGAACGGGGCGGAGATTTCCTGGAAGATGCCAGGAAGATACTTAACAGGGAAGTGAGAGGGCCGCGGCAAA
GCCGTTTTTCCATAGGCTCCGCCCCCCTGACAAGCATCACGAAATCTGACGCTCAAATCAGTGGTGGCGAAACCC
GACAGGACTATAAAGATACCAGGCGTTTCCCCTGGCGGCTCCCTCGTGCGCTCTCCTGTTCCTGCCTTTCGGTTT
ACCGGTGTCATTCCGCTGTTATGGCCGCGTTTGTCTCATTCCACGCCTGACACTCAGTTCCGGGTAGGCAGTTCG
CTCCAAGCTGGACTGTATGCACGAACCCCCCGTTCAGTCCGACCGCTGCGCCTTATCCGGTAACTATCGTCTTGA
GTCCAACCCGGAAAGACATGCAAAAGCACCACTGGCAGCAGCCACTGGTAATTGATTTAGAGGAGTTAGTCTTGA
AGTCATGCGCCGGTTAAGGCTAAACTGAAAGGACAAGTTTTGGTGACTGCGCTCCTCCAAGCCAGTTACCTCGGT
TCAAAGAGTTGGTAGCTCAGAGAACCTTCGAAAAACCGCCCTGCAAGGCGGTTTTTTCGTTTTCAGAGCAAGAGA
TTACGCGCAGACCAAAACGATCTCAAGAAGATCATCTTATTAAGGGGTCTGACGCTCAGTGGAACGAAAACTCAC
GTTAAGGGATTTTGGTCATGAGATTATCAAAAAGGATCTTCACCTAGATCCTTTTAAATTAAAAATGAAGTTTTA
AATCAATCTAAAGTATATATGAGTAAACTTGGTCTGACAGTTATTAGAAAAATTCATCCAGCAGACGATAAAACG
CAATACGCTGGCTATCCGGTGCCGCAATGCCATACAGCACCAGAAAACGATCCGCCCATTCGCCGCCCAGTTCTT
CCGCAATATCACGGGTGGCCAGCGCAATATCCTGATAACGATCCGCCACGCCCAGACGGCCGCAATCAATAAAGC
CGCTAAAACGGCCATTTTCCACCATAATGTTCGGCAGGCACGCATCACCATGGGTCACCACCAGATCTTCGCCAT
CCGGCATGCTCGCTTTCAGACGCGCAAACAGCTCTGCCGGTGCCAGGCCCTGATGTTCTTCATCCAGATCATCCT
GATCCACCAGGCCCGCTTCCATACGGGTACGCGCACGTTCAATACGATGTTTCGCCTGATGATCAAACGGACAGG
TCGCCGGGTCCAGGGTATGCAGACGACGCATGGCATCCGCCATAATGCTCACTTTTTCTGCCGGCGCCAGATGGC
TAGACAGCAGATCCTGACCCGGCACTTCGCCCAGCAGCAGCCAATCACGGCCCGCTTCGGTCACCACATCCAGCA
CCGCCGCACACGGAACACCGGTGGTGGCCAGCCAGCTCAGACGCGCCGCTTCATCCTGCAGCTCGTTCAGCGCAC
CGCTCAGATCGGTTTTCACAAACAGCACCGGACGACCCTGCGCGCTCAGACGAAACACCGCCGCATCAGAGCAGC
CAATGGTCTGCTGCGCCCAATCATAGCCAAACAGACGTTCCACCCACGCTGCCGGGCTACCCGCATGCAGGCCAT
CCTGTTCAATCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTAAATTGT
AAGCGTTAATATTTTGTTAAAATTCGCGTTAAATTTTTGTTAAATCAGCTCATTTTTTAACCAATAGGCCGAAAT
CGGCAAAATCCCTTATAAATCAAAAGAATAGACCGAGATAGGGTTGAGTGGCCGCTACAGGGCGCTCCCATTCGC
CATTCAGGCTGCGCAACTGTTGGGAAGGGCGTTTCGGTGCGGGCCTCTTCGCTATTACGCCAGCTGGCGAAAGGG
GGATGTGCTGCAAGGCGATTAAGTTGGGTAACGCCAGGGTTTTCCCAGTCACACGCGTAATACGACTCACTATAG
A555 Vector: SGP-gHsol-SGP-gL-SGP-UL128-SGP-UL130-SGP-UL131
(SEQ ID NO: 40)
ATAGGCGGCGCATGAGAGAAGCCCAGACCAATTACCTACCCAAAATGGAGAAAGTTCACGTTGACATCGAGGAAG
ACAGCCCATTCCTCAGAGCTTTGCAGCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAAACGGAGGTGGACCCATCCGACACGA
TCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGG
AATTGGACAAGAAAATGAAGGAGCTCGCCGCCGTCATGAGCGACCCTGACCTGGAAACTGAGACTATGTGCCTCC
ACGACGACGAGTCGTGTCGCTACGAAGGGCAAGTCGCTGTTTACCAGGATGTATACGCGGTTGACGGACCGACAA
GTCTCTATCACCAAGCCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAACGGCTCGTAACATAG
GCCTATGCAGCTCTGACGTTATGGAGCGGTCACGTAGAGGGATGTCCATTCTTAGAAAGAAGTATTTGAAACCAT
CCAACAATGTTCTATTCTCTGTTGGCTCGACCATCTACCACGAGAAGAGGGACTTACTGAGGAGCTGGCACCTGC
CGTCTGTATTTCACTTACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTACGATGCACCGCGAGG
GATTCTTGTGCTGCAAAGTGACAGACACATTGAACGGGGAGAGGGTCTCTTTTCCCGTGTGCACGTATGTGCCAG
CTACATTGTGTGACCAAATGACTGGCATACTGGCAACAGATGTCAGTGCGGACGACGCGCAAAAACTGCTGGTTG
GGCTCAACCAGCGTATAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAAGGCCACTAGGACTAC
GAGATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACATTGG
AGATCGGGCTGAGAACAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGTTGCGCGCAGCTCTAC
CACCTTTGGCAGCTGATGTTGAGGAGCCCACTCTGGAAGCCGATGTAGACTTGATGTTACAAGAGGCTGGGGCCG
GCTCAGTGGAGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGACATG
CAATACCCGTCCAGGACTTTCAAGCTCTGAGTGAAAGTGCCACCATTGTGTACAACGAACGTGAGTTCGTAAACA
GGTACCTGCACCATATTGCCACACATGGAGGAGCGCTGAACACTGATGAAGAATATTACAAAACTGTCAAGCCCA
GCGAGCACGACGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGACCAGCCGCTC
CTTACCAAGTACCAACCATAGGGGTGTATGGCGTGCCAGGATCAGGCAAGTCTGGCATCATTAAAAGCGCAGTCA
CCAAAAAAGATCTAGTGGTGAGCGCCAAGAAAGAAAACTGTGCAGAAATTATAAGGGACGTCAAGAAAATGAAAG
GGCTGGACGTCAATGCCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTTAACCACGAGATTTGCA
CACAAGTCTTCCACAAAAGCATCTCTCGCCGTTGCACTAAATCTGTGACTTCGGTCGTCTCAACCTTGTTTTACG
ACAAAAAAATGAGAACGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACAAAGGCAACGAAATAA
TGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAG
CCGGCGACCCATGGATAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCCAGAATAAGGCAAACG
TGTGTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTGGAC
TCGATCTGGACTCCGGTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACCCACAACTGCCTCGGG
CAGTTGCCACTGGAAGAGTCTATGACATGAACACTGGTACACTGCGCAATTATGATCCGCGCATAAACCTAGTAC
CTGTAAACAGAAGACTGCCTCATGCTTTAGTCCTCCACCATAATGAACACCCACAGAGTGACTTTTCTTCATTCG
TCAGCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATGACA
TAATATTTGTTAATGTGAGGACCCCATATAAATACCATCACTATCAGCAGTGTGAAGACCATGCCATTAAGCTTA
GCATGTTGACCAAGAAAGCTTGTCTGCATCTGAATCCCGGCGGAACCTGTGTCAGCATAGGTTATGGTTACGCTG
ACAGGGCCAGCGAAAGCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGCACAATCCTTACAAGC
TTTCATCAACCTTGACCAACATTTATACAGGTTCCAGACTCCACGAAGCCGGATGTGCACCCTCATATCATGTGG
TGCGAGGGGATATTGCCACGGCCACCGAAGGAGTGATTATAAATGCTGCTAACAGCAAAGGACAACCTGGCGGAG
GGGTGTGCGGAGCGCTGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTTCGGAGGTTGAAGGTG
ACAAACAGTTGGCAGAGGCTTATGAGTCCATCGCTAAGATTGTCAACGATAACAATTACAAGTCAGTAGCGATTC
CACTGTTGTCCACCGGCATCTTTTCCGGGAACAAAGATCGACTAACCCAATCATTGAACCATTTGCTGACAGCTT
TAGACACCACTGATGCAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTGATGCAGAGCTGGTGA
GGGTGCATCCGAAGAGTTCTTTGGCTGGAAGGAAGGGCTACAGCACAAGCGATGGCAAAACTTTCTCATATTTGG
AAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAAGAGTACAGCGCCTAA
AAGCCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCGTGG
AAACACCACCGGTAGACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGGAAGAAGAGGATAGCA
TAAGTTTGCTGTCAGATGGCCCGACCCACCAGGTGCTGCAAGTCGAGGCAGACATTCACGGGCCGCCCTCTGTAT
CTAGCTCATCCTGGTCCATTCCTCATGCATCCGACTTTGATGTGGACAGTTTATCCATACTTGACACCCTGGAGG
GAGCTAGCGTGACCAGCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAAGAACACCGTCACTTG
CACCCAGCAGGGCCTGCTCGAGAACCAGCCTAGTTTCCACCCCGCCAGGCGTGAATAGGGTGATCACTAGAGAGG
AGCTCGAGGCGCTTACCCCGTCACGCACTCCTAGCAGGTCGGTCTCGAGAACCAGCCTGGTCTCCAACCCGCCAG
GCGTAAATAGGGTGATTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAACGGTGCTATCCGAAG
TGGTGTTGGAGAGGACCGAATTGGAGATTTCGTATGCCCCGCGCCTCGACCAAGAAAAAGAAGAATTACTACGCA
AGAAATTACAGTTAAATCCCACACCTGCTAACAGAAGCAGATACCAGTCCAGGAAGGTGGAGAACATGAAAGCCA
TAACAGCTAGACGTATTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCGCAGTGGAAGCCTGTA
ACGCCATGTTGAAAGAGAACTTTCCGACTGTGGCTTCTTACTGTATTATTCCAGAGTACGATGCCTATTTGGACA
TGGTTGACGGAGCTTCATGCTGCTTAGACACTGCCAGTTTTTGCCCTGCAAAGCTGCGCAGCTTTCCAAAGAAAC
ACTCCTATTTGGAACCCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGGCGGCCTTTAATGTGG
AATGCTTCAAGAAATATGCGTGTAATAATGAATATTGGGAAACGTTTAAAGAAAACCCCATCAGGCTTACTGAAG
AAAACGTGGTAAATTACATTACCAAATTAAAAGGACCAAAAGCTGCTGCTCTTTTTGCGAAGACACATAATTTGA
ATATGTTGCAGGACATACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAGCGTATCTGTGCGGAA
TCCACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGTTTG
ATAAAAGTGAGGACGACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTAAATTTAAATTCGGAG
CCATGATGAAATCTGGAATGTTCCTCACACTGTTTGTGAACACAGTCATTAACATTGTAATCGCAAGCAGAGTGT
TGAGAGAACGGCTAACCGGATCACCATGTGCAGCATTCATTGGAGATGACAATATCGTGAAAGGAGTCAAATCGG
ACAAATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACCCCC
TAAAAAGGCTGTTTAAGCTTGGCAAACCTCTGGCAGCAGACGATGAACATGATGATGACAGGAGAAGGGCATTGC
ATGAAGAGTCAACACGCTGGAACCGAGTGGGTATTCTTTCAGAGCTGTGCAAGGCAGTAGAATCAAGGTATGAAA
CCGTAGGAACTTCCATCATAGTTATGGCCATGACTACTCTAGCTAGCAGTGTTAAATCATTCAGCTACCTGAGAG

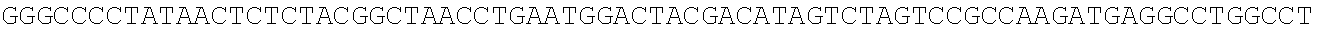

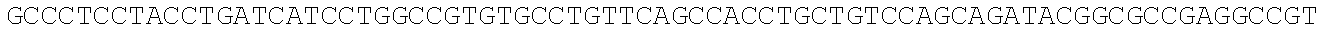

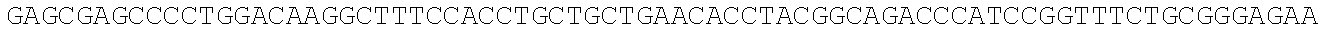

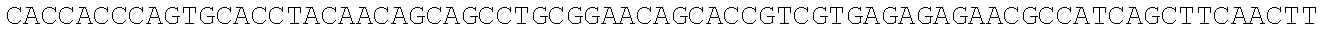

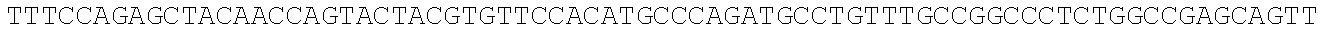

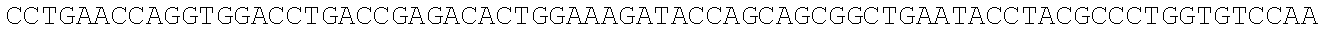

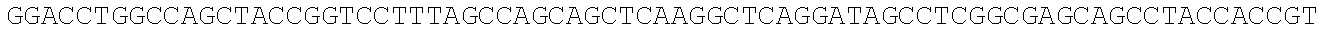

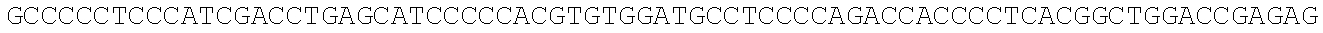

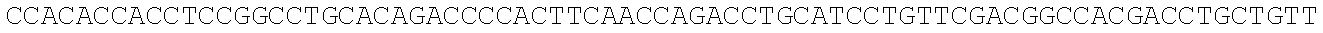

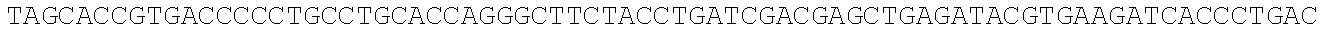

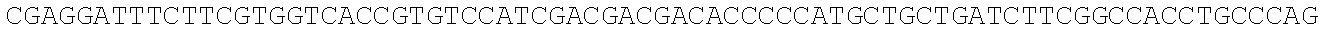

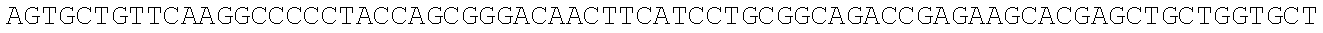

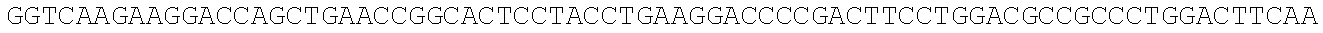

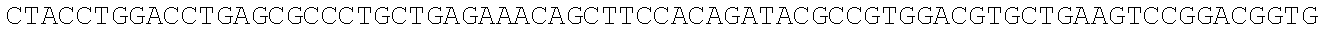

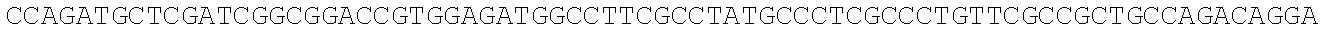

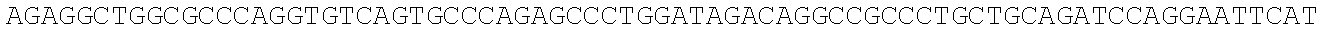

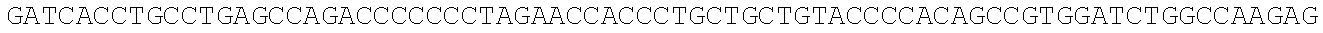

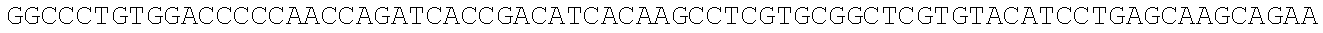

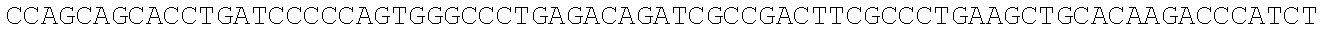

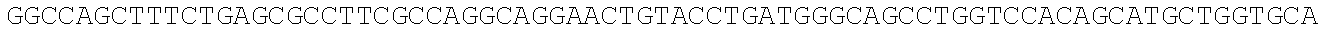

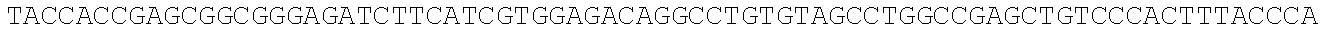

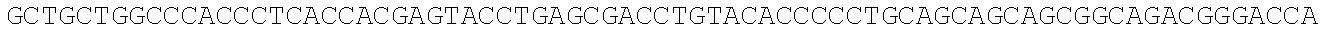

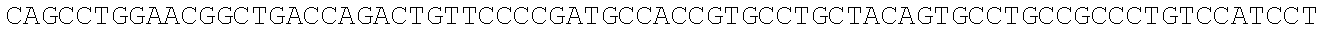

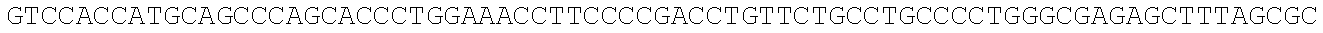

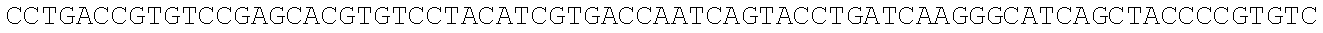

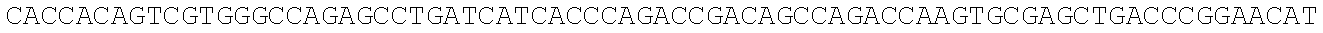

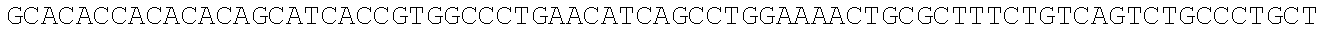

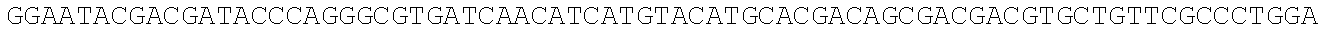

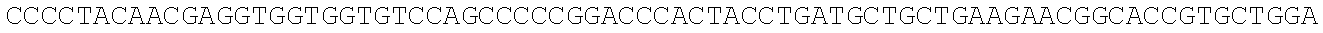

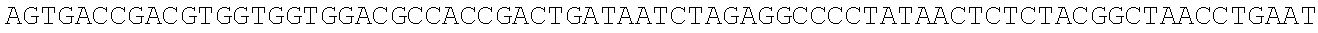

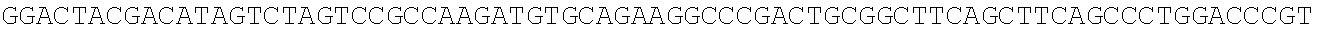

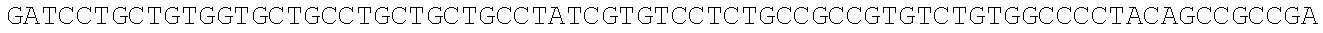

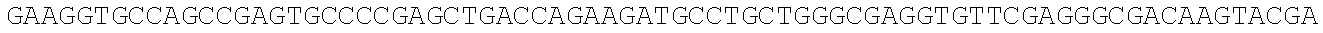

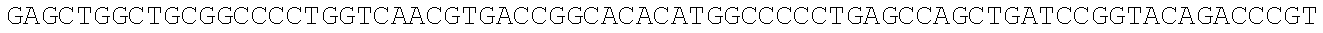

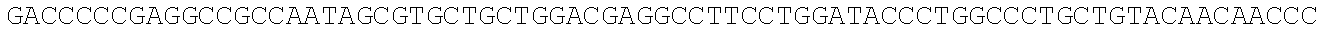

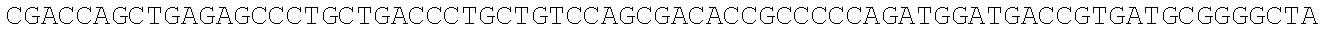

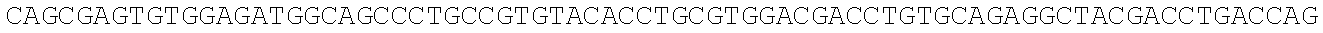

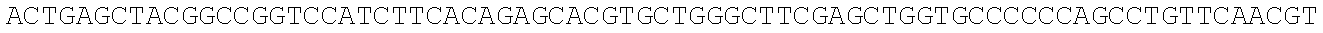

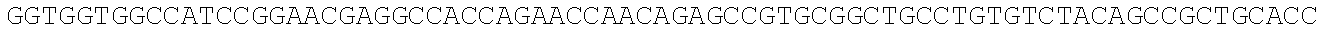

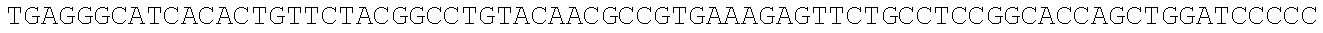

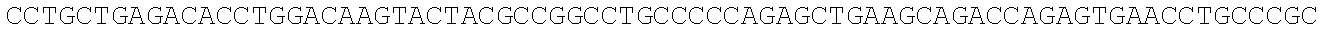

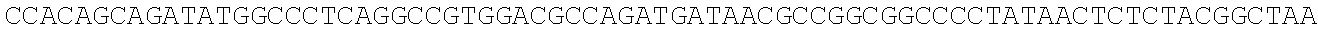

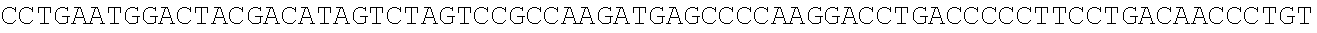

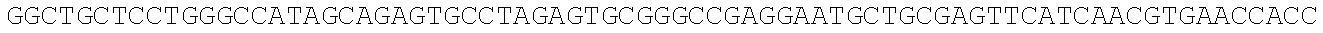

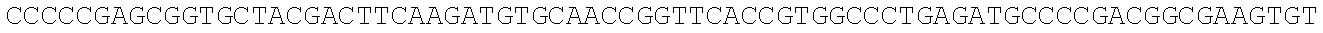

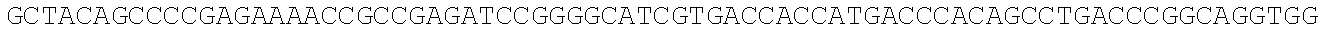

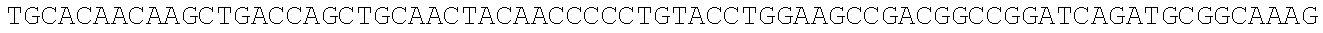

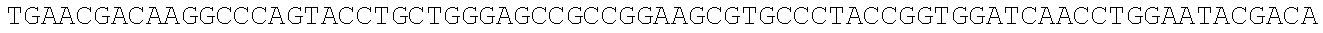

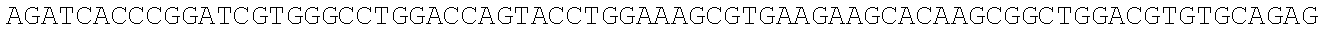

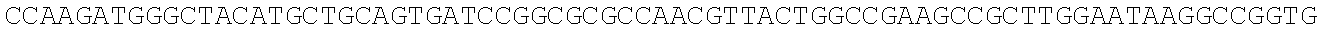

TGCGTTTGTCTATATGTTATTTTCCACCATATTGCCGTCTTTTGGCAATGTGAGGGCCCGGAAACCTGGCCCTGT
CTTCTTGACGAGCATTCCTAGGGGTCTTTCCCCTCTCGCCAAAGGAATGCAAGGTCTGTTGAATGTCGTGAAGGA
AGCAGTTCCTCTGGAAGCTTCTTGAAGACAAACAACGTCTGTAGCGACCCTTTGCAGGCAGCGGAACCCCCCACC
TGGCGACAGGTGCCTCTGCGGCCAAAAGCCACGTGTATAAGATACACCTGCAAAGGCGGCACAACCCCAGTGCCA
CGTTGTGAGTTGGATAGTTGTGGAAAGAGTCAAATGGCTCTCCTCAAGCGTATTCAACAAGGGGCTGAAGGATGC
CCAGAAGGTACCCCATTGTATGGGATCTGATCTGGGGCCTCGGTGCACATGCTTTACATGTGTTTAGTCGAGGTT

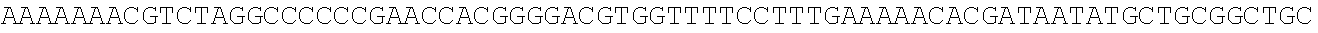

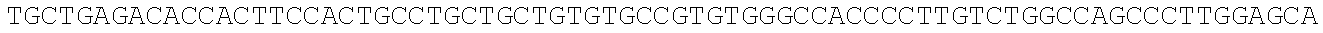

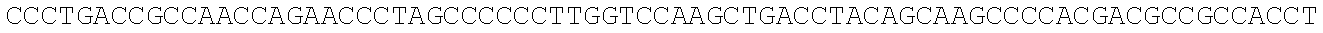

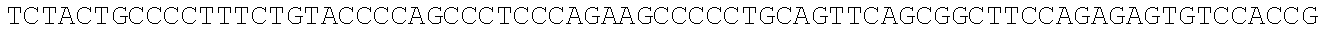

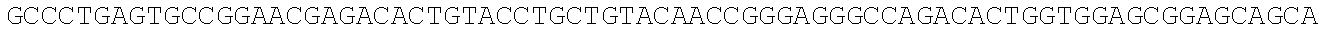

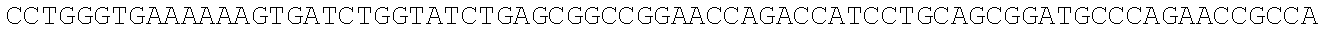

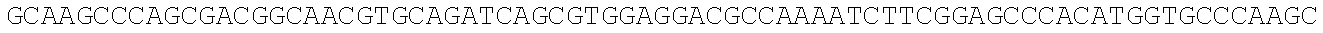

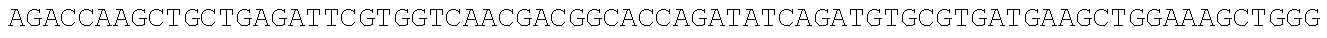

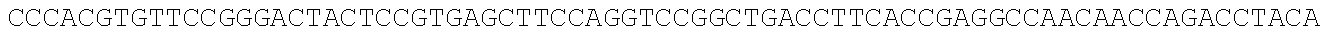

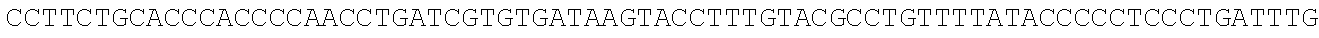

CAACTTAGAAGCAACGCAAACCAGATCAATAGTAGGTGTGACATACCAGTCGCATCTTGATCAAGCACTTCTGTA
TCCCCGGACCGAGTATCAATAGACTGTGCACACGGTTGAAGGAGAAAACGTCCGTTACCCGGCTAACTACTTCGA
GAAGCCTAGTAACGCCATTGAAGTTGCAGAGTGTTTCGCTCAGCACTCCCCCCGTGTAGATCAGGTCGATGAGTC
ACCGCATTCCCCACGGGCGACCGTGGCGGTGGCTGCGTTGGCGGCCTGCCTATGGGGTAACCCATAGGACGCTCT
AATACGGACATGGCGTGAAGAGTCTATTGAGCTAGTTAGTAGTCCTCCGGCCCCTGAATGCGGCTAATCCTAACT
GCGGAGCACATACCCTTAATCCAAAGGGCAGTGTGTCGTAACGGGCAACTCTGCAGCGGAACCGACTACTTTGGG
TGTCCGTGTTTCTTTTTATTCTTGTATTGGCTGCTTATGGTGACAATTAAAGAATTGTTACCATATAGCTATTGG
ATTGGCCATCCAGTGTCAAACAGAGCTATTGTATATCTCTTTGTTGGATTCACACCTCTCACTCTTGAAACGTTA

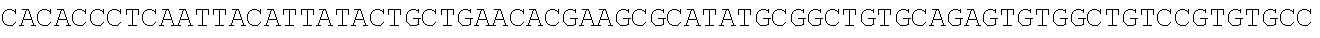

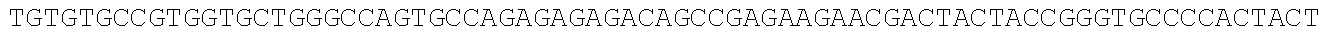

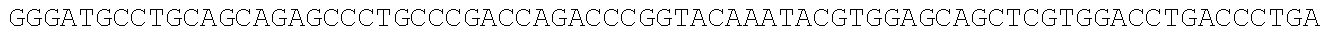

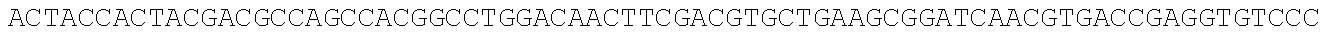

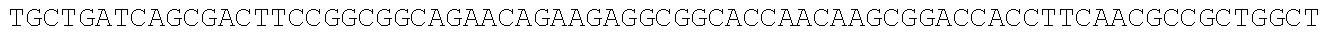

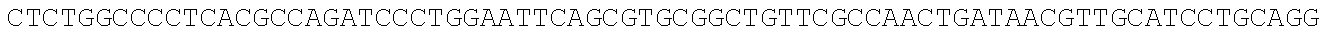

ATACAGCAGCAATTGGCAAGCTGCTTACATAGAACTCGCGGCGATTGGCATGCCGCCTTAAAATTTTTATTTTAT
TTTTCTTTTCTTTTCCGAATCGGATTTTGTTTTTAATATTTCAAAAAAAAAAAAAAAAAAAAAAAAAAAAAAAAA
AAGGGTCGGCATGGCATCTCCACCTCCTCGCGGTCCGACCTGGGCATCCGAAGGAGGACGCACGTCCACTCGGAT
GGCTAAGGGAGAGCCACGTTTAAACGCTAGAGCAAGACGTTTCCCGTTGAATATGGCTCATAACACCCCTTGTAT
TACTGTTTATGTAAGCAGACAGTTTTATTGTTCATGATGATATATTTTTATCTTGTGCAATGTAACATCAGAGAT
TTTGAGACACAACGTGGCTTTGTTGAATAAATCGAACTTTTGCTGAGTTGAAGGATCAGATCACGCATCTTCCCG
ACAACGCAGACCGTTCCGTGGCAAAGCAAAAGTTCAAAATCACCAACTGGTCCACCTACAACAAAGCTCTCATCA
ACCGTGGCTCCCTCACTTTCTGGCTGGATGATGGGGCGATTCAGGCCTGGTATGAGTCAGCAACACCTTCTTCAC
GAGGCAGACCTCAGCGCTAGCGGAGTGTATACTGGCTTACTATGTTGGCACTGATGAGGGTGTCAGTGAAGTGCT
TCATGTGGCAGGAGAAAAAAGGCTGCACCGGTGCGTCAGCAGAATATGTGATACAGGATATATTCCGCTTCCTCG
CTCACTGACTCGCTACGCTCGGTCGTTCGACTGCGGCGAGCGGAAATGGCTTACGAACGGGGCGGAGATTTCCTG
GAAGATGCCAGGAAGATACTTAACAGGGAAGTGAGAGGGCCGCGGCAAAGCCGTTTTTCCATAGGCTCCGCCCCC
CTGACAAGCATCACGAAATCTGACGCTCAAATCAGTGGTGGCGAAACCCGACAGGACTATAAAGATACCAGGCGT
TTCCCCTGGCGGCTCCCTCGTGCGCTCTCCTGTTCCTGCCTTTCGGTTTACCGGTGTCATTCCGCTGTTATGGCC
GCGTTTGTCTCATTCCACGCCTGACACTCAGTTCCGGGTAGGCAGTTCGCTCCAAGCTGGACTGTATGCACGAAC
CCCCCGTTCAGTCCGACCGCTGCGCCTTATCCGGTAACTATCGTCTTGAGTCCAACCCGGAAAGACATGCAAAAG
CACCACTGGCAGCAGCCACTGGTAATTGATTTAGAGGAGTTAGTCTTGAAGTCATGCGCCGGTTAAGGCTAAACT
GAAAGGACAAGTTTTGGTGACTGCGCTCCTCCAAGCCAGTTACCTCGGTTCAAAGAGTTGGTAGCTCAGAGAACC
TTCGAAAAACCGCCCTGCAAGGCGGTTTTTTCGTTTTCAGAGCAAGAGATTACGCGCAGACCAAAACGATCTCAA
GAAGATCATCTTATTAAGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATATATGAGTAA
ACTTGGTCTGACAGTTATTAGAAAAATTCATCCAGCAGACGATAAAACGCAATACGCTGGCTATCCGGTGCCGCA
ATGCCATACAGCACCAGAAAACGATCCGCCCATTCGCCGCCCAGTTCTTCCGCAATATCACGGGTGGCCAGCGCA
ATATCCTGATAACGATCCGCCACGCCCAGACGGCCGCAATCAATAAAGCCGCTAAAACGGCCATTTTCCACCATA
ATGTTCGGCAGGCACGCATCACCATGGGTCACCACCAGATCTTCGCCATCCGGCATGCTCGCTTTCAGACGCGCA
AACAGCTCTGCCGGTGCCAGGCCCTGATGTTCTTCATCCAGATCATCCTGATCCACCAGGCCCGCTTCCATACGG
GTACGCGCACGTTCAATACGATGTTTCGCCTGATGATCAAACGGACAGGTCGCCGGGTCCAGGGTATGCAGACGA
CGCATGGCATCCGCCATAATGCTCACTTTTTCTGCCGGCGCCAGATGGCTAGACAGCAGATCCTGACCCGGCACT
TCGCCCAGCAGCAGCCAATCACGGCCCGCTTCGGTCACCACATCCAGCACCGCCGCACACGGAACACCGGTGGTG
GCCAGCCAGCTCAGACGCGCCGCTTCATCCTGCAGCTCGTTCAGCGCACCGCTCAGATCGGTTTTCACAAACAGC
ACCGGACGACCCTGCGCGCTCAGACGAAACACCGCCGCATCAGAGCAGCCAATGGTCTGCTGCGCCCAATCATAG
CCAAACAGACGTTCCACCCACGCTGCCGGGCTACCCGCATGCAGGCCATCCTGTTCAATCATACTCTTCCTTTTT
CAATATTATTGAAGCATTTATCAGGGTTATTGTCTCATGAGCGGATACATATTTGAATGTATTTAGAAAAATAAA
CAAATAGGGGTTCCGCGCACATTTCCCCGAAAAGTGCCACCTAAATTGTAAGCGTTAATATTTTGTTAAAATTCG
CGTTAAATTTTTGTTAAATCAGCTCATTTTTTAACCAATAGGCCGAAATCGGCAAAATCCCTTATAAATCAAAAG
AATAGACCGAGATAGGGTTGAGTGGCCGCTACAGGGCGCTCCCATTCGCCATTCAGGCTGCGCAACTGTTGGGAA
GGGCGTTTCGGTGCGGGCCTCTTCGCTATTACGCCAGCTGGCGAAAGGGGGATGTGCTGCAAGGCGATTAAGTTG
GGTAACGCCAGGGTTTTCCCAGTCACACGCGTAATACGACTCACTATAG
A556 Vector: SGP-gHsol6His-SGP-gL-SGP-UL128-SGP-UL130-SGP-UL131 (“6His”
disclosed as SEQ ID NO: 45)
(SEQ ID NO: 41)
ATAGGCGGCGCATGAGAGAAGCCCAGACCAATTACCTACCCAAAATGGAGAAAGTTCACGTTGACATCGAGGAAG
ACAGCCCATTCCTCAGAGCTTTGCAGCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAAACGGAGGTGGACCCATCCGACACGA
TCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGG
AATTGGACAAGAAAATGAAGGAGCTCGCCGCCGTCATGAGCGACCCTGACCTGGAAACTGAGACTATGTGCCTCC
ACGACGACGAGTCGTGTCGCTACGAAGGGCAAGTCGCTGTTTACCAGGATGTATACGCGGTTGACGGACCGACAA
GTCTCTATCACCAAGCCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAACGGCTCGTAACATAG
GCCTATGCAGCTCTGACGTTATGGAGCGGTCACGTAGAGGGATGTCCATTCTTAGAAAGAAGTATTTGAAACCAT
CCAACAATGTTCTATTCTCTGTTGGCTCGACCATCTACCACGAGAAGAGGGACTTACTGAGGAGCTGGCACCTGC
CGTCTGTATTTCACTTACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTACGATGCACCGCGAGG
GATTCTTGTGCTGCAAAGTGACAGACACATTGAACGGGGAGAGGGTCTCTTTTCCCGTGTGCACGTATGTGCCAG
CTACATTGTGTGACCAAATGACTGGCATACTGGCAACAGATGTCAGTGCGGACGACGCGCAAAAACTGCTGGTTG
GGCTCAACCAGCGTATAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAAGGCCACTAGGACTAC
GAGATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACATTGG
AGATCGGGCTGAGAACAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGTTGCGCGCAGCTCTAC
CACCTTTGGCAGCTGATGTTGAGGAGCCCACTCTGGAAGCCGATGTAGACTTGATGTTACAAGAGGCTGGGGCCG
GCTCAGTGGAGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGACATG
CAATACCCGTCCAGGACTTTCAAGCTCTGAGTGAAAGTGCCACCATTGTGTACAACGAACGTGAGTTCGTAAACA
GGTACCTGCACCATATTGCCACACATGGAGGAGCGCTGAACACTGATGAAGAATATTACAAAACTGTCAAGCCCA
GCGAGCACGACGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGACCAGCCGCTC
CTTACCAAGTACCAACCATAGGGGTGTATGGCGTGCCAGGATCAGGCAAGTCTGGCATCATTAAAAGCGCAGTCA
CCAAAAAAGATCTAGTGGTGAGCGCCAAGAAAGAAAACTGTGCAGAAATTATAAGGGACGTCAAGAAAATGAAAG
GGCTGGACGTCAATGCCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTTAACCACGAGATTTGCA
CACAAGTCTTCCACAAAAGCATCTCTCGCCGTTGCACTAAATCTGTGACTTCGGTCGTCTCAACCTTGTTTTACG
ACAAAAAAATGAGAACGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACAAAGGCAACGAAATAA
TGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAG
CCGGCGACCCATGGATAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCCAGAATAAGGCAAACG
TGTGTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTGGAC
TCGATCTGGACTCCGGTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACCCACAACTGCCTCGGG
CAGTTGCCACTGGAAGAGTCTATGACATGAACACTGGTACACTGCGCAATTATGATCCGCGCATAAACCTAGTAC
CTGTAAACAGAAGACTGCCTCATGCTTTAGTCCTCCACCATAATGAACACCCACAGAGTGACTTTTCTTCATTCG
TCAGCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATGACA
TAATATTTGTTAATGTGAGGACCCCATATAAATACCATCACTATCAGCAGTGTGAAGACCATGCCATTAAGCTTA
GCATGTTGACCAAGAAAGCTTGTCTGCATCTGAATCCCGGCGGAACCTGTGTCAGCATAGGTTATGGTTACGCTG
ACAGGGCCAGCGAAAGCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGCACAATCCTTACAAGC
TTTCATCAACCTTGACCAACATTTATACAGGTTCCAGACTCCACGAAGCCGGATGTGCACCCTCATATCATGTGG
TGCGAGGGGATATTGCCACGGCCACCGAAGGAGTGATTATAAATGCTGCTAACAGCAAAGGACAACCTGGCGGAG
GGGTGTGCGGAGCGCTGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTTCGGAGGTTGAAGGTG
ACAAACAGTTGGCAGAGGCTTATGAGTCCATCGCTAAGATTGTCAACGATAACAATTACAAGTCAGTAGCGATTC
CACTGTTGTCCACCGGCATCTTTTCCGGGAACAAAGATCGACTAACCCAATCATTGAACCATTTGCTGACAGCTT
TAGACACCACTGATGCAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTGATGCAGAGCTGGTGA
GGGTGCATCCGAAGAGTTCTTTGGCTGGAAGGAAGGGCTACAGCACAAGCGATGGCAAAACTTTCTCATATTTGG
AAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAAGAGTACAGCGCCTAA
AAGCCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCGTGG
AAACACCACCGGTAGACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGGAAGAAGAGGATAGCA
TAAGTTTGCTGTCAGATGGCCCGACCCACCAGGTGCTGCAAGTCGAGGCAGACATTCACGGGCCGCCCTCTGTAT
CTAGCTCATCCTGGTCCATTCCTCATGCATCCGACTTTGATGTGGACAGTTTATCCATACTTGACACCCTGGAGG
GAGCTAGCGTGACCAGCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAAGAACACCGTCACTTG
CACCCAGCAGGGCCTGCTCGAGAACCAGCCTAGTTTCCACCCCGCCAGGCGTGAATAGGGTGATCACTAGAGAGG
AGCTCGAGGCGCTTACCCCGTCACGCACTCCTAGCAGGTCGGTCTCGAGAACCAGCCTGGTCTCCAACCCGCCAG
GCGTAAATAGGGTGATTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAACGGTGCTATCCGAAG
TGGTGTTGGAGAGGACCGAATTGGAGATTTCGTATGCCCCGCGCCTCGACCAAGAAAAAGAAGAATTACTACGCA
AGAAATTACAGTTAAATCCCACACCTGCTAACAGAAGCAGATACCAGTCCAGGAAGGTGGAGAACATGAAAGCCA
TAACAGCTAGACGTATTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCGCAGTGGAAGCCTGTA
ACGCCATGTTGAAAGAGAACTTTCCGACTGTGGCTTCTTACTGTATTATTCCAGAGTACGATGCCTATTTGGACA
TGGTTGACGGAGCTTCATGCTGCTTAGACACTGCCAGTTTTTGCCCTGCAAAGCTGCGCAGCTTTCCAAAGAAAC
ACTCCTATTTGGAACCCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGGCGGCCTTTAATGTGG
AATGCTTCAAGAAATATGCGTGTAATAATGAATATTGGGAAACGTTTAAAGAAAACCCCATCAGGCTTACTGAAG
AAAACGTGGTAAATTACATTACCAAATTAAAAGGACCAAAAGCTGCTGCTCTTTTTGCGAAGACACATAATTTGA
ATATGTTGCAGGACATACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAGCGTATCTGTGCGGAA
TCCACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGTTTG
ATAAAAGTGAGGACGACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTAAATTTAAATTCGGAG
CCATGATGAAATCTGGAATGTTCCTCACACTGTTTGTGAACACAGTCATTAACATTGTAATCGCAAGCAGAGTGT
TGAGAGAACGGCTAACCGGATCACCATGTGCAGCATTCATTGGAGATGACAATATCGTGAAAGGAGTCAAATCGG
ACAAATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACCCCC
TAAAAAGGCTGTTTAAGCTTGGCAAACCTCTGGCAGCAGACGATGAACATGATGATGACAGGAGAAGGGCATTGC
ATGAAGAGTCAACACGCTGGAACCGAGTGGGTATTCTTTCAGAGCTGTGCAAGGCAGTAGAATCAAGGTATGAAA
CCGTAGGAACTTCCATCATAGTTATGGCCATGACTACTCTAGCTAGCAGTGTTAAATCATTCAGCTACCTGAGAG

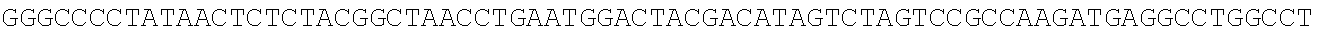

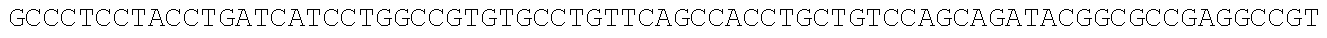

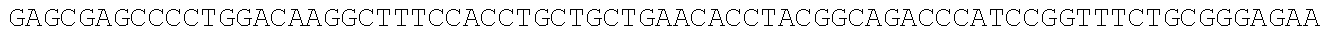

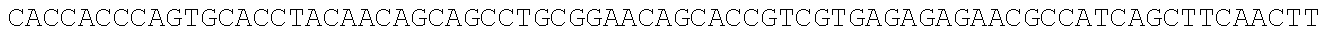

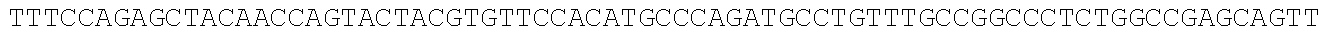

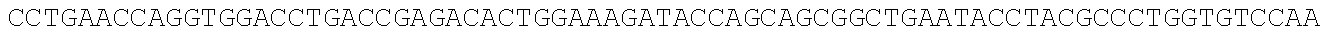

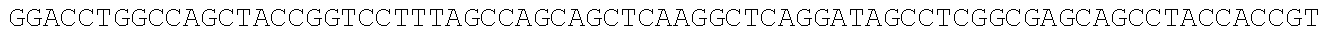

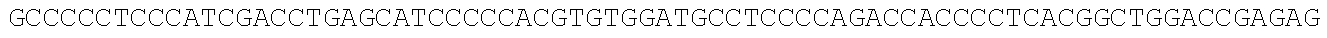

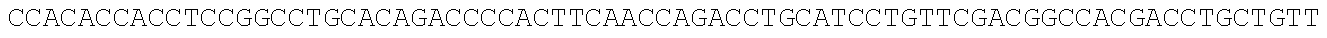

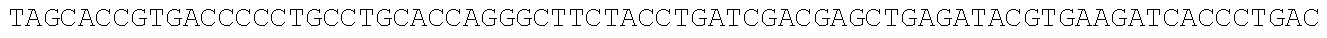

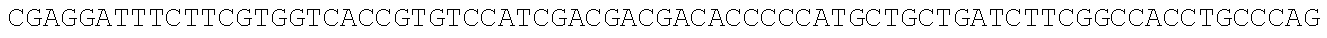

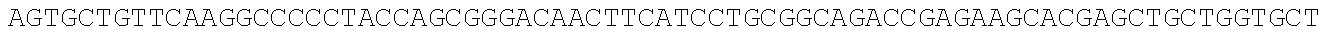

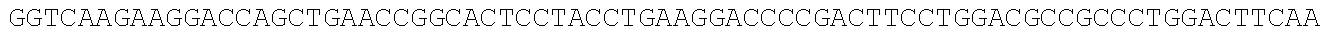

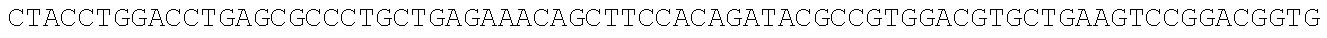

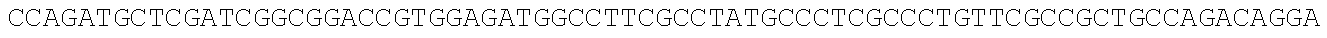

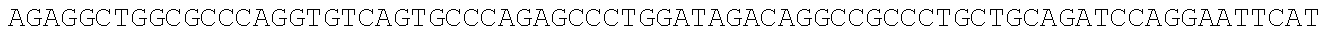

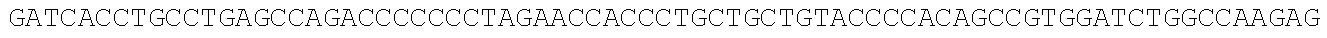

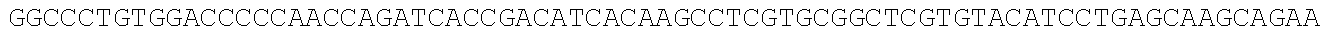

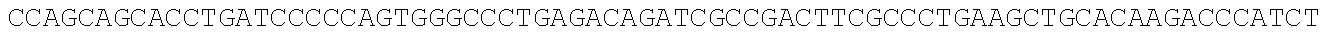

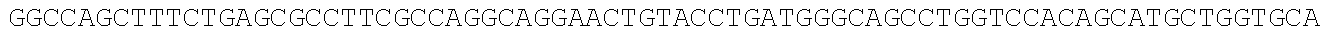

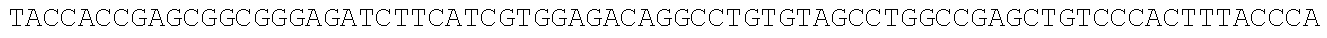

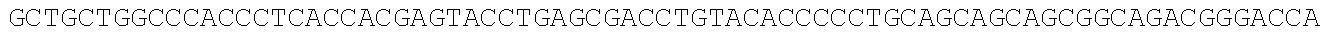

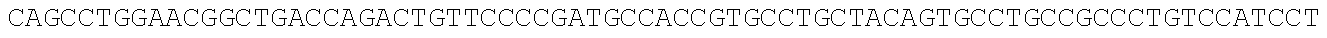

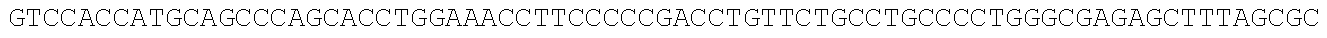

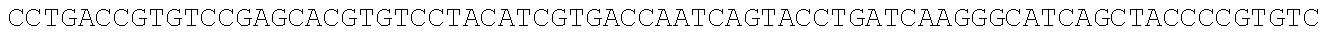

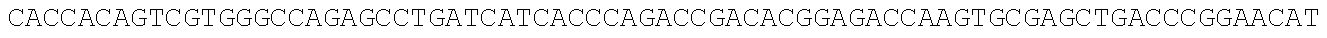

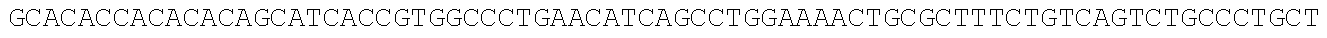

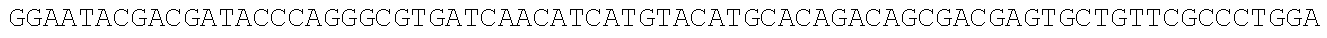

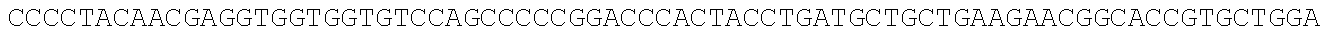

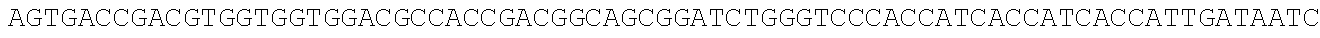

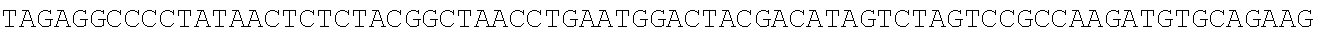

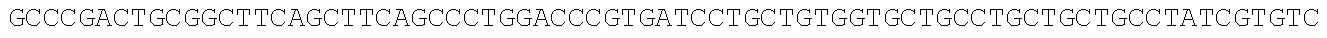

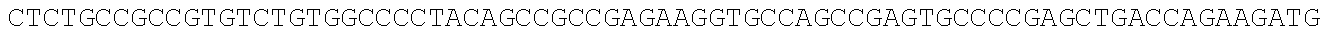

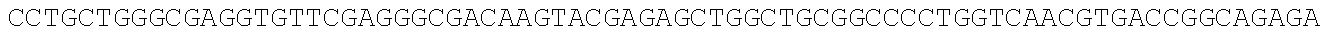

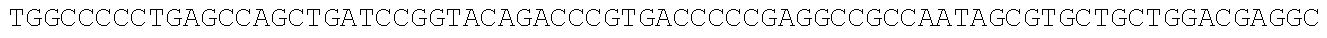

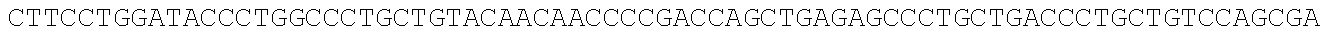

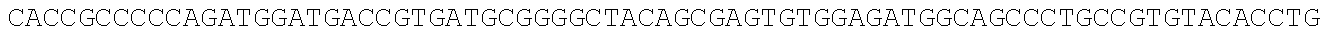

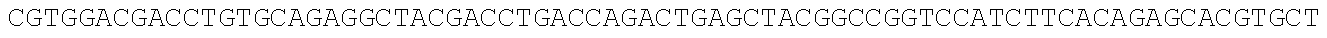

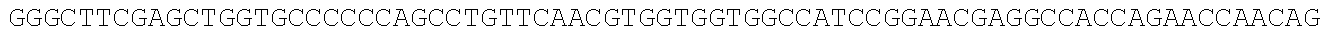

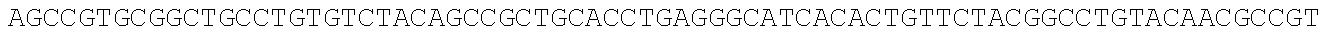

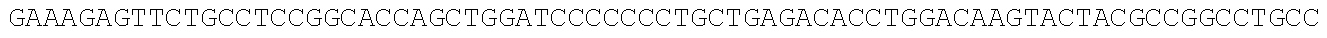

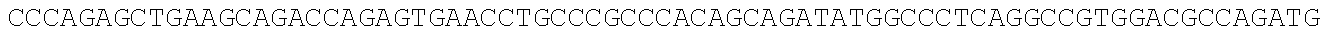

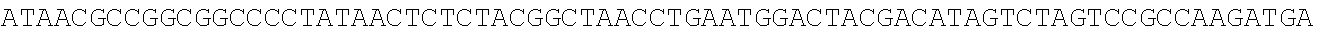

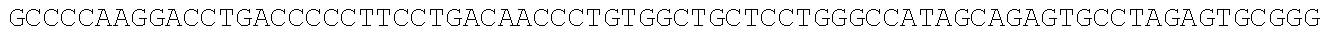

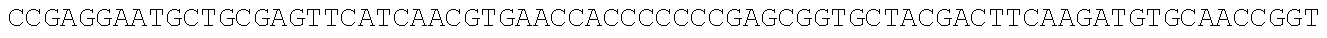

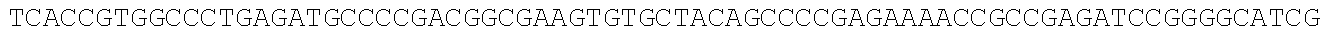

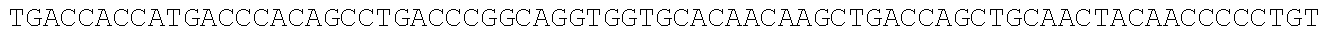

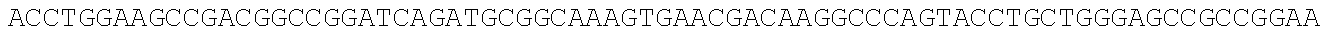

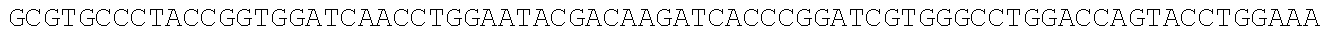

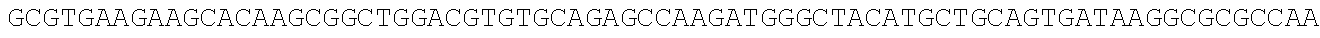

CGTTACTGGCCGAAGCCGCTTGGAATAAGGCCGGTGTGCGTTTGTCTATATGTTATTTTCCACCATATTGCCGTC
TTTTGGCAATGTGAGGGCCCGGAAACCTGGCCCTGTCTTCTTGACGAGCATTCCTAGGGGTCTTTCCCCTCTCGC
CAAAGGAATGCAAGGTCTGTTGAATGTCGTGAAGGAAGCAGTTCCTCTGGAAGCTTCTTGAAGACAAACAACGTC
TGTAGCGACCCTTTGCAGGCAGCGGAACCCCCCACCTGGCGACAGGTGCCTCTGCGGCCAAAAGCCACGTGTATA
AGATACACCTGCAAAGGCGGCACAACCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCCATTGTATGGGATCTGATCTGGGGCC
TCGGTGCACATGCTTTACATGTGTTTAGTCGAGGTTAAAAAAACGTCTAGGCCCCCCGAACCACGGGGACGTGGT

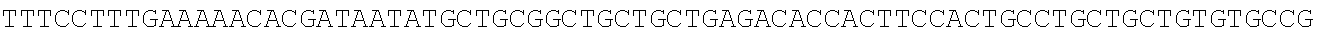

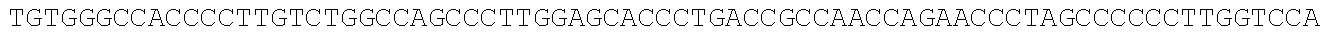

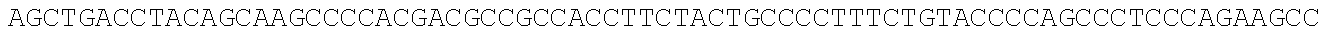

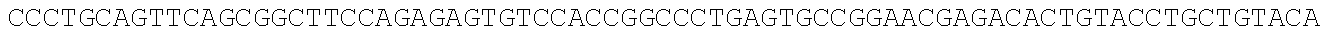

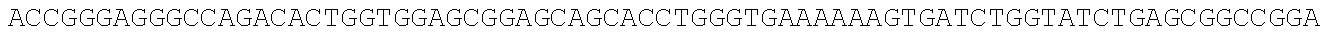

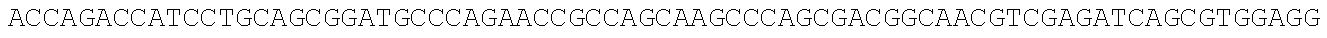

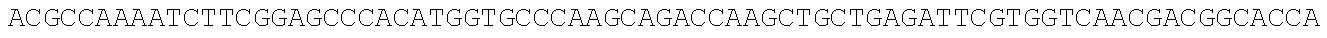

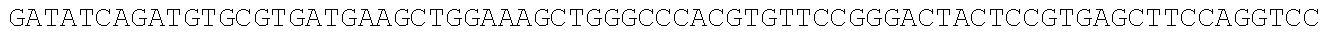

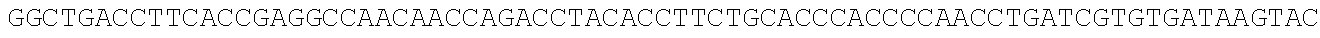

CTTTGTACGCCTGTTTTATACCCCCTCCCTGATTTGCAACTTAGAAGCAACGCAAACCAGATCAATAGTAGGTGT
GACATACCAGTCGCATCTTGATCAAGCACTTCTGTATCCCCGGACCGAGTATCAATAGACTGTGCACACGGTTGA
AGGAGAAAACGTCCGTTACCCGGCTAACTACTTCGAGAAGCCTAGTAACGCCATTGAAGTTGCAGAGTGTTTCGC
TCAGCACTCCCCCCGTGTAGATCAGGTCGATGAGTCACCGCATTCCCCACGGGCGACCGTGGCGGTGGCTGCGTT
GGCGGCCTGCCTATGGGGTAACCCATAGGACGCTCTAATACGGACATGGCGTGAAGAGTCTATTGAGCTAGTTAG
TAGTCCTCCGGCCCCTGAATGCGGCTAATCCTAACTGCGGAGCACATACCCTTAATCCAAAGGGCAGTGTGTCGT
AACGGGCAACTCTGCAGCGGAACCGACTACTTTGGGTGTCCGTGTTTCTTTTTATTCTTGTATTGGCTGCTTATG
GTGACAATTAAAGAATTGTTACCATATAGCTATTGGATTGGCCATCCAGTGTCAAACAGAGCTATTGTATATCTC
TTTGTTGGATTCACACCTCTCACTCTTGAAACGTTACACACCCTCAATTACATTATACTGCTGAACACGAAGCGC

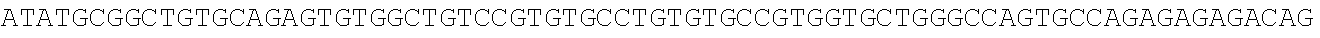

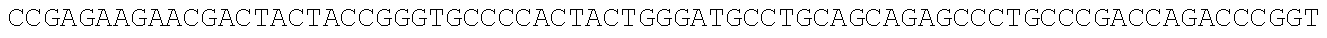

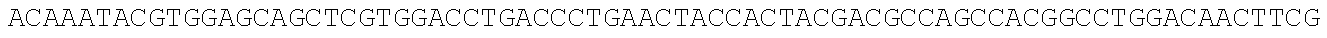

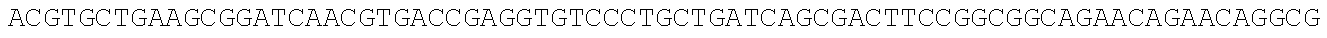

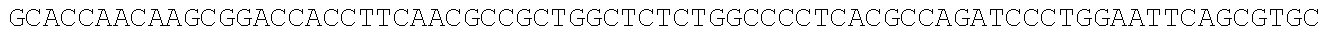

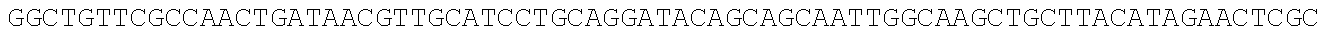

GGCGATTGGCATGCCGCCTTAAAATTTTTATTTTATTTTTCTTTTCTTTTCCGAATCGGATTTTGTTTTTAATAT
TTCAAAAAAAAAAAAAAAAAAAAAAAAAAAAAAAAAAAGGGTCGGCATGGCATCTCCACCTCCTCGCGGTCCGAC
CTGGGCATCCGAAGGAGGACGCACGTCCACTCGGATGGCTAAGGGAGAGCCACGTTTAAACGCTAGAGCAAGACG
TTTCCCGTTGAATATGGCTCATAACACCCCTTGTATTACTGTTTATGTAAGCAGACAGTTTTATTGTTCATGATG
ATATATTTTTATCTTGTGCAATGTAACATCAGAGATTTTGAGACACAACGTGGCTTTGTTGAATAAATCGAACTT
TTGCTGAGTTGAAGGATCAGATCACGCATCTTCCCGACAACGCAGACCGTTCCGTGGCAAAGCAAAAGTTCAAAA
TCACCAACTGGTCCACCTACAACAAAGCTCTCATCAACCGTGGCTCCCTCACTTTCTGGCTGGATGATGGGGCGA
TTCAGGCCTGGTATGAGTCAGCAACACCTTCTTCACGAGGCAGACCTCAGCGCTAGCGGAGTGTATACTGGCTTA
CTATGTTGGCACTGATGAGGGTGTCAGTGAAGTGCTTCATGTGGCAGGAGAAAAAAGGCTGCACCGGTGCGTCAG
CAGAATATGTGATACAGGATATATTCCGCTTCCTCGCTCACTGACTCGCTACGCTCGGTCGTTCGACTGCGGCGA
GCGGAAATGGCTTACGAACGGGGCGGAGATTTCCTGGAAGATGCCAGGAAGATACTTAACAGGGAAGTGAGAGGG
CCGCGGCAAAGCCGTTTTTCCATAGGCTCCGCCCCCCTGACAAGCATCACGAAATCTGACGCTCAAATCAGTGGT
GGCGAAACCCGACAGGACTATAAAGATACCAGGCGTTTCCCCTGGCGGCTCCCTCGTGCGCTCTCCTGTTCCTGC
CTTTCGGTTTACCGGTGTCATTCCGCTGTTATGGCCGCGTTTGTCTCATTCCACGCCTGACACTCAGTTCCGGGT
AGGCAGTTCGCTCCAAGCTGGACTGTATGCACGAACCCCCCGTTCAGTCCGACCGCTGCGCCTTATCCGGTAACT
ATCGTCTTGAGTCCAACCCGGAAAGACATGCAAAAGCACCACTGGCAGCAGCCACTGGTAATTGATTTAGAGGAG
TTAGTCTTGAAGTCATGCGCCGGTTAAGGCTAAACTGAAAGGACAAGTTTTGGTGACTGCGCTCCTCCAAGCCAG
TTACCTCGGTTCAAAGAGTTGGTAGCTCAGAGAACCTTCGAAAAACCGCCCTGCAAGGCGGTTTTTTCGTTTTCA
GAGCAAGAGATTACGCGCAGACCAAAACGATCTCAAGAAGATCATCTTATTAAGGGGTCTGACGCTCAGTGGAAC
GAAAACTCACGTTAAGGGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTATTAGAAAAATTCATCCAGCAGA
CGATAAAACGCAATACGCTGGCTATCCGGTGCCGCAATGCCATACAGCACCAGAAAACGATCCGCCCATTCGCCG
CCCAGTTCTTCCGCAATATCACGGGTGGCCAGCGCAATATCCTGATAACGATCCGCCACGCCCAGACGGCCGCAA
TCAATAAAGCCGCTAAAACGGCCATTTTCCACCATAATGTTCGGCAGGCACGCATCACCATGGGTCACCACCAGA
TCTTCGCCATCCGGCATGCTCGCTTTCAGACGCGCAAACAGCTCTGCCGGTGCCAGGCCCTGATGTTCTTCATCC
AGATCATCCTGATCCACCAGGCCCGCTTCCATACGGGTACGCGCACGTTCAATACGATGTTTCGCCTGATGATCA
AACGGACAGGTCGCCGGGTCCAGGGTATGCAGACGACGCATGGCATCCGCCATAATGCTCACTTTTTCTGCCGGC
GCCAGATGGCTAGACAGCAGATCCTGACCCGGCACTTCGCCCAGCAGCAGCCAATCACGGCCCGCTTCGGTCACC
ACATCCAGCACCGCCGCACACGGAACACCGGTGGTGGCCAGCCAGCTCAGACGCGCCGCTTCATCCTGCAGCTCG
TTCAGCGCACCGCTCAGATCGGTTTTCACAAACAGCACCGGACGACCCTGCGCGCTCAGACGAAACACCGCCGCA
TCAGAGCAGCCAATGGTCTGCTGCGCCCAATCATAGCCAAACAGACGTTCCACCCACGCTGCCGGGCTACCCGCA
TGCAGGCCATCCTGTTCAATCATACTCTTCCTTTTTCAATATTATTGAAGCATTTATCAGGGTTATTGTCTCATG
AGCGGATACATATTTGAATGTATTTAGAAAAATAAACAAATAGGGGTTCCGCGCACATTTCCCCGAAAAGTGCCA
CCTAAATTGTAAGCGTTAATATTTTGTTAAAATTCGCGTTAAATTTTTGTTAAATCAGCTCATTTTTTAACCAAT
AGGCCGAAATCGGCAAAATCCCTTATAAATCAAAAGAATAGACCGAGATAGGGTTGAGTGGCCGCTACAGGGCGC
TCCCATTCGCCATTCAGGCTGCGCAACTGTTGGGAAGGGCGTTTCGGTGCGGGCCTCTTCGCTATTACGCCAGCT
GGCGAAAGGGGGATGTGCTGCAAGGCGATTAAGTTGGGTAACGCCAGGGTTTTCCCAGTCACACGCGTAATACGA
CTCACTATAG
VEE-based replicon encoding eGFP
(SEQ ID NO: 42)

| | nsP1 |
|---|---|
| | ~~~~~~~~~~~~~~~~ |
| 1 | ATAGGCGGCG CATGAGAGAA GCCCAGACCA ATTACCTACC CAAAATGGAG AAAGTTCACG |
| | nsP1 |
| | ~~~~~~~~~~~~~~~~~~~~~~~~~~~~~~~~~~~~~~~~~~~~~~~~~~~~~~~~~~~~~~~~~ |
| 61 | TTGACATCGA GGAAGACAGC CCATTCCTCA GAGCTTTGCA GCGGAGCTTC CCGCAGTTTG |
| | nsP1 |
| | ~~~~~~~~~~~~~~~~~~~~~~~~~~~~~~~~~~~~~~~~~~~~~~~~~~~~~~~~~~~~~~~~~ |
| 121 | AGGTAGAAGC CAAGCAGGTC ACTGATAATG ACCATGCTAA TGCCAGAGCG TTTTCGCATC |
| | nsP1 |
| | ~~~~~~~~~~~~~~~~~~~~~~~~~~~~~~~~~~~~~~~~~~~~~~~~~~~~~~~~~~~~~~~~~ |
| 181 | TGGCTTCAAA ACTGATCGAA ACGGAGGTGG ACCCATCCGA CACGATCCTT GACATTGGAA |
| | nsP1 |
| | ~~~~~~~~~~~~~~~~~~~~~~~~~~~~~~~~~~~~~~~~~~~~~~~~~~~~~~~~~~~~~~~~~ |
| 241 | GTGCGCCCGC CCGCAGAATG TATTCTAAGC ACAAGTATCA TTGTATCTGT CCGATGAGAT |
| | nsP1 |
| | ~~~~~~~~~~~~~~~~~~~~~~~~~~~~~~~~~~~~~~~~~~~~~~~~~~~~~~~~~~~~~~~~~ |
| 301 | GTGCGGAAGA TCCGGACAGA TTGTATAAGT ATGCAACTAA GCTGAAGAAA AACTGTAAGG |
| | nsP1 |
| | ~~~~~~~~~~~~~~~~~~~~~~~~~~~~~~~~~~~~~~~~~~~~~~~~~~~~~~~~~~~~~~~~~ |
| 361 | AAATAACTGA TAAGGAATTG GACAAGAAAA TGAAGGAGCT CGCCGCCGTC ATGAGCGACC |
| | nsP1 |
| | ~~~~~~~~~~~~~~~~~~~~~~~~~~~~~~~~~~~~~~~~~~~~~~~~~~~~~~~~~~~~~~~~~ |
| 421 | CTGACCTGGA AACTGAGACT ATGTGCCTCC ACGACGACGA GTCGTGTCGC TACGAAGGGC |
| | nsP1 |
| | ~~~~~~~~~~~~~~~~~~~~~~~~~~~~~~~~~~~~~~~~~~~~~~~~~~~~~~~~~~~~~~~~~ |
| 481 | AAGTCGCTGT TTACCAGGAT GTATACGCGG TTGACGGACC GACAAGTCTC TATCACCAAG |
| | nsP1 |
| | ~~~~~~~~~~~~~~~~~~~~~~~~~~~~~~~~~~~~~~~~~~~~~~~~~~~~~~~~~~~~~~~~~ |
| 541 | CCAATAAGGG AGTTAGAGTC GCCTACTGGA TAGGCTTTGA CACCACCCCT TTTATGTTTA |
| | nsP1 |
| | ~~~~~~~~~~~~~~~~~~~~~~~~~~~~~~~~~~~~~~~~~~~~~~~~~~~~~~~~~~~~~~~~~ |
| 601 | AGAACTTGGC TGGAGCATAT CCATCATACT CTACCAACTG GGCCGACGAA ACCGTGTTAA |
| | nsP1 |
| | ~~~~~~~~~~~~~~~~~~~~~~~~~~~~~~~~~~~~~~~~~~~~~~~~~~~~~~~~~~~~~~~~~ |
| 661 | CGGCTCGTAA CATAGGCCTA TGCAGCTCTG ACGTTATGGA GCGGTCACGT AGAGGGATGT |
| | nsP1 |
| | ~~~~~~~~~~~~~~~~~~~~~~~~~~~~~~~~~~~~~~~~~~~~~~~~~~~~~~~~~~~~~~~~~ |
| 721 | CCATTCTTAG AAAGAAGTAT TTGAAACCAT CCAACAATGT TCTATTCTCT GTTGGCTCGA |
| | nsP1 |
| | ~~~~~~~~~~~~~~~~~~~~~~~~~~~~~~~~~~~~~~~~~~~~~~~~~~~~~~~~~~~~~~~~~ |
| 781 | CCATCTACCA CGAGAAGAGG GACTTACTGA GGAGCTGGCA CCTGCCGTCT GTATTTCACT |
| | nsP1 |
| | ~~~~~~~~~~~~~~~~~~~~~~~~~~~~~~~~~~~~~~~~~~~~~~~~~~~~~~~~~~~~~~~~~ |
| 841 | TACGTGGCAA GCAAAATTAC ACATGTCGGT GTGAGACTAT AGTTAGTTGC GACGGGTACG |
| | nsP1 |
| | ~~~~~~~~~~~~~~~~~~~~~~~~~~~~~~~~~~~~~~~~~~~~~~~~~~~~~~~~~~~~~~~~~ |
| 901 | TCGTTAAAAG AATAGCTATC AGTCCAGGCC TGTATGGGAA GCCTTCAGGC TATGCTGCTA |
| | nsP1 |
| | ~~~~~~~~~~~~~~~~~~~~~~~~~~~~~~~~~~~~~~~~~~~~~~~~~~~~~~~~~~~~~~~~~ |
| 961 | CGATGCACCG CGAGGGATTC TTGTGCTGCA AAGTGACAGA CACATTGAAC GGGGAGAGGG |
| | nsP1 |
| | ~~~~~~~~~~~~~~~~~~~~~~~~~~~~~~~~~~~~~~~~~~~~~~~~~~~~~~~~~~~~~~~~~ |
| 1021 | TCTCTTTTCC CGTGTGCACG TATGTGCCAG CTACATTGTG TGACCAAATG ACTGGCATAC |
| | nsP1 |
| | ~~~~~~~~~~~~~~~~~~~~~~~~~~~~~~~~~~~~~~~~~~~~~~~~~~~~~~~~~~~~~~~~~ |
| 1081 | TGGCAACAGA TGTCAGTGCG GACGACGCGC AAAAACTGCT GGTTGGGCTC AACCAGCGTA |
| | nsP1 |
| | ~~~~~~~~~~~~~~~~~~~~~~~~~~~~~~~~~~~~~~~~~~~~~~~~~~~~~~~~~~~~~~~~~ |
| 1141 | TAGTCGTCAA CGGTCGCACC CAGAGAAACA CCAATACCAT GAAAAATTAC CTTTTGCCCG |
| | nsP1 |
| | ~~~~~~~~~~~~~~~~~~~~~~~~~~~~~~~~~~~~~~~~~~~~~~~~~~~~~~~~~~~~~~~~~ |
| 1201 | TAGTGGCCCA GGCATTTGCT AGGTGGGCAA AGGAATATAA GGAAGATCAA GAAGATGAAA |
| | nsP1 |
| | ~~~~~~~~~~~~~~~~~~~~~~~~~~~~~~~~~~~~~~~~~~~~~~~~~~~~~~~~~~~~~~~~~ |
| 1261 | GGCCACTAGG ACTACGAGAT AGACAGTTAG TCATGGGGTG TTGTTGGGCT TTTAGAAGGC |
| | nsP1 |
| | ~~~~~~~~~~~~~~~~~~~~~~~~~~~~~~~~~~~~~~~~~~~~~~~~~~~~~~~~~~~~~~~~~ |
| 1321 | ACAAGATAAC ATCTATTTAT AAGCGCCCGG ATACCCAAAC CATCATCAAA GTGAACAGCG |
| | nsP1 |
| | ~~~~~~~~~~~~~~~~~~~~~~~~~~~~~~~~~~~~~~~~~~~~~~~~~ |
| 1381 | ATTTCCACTC ATTCGTGCTG CCCAGGATAG GCAGTAACAC ATTGGAGATC GGGCTGAGAA |
| | nsP1 |
| | ~~~~~~~~~~~~~~~~~~~~~~~~~~~~~~~~~~~~~~~~~~~~~~~~~ |
| 1441 | CAAGAATCAG GAAAATGTTA GAGGAGCACA AGGAGCCGTC ACCTCTCATT ACCGCCGAGG |
| | nsP1 |
| | ~~~~~~~~~~~~~~~~~~~~~~~~~~~~~~~~~~~~~~~~~~~~~~~~~~~~~~~~~~~~~~~~~ |
| 1501 | ACGTACAAGA AGCTAAGTGC GCAGCCGATG AGGCTAAGGA GGTGCGTGAA GCCGAGGAGT |
| | nsP1 |
| | ~~~~~~~~~~~~~~~~~~~~~~~~~~~~~~~~~~~~~~~~~~~~~~~~~~~~~~~~~~~~~~~~~ |
| 1561 | TGCGCGCAGC TCTACCACCT TTGGCAGCTG ATGTTGAGGA GCCCACTCTG GAAGCCGATG |
| | nsP2 |
| | ~~~~~~~~~~~~~~~~~~~~~~~~~~~~~ |
| | nsP1 |
| | ~~~~~~~~~~~~~~~~~~~~~~~ |
| 1621 | TAGACTTGAT GTTACAAGAG GCTGGGGCCG GCTCAGTGGA GACACCTCGT GGCTTGATAA |
| | nsP2 |
| | ~~~~~~~~~~~~~~~~~~~~~~~~~~~~~~~~~~~~~~~~~~~~~~~~~~~~~~~~~~~~~~~~~ |
| 1681 | AGGTTACCAG CTACGATGGC GAGGACAAGA TCGGCTCTTA CGCTGTGCTT TCTCCGCAGG |
| | nsP2 |
| | ~~~~~~~~~~~~~~~~~~~~~~~~~~~~~~~~~~~~~~~~~~~~~~~~~~~~~~~~~~~~~~~~~ |
| 1741 | CTGTACTCAA GAGTGAAAAA TTATCTTGCA TCCACCCTCT CGCTGAACAA GTCATAGTGA |
| | nsP2 |
| | ~~~~~~~~~~~~~~~~~~~~~~~~~~~~~~~~~~~~~~~~~~~~~~~~~~~~~~~~~~~~~~~~~ |
| 1801 | TAACACACTC TGGCCGAAAA GGGCGTTATG CCGTGGAACC ATACCATGGT AAAGTAGTGG |
| | nsP2 |
| | ~~~~~~~~~~~~~~~~~~~~~~~~~~~~~~~~~~~~~~~~~~~~~~~~~~~~~~~~~~~~~~~~~ |
| 1861 | TGCCAGAGGG ACATGCAATA CCCGTCCAGG ACTTTCAAGC TCTGAGTGAA AGTGCCACCA |
| | nsP2 |
| | ~~~~~~~~~~~~~~~~~~~~~~~~~~~~~~~~~~~~~~~~~~~~~~~~~~~~~~~~~~~~~~~~~ |
| 1921 | TTGTGTACAA CGAACGTGAG TTCGTAAACA GGTACCTGCA CCATATTGCC ACACATGGAG |
| | nsP2 |
| | ~~~~~~~~~~~~~~~~~~~~~~~~~~~~~~~~~~~~~~~~~~~~~~~~~~~~~~~~~~~~~~~~~ |
| 1981 | GAGCGCTGAA CACTGATGAA GAATATTACA AAACTGTCAA GCCCAGCGAG CACGACGGCG |
| | nsP2 |
| | ~~~~~~~~~~~~~~~~~~~~~~~~~~~~~~~~~~~~~~~~~~~~~~~~~~~~~~~~~~~~~~~~~ |
| 2041 | AATACCTGTA CGACATCGAC AGGAAACAGT GCGTCAAGAA AGAACTAGTC ACTGGGCTAG |
| | nsP2 |
| | ~~~~~~~~~~~~~~~~~~~~~~~~~~~~~~~~~~~~~~~~~~~~~~~~~~~~~~~~~~~~~~~~~ |
| 2101 | GGCTCACAGG CGAGCTGGTG GATCCTCCCT TCCATGAATT CGCCTACGAG AGTCTGAGAA |
| | nsP2 |
| | ~~~~~~~~~~~~~~~~~~~~~~~~~~~~~~~~~~~~~~~~~~~~~~~~~~~~~~~~~~~~~~~~~ |
| 2161 | CACGACCAGC CGCTCCTTAC CAAGTACCAA CCATAGGGGT GTATGGCGTG CCAGGATCAG |
| | nsP2 |
| | ~~~~~~~~~~~~~~~~~~~~~~~~~~~~~~~~~~~~~~~~~~~~~~~~~~~~~~~~~~~~~~~~~ |
| 2221 | GCAAGTCTGG CATCATTAAA AGCGCAGTCA CCAAAAAAGA TCTAGTGGTG AGCGCCAAGA |
| | nsP2 |
| | ~~~~~~~~~~~~~~~~~~~~~~~~~~~~~~~~~~~~~~~~~~~~~~~~~~~~~~~~~~~~~~~~~ |
| 2281 | AAGAAAACTG TGCAGAAATT ATAAGGGACG TCAAGAAAAT GAAAGGGCTG GACGTCAATG |
| | nsP2 |
| | ~~~~~~~~~~~~~~~~~~~~~~~~~~~~~~~~~~~~~~~~~~~~~~~~~~~~~~~~~~~~~~~~~ |
| 2341 | CCAGAACTGT GGACTCAGTG CTCTTGAATG GATGCAAACA CCCCGTAGAG ACCCTGTATA |
| | nsP2 |
| | ~~~~~~~~~~~~~~~~~~~~~~~~~~~~~~~~~~~~~~~~~~~~~~~~~~~~~~~~~~~~~~~~~ |
| 2401 | TTGACGAAGC TTTTGCTTGT CATGCAGGTA CTCTCAGAGC GCTCATAGCC ATTATAAGAC |
| | nsP2 |
| | ~~~~~~~~~~~~~~~~~~~~~~~~~~~~~~~~~~~~~~~~~~~~~~~~~~~~~~~~~~~~~~~~~ |
| 2461 | CTAAAAAGGC AGTGCTCTGC GGGGATCCCA AACAGTGCGG TTTTTTTAAC ATGATGTGCC |
| | nsP2 |
| | ~~~~~~~~~~~~~~~~~~~~~~~~~~~~~~~~~~~~~~~~~~~~~~~~~~~~~~~~~~~~~~~~~ |
| 2521 | TGAAAGTGCA TTTTAACCAC GAGATTTGCA CACAAGTCTT CCACAAAAGC ATCTCTCGCC |
| | nsP2 |
| | ~~~~~~~~~~~~~~~~~~~~~~~~~~~~~~~~~~~~~~~~~~~~~~~~~~~~~~~~~~~~~~~~~ |
| 2581 | GTTGCACTAA ATCTGTGACT TCGGTCGTCT CAACCTTGTT TTACGACAAA AAAATGAGAA |
| | nsP2 |
| | ~~~~~~~~~~~~~~~~~~~~~~~~~~~~~~~~~~~~~~~~~~~~~~~~~~~~~~~~~~~~~~~~~ |
| 2641 | CGACGAATCC GAAAGAGACT AAGATTGTGA TTGACACTAC CGGCAGTACC AAACCTAAGC |
| | nsP2 |
| | ~~~~~~~~~~~~~~~~~~~~~~~~~~~~~~~~~~~~~~~~~~~~~~~~~~~~~~~~~~~~~~~~~ |
| 2701 | AGGACGATCT CATTCTCACT TGTTTCAGAG GGTGGGTGAA GCAGTTGCAA ATAGATTACA |
| | nsP2 |
| | ~~~~~~~~~~~~~~~~~~~~~~~~~~~~~~~~~~~~~~~~~~~~~~~~~~~~~~~~~~~~~~~~~ |
| 2761 | AAGGCAACGA AATAATGACG GCAGCTGCCT CTCAAGGGCT GACCCGTAAA GGTGTGTATG |
| | nsP2 |
| | ~~~~~~~~~~~~~~~~~~~~~~~~~~~~~~~~~~~~~~~~~~~~~~~~~~~~~~~~~~~~~~~~~ |
| 2821 | CCGTTCGGTA CAAGGTGAAT GAAAATCCTC TGTACGCACC CACCTCAGAA CATGTGAACG |
| | nsP2 |
| | ~~~~~~~~~~~~~~~~~~~~~~~~~~~~~~~~~~~~~~~~~~~~~~~~~~~~~~~~~~~~~~~~~ |
| 2881 | TCCTACTGAC CCGCACGGAG GACCGCATCG TGTGGAAAAC ACTAGCCGGC GACCCATGGA |
| | nsP2 |
| | ~~~~~~~~~~~~~~~~~~~~~~~~~~~~~~~~~~~~~~~~~~~~~~~~~~~~~~~~~~~~~~~~~ |
| 2941 | TAAAAACACT GACTGCCAAG TACCCTGGGA ATTTCACTGC CACGATAGAG GAGTGGCAAG |
| | nsP2 |
| | ~~~~~~~~~~~~~~~~~~~~~~~~~~~~~~~~~~~~~~~~~~~~~~~~~~~~~~~~~~~~~~~~~ |
| 3001 | CAGAGCATGA TGCCATCATG AGGCACATCT TGGAGAGACC GGACCCTACC GACGTCTTCC |
| | nsP2 |
| | ~~~~~~~~~~~~~~~~~~~~~~~~~~~~~~~~~~~~~~~~~~~~~~~~~~~~~~~~~~~~~~~~~ |
| 3061 | AGAATAAGGC AAACGTGTGT TGGGCCAAGG CTTTAGTGCC GGTGCTGAAG ACCGCTGGCA |
| | nsP2 |
| | ~~~~~~~~~~~~~~~~~~~~~~~~~~~~~~~~~~~~~~~~~~~~~~~~~~~~~~~~~~~~~~~~~ |
| 3121 | TAGACATGAC CACTGAACAA TGGAACACTG TGGATTATTT TGAAACGGAC AAAGCTCACT |
| | nsP2 |
| | ~~~~~~~~~~~~~~~~~~~~~~~~~~~~~~~~~~~~~~~~~~~~~~~~~~~~~~~~~~~~~~~~~ |
| 3181 | CAGCAGAGAT AGTATTGAAC CAACTATGCG TGAGGTTCTT TGGACTCGAT CTGGACTCCG |
| | nsP2 |
| | ~~~~~~~~~~~~~~~~~~~~~~~~~~~~~~~~~~~~~~~~~~~~~~~~~~~~~~~~~~~~~~~~~ |
| 3241 | GTCTATTTTC TGCACCCACT GTTCCGTTAT CCATTAGGAA TAATCACTGG GATAACTCCC |
| | nsP2 |
| | ~~~~~~~~~~~~~~~~~~~~~~~~~~~~~~~~~~~~~~~~~~~~~~~~~~~~~~~~~~~~~~~~~ |
| 3301 | CGTCGCCTAA CATGTACGGG CTGAATAAAG AAGTGGTCCG TCAGCTCTCT CGCAGGTACC |
| | nsP2 |
| | ~~~~~~~~~~~~~~~~~~~~~~~~~~~~~~~~~~~~~~~~~~~~~~~~~~~~~~~~~~~~~~~~~ |
| 3361 | CACAACTGCC TCGGGCAGTT GCCACTGGAA GAGTCTATGA CATGAACACT GGTACACTGC |
| | nsP2 |
| | ~~~~~~~~~~~~~~~~~~~~~~~~~~~~~~~~~~~~~~~~~~~~~~~~~~~~~~~~~~~~~~~~~ |
| 3421 | GCAATTATGA TCCGCGCATA AACCTAGTAC CTGTAAACAG AAGACTGCCT CATGCTTTAG |
| | nsP2 |
| | ~~~~~~~~~~~~~~~~~~~~~~~~~~~~~~~~~~~~~~~~~~~~~~~~~~~~~~~~~~~~~~~~~ |
| 3481 | TCCTCCACCA TAATGAACAC CCACAGAGTG ACTTTTCTTC ATTCGTCAGC AAATTGAAGG |
| | nsP2 |
| | ~~~~~~~~~~~~~~~~~~~~~~~~~~~~~~~~~~~~~~~~~~~~~~~~~~~~~~~~~~~~~~~~~ |
| 3541 | GCAGAACTGT CCTGGTGGTC GGGGAAAAGT TGTCCGTCCC AGGCAAAATG GTTGACTGGT |
| | nsP2 |
| | ~~~~~~~~~~~~~~~~~~~~~~~~~~~~~~~~~~~~~~~~~~~~~~~~~~~~~~~~~~~~~~~~~ |
| 3601 | TGTCAGACCG GCCTGAGGCT ACCTTCAGAG CTCGGCTGGA TTTAGGCATC CCAGGTGATG |
| | nsP2 |
| | ~~~~~~~~~~~~~~~~~~~~~~~~~~~~~~~~~~~~~~~~~~~~~~~~~~~~~~~~~~~~~~~~~ |
| 3661 | TGCCCAAATA TGACATAATA TTTGTTAATG TGAGGACCCC ATATAAATAC CATCACTATC |
| | nsP2 |
| | ~~~~~~~~~~~~~~~~~~~~~~~~~~~~~~~~~~~~~~~~~~~~~~~~~~~~~~~~~~~~~~~~~ |
| 3721 | AGCAGTGTGA AGACCATGCC ATTAAGCTTA GCATGTTGAC CAAGAAAGCT TGTCTGCATC |
| | nsP2 |
| | ~~~~~~~~~~~~~~~~~~~~~~~~~~~~~~~~~~~~~~~~~~~~~~~~~~~~~~~~~~~~~~~~~ |
| 3781 | TGAATCCCGG CGGAACCTGT GTCAGCATAG GTTATGGTTA CGCTGACAGG GCCAGCGAAA |
| | nsP2 |
| | ~~~~~~~~~~~~~~~~~~~~~~~~~~~~~~~~~~~~~~~~~~~~~~~~~~~~~~~~~~~~~~~~~ |
| 3841 | GCATCATTGG TGCTATAGCG CGGCAGTTCA AGTTTTCCCG GGTATGCAAA CCGAAATCCT |
| | nsP2 |
| | ~~~~~~~~~~~~~~~~~~~~~~~~~~~~~~~~~~~~~~~~~~~~~~~~~~~~~~~~~~~~~~~~~ |
| 3901 | CACTTGAAGA GACGGAAGTT CTGTTTGTAT TCATTGGGTA CGATCGCAAG GCCCGTACGC |
| | nsP2 |
| | ~~~~~~~~~~~~~~~~~~~~~~~~~~~~~~~~~~~~~~~~~~~~~~~~~~~~~~~~~~~~~~~~~ |
| 3961 | ACAATCCTTA CAAGCTTTCA TCAACCTTGA CCAACATTTA TACAGGTTCC AGACTCCACG |
| | nsP3 |
| | ~~~~~~~~~~~~~~~~~~~~~~~~~~~~~~~~~~~~~~~~~~~~~~~~~~ |
| | nsP2 |
| | ~~~~~~~~~~~~ |
| 4021 | AAGCCGGATG TGCACCCTCA TATCATGTGG TGCGAGGGGA TATTGCCACG GCCACCGAAG |
| | nsP3 |
| | ~~~~~~~~~~~~~~~~~~~~~~~~~~~~~~~~~~~~~~~~~~~~~~~~~~~~~~~~~~~~~~~~~ |
| 4081 | GAGTGATTAT AAATGCTGCT AACAGCAAAG GACAACCTGG CGGAGGGGTG TGCGGAGCGC |
| | nsP3 |
| | ~~~~~~~~~~~~~~~~~~~~~~~~~~~~~~~~~~~~~~~~~~~~~~~~~~~~~~~~~~~~~~~~~ |
| 4141 | TGTATAAGAA ATTCCCGGAA AGCTTCGATT TACAGCCGAT CGAAGTAGGA AAAGCGCGAC |
| | nsP3 |
| | ~~~~~~~~~~~~~~~~~~~~~~~~~~~~~~~~~~~~~~~~~~~~~~~~~~~~~~~~~~~~~~~~~ |
| 4201 | TGGTCAAAGG TGCAGCTAAA CATATCATTC ATGCCGTAGG ACCAAACTTC AACAAAGTTT |
| | nsP3 |
| | ~~~~~~~~~~~~~~~~~~~~~~~~~~~~~~~~~~~~~~~~~~~~~~~~~~~~~~~~~~~~~~~~~ |
| 4261 | CGGAGGTTGA AGGTGACAAA CAGTTGGCAG AGGCTTATGA GTCCATCGCT AAGATTGTCA |
| | nsP3 |
| | ~~~~~~~~~~~~~~~~~~~~~~~~~~~~~~~~~~~~~~~~~~~~~~~~~~~~~~~~~~~~~~~~~ |
| 4321 | ACGATAACAA TTACAAGTCA GTAGCGATTC CACTGTTGTC CACCGGCATC TTTTCCGGGA |
| | nsP3 |
| | ~~~~~~~~~~~~~~~~~~~~~~~~~~~~~~~~~~~~~~~~~~~~~~~~~~~~~~~~~~~~~~~~~ |
| 4381 | ACAAAGATCG ACTAACCCAA TCATTGAACC ATTTGCTGAC AGCTTTAGAC ACCACTGATG |
| | nsP3 |
| | ~~~~~~~~~~~~~~~~~~~~~~~~~~~~~~~~~~~~~~~~~~~~~~~~~~~~~~~~~~~~~~~~~ |
| 4441 | CAGATGTAGC CATATACTGC AGGGACAAGA AATGGGAAAT GACTCTCAAG GAAGCAGTGG |
| | nsP3 |
| | ~~~~~~~~~~~~~~~~~~~~~~~~~~~~~~~~~~~~~~~~~~~~~~~~~~~~~~~~~~~~~~~~~ |
| 4501 | CTAGGAGAGA AGCAGTGGAG GAGATATGCA TATCCGACGA CTCTTCAGTG ACAGAACCTG |
| | nsP3 |
| | ~~~~~~~~~~~~~~~~~~~~~~~~~~~~~~~~~~~~~~~~~~~~~~~~~~~~~~~~~~~~~~~~~ |
| 4561 | ATGCAGAGCT GGTGAGGGTG CATCCGAAGA GTTCTTTGGC TGGAAGGAAG GGCTACAGCA |
| | nsP3 |
| | ~~~~~~~~~~~~~~~~~~~~~~~~~~~~~~~~~~~~~~~~~~~~~~~~~~~~~~~~~~~~~~~~~ |
| 4621 | CAAGCGATGG CAAAACTTTC TCATATTTGG AAGGGACCAA GTTTCACCAG GCGGCCAAGG |
| | nsP3 |
| | ~~~~~~~~~~~~~~~~~~~~~~~~~~~~~~~~~~~~~~~~~~~~~~~~~~~~~~~~~~~~~~~~~ |
| 4681 | ATATAGCAGA AATTAATGCC ATGTGGCCCG TTGCAACGGA GGCCAATGAG CAGGTATGCA |
| | nsP3 |
| | ~~~~~~~~~~~~~~~~~~~~~~~~~~~~~~~~~~~~~~~~~~~~~~~~~~~~~~~~~~~~~~~~~ |
| 4741 | TGTATATCCT CGGAGAAAGC ATGAGCAGTA TTAGGTCGAA ATGCCCCGTC GAAGAGTCGG |
| | nsP3 |
| | ~~~~~~~~~~~~~~~~~~~~~~~~~~~~~~~~~~~~~~~~~~~~~~~~~~~~~~~~~~~~~~~~~ |
| 4801 | AAGCCTCCAC ACCACCTAGC ACGCTGCCTT GCTTGTGCAT CCATGCCATG ACTCCAGAAA |
| | nsP3 |
| | ~~~~~~~~~~~~~~~~~~~~~~~~~~~~~~~~~~~~~~~~~~~~~~~~~~~~~~~~~~~~~~~~~ |
| 4861 | GAGTACAGCG CCTAAAAGCC TCACGTCCAG AACAAATTAC TGTGTGCTCA TCCTTTCCAT |
| | nsP3 |
| | ~~~~~~~~~~~~~~~~~~~~~~~~~~~~~~~~~~~~~~~~~~~~~~~~~~~~~~~~~~~~~~~~~ |
| 4921 | TGCCGAAGTA TAGAATCACT GGTGTGCAGA AGATCCAATG CTCCCAGCCT ATATTGTTCT |
| | nsP3 |
| | ~~~~~~~~~~~~~~~~~~~~~~~~~~~~~~~~~~~~~~~~~~~~~~~~~~~~~~~~~~~~~~~~~ |
| 4981 | CACCGAAAGT GCCTGCGTAT ATTCATCCAA GGAAGTATCT CGTGGAAACA CCACCGGTAG |
| | nsP3 |
| | ~~~~~~~~~~~~~~~~~~~~~~~~~~~~~~~~~~~~~~~~~~~~~~~~~~~~~~~~~~~~~~~~~ |
| 5041 | ACGAGACTCC GGAGCCATCG GCAGAGAACC AATCCACAGA GGGGACACCT GAACAACCAC |
| | nsP3 |
| | ~~~~~~~~~~~~~~~~~~~~~~~~~~~~~~~~~~~~~~~~~~~~~~~~~~~~~~~~~~~~~~~~~ |
| 5101 | CACTTATAAC CGAGGATGAG ACCAGGACTA GAACGCCTGA GCCGATCATC ATCGAAGAGG |
| | nsP3 |
| | ~~~~~~~~~~~~~~~~~~~~~~~~~~~~~~~~~~~~~~~~~~~~~~~~~~~~~~~~~~~~~~~~~ |
| 5161 | AAGAAGAGGA TAGCATAAGT TTGCTGTCAG ATGGCCCGAC CCACCAGGTG CTGCAAGTCG |
| | nsP3 |
| | ~~~~~~~~~~~~~~~~~~~~~~~~~~~~~~~~~~~~~~~~~~~~~~~~~~~~~~~~~~~~~~~~~ |
| 5221 | AGGCAGACAT TCACGGGCCG CCCTCTGTAT CTAGCTCATC CTGGTCCATT CCTCATGCAT |
| | nsP3 |
| | ~~~~~~~~~~~~~~~~~~~~~~~~~~~~~~~~~~~~~~~~~~~~~~~~~~~~~~~~~~~~~~~~~ |
| 5281 | CCGACTTTGA TGTGGACAGT TTATCCATAC TTGACACCCT GGAGGGAGCT AGCGTGACCA |
| | nsP3 |
| | ~~~~~~~~~~~~~~~~~~~~~~~~~~~~~~~~~~~~~~~~~~~~~~~~~~~~~~~~~~~~~~~~~ |
| 5341 | GCGGGGCAAC GTCAGCCGAG ACTAACTCTT ACTTCGCAAA GAGTATGGAG TTTCTGGCGC |
| | nsP3 |
| | ~~~~~~~~~~~~~~~~~~~~~~~~~~~~~~~~~~~~~~~~~~~~~~~~~~~~~~~~~~~~~~~~~ |
| 5401 | GACCGGTGCC TGCGCCTCGA ACAGTATTCA GGAACCCTCC ACATCCCGCT CCGCGCACAA |
| | nsP3 |
| | ~~~~~~~~~~~~~~~~~~~~~~~~~~~~~~~~~~~~~~~~~~~~~~~~~~~~~~~~~~~~~~~~~ |
| 5461 | GAACACCGTC ACTTGCACCC AGCAGGGCCT GCTCGAGAAC CAGCCTAGTT TCCACCCCGC |
| | nsP3 |
| | ~~~~~~~~~~~~~~~~~~~~~~~~~~~~~~~~~~~~~~~~~~~~~~~~~~~~~~~~~~~~~~~~~ |
| 5521 | CAGGCGTGAA TAGGGTGATC ACTAGAGAGG AGCTCGAGGC GCTTACCCCG TCACGCACTC |
| | nsP3 |
| | ~~~~~~~~~~~~~~~~~~~~~~~~~~~~~~~~~~~~~~~~~~~~~~~~~~~~~~~~~~~~~~~~~ |
| 5581 | CTAGCAGGTC GGTCTCGAGA ACCAGCCTGG TCTCCAACCC GCCAGGCGTA AATAGGGTGA |
| | nsP4 |
| | ~~~~~~~~~~~~~~~~ |
| | nsP3 |
| | ~~~~~~~~~~~~~~~~~~~~~~~~~~~~~~~~~~~~~~~~~~~~~ |
| 5641 | TTACAAGAGA GGAGTTTGAG GCGTTCGTAG CACAACAACA ATGACGGTTT GATGCGGGTG |
| | nsP4 |
| | ~~~~~~~~~~~~~~~~~~~~~~~~~~~~~~~~~~~~~~~~~~~~~~~~~~~~~~~~~~~~~~~~~ |
| 5701 | CATACATCTT TTCCTCCGAC ACCGGTCAAG GGCATTTACA ACAAAAATCA GTAAGGCAAA |
| | nsP4 |
| | ~~~~~~~~~~~~~~~~~~~~~~~~~~~~~~~~~~~~~~~~~~~~~~~~~~~~~~~~~~~~~~~~~ |
| 5761 | CGGTGCTATC CGAAGTGGTG TTGGAGAGGA CCGAATTGGA GATTTCGTAT GCCCCGCGCC |
| | nsP4 |
| | ~~~~~~~~~~~~~~~~~~~~~~~~~~~~~~~~~~~~~~~~~~~~~~~~~~~~~~~~~~~~~~~~~ |
| 5821 | TCGACCAAGA AAAAGAAGAA TTACTACGCA AGAAATTACA GTTAAATCCC ACACCTGCTA |
| | nsP4 |
| | ~~~~~~~~~~~~~~~~~~~~~~~~~~~~~~~~~~~~~~~~~~~~~~~~~~~~~~~~~~~~~~~~~ |
| 5881 | ACAGAAGCAG ATACCAGTCC AGGAAGGTGG AGAACATGAA AGCCATAACA GCTAGACGTA |
| | nsP4 |
| | ~~~~~~~~~~~~~~~~~~~~~~~~~~~~~~~~~~~~~~~~~~~~~~~~~~~~~~~~~~~~~~~~~ |
| 5941 | TTCTGCAAGG CCTAGGGCAT TATTTGAAGG CAGAAGGAAA AGTGGAGTGC TACCGAACCC |
| | nsP4 |
| | ~~~~~~~~~~~~~~~~~~~~~~~~~~~~~~~~~~~~~~~~~~~~~~~~~~~~~~~~~~~~~~~~~ |
| 6001 | TGCATCCTGT TCCTTTGTAT TCATCTAGTG TGAACCGTGC CTTTTCAAGC CCCAAGGTCG |
| | nsP4 |
| | ~~~~~~~~~~~~~~~~~~~~~~~~~~~~~~~~~~~~~~~~~~~~~~~~~~~~~~~~~~~~~~~~~ |
| 6061 | CAGTGGAAGC CTGTAACGCC ATGTTGAAAG AGAACTTTCC GACTGTGGCT TCTTACTGTA |
| | nsP4 |
| | ~~~~~~~~~~~~~~~~~~~~~~~~~~~~~~~~~~~~~~~~~~~~~~~~~~~~~~~~~~~~~~~~~ |
| 6121 | TTATTCCAGA GTACGATGCC TATTTGGACA TGGTTGACGG AGCTTCATGC TGCTTAGACA |
| | nsP4 |
| | ~~~~~~~~~~~~~~~~~~~~~~~~~~~~~~~~~~~~~~~~~~~~~~~~~~~~~~~~~~~~~~~~~ |
| 6181 | CTGCCAGTTT TTGCCCTGCA AAGCTGCGCA GCTTTCCAAA GAAACACTCC TATTTGGAAC |
| | nsP4 |
| | ~~~~~~~~~~~~~~~~~~~~~~~~~~~~~~~~~~~~~~~~~~~~~~~~~~~~~~~~~~~~~~~~~ |
| 6241 | CCACAATACG ATCGGCAGTG CCTTCAGCGA TCCAGAACAC GCTCCAGAAC GTCCTGGCAG |
| | nsP4 |
| | ~~~~~~~~~~~~~~~~~~~~~~~~~~~~~~~~~~~~~~~~~~~~~~~~~~~~~~~~~~~~~~~~~ |
| 6301 | CTGCCACAAA AAGAAATTGC AATGTCACGC AAATGAGAGA ATTGCCCGTA TTGGATTCGG |
| | nsP4 |
| | ~~~~~~~~~~~~~~~~~~~~~~~~~~~~~~~~~~~~~~~~~~~~~~~~~~~~~~~~~~~~~~~~~ |
| 6361 | CGGCCTTTAA TGTGGAATGC TTCAAGAAAT ATGCGTGTAA TAATGAATAT TGGGAAACGT |
| | nsP4 |
| | ~~~~~~~~~~~~~~~~~~~~~~~~~~~~~~~~~~~~~~~~~~~~~~~~~~~~~~~~~~~~~~~~~ |
| 6421 | TTAAAGAAAA CCCCATCAGG CTTACTGAAG AAAACGTGGT AAATTACATT ACCAAATTAA |
| | nsP4 |
| | ~~~~~~~~~~~~~~~~~~~~~~~~~~~~~~~~~~~~~~~~~~~~~~~~~~~~~~~~~~~~~~~~~ |
| 6481 | AAGGACCAAA AGCTGCTGCT CTTTTTGCGA AGACACATAA TTTGAATATG TTGCAGGACA |
| | nsP4 |
| | ~~~~~~~~~~~~~~~~~~~~~~~~~~~~~~~~~~~~~~~~~~~~~~~~~~~~~~~~~~~~~~~~~ |
| 6541 | TACCAATGGA CAGGTTTGTA ATGGACTTAA AGAGAGACGT GAAAGTGACT CCAGGAACAA |
| | nsP4 |
| | ~~~~~~~~~~~~~~~~~~~~~~~~~~~~~~~~~~~~~~~~~~~~~~~~~~~~~~~~~~~~~~~~~ |
| 6601 | AACATACTGA AGAACGGCCC AAGGTACAGG TGATCCAGGC TGCCGATCCG CTAGCAACAG |
| | nsP4 |
| | ~~~~~~~~~~~~~~~~~~~~~~~~~~~~~~~~~~~~~~~~~~~~~~~~~~~~~~~~~~~~~~~~~ |
| 6661 | CGTATCTGTG CGGAATCCAC CGAGAGCTGG TTAGGAGATT AAATGCGGTC CTGCTTCCGA |
| | nsP4 |
| | ~~~~~~~~~~~~~~~~~~~~~~~~~~~~~~~~~~~~~~~~~~~~~~~~~~~~~~~~~~~~~~~~~ |
| 6721 | ACATTCATAC ACTGTTTGAT ATGTCGGCTG AAGACTTTGA CGCTATTATA GCCGAGCACT |
| | nsP4 |
| | ~~~~~~~~~~~~~~~~~~~~~~~~~~~~~~~~~~~~~~~~~~~~~~~~~~~~~~~~~~~~~~~~~ |
| 6781 | TCCAGCCTGG GGATTGTGTT CTGGAAACTG ACATCGCGTC GTTTGATAAA AGTGAGGACG |
| | nsP4 |
| | ~~~~~~~~~~~~~~~~~~~~~~~~~~~~~~~~~~~~~~~~~~~~~~~~~~~~~~~~~~~~~~~~~ |
| 6841 | ACGCCATGGC TCTGACCGCG TTAATGATTC TGGAAGACTT AGGTGTGGAC GCAGAGCTGT |
| | nsP4 |
| | ~~~~~~~~~~~~~~~~~~~~~~~~~~~~~~~~~~~~~~~~~~~~~~~~~~~~~~~~~~~~~~~~~ |
| 6901 | TGACGCTGAT TGAGGCGGCT TTCGGCGAAA TTTCATCAAT ACATTTGCCC ACTAAAACTA |
| | nsP4 |
| | ~~~~~~~~~~~~~~~~~~~~~~~~~~~~~~~~~~~~~~~~~~~~~~~~~~~~~~~~~~~~~~~~~ |
| 6961 | AATTTAAATT CGGAGCCATG ATGAAATCTG GAATGTTCCT CACACTGTTT GTGAACACAG |
| | nsP4 |
| | ~~~~~~~~~~~~~~~~~~~~~~~~~~~~~~~~~~~~~~~~~~~~~~~~~~~~~~~~~~~~~~~~~ |
| 7021 | TCATTAACAT TGTAATCGCA AGCAGAGTGT TGAGAGAACG GCTAACCGGA TCACCATGTG |
| | nsP4 |
| | ~~~~~~~~~~~~~~~~~~~~~~~~~~~~~~~~~~~~~~~~~~~~~~~~~~~~~~~~~~~~~~~~~ |
| 7081 | CAGCATTCAT TGGAGATGAC AATATCGTGA AAGGAGTCAA ATCGGACAAA TTAATGGCAG |
| | nsP4 |
| | ~~~~~~~~~~~~~~~~~~~~~~~~~~~~~~~~~~~~~~~~~~~~~~~~~~~~~~~~~~~~~~~~~ |
| 7141 | ACAGGTGCGC CACCTGGTTG AATATGGAAG TCAAGATTAT AGATGCTGTG GTGGGCGAGA |
| | nsP4 |
| | ~~~~~~~~~~~~~~~~~~~~~~~~~~~~~~~~~~~~~~~~~~~~~~~~~~~~~~~~~~~~~~~~~ |
| 7201 | AAGCGCCTTA TTTCTGTGGA GGGTTTATTT TGTGTGACTC CGTGACCGGC ACAGCGTGCC |
| | nsP4 |
| | ~~~~~~~~~~~~~~~~~~~~~~~~~~~~~~~~~~~~~~~~~~~~~~~~~~~~~~~~~~~~~~~~~ |
| 7261 | GTGTGGCAGA CCCCCTAAAA AGGCTGTTTA AGCTTGGCAA ACCTCTGGCA GCAGACGATG |
| | nsP4 |
| | ~~~~~~~~~~~~~~~~~~~~~~~~~~~~~~~~~~~~~~~~~~~~~~~~~~~~~~~~~~~~~~~~~ |
| 7321 | AACATGATGA TGACAGGAGA AGGGCATTGC ATGAAGAGTC AACACGCTGG AACCGAGTGG |
| | nsP4 |
| | ~~~~~~~~~~~~~~~~~~~~~~~~~~~~~~~~~~~~~~~~~~~~~~~~~~~~~~~~~~~~~~~~~ |
| 7381 | GTATTCTTTC AGAGCTGTGC AAGGCAGTAG AATCAAGGTA TGAAACCGTA GGAACTTCCA |
| | nsP4 |
| | ~~~~~~~~~~~~~~~~~~~~~~~~~~~~~~~~~~~~~~~~~~~~~~~~~~~~~~~~~~~~~~~~~ |
| 7441 | TCATAGTTAT GGCCATGACT ACTCTAGCTA GCAGTGTTAA ATCATTCAGC TACCTGAGAG |
| | subgenomic promoter |
| | ~~~~~~~~~~~~~~~~~~~~~~~~~ |
| | nsP4 |
| | ~~~~~~~~~~~~~~~~~~~~~~~~~~~~ |
| 7501 | GGGCCCCTAT AACTCTCTAC GGCTAACCTG AATGGACTAC GACATAGTCT AGTCGACGCC |
| | eGFP |
| | ~~~~~~~~~~~~~~~~~~~~~~~~~~~~~~~~~~~~~~~~~~~~~~~~~~~~~~~~~~~~~~~~~ |
| 7561 | ACCATGGTGA GCAAGGGCGA GGAGCTGTTC ACCGGGGTGG TGCCCATCCT GGTCGAGCTG |
| | eGFP |
| | ~~~~~~~~~~~~~~~~~~~~~~~~~~~~~~~~~~~~~~~~~~~~~~~~~~~~~~~~~~~~~~~~~ |
| 7621 | GACGGCGACG TAAACGGCCA CAAGTTCAGC GTGTCCGGCG AGGGCGAGGG CGATGCCACC |
| | eGFP |
| | ~~~~~~~~~~~~~~~~~~~~~~~~~~~~~~~~~~~~~~~~~~~~~~~~~~~~~~~~~~~~~~~~~ |
| 7681 | TACGGCAAGC TGACCCTGAA GTTCATCTGC ACCACCGGCA AGCTGCCCGT GCCCTGGCCC |
| | eGFP |
| | ~~~~~~~~~~~~~~~~~~~~~~~~~~~~~~~~~~~~~~~~~~~~~~~~~~~~~~~~~~~~~~~~~ |
| 7741 | ACCCTCGTGA CCACCCTGAC CTACGGCGTG CAGTGCTTCA GCCGCTACCC CGACCACATG |
| | eGFP |
| | ~~~~~~~~~~~~~~~~~~~~~~~~~~~~~~~~~~~~~~~~~~~~~~~~~~~~~~~~~~~~~~~~~ |
| 7801 | AAGCAGCACG ACTTCTTCAA GTCCGCCATG CCCGAAGGCT ACGTCCAGGA GCGCACCATC |
| | eGFP |
| | ~~~~~~~~~~~~~~~~~~~~~~~~~~~~~~~~~~~~~~~~~~~~~~~~~~~~~~~~~~~~~~~~~ |
| 7861 | TTCTTCAAGG ACGACGGCAA CTACAAGACC CGCGCCGAGG TGAAGTTCGA GGGCGACACC |
| | eGFP |
| | ~~~~~~~~~~~~~~~~~~~~~~~~~~~~~~~~~~~~~~~~~~~~~~~~~~~~~~~~~~~~~~~~~ |
| 7921 | CTGGTGAACC GCATCGAGCT GAAGGGCATC GACTTCAAGG AGGACGGCAA CATCCTGGGG |
| | eGFP |
| | ~~~~~~~~~~~~~~~~~~~~~~~~~~~~~~~~~~~~~~~~~~~~~~~~~~~~~~~~~~~~~~~~~ |
| 7981 | CACAAGCTGG AGTACAACTA CAACAGCCAC AACGTCTATA TCATGGCCGA CAAGCAGAAG |
| | eGFP |
| | ~~~~~~~~~~~~~~~~~~~~~~~~~~~~~~~~~~~~~~~~~~~~~~~~~~~~~~~~~~~~~~~~~ |
| 8041 | AACGGCATCA AGGTGAACTT CAAGATCCGC CACAACATCG AGGACGGCAG CGTGCAGCTC |
| | eGFP |
| | ~~~~~~~~~~~~~~~~~~~~~~~~~~~~~~~~~~~~~~~~~~~~~~~~~~~~~~~~~~~~~~~~~ |
| 8101 | GCCGACCACT ACCAGCAGAA CACCCCCATC GGCGACGGCC CCGTGCTGCT GCCCGACAAC |
| | eGFP |
| | ~~~~~~~~~~~~~~~~~~~~~~~~~~~~~~~~~~~~~~~~~~~~~~~~~~~~~~~~~~~~~~~~~ |
| 8161 | CACTACCTGA GCACCCAGTC CGCCCTGAGC AAAGACCCCA ACGAGAAGCG CGATCACATG |
| | eGFP |
| | ~~~~~~~~~~~~~~~~~~~~~~~~~~~~~~~~~~~~~~~~~~~~~~~~~~~~~~~~~~~~~~~~~ |
| 8221 | GTCCTGCTGG AGTTCGTGAC CGCCGCCGGG ATCACTCTCG GCATGGACGA GCTGTACAAG |
| | eGFP 3′UTR |
| | ~~~~~~ ~~~~~~~~~~~~~~~~~~~~~~~~~~ |
| 8281 | TGATAATCTA GACGGCGCGC CCACCCAGCG GCCGCATACA GCAGCAATTG GCAAGCTGCT |
| | 3′UTR |
| | ~~~~~~~~~~~~~~~~~~~~~~~~~~~~~~~~~~~~~~~~~~~~~~~~~~~~~~~~~~~~~~~~~ |
| 8341 | TACATAGAAC TCGCGGCGAT TGGCATGCCG CCTTAAAATT TTTATTTTAT TTTTCTTTTC |
| | 3′UTR |
| | ~~~~~~~~~~~~~~~~~~~~~~~~~~~~~~~~~~~ |
| 8401 | TTTTCCGAAT CGGATTTTGT TTTTAATATT TCAAAAAAAA AAAAAAAAAA AAAAAAAAAA |
| | HDV ribozyme |
| | ~~~~~~~~~~~~~~~~~~~~~~~~~~~~~~~~~~~~~~~~~~~~~~~~~~~~~~~~~ |
| 8461 | AAAAAAAGGG TCGGCATGGC ATCTCCACCT CCTCGCGGTC CGACCTGGGC ATCCGAAGGA |
| | HDV ribozyme |
| | ~~~~~~~~~~~~~~~~~~~~~~~~~~~~~~~~~~~~~~~ |
| 8521 | GGACGCACGT CCACTCGGAT GGCTAAGGGA GAGCCACGTT TAAACCAGCT CCAATTCGCC |
| 8581 | CTATAGTGAG TCGTATTACG CGCGCTCACT GGCCGTCGTT TTACAACGTC GTGACTGGGA |
| 8641 | AAACCCTGGC GTTACCCAAC TTAATCGCCT TGCAGCACAT CCCCCTTTCG CCAGCTGGCG |
| 8701 | TAATAGCGAA GAGGCCCGCA CCGATCGCCC TTCCCAACAG TTGCGCAGCC TGAATGGCGA |
| 8761 | ATGGGACGCG CCCTGTAGCG GCGCATTAAG CGCGGCGGGT GTGGTGGTTA CGCGCAGCGT |
| 8821 | GACCGCTACA CTTGCCAGCG CCCTAGCGCC CGCTCCTTTC GCTTTCTTCC CTTCCTTTCT |
| 8881 | CGCCACGTTC GCCGGCTTTC CCCGTCAAGC TCTAAATCGG GGGCTCCCTT TAGGGTTCCG |
| 8941 | ATTTAGTGCT TTACGGCACC TCGACCCCAA AAAACTTGAT TAGGGTGATG GTTCACGTAG |
| 9001 | TGGGCCATCG CCCTGATAGA CGGTTTTTCG CCCTTTGACG TTGGAGTCCA CGTTCTTTAA |
| 9061 | TAGTGGACTC TTGTTCCAAA CTGGAACAAC ACTCAACCCT ATCTCGGTCT ATTCTTTTGA |
| 9121 | TTTATAAGGG ATTTTGCCGA TTTCGGCCTA TTGGTTAAAA AATGAGCTGA TTTAACAAAA |
| 9181 | ATTTAACGCG AATTTTAACA AAATATTAAC GCTTACAATT TAGGTGGCAC TTTTCGGGGA |
| 9241 | AATGTGCGCG GAACCCCTAT TTGTTTATTT TTCTAAATAC ATTCAAATAT GTATCCGCTC |
| | bla |
| | ~~~~~~~~~~~ |
| 9301 | ATGAGACAAT AACCCTGATA AATGCTTCAA TAATATTGAA AAAGGAAGAG TATGAGTATT |
| | bla |
| | ~~~~~~~~~~~~~~~~~~~~~~~~~~~~~~~~~~~~~~~~~~~~~~~~~~~~~~~~~~~~~~~~~ |
| 9361 | CAACATTTCC GTGTCGCCCT TATTCCCTTT TTTGCGGCAT TTTGCCTTCC TGTTTTTGCT |
| | bla |
| | ~~~~~~~~~~~~~~~~~~~~~~~~~~~~~~~~~~~~~~~~~~~~~~~~~~~~~~~~~~~~~~~~~ |
| 9421 | CACCCAGAAA CGCTGGTGAA AGTAAAAGAT GCTGAAGATC AGTTGGGTGC ACGAGTGGGT |
| | bla |
| | ~~~~~~~~~~~~~~~~~~~~~~~~~~~~~~~~~~~~~~~~~~~~~~~~~~~~~~~~~~~~~~~~~ |
| 9481 | TACATCGAAC TGGATCTCAA CAGCGGTAAG ATCCTTGAGA GTTTTCGCCC CGAAGAACGT |
| | bla |
| | ~~~~~~~~~~~~~~~~~~~~~~~~~~~~~~~~~~~~~~~~~~~~~~~~~~~~~~~~~~~~~~~~~ |
| 9541 | TTTCCAATGA TGAGCACTTT TAAAGTTCTG CTATGTGGCG CGGTATTATC CCGTATTGAC |
| | bla |
| | ~~~~~~~~~~~~~~~~~~~~~~~~~~~~~~~~~~~~~~~~~~~~~~~~~~~~~~~~~~~~~~~~~ |
| 9601 | GCCGGGCAAG AGCAACTCGG TCGCCGCATA CACTATTCTC AGAATGACTT GGTTGAGTAC |
| | bla |
| | ~~~~~~~~~~~~~~~~~~~~~~~~~~~~~~~~~~~~~~~~~~~~~~~~~~~~~~~~~~~~~~~~~ |
| 9661 | TCACCAGTCA CAGAAAAGCA TCTTACGGAT GGCATGACAG TAAGAGAATT ATGCAGTGCT |
| | bla |
| | ~~~~~~~~~~~~~~~~~~~~~~~~~~~~~~~~~~~~~~~~~~~~~~~~~~~~~~~~~~~~~~~~~ |
| 9721 | GCCATAACCA TGAGTGATAA CACTGCGGCC AACTTACTTC TGACAACGAT CGGAGGACCG |
| | bla |
| | ~~~~~~~~~~~~~~~~~~~~~~~~~~~~~~~~~~~~~~~~~~~~~~~~~~~~~~~~~~~~~~~~~ |
| 9781 | AAGGAGCTAA CCGCTTTTTT GCACAACATG GGGGATCATG TAACTCGCCT TGATCGTTGG |
| | bla |
| | ~~~~~~~~~~~~~~~~~~~~~~~~~~~~~~~~~~~~~~~~~~~~~~~~~~~~~~~~~~~~~~~~~ |
| 9841 | GAACCGGAGC TGAATGAAGC CATACCAAAC GACGAGCGTG ACACCACGAT GCCTGTAGCA |
| | bla |
| | ~~~~~~~~~~~~~~~~~~~~~~~~~~~~~~~~~~~~~~~~~~~~~~~~~~~~~~~~~~~~~~~~~ |
| 9901 | ATGGCAACAA CGTTGCGCAA ACTATTAACT GGCGAACTAC TTACTCTAGC TTCCCGGCAA |
| | bla |
| | ~~~~~~~~~~~~~~~~~~~~~~~~~~~~~~~~~~~~~~~~~~~~~~~~~~~~~~~~~~~~~~~~~ |
| 9961 | CAATTAATAG ACTGGATGGA GGCGGATAAA GTTGCAGGAC CACTTCTGCG CTCGGCCCTT |
| | bla |
| | ~~~~~~~~~~~~~~~~~~~~~~~~~~~~~~~~~~~~~~~~~~~~~~~~~~~~~~~~~~~~~~~~~ |
| 10021 | CCGGCTGGCT GGTTTATTGC TGATAAATCT GGAGCCGGTG AGCGTGGGTC TCGCGGTATC |
| | bla |
| | ~~~~~~~~~~~~~~~~~~~~~~~~~~~~~~~~~~~~~~~~~~~~~~~~~~~~~~~~~~~~~~~~~ |
| 10081 | ATTGCAGCAC TGGGGCCAGA TGGTAAGCCC TCCCGTATCG TAGTTATCTA CACGACGGGG |
| | bla |
| | ~~~~~~~~~~~~~~~~~~~~~~~~~~~~~~~~~~~~~~~~~~~~~~~~~~~~~~~~~~~~~~~~~ |
| 10141 | AGTCAGGCAA CTATGGATGA ACGAAATAGA CAGATCGCTG AGATAGGTGC CTCACTGATT |
| | bla |
| | ~~~~~~~~~~ |
| 10201 | AAGCATTGGT AACTGTCAGA CCAAGTTTAC TCATATATAC TTTAGATTGA TTTAAAACTT |
| 10261 | CATTTTTAAT TTAAAAGGAT CTAGGTGAAG ATCCTTTTTG ATAATCTCAT GACCAAAATC |
| 10321 | CCTTAACGTG AGTTTTCGTT CCACTGAGCG TCAGACCCCG TAGAAAAGAT CAAAGGATCT |
| 10381 | TCTTGAGATC CTTTTTTTCT GCGCGTAATC TGCTGCTTGC AAACAAAAAA ACCACCGCTA |
| 10441 | CCAGCGGTGG TTTGTTTGCC GGATCAAGAG CTACCAACTC TTTTTCCGAA GGTAACTGGC |
| 10501 | TTCAGCAGAG CGCAGATACC AAATACTGTT CTTCTAGTGT AGCCGTAGTT AGGCCACCAC |
| 10561 | TTCAAGAACT CTGTAGCACC GCCTACATAC CTCGCTCTGC TAATCCTGTT ACCAGTGGCT |
| 10621 | GCTGCCAGTG GCGATAAGTC GTGTCTTACC GGGTTGGACT CAAGACGATA GTTACCGGAT |
| 10681 | AAGGCGCAGC GGTCGGGCTG AACGGGGGGT TCGTGCACAC AGCCCAGCTT GGAGCGAACG |
| 10741 | ACCTACACCG AACTGAGATA CCTACAGCGT GAGCTATGAG AAAGCGCCAC GCTTCCCGAA |
| 10801 | GGGAGAAAGG CGGACAGGTA TCCGGTAAGC GGCAGGGTCG GAACAGGAGA GCGCACGAGG |
| 10861 | GAGCTTCCAG GGGGAAACGC CTGGTATCTT TATAGTCCTG TCGGGTTTCG CCACCTCTGA |
| 10921 | CTTGAGCGTC GATTTTTGTG ATGCTCGTCA GGGGGGCGGA GCCTATGGAA AAACGCCAGC |
| 10981 | AACGCGGCCT TTTTACGGTT CCTGGCCTTT TGCTGGCCTT TTGCTCACAT GTTCTTTCCT |
| 11041 | GCGTTATCCC CTGATTCTGT GGATAACCGT ATTACCGCCT TTGAGTGAGC TGATACCGCT |
| 11101 | CGCCGCAGCC GAACGACCGA GCGCAGCGAG TCAGTGAGCG AGGAAGCGGA AGAGCGCCCA |
| 11161 | ATACGCAAAC CGCCTCTCCC CGCGCGTTGG CCGATTCATT AATGCAGCTG GCACGACAGG |
| 11221 | TTTCCCGACT GGAAAGCGGG CAGTGAGCGC AACGCAATTA ATGTGAGTTA GCTCACTCAT |
| 11281 | TAGGCACCCC AGGCTTTACA CTTTATGCTC CCGGCTCGTA TGTTGTGTGG AATTGTGAGC |
| 11341 | GGATAACAAT TTCACACAGG AAACAGCTAT GACCATGATT ACGCCAAGCG CGCAATTAAC |
| 11401 | CCTCACTAAA GGGAACAAAA GCTGGGTACC GGGCCCACGC GTAATACGAC TCACTATAG |

VEE cap helper
(SEQ ID NO: 43)

| | |
|---|---|
| | 5′UTR |
| | ~~~~~~~~~~~~~~~~~~~~~~~~~~~~~~~~~~~~~ |
| | nsP1 |
| | ~~~~~~~~~~~~~~~~~~~~ |
| 1 | ATAGGCGGCG CATGAGAGAA GCCCAGACCA ATTACCTACC CAAATAGGAG AAAGTTCACG |
| | nsP1 |
| | ~~~~~~~~~~~~~~~~~~~~~~~~~~~~~~~~~~~~~~~~~~~~~~~~~~~~~~~~~~~~~~~~~ |
| 61 | TTGACATCGA GGAAGACAGC CCATTCCTCA GAGCTTTGCA GCGGAGCTTC CCGCAGTTTG |
| | nsP1 |
| | ~~~~~~~~~~~~~~~~~~~~~~~~~~~~~~~~~~~~~~~~~~~~~~~~~~~~~~~~~~~~~~~~~ |
| 121 | AGGTAGAAGC CAAGCAGGTC ACTGATAATG ACCATGCTAA TGCCAGAGCG TTTTCGCATC |
| | nsP1 |
| | ~~~~~~~~~~~~~~~~~~~~~~~~~~~~~~~~~~~~~~~~~~~~~~~~~~~~~~~~~~~~~~~~~ |
| 181 | TGGCTTCAAA ACTGATCGAA ACGGAGGTGG ACCCATCCGA CACGATCCTT GACATTGGAC |
| | VEECAP |
| | ~~~~~~~~~~~~~~~~~~~~~~~~~~~~~~~~~~~~~~~~~~~~~~~~~~~~~~~~~~~~~~~~~ |
| 241 | GGACCGACCA TGTTCCCGTT CCAGCCAATG TATCCGATGC AGCCAATGCC CTATCGCAAC |
| | VEECAP |
| | ~~~~~~~~~~~~~~~~~~~~~~~~~~~~~~~~~~~~~~~~~~~~~~~~~~~~~~~~~~~~~~~~~ |
| 301 | CCGTTCGCGG CCCCGCGCAG GCCCTGGTTC CCCAGAACCG ACCCTTTTCT GGCGATGCAG |
| | VEECAP |
| | ~~~~~~~~~~~~~~~~~~~~~~~~~~~~~~~~~~~~~~~~~~~~~~~~~~~~~~~~~~~~~~~~~ |
| 361 | GTGCAGGAAT TAACCCGCTC GATGGCTAAC CTGACGTTCA AGCAACGCCG GGACGCGCCA |
| | VEECAP |
| | ~~~~~~~~~~~~~~~~~~~~~~~~~~~~~~~~~~~~~~~~~~~~~~~~~~~~~~~~~~~~~~~~~ |
| 421 | CCTGAGGGGC CATCCGCTAA GAAACCGAAG AAGGAGGCCT CGCAAAAACA GAAAGGGGGA |
| | VEECAP |
| | ~~~~~~~~~~~~~~~~~~~~~~~~~~~~~~~~~~~~~~~~~~~~~~~~~~~~~~~~~~~~~~~~~ |
| 481 | GGCCAAGGGA AGAAGAAGAA GAACCAAGGG AAGAAGAAGG CTAAGACAGG GCCGCCTAAT |
| | VEECAP |
| | ~~~~~~~~~~~~~~~~~~~~~~~~~~~~~~~~~~~~~~~~~~~~~~~~~~~~~~~~~~~~~~~~~ |
| 541 | CCGAAGGCAC AGAATGGAAA CAAGAAGAAG ACCAACAAGA AACCAGGCAA GAGACAGCGC |
| | VEECAP |
| | ~~~~~~~~~~~~~~~~~~~~~~~~~~~~~~~~~~~~~~~~~~~~~~~~~~~~~~~~~~~~~~~~~ |
| 601 | ATGGTCATGA AATTGGAATC TGACAAGACG TTCCCAATCA TGTTGGAAGG GAAGATAAAC |
| | VEECAP |
| | ~~~~~~~~~~~~~~~~~~~~~~~~~~~~~~~~~~~~~~~~~~~~~~~~~~~~~~~~~~~~~~~~~ |
| | H152G |
| | ~~~ |
| 661 | GGCTACGCTT GTGTGGTCGG AGGGAAGTTA TTCAGGCCGA TGGGTGTGGA AGGCAAGATC |
| | VEECAP |
| | ~~~~~~~~~~~~~~~~~~~~~~~~~~~~~~~~~~~~~~~~~~~~~~~~~~~~~~~~~~~~~~~~~ |
| 721 | GACAACGACG TTCTGGCCGC GCTTAAGACG AAGAAAGCAT CCAAATACGA TCTTGAGTAT |
| | VEECAP |
| | ~~~~~~~~~~~~~~~~~~~~~~~~~~~~~~~~~~~~~~~~~~~~~~~~~~~~~~~~~~~~~~~~~ |
| 781 | GCAGATGTGC CACAGAACAT GCGGGCCGAT ACATTCAAAT ACACCCATGA GAAACCCCAA |
| | VEECAP |
| | ~~~~~~~~~~~~~~~~~~~~~~~~~~~~~~~~~~~~~~~~~~~~~~~~~~~~~~~~~~~~~~~~~ |
| 841 | GGCTATTACA GCTGGCATCA TGGAGCAGTC CAATATGAAA ATGGGCGTTT CACGGTGCCG |
| | VEECAP |
| | ~~~~~~~~~~~~~~~~~~~~~~~~~~~~~~~~~~~~~~~~~~~~~~~~~~~~~~~~~~~~~~~~~ |
| 901 | AAAGGAGTTG GGGCCAAGGG AGACAGCGGA CGACCCATTC TGGATAACCA GGGACGGGTG |
| | VEECAP |
| | ~~~~~~~~~~~~~~~~~~~~~~~~~~~~~~~~~~~~~~~~~~~~~~~~~~~~~~~~~~~~~~~~~ |
| 961 | GTCGCTATTG TGCTGGGAGG TGTGAATGAA GGATCTAGGA CAGCCCTTTC AGTCGTCATG |
| | VEECAP |
| | ~~~~~~~~~~~~~~~~~~~~~~~~~~~~~~~~~~~~~~~~~~~~~~~~~~~~~~~~~~~~~~~~~ |
| 1021 | TGGAACGAGA AGGGAGTTAC CGTGAAGTAT ACTCCGGAGA ACTGCGAGCA ATGGTAATAG |
| | VEECAP 3′UTR |
| | ~~~~ ~~~~~~~~~~~~~~~~~~~~~~~~~~~~~~~~~~~~~~~~~~~~~~~~~~ |
| 1081 | TAAGCGGCCG CATACAGCAG CAATTGGCAA GCTGCTTACA TAGAACTCGC GGCGATTGGC |
| | 3′UTR |
| | ~~~~~~~~~~~~~~~~~~~~~~~~~~~~~~~~~~~~~~~~~~~~~~~~~~~~~~~~~~~~~~~~~ |
| 1141 | ATGCCGCCTT AAAATTTTTA TTTTATTTTT CTTTTCTTTT CCGAATCGGA TTTTGTTTTT |
| | 3′UTR HDV ribozyme |
| | ~~~~~~~ ~~~~~~~~~~~~~~~~~~~~~~~~~~~~~~~~~~~ |
| 1201 | AATATTTCAA AAAAAAAAAA AAAAAAAAAA AAAAAAAAAA AAAGGGTCGG CATGGCATCT |
| | HDV ribozyme |
| | ~~~~~~~~~~~~~~~~~~~~~~~~~~~~~~~~~~~~~~~~~~~~~~~~~~~~~~~~~~~~~~~~~ |
| 1261 | CCACCTCCTC GCGGTCCGAC CTGGGCATCC GAAGGAGGAC GCACGTCCAC TCGGATGGCT |
| | HDV ribozyme |
| | ~~~~~~~~~~~~~ |
| 1321 | AAGGGAGAGC CACGTTTAAA CACGTGATAT CTGGCCTCAT GGGCCTTCCT TTCACTGCCC |
| 1381 | GCTTTCCAGT CGGGAAACCT GTCGTGCCAG CTGCATTAAC ATGGTCATAG CTGTTTCCTT |
| 1441 | GCGTATTGGG CGCTCTCCGC TTCCTCGCTC ACTGACTCGC TGCGCTCGGT CGTTCGGGTA |
| | colE1 |
| | ~~~~~~~~~~~~~~~~~~~~~~~~~~~~~~~~~~~~~~~~~~~~ |
| 1501 | AAGCCTGGGG TGCCTAATGA GCAAAAGGCC AGCAAAAGGC CAGGAACCGT AAAAAGGCCG |
| | colE1 |
| | ~~~~~~~~~~~~~~~~~~~~~~~~~~~~~~~~~~~~~~~~~~~~~~~~~~~~~~~~~~~~~~~~~ |
| 1561 | CGTTGCTGGC GTTTTTCCAT AGGCTCCGCC CCCCTGACGA GCATCACAAA AATCGACGCT |
| | colE1 |
| | ~~~~~~~~~~~~~~~~~~~~~~~~~~~~~~~~~~~~~~~~~~~~~~~~~~~~~~~~~~~~~~~~~ |
| 1621 | CAAGTCAGAG GTGGCGAAAC CCGACAGGAC TATAAAGATA CCAGGCGTTT CCCCCTGGAA |
| | colE1 |
| | ~~~~~~~~~~~~~~~~~~~~~~~~~~~~~~~~~~~~~~~~~~~~~~~~~~~~~~~~~~~~~~~~~ |
| 1681 | GCTCCCTCGT GCGCTCTCCT GTTCCGACCC TGCCGCTTAC CGGATACCTG TCCGCCTTTC |
| | colE1 |
| | ~~~~~~~~~~~~~~~~~~~~~~~~~~~~~~~~~~~~~~~~~~~~~~~~~~~~~~~~~~~~~~~~~ |
| 1741 | TCCCTTCGGG AAGCGTGGCG CTTTCTCATA GCTCACGCTG TAGGTATC7C AGTTCGGTGT |
| | colE1 |
| | ~~~~~~~~~~~~~~~~~~~~~~~~~~~~~~~~~~~~~~~~~~~~~~~~~~~~~~~~~~~~~~~~~ |
| 1801 | AGGTCGTTCG CTCCAAGCTG GGCTGTGTGC ACGAACCCCC CGTTCAGCCC GACCGCTGCG |
| | colE1 |
| | ~~~~~~~~~~~~~~~~~~~~~~~~~~~~~~~~~~~~~~~~~~~~~~~~~~~~~~~~~~~~~~~~~ |
| 1861 | CCTTATCCGG TAACTATCGT CTTGAGTCCA ACCCGGTAAG ACACGACTTA TCGCCACTGG |
| | colE1 |
| | ~~~~~~~~~~~~~~~~~~~~~~~~~~~~~~~~~~~~~~~~~~~~~~~~~~~~~~~~~~~~~~~~~ |
| 1921 | CAGCAGCCAC TGGTAACAGG ATTAGCAGAG CGAGGTATGT AGGCGGTGCT ACAGAGTTCT |
| | colE1 |
| | ~~~~~~~~~~~~~~~~~~~~~~~~~~~~~~~~~~~~~~~~~~~~~~~~~~~~~~~~~~~~~~~~~ |
| 1981 | TGAAGTGGTG GCCTAACTAC GGCTACACTA GAAGAACAGT ATTTGGTATC TGCGCTCTGC |
| | colE1 |
| | ~~~~~~~~~~~~~~~~~~~~~~~~~~~~~~~~~~~~~~~~~~~~~~~~~~~~~~~~~~~~~~~~~ |
| 2041 | TGAAGCCAGT TACCTTCGGA AAAAGAGTTG GTAGCTCTTG ATCCGGCAAA CAAACCACCG |
| | colE1 |
| | ~~~~~~~~~~~~~~~~~~~~~~~~~~~~~~~~~~~~~~~~~~~~~~~~~~~~~~~~~~~~~~~~~ |
| 2101 | CTGGTAGCGG TGGTTTTTTT GTTTGCAAGC AGCAGATTAC GCGCAGAAAA AAAGGATCTC |
| | colE1 |
| | ~~~~~~~~~~~~~~~~~~~~~~~~~~~~ |
| 2161 | AAGAAGATCC TTTGATCTTT TCTACGGGGT CTGACGCTCA GTGGAACGAA AACTCACGTT |
| 2221 | AAGGGATTTT GGTCATGAGA TTATCAAAAA GGATCTTCAC CTAGATCCTT TTAAATTAAA |
| 2281 | AATGAAGTTT TAAATCAATC TAAAGTATAT ATGAGTAAAC TTGGTCTGAC AGTTATTAGA |
| | ~~~ |
| | KanR |
| 2341 | AAAATTCATC CAGCAGACGA TAAAACGCAA TACGCTGGCT ATCCGGTGCC GCAATGCCAT |
| | ~~~~~~~~~~~~~~~~~~~~~~~~~~~~~~~~~~~~~~~~~~~~~~~~~~~~~~~~~~~~~~~~~ |
| | KanR |
| 2401 | ACAGCACCAG AAAACGATCC GCCCATTCGC CGCCCAGTTC TTCCGCAATA TCACGGGTGG |
| | ~~~~~~~~~~~~~~~~~~~~~~~~~~~~~~~~~~~~~~~~~~~~~~~~~~~~~~~~~~~~~~~~~ |
| | KanR |
| 2461 | CCAGCGCAAT ATCCTGATAA CGATCCGCCA CGCCCAGACG GCCGCAATCA ATAAAGCCGC |
| | ~~~~~~~~~~~~~~~~~~~~~~~~~~~~~~~~~~~~~~~~~~~~~~~~~~~~~~~~~~~~~~~~~ |
| | KanR |
| 2521 | TAAAACGGCC ATTTTCCACC ATAATGTTCG GCAGGCACGC ATCACCATGG GTCACCACCA |
| | ~~~~~~~~~~~~~~~~~~~~~~~~~~~~~~~~~~~~~~~~~~~~~~~~~~~~~~~~~~~~~~~~~ |
| | KanR |
| 2581 | GATCTTCGCC ATCCGGCATG CTCGCTTTCA GACGCGCAAA CAGCTCTGCC GGTGCCAGGC |
| | ~~~~~~~~~~~~~~~~~~~~~~~~~~~~~~~~~~~~~~~~~~~~~~~~~~~~~~~~~~~~~~~~~ |
| | KanR |
| 2641 | CCTGATGTTC TTCATCCAGA TCATCCTGAT CCACCAGGCC CGCTTCCATA CGGGTACGCG |
| | ~~~~~~~~~~~~~~~~~~~~~~~~~~~~~~~~~~~~~~~~~~~~~~~~~~~~~~~~~~~~~~~~~ |
| | KanR |
| 2701 | CACGTTCAAT ACGATGTTTC GCCTGATGAT CAAACGGACA GGTCGCCGGG TCCAGGGTAT |
| | ~~~~~~~~~~~~~~~~~~~~~~~~~~~~~~~~~~~~~~~~~~~~~~~~~~~~~~~~~~~~~~~~~ |
| | KanR |
| 2761 | GCAGACGACG CATGGCATCC GCCATAATGC TCACTTTTTC TGCCGGCGCC AGATGGCTAG |
| | ~~~~~~~~~~~~~~~~~~~~~~~~~~~~~~~~~~~~~~~~~~~~~~~~~~~~~~~~~~~~~~~~~ |
| | KanR |
| 2821 | ACAGCAGATC CTGACCCGGC ACTTCGCCCA GCAGCAGCCA ATCACGGCCC GCTTCGGTCA |
| | ~~~~~~~~~~~~~~~~~~~~~~~~~~~~~~~~~~~~~~~~~~~~~~~~~~~~~~~~~~~~~~~~~ |
| | KanR |
| 2881 | CCACATCCAG CACCGCCGCA CACGGAACAC CGGTGGTGGC CAGCCAGCTC AGACGCGCCG |
| | ~~~~~~~~~~~~~~~~~~~~~~~~~~~~~~~~~~~~~~~~~~~~~~~~~~~~~~~~~~~~~~~~~ |
| | KanR |
| 2941 | CTTCATCCTG CAGCTCGTTC AGCGCACCGC TCAGATCGGT TTTCACAAAC AGCACCGGAC |
| | ~~~~~~~~~~~~~~~~~~~~~~~~~~~~~~~~~~~~~~~~~~~~~~~~~~~~~~~~~~~~~~~~~ |
| | KanR |
| 3001 | GACCCTGCGC GCTCAGACGA AACACCGCCG CATCAGAGCA GCCAATGGTC TGCTGCGCCC |
| | ~~~~~~~~~~~~~~~~~~~~~~~~~~~~~~~~~~~~~~~~~~~~~~~~~~~~~~~~~~~~~~~~~ |
| | KanR |
| 3061 | AATCATAGCC AAACAGACGT TCCACCCACG CTGCCGGGCT ACCCGCATGC AGGCCATCCT |
| | ~~~~~~~~~~~~~~~~~~~~~~~~~~~~~~~~~~~~~~~~~~~~~~~~~~~~~~~~~~~~~~~~~ |
| | KanR |
| 3121 | GTTCAATCAT ACTCTTCCTT TTTCAATATT ATTGAAGCAT TTATCAGGGT TATTGTCTCA |
| | ~~~~~~~~ |
| | KanR |
| 3181 | TGAGCGGATA CATATTTGAA TGTATTTAGA AAAATAAACA AATAGGGGTT CCGCGCACAT |
| 3241 | TTCCCCGAAA AGTGCCACCT AAATTGTAAG CGTTAATATT TTGTTAAAAT TCGCGTTAAA |
| 3301 | TTTTTGTTAA ATCAGCTCAT TTTTTAACCA ATAGGCCGAA ATCGGCAAAA TCCCTTATAA |
| 3361 | ATCAAAAGAA TAGACCGAGA TAGGGTTGAG TGGCCGCTAC AGGGCGCTCC CATTCGCCAT |
| 3421 | TCAGGCTGCG CAACTGTTGG GAAGGGCGTT TCGGTGCGGG CCTCTTCGCT ATTACGCCAG |
| 3481 | CTGGCGAAAG GGGGATGTGC TGCAAGGCGA TTAAGTTGGG TAACGCCAGG GTTTTCCCAG |
| | T7 promoter |
| | ~~~~~~~~~~~~~~~~~~~ |
| 3541 | TCACACGCGT AATACGACTC ACTATAG |

VEE gly helper
(SEQ ID NO: 44)

| | |
|---|---|
| | 5′UTR |
| | ~~~~~~~~~~~~~~~~~~~~~~~~~~~~~~~~~~~~~~ |
| | nsP1 |
| | ~~~~~~~~~~~~~~~ |
| 1 | ATAGGCGGCG CATGAGAGAA GCCCAGACCA ATTACCTACC CAAATAGGAG AAAGTTCACG |
| | nsP1 |
| | ~~~~~~~~~~~~~~~~~~~~~~~~~~~~~~~~~~~~~~~~~~~~~~~~~~~~~~~~~~~~~~~~~ |
| 61 | TTGACATCGA GGAAGACAGC CCATTCCTCA GAGCTTTGCA GCGGAGCTTC CCGCAGTTTG |
| | nsP1 |
| | ~~~~~~~~~~~~~~~~~~~~~~~~~~~~~~~~~~~~~~~~~~~~~~~~~~~~~~~~~~~~~~~~~ |
| 121 | AGGTAGAAGC CAAGCAGGTC ACTGATAATG ACCATGCTAA TGCCAGAGCG TTTTCGCATC |
| | nsP1 |
| | ~~~~~~~~~~~~~~~~~~~~~~~~~~~~~~~~~~~~~~~~~~~~~~~~~~~~~~~~~~~~~~~~~ |
| 181 | TGGCTTCAAA ACTGATCGAA ACGGAGGTGG ACCCATCCGA CACGATCCTT GACATTGGAC |
| | VEE GLY |
| | ~~~~~~~~~~~~~~~~~~~~~~~~~~~~~~~~~~~~~~~~~~~~~~~~~~~~~~~~~~~~~~~~~ |
| 241 | GGACCGACCA TGTCACTAGT GACCACCATG TGTCTGCTCG CCAATGTGAC GTTCCCATGT |
| | VEE GLY |
| | ~~~~~~~~~~~~~~~~~~~~~~~~~~~~~~~~~~~~~~~~~~~~~~~~~~~~~~~~~~~~~~~~~ |
| 301 | GCTCAACCAC CAATTTGCTA CGACAGAAAA CCAGCAGAGA CTTTGGCCAT GCTCAGCGTT |
| | VEE GLY |
| | ~~~~~~~~~~~~~~~~~~~~~~~~~~~~~~~~~~~~~~~~~~~~~~~~~~~~~~~~~~~~~~~~~ |
| 361 | AACGTTGACA ACCCGGGCTA CGATGAGCTG CTGGAAGCAG CTGTTAAGTG CCCCGGAAGG |
| | VEE GLY |
| | ~~~~~~~~~~~~~~~~~~~~~~~~~~~~~~~~~~~~~~~~~~~~~~~~~~~~~~~~~~~~~~~~~ |
| 421 | AAAAGGAGAT CCACCGAGGA GCTGTTTAAT GAGTATAAGC TAACGCGCCC TTACATGGCC |
| | VEE GLY |
| | ~~~~~~~~~~~~~~~~~~~~~~~~~~~~~~~~~~~~~~~~~~~~~~~~~~~~~~~~~~~~~~~~~ |
| 481 | AGATGCATCA GATGTGCAGT TGGGAGCTGC CATAGTCCAA TAGCAATCGA GGCAGTAAAG |
| | VEE GLY |
| | ~~~~~~~~~~~~~~~~~~~~~~~~~~~~~~~~~~~~~~~~~~~~~~~~~~~~~~~~~~~~~~~~~ |
| 541 | AGCGACGGGC ACGACGGTTA TGTTAGACTT CAGACTTCCT CGCAGTATGG CCTGGATTCC |
| | VEE GLY |
| | ~~~~~~~~~~~~~~~~~~~~~~~~~~~~~~~~~~~~~~~~~~~~~~~~~~~~~~~~~~~~~~~~~ |
| 601 | TCCGGCAACT TAAAGGGCAG GACCATGCGG TATGACATGC ACGGGACCAT TAAAGAGATA |
| | VEE GLY |
| | ~~~~~~~~~~~~~~~~~~~~~~~~~~~~~~~~~~~~~~~~~~~~~~~~~~~~~~~~~~~~~~~~~ |
| 661 | CCACTACATC AAGTGTCACT CTATACATCT CGCCCGTGTC ACATTGTGGA TGGGCACGGT |
| | VEE GLY |
| | ~~~~~~~~~~~~~~~~~~~~~~~~~~~~~~~~~~~~~~~~~~~~~~~~~~~~~~~~~~~~~~~~~ |
| 721 | TATTTCCTGC TTGCCAGGTG CCCGGCAGGG GACTCCATCA CCATGGAATT TAAGAAAGAT |
| | VEE GLY |
| | ~~~~~~~~~~~~~~~~~~~~~~~~~~~~~~~~~~~~~~~~~~~~~~~~~~~~~~~~~~~~~~~~~ |
| 781 | TCCGTCAGAC ACTCCTGCTC GGTGCCGTAT GAAGTGAAAT TTAATCCTGT AGGCAGAGAA |
| | VEE GLY |
| | ~~~~~~~~~~~~~~~~~~~~~~~~~~~~~~~~~~~~~~~~~~~~~~~~~~~~~~~~~~~~~~~~~ |
| 841 | CTCTATACT ATCCCCCAGA ACACGGAGTA GAGCAAGCGT GCCAAGTCTA CGCACATGAT |
| | VEE GLY |
| | ~~~~~~~~~~~~~~~~~~~~~~~~~~~~~~~~~~~~~~~~~~~~~~~~~~~~~~~~~~~~~~~~~ |
| 901 | GCACAGAACA GAGGAGCTTA TGTCGAGATG CACCTCCCGG GCTCAGAAGT GGACAGCAGT |
| | VEE GLY |
| | ~~~~~~~~~~~~~~~~~~~~~~~~~~~~~~~~~~~~~~~~~~~~~~~~~~~~~~~~~~~~~~~~~ |
| 961 | TTGGTTTCCT TGAGCGGCAG TTCAGTCACC GTGACACCTC CTGATGGGAC TAGCGCCCTG |
| | VEE GLY |
| | ~~~~~~~~~~~~~~~~~~~~~~~~~~~~~~~~~~~~~~~~~~~~~~~~~~~~~~~~~~~~~~~~~ |
| 1021 | GTGGAATGCG AGTGTGGCGG CACAAAGATC TCCGAGACCA TCAACAAGAC AAAACAGTTC |
| | VEE GLY |
| | ~~~~~~~~~~~~~~~~~~~~~~~~~~~~~~~~~~~~~~~~~~~~~~~~~~~~~~~~~~~~~~~~~ |
| 1081 | AGCCAGTGCA CAAAGAAGGA GCAGTGCAGA GCATATCGGC TGCAGAACGA TAAGTGGGTG |
| | VEE GLY |
| | ~~~~~~~~~~~~~~~~~~~~~~~~~~~~~~~~~~~~~~~~~~~~~~~~~~~~~~~~~~~~~~~~~ |
| 1141 | TATAATTCTG ACAAACTGCC CAAAGCAGCG GGAGCCACCT TAAAAGGAAA ACTGCATGTC |
| | VEE GLY |
| | ~~~~~~~~~~~~~~~~~~~~~~~~~~~~~~~~~~~~~~~~~~~~~~~~~~~~~~~~~~~~~~~~~ |
| 1201 | CCATTCTTGC TGGCAGACGG CAAATGCACC GTGCCTCTAG CACCAGAACC TATGATAACC |
| | VEE GLY |
| | ~~~~~~~~~~~~~~~~~~~~~~~~~~~~~~~~~~~~~~~~~~~~~~~~~~~~~~~~~~~~~~~~~ |
| 1261 | TTCGGTTTCA GATCAGTGTC ACTGAAACTG CACCCTAAGA ATCCCACATA TCTAATCACC |
| | VEE GLY |
| | ~~~~~~~~~~~~~~~~~~~~~~~~~~~~~~~~~~~~~~~~~~~~~~~~~~~~~~~~~~~~~~~~~ |
| 1321 | CGCCAACTTG CTGATGAGCC TCACTACACG CACGAGCTCA TATCTGAACC AGCTGTTAGG |
| | VEE GLY |
| | ~~~~~~~~~~~~~~~~~~~~~~~~~~~~~~~~~~~~~~~~~~~~~~~~~~~~~~~~~~~~~~~~~ |
| 1381 | AATTTTACCG TCACCGAAAA AGGGTGGGAG TTTGTATGGG GAAACCACCC GCCGAAAAGG |
| | VEE GLY |
| | ~~~~~~~~~~~~~~~~~~~~~~~~~~~~~~~~~~~~~~~~~~~~~~~~~~~~~~~~~~~~~~~~~ |
| 1441 | TTTTGGGCAC AGGAAACAGC ACCCGGAAAT CCACATGGGC TACCGCACGA GGTGATAACT |
| | VEE GLY |
| | ~~~~~~~~~~~~~~~~~~~~~~~~~~~~~~~~~~~~~~~~~~~~~~~~~~~~~~~~~~~~~~~~~ |
| 1501 | CATTATTACC ACAGATACCC TATGTCCACC ATCCTGGGTT TGTCAATTTG TGCCGCCATT |
| | VEE GLY |
| | ~~~~~~~~~~~~~~~~~~~~~~~~~~~~~~~~~~~~~~~~~~~~~~~~~~~~~~~~~~~~~~~~~ |
| 1561 | GCAACCGTTT CCGTTGCAGC GTCTACCTGG CTGTTTTGCA GATCTAGAGT TGCGTGCCTA |
| | VEE GLY |
| | ~~~~~~~~~~~~~~~~~~~~~~~~~~~~~~~~~~~~~~~~~~~~~~~~~~~~~~~~~~~~~~~~~ |
| 1621 | ACTCCTTACC GGCTAACACC TAACGCTAGG ATACCATTTT GTCTGGCTGT GCTTTGCTGC |
| | VEE GLY |
| | ~~~~~~~~~~~~~~~~~~~~~~~~~~~~~~~~~~~~~~~~~~~~~~~~~~~~~~~~~~~~~~~~~ |
| 1681 | GCCCGCACTG CCCGGGCCGA GACCACCTGG GAGTCCTTGG ATCACCTATG GAACAATAAC |
| | VEE GLY |
| | ~~~~~~~~~~~~~~~~~~~~~~~~~~~~~~~~~~~~~~~~~~~~~~~~~~~~~~~~~~~~~~~~~ |
| 1741 | CAACAGATGT TCTGGATTCA ATTGCTGATC CCTCTGGCCG CCTTGATCGT AGTGACTCGC |
| | VEE GLY |
| | ~~~~~~~~~~~~~~~~~~~~~~~~~~~~~~~~~~~~~~~~~~~~~~~~~~~~~~~~~~~~~~~~~ |
| 1801 | CTGCTCAGGT GCGTGTGCTG TGTCGTGCCT TTTTTAGTCA TGGCCGGCGC CGCAGGCGCC |
| | VEE GLY |
| | ~~~~~~~~~~~~~~~~~~~~~~~~~~~~~~~~~~~~~~~~~~~~~~~~~~~~~~~~~~~~~~~~~ |
| 1861 | GGCGCCTACG AGCACGCGAC CACGATGCCG AGCCAAGCGG GAATCTCGTA TAACACTATA |
| | VEE GLY |
| | ~~~~~~~~~~~~~~~~~~~~~~~~~~~~~~~~~~~~~~~~~~~~~~~~~ |
| 1921 | GTCAACAGAG CAGGCTACGC ACCACTCCCT ATCAGCATAA CACCAACAAA GATCAAGCTG |
| | VEE GLY |
| | ~~~~~~~~~~~~~~~~~~~~~~~~~~~~~~~~~~~~~~~~~~~~~~~~~~~~~~~~~~~~~~~~~ |
| 1981 | ATACCTACAG TGAACTTGGA GTACGTCACC TGCCACTACA AAACAGGAAT GGATTCACCA |
| | VEE GLY |
| | ~~~~~~~~~~~~~~~~~~~~~~~~~~~~~~~~~~~~~~~~~~~~~~~~~~~~~~~~~~~~~~~~~ |
| 2041 | GCCATCAAAT GCTGCGGATC TCAGGAATGC ACTCCAACTT ACAGGCCTGA TGAACAGTGC |
| | VEE GLY |
| | ~~~~~~~~~~~~~~~~~~~~~~~~~~~~~~~~~~~~~~~~~~~~~~~~~~~~~~~~~~~~~~~~~ |
| 2101 | AAAGTCTTCA CAGGGGTTTA CCCGTTCATG TGGGGTGGTG CATATTGCTT TTGCGACACT |
| | VEE GLY |
| | ~~~~~~~~~~~~~~~~~~~~~~~~~~~~~~~~~~~~~~~~~~~~~~~~~~~~~~~~~~~~~~~~~ |
| 2161 | GAGAACACCC AAGTCAGCAA GGCCTACGTA ATGAAATCTG ACGACTGCCT TGCGGATCAT |
| | VEE GLY |
| | ~~~~~~~~~~~~~~~~~~~~~~~~~~~~~~~~~~~~~~~~~~~~~~~~~~~~~~~~~~~~~~~~~ |
| 2221 | GCTGAAGCAT ATAAAGCGCA CACAGCCTCA GTGCAGGCGT TCCTCAACAT CACAGTGGGA |
| | VEE GLY |
| | ~~~~~~~~~~~~~~~~~~~~~~~~~~~~~~~~~~~~~~~~~~~~~~~~~~~~~~~~~~~~~~~~~ |
| 2281 | GAACACTCTA TTGTGACTAC CGTGTATGTG AATGGAGAAA CTCCTGTGAA TTTCAATGGG |
| | VEE GLY |
| | ~~~~~~~~~~~~~~~~~~~~~~~~~~~~~~~~~~~~~~~~~~~~~~~~~~~~~~~~~~~~~~~~~ |
| 2341 | GTCAAAATAA CTGCAGGTCC GCTTTCCACA GCTTGGACAC CCTTTGATCG CAAAATCGTG |
| | VEE GLY |
| | ~~~~~~~~~~~~~~~~~~~~~~~~~~~~~~~~~~~~~~~~~~~~~~~~~~~~~~~~~~~~~~~~~ |
| 2401 | CAGTATGCCG GGGAGATCTA TAATTATGAT TTTCCTGAGT ATGGGGCAGG ACAACCAGGA |
| | VEE GLY |
| | ~~~~~~~~~~~~~~~~~~~~~~~~~~~~~~~~~~~~~~~~~~~~~~~~~~~~~~~~~~~~~~~~~ |
| 2461 | GCATTTGGAG ATATACAATC CAGAACAGTC TCAAGCTCTG ATCTGTATGC CAATACCAAC |
| | VEE GLY |
| | ~~~~~~~~~~~~~~~~~~~~~~~~~~~~~~~~~~~~~~~~~~~~~~~~~~~~~~~~~~~~~~~~~ |
| 2521 | CTAGTGCTGC AGAGACCCAA AGCAGGAGCG ATCCACGTGC CATACACTCA GGCACCTTCG |
| | VEE GLY |
| | ~~~~~~~~~~~~~~~~~~~~~~~~~~~~~~~~~~~~~~~~~~~~~~~~~~~~~~~~~~~~~~~~~ |
| 2581 | GGTTTTGAGC AATGGAAGAA AGATAAAGCT CCATCATTGA AATTTACCGC CCCTTTCGGA |
| | VEE GLY |
| | ~~~~~~~~~~~~~~~~~~~~~~~~~~~~~~~~~~~~~~~~~~~~~~~~~~~~~~~~~~~~~~~~~ |
| 2641 | TGCGAAATAT ATACAAACCC CATTCGCGCC GAAAACTGTG CTGTAGGGTC AATTCCATTA |
| | VEE GLY |
| | ~~~~~~~~~~~~~~~~~~~~~~~~~~~~~~~~~~~~~~~~~~~~~~~~~~~~~~~~~~~~~~~~~ |
| 2701 | GCCTTTGACA TTCCCGACGC CTTGTTCACC AGGGTGTCAG AAACACCGAC ACTTTCAGCG |
| | VEE GLY |
| | ~~~~~~~~~~~~~~~~~~~~~~~~~~~~~~~~~~~~~~~~~~~~~~~~~ |
| 2761 | GCCGAATGCA CTCTTAACGA GTGCGTGTAT TCTTCCGACT TTGGTGGGAT CGCCACGGTC |
| | VEE GLY |
| | ~~~~~~~~~~~~~~~~~~~~~~~~~~~~~~~~~~~~~~~~~~~~~~~~~~~~~~~~~~~~~~~~~ |
| 2821 | AAGTACTCGG CCAGCAAGTC AGGCAAGTGC GCAGTCCATG TGCCATCAGG GACTGCTACC |
| | VEE GLY |
| | ~~~~~~~~~~~~~~~~~~~~~~~~~~~~~~~~~~~~~~~~~~~~~~~~~~~~~~~~~~~~~~~~~ |
| 2881 | CTAAAAGAAG CAGCAGTCGA GCTAACCGAG CAAGGGTCGG CGACTATCCA TTTCTCGACC |
| | VEE GLY |
| | ~~~~~~~~~~~~~~~~~~~~~~~~~~~~~~~~~~~~~~~~~~~~~~~~~~~~~~~~~~~~~~~~~ |
| 2941 | GCAAATATCC ACCCGGAGTT CAGGCTCCAA ATATGCACAT CATATGTTAC GTGCAAAGGT |
| | VEE GLY |
| | ~~~~~~~~~~~~~~~~~~~~~~~~~~~~~~~~~~~~~~~~~~~~~~~~~~~~~~~~~~~~~~~~~ |
| 3001 | GATTGTCACC CCCCGAAAGA CCATATTGTG ACACACCCTC AGTATCACGC CCAAACATTT |
| | VEE GLY |
| | ~~~~~~~~~~~~~~~~~~~~~~~~~~~~~~~~~~~~~~~~~~~~~~~~~~~~~~~~~~~~~~~~~ |
| 3061 | ACAGCCGCGG TGTCAAAAAC CGCGTGGACG TGGTTAACAT CCCTGCTGGG AGGATCAGCC |
| | VEE GLY |
| | ~~~~~~~~~~~~~~~~~~~~~~~~~~~~~~~~~~~~~~~~~~~~~~~~~~~~~~~~~~~~~~~~~ |
| 3121 | GTAATTATTA TAATTGGCTT GGTGCTGGCT ACTATTGTGG CCATGTACGT GCTGACCAAC |
| | VEE GLY 3′UTR |
| | ~~~~~~~~~~~~~~~~~ ~~~~~~~~~~~~~~~~~~~~~~~~~~~~~~~~ |
| 3181 | CAGAAACATA ATTAATAGTA AGCGGCCGCA TACAGCAGCA ATTGGCAAGC TGCTTACATA |
| | 3′UTR |
| | ~~~~~~~~~~~~~~~~~~~~~~~~~~~~~~~~~~~~~~~~~~~~~~~~~~~~~~~~~~~~~~~~~ |
| 3241 | GAACTCGCGG CGATTGGCAT GCCGCCTTAA AATTTTTATT TTATTTTTCT TTTCTTTTCC |
| | 3′UTR |
| | ~~~~~~~~~~~~~~~~~~~~~~~ |
| 3301 | GAATCGGATT TTGTTTTTAA TATTTCAAAA AAAAAAAAAA AAAAAAAAAA AAAAAAAAAA |
| | HDV ribozyme |
| | ~~~~~~~~~~~~~~~~~~~~~~~~~~~~~~~~~~~~~~~~~~~~~~~~~~~~~~~~~~~~~~~~~ |
| 3361 | AGGGTCGGCA TGGCATCTCC ACCTCCTCGC GGTCCGACCT GGGCATCCGA AGGAGGACGC |
| | HDV ribozyme |
| | ~~~~~~~~~~~~~~~~~~~~~~~~~~~~~~~~~~~~ |
| 3421 | ACGTCCACTC GGATGGCTAA GGGAGAGCCA CGTTTAAACA CGTGATATCT GGCCTCATGG |
| 3481 | GCCTTCCTTT CACTGCCCGC TTTCCAGTCG GGAAACCTGT CGTGCCAGCT GCATTAACAT |
| 3541 | GGTCATAGCT GTTTCCTTGC GTATTGGGCG CTCTCCGCTT CCTCGCTCAC TGACTCGCTG |
| | colE1 |
| | ~~~~~~~~~~~~~~~~~~~ |
| 3601 | CGCTCGGTCG TTCGGGTAAA GCCTGGGGTG CCTAATGAGC AAAAGGCCAG CAAAAGGCCA |
| | colE1 |
| | ~~~~~~~~~~~~~~~~~~~~~~~~~~~~~~~~~~~~~~~~~~~~~~~~~~~~~~~~~~~~~~~~~ |
| 3661 | GGAACCGTAA AAAGGCCGCG TTGCTGGCGT TTTTCCATAG GCTCCGCCCC CCTGACGAGC |
| | colE1 |
| | ~~~~~~~~~~~~~~~~~~~~~~~~~~~~~~~~~~~~~~~~~~~~~~~~~~~~~~~~~~~~~~~~~ |
| 3721 | ATCACAAAAA TCGACGCTCA AGTCAGAGGT GGCGAAACCC GACAGGACTA TAAAGATACC |
| | colE1 |
| | ~~~~~~~~~~~~~~~~~~~~~~~~~~~~~~~~~~~~~~~~~~~~~~~~~~~~~~~~~~~~~~~~~ |
| 3781 | AGGCGTTTCC CCCTGGAAGC TCCCTCGTGC GCTCTCCTGT TCCGACCCTG CCGCTTACCG |
| | colE1 |
| | ~~~~~~~~~~~~~~~~~~~~~~~~~~~~~~~~~~~~~~~~~~~~~~~~~~~~~~~~~~~~~~~~~ |
| 3841 | GATACCTGTC CGCCTTTCTC CCTTCGGGAA GCGTGGCGCT TTCTCATAGC TCACGCTGTA |
| | colE1 |
| | ~~~~~~~~~~~~~~~~~~~~~~~~~~~~~~~~~~~~~~~~~~~~~~~~~~~~~~~~~~~~~~~~~ |
| 3901 | GGTATCTCAG TTCGGTGTAG GTCGTTCGCT CCAAGCTGGG CTGTGTGCAC GAACCCCCCG |
| | colE1 |
| | ~~~~~~~~~~~~~~~~~~~~~~~~~~~~~~~~~~~~~~~~~~~~~~~~~~~~~~~~~~~~~~~~~ |
| 3961 | TTCAGCCCGA CCGCTGCGCC TTATCCGGTA ACTATCGTCT TGAGTCCAAC CCGGTAAGAC |
| | colE1 |
| | ~~~~~~~~~~~~~~~~~~~~~~~~~~~~~~~~~~~~~~~~~~~~~~~~~~~~~~~~~~~~~~~~~ |
| 4021 | ACGACTTATC GCCACTGGCA GCAGCCACTG GTAACAGGAT TAGCAGAGCG AGGTATGTAG |
| | colE1 |
| | ~~~~~~~~~~~~~~~~~~~~~~~~~~~~~~~~~~~~~~~~~~~~~~~~~~~~~~~~~~~~~~~~~ |
| 4081 | GCGGTGCTAC AGAGTTCTTG AAGTGGTGGC CTAACTACGG CTACACTAGA AGAACAGTAT |
| | colE1 |
| | ~~~~~~~~~~~~~~~~~~~~~~~~~~~~~~~~~~~~~~~~~~~~~~~~~~~~~~~~~~~~~~~~~ |
| 4141 | TTGGTATCTG CGCTCTGCTG AAGCCAGTTA CCTTCGGAAA AAGAGTTGGT AGCTCTTGAT |
| | colE1 |
| | ~~~~~~~~~~~~~~~~~~~~~~~~~~~~~~~~~~~~~~~~~~~~~~~~~~~~~~~~~~~~~~~~~ |
| 4201 | CCGGCAAACA AACCACCGCT GGTAGCGGTG GTTTTTTTGT TTGCAAGCAG CAGATTACGC |
| | colE1 |
| | ~~~~~~~~~~~~~~~~~~~~~~~~~~~~~~~~~~~~~ |
| 4261 | GCAGAAAAAA AGGATCTCAA GAAGATCCTT TGATCTTTTC TACGGGGTCT GACGCTCAGT |
| 4321 | GGAACGAAAA CTCACGTTAA GGGATTTTGG TCATGAGATT ATCAAAAAGG ATCTTCACCT |
| 4381 | AGATCCTTTT AAATTAAAAA TGAAGTTTTA AATCAATCTA AAGTATATAT GAGTAAACTT |
| 4441 | GGTCTGACAG TTATTAGAAA AATTCATCCA GCAGACGATA AAACGCAATA CGCTGGCTAT |
| | ~~~~~~~~~~~~~~~~~~~~~~~~~~~~~~~~~~~~~~~~~~~~~~~~~~~~ |
| | KanR |
| 4501 | CCGGTGCCGC AATGCCATAC AGCACCAGAA AACGATCCGC CCATTCGCCG CCCAGTTCTT |
| | ~~~~~~~~~~~~~~~~~~~~~~~~~~~~~~~~~~~~~~~~~~~~~~~~~~~~~~~~~~~~~~~~~ |
| | KanR |
| 4561 | CCGCAATATC ACGGGTGGCC AGCGCAATAT CCTGATAACG ATCCGCCACG CCCAGACGGC |
| | ~~~~~~~~~~~~~~~~~~~~~~~~~~~~~~~~~~~~~~~~~~~~~~~~~~~~~~~~~~~~~~~~~ |
| | KanR |
| 4621 | CGCAATCAAT AAAGCCGCTA AAACGGCCAT TTTCCACCAT AATGTTCGGC AGGCACGCAT |
| | ~~~~~~~~~~~~~~~~~~~~~~~~~~~~~~~~~~~~~~~~~~~~~~~~~~~~~~~~~~~~~~~~~ |
| | KanR |
| 4681 | CACCATGGGT CACCACCAGA TCTTCGCCAT CCGGCATGCT CGCTTTCAGA CGCGCAAACA |
| | ~~~~~~~~~~~~~~~~~~~~~~~~~~~~~~~~~~~~~~~~~~~~~~~~~~~~~~~~~~~~~~~~~ |
| | KanR |
| 4741 | GCTCTGCCGG TGCCAGGCCC TGATGTTCTT CATCCAGATC ATCCTGATCC ACCAGGCCCG |
| | ~~~~~~~~~~~~~~~~~~~~~~~~~~~~~~~~~~~~~~~~~~~~~~~~~~~~~~~~~~~~~~~~~ |
| | KanR |
| 4801 | CTTCCATACG GGTACGCGCA CGTTCAATAC GATGTTTCGC CTGATGATCA AACGGACAGG |
| | ~~~~~~~~~~~~~~~~~~~~~~~~~~~~~~~~~~~~~~~~~~~~~~~~~~~~~~~~~~~~~~~~~ |
| | KanR |
| 4861 | TCGCCGGGTC CAGGGTATGC AGACGACGCA TGGCATCCGC CATAATGCTC ACTTTTTCTG |
| | ~~~~~~~~~~~~~~~~~~~~~~~~~~~~~~~~~~~~~~~~~~~~~~~~~~~~~~~~~~~~~~~~~ |
| | KanR |
| 4921 | CCGGCGCCAG ATGGCTAGAC AGCAGATCCT GACCCGGCAC TTCGCCCAGC AGCAGCCAAT |
| | ~~~~~~~~~~~~~~~~~~~~~~~~~~~~~~~~~~~~~~~~~~~~~~~~~~~~~~~~~~~~~~~~~ |
| | KanR |
| 4981 | CACGGCCCGC TTCGGTCACC ACATCCAGCA CCGCCGCACA CGGAACACCG GTGGTGGCCA |
| | ~~~~~~~~~~~~~~~~~~~~~~~~~~~~~~~~~~~~~~~~~~~~~~~~~~~~~~~~~~~~~~~~~ |
| | KanR |
| 5041 | GCCAGCTCAG ACGCGCCGCT TCATCCTGCA GCTCGTTCAG CGCACCGCTC AGATCGGTTT |
| | ~~~~~~~~~~~~~~~~~~~~~~~~~~~~~~~~~~~~~~~~~~~~~~~~~~~~~~~~~~~~~~~~~ |
| | KanR |
| 5101 | TCACAAACAG CACCGGACGA CCCTGCGCGC TCAGACGAAA CACCGCCGCA TCAGAGCAGC |
| | ~~~~~~~~~~~~~~~~~~~~~~~~~~~~~~~~~~~~~~~~~~~~~~~~~~~~~~~~~~~~~~~~~ |
| | KanR |
| 5161 | CAATGGTCTG CTGCGCCCAA TCATAGCCAA ACAGACGTTC CACCCACGCT GCCGGGCTAC |
| | ~~~~~~~~~~~~~~~~~~~~~~~~~~~~~~~~~~~~~~~~~~~~~~~~~~~~~~~~~~~~~~~~~ |
| | KanR |
| 5221 | CCGCATGCAG GCCATCCTGT TCAATCATAC TCTTCCTTTT TCAATATTAT TGAAGCATTT |
| | ~~~~~~~~~~~~~~~~~~~~~~ |
| | KanR |
| 5281 | ATCAGGGTTA TTGTCTCATG AGCGGATACA TATTTGAATG TATTTAGAAA AATAAACAAA |
| 5341 | TAGGGGTTCC GCGCACATTT CCCCGAAAAG TGCCACCTAA ATTGTAAGCG TTAATATTTT |
| 5401 | GTTAAAATTC GCGTTAAATT TTTGTTAAAT CAGCTCATTT TTTAACCAAT AGGCCGAAAT |
| 5461 | CGGCAAAATC CCTTATAAAT CAAAAGAATA GACCGAGATA GGGTTGAGTG GCCGCTACAG |
| 5521 | GGCGCTCCCA TTCGCCATTC AGGCTGCGCA ACTGTTGGGA AGGGCGTTTC GGTGCGGGCC |
| 5581 | TCTTCGCTAT TACGCCAGCT GGCGAAAGGG GGATGTGCTG CAAGGCGATT AAGTTGGGTA |
| | T7 promoter |
| | ~~~~~~~~~~~~~~~~~~~~ |
| 5641 | ACGCCAGGGT TTTCCCAGTC ACACGCGTAA TACGACTCAC TATAG |

### REFERENCES

Britt W J, Alford C A. Cytomegalovirus. In Fields B N, Knipe D M, Howley P M (ed.). Fields Virology, 3^{rd} edition, Philadelphia, Pa.: Lippincott/Raven; 1996. p. 2493-523.
Chee M S, Bankier A T, Beck S, Bohni R, Brown C M, Cerny R, Horsnell T, Hutchinson C A, Kouzarides T, Martignetti J A, Preddie E, Satchwell S C, Tomlinson P, Weston K M and Barrell B G. 1990. Analysis of the protein-coding content of the sequence of human cytomegalovirus strain AD169. Curr. Top. Microbiol. Immunol. 154:125-70.
Davison A J, Dolan A, Akter P, Addison C, Dargan D J, Alcendor D J, McGeoch D J and Hayward G S. 2003. The human cytomegalovirus genome revisited: comparison with the chimpanzee cytomegalovirus genome. J. Gen. Virol. 84:17-28. (Erratum, 84:1053).
Crumpacker C S and Wadhwa S. 2005. Cytomegalovirus, p 1786-1800. In G. L. Mandell, J. E. Bennett, and R. Dolin (ed.), Principles and practice of infectious diseases, vol 2. Elsevier, Philadelphia, Pa.
Pomeroy C and Englund J A. 1987. Cyotmegalovirus: epidemiology and infection control. Am J Infect Control 15: 107-119.
Murphy E, Yu D, Grimwood J, Schmutz J, Dickson M, Jarvis M A, Nelson J A, Myers R M and Shenk T E. 2003. Coding potential of laboratory and clinical strains of cytomegalovirus. Proc. Natl. Acad. Sci. USA 100:14976-81.
Mocarski E S and Tan Courcelle C. 2001. Cytomegalovirus and their replication, p. 2629-73. In D M Knipe and P M Howley (ed.) Fields Virology, 4^{th} edition, vol. 2. Lippincott Williams and Wilkins, Philadelphia, Pa.
Compton T. 2004. Receptors and immune sensors: the complex entry path of human cytomegalovirus. Trends Cell. Bio. 14(1): 5-8.
Britt W J and Alford C A. 2004. Human cytomegalovirus virion proteins. Hum. Immunol. 65:395-402.
Varnum S M, Streblow D N, Monroe M E, Smith P, Auberry K J, Pasa-Tolic L, Wang D, Camp II D G, Rodland K, Wiley, Britt W, Shenk T, Smith R D and Nelson J A. 2004. Identification of proteins in human cytomegalovirus (HCMV) particles: the HCMV proteome. J. Virol. 78:10960-66. (Erratum, 78:13395).
Ljungman P, Griffiths P and Paya C. 2002. Definitions of cytomegalovirus infection and disease in transplant recipients. Clin. Infect. Dis. 34:1094-97.
Rubin R. 2002. Clinical approach to infection in the compromised host, p. 573-679. In R. Rubin and L S Young (ed), Infection in the organ transplant recipient. Kluwer Academic Press, New York, N.Y.
Stagno S and Britt W J. 2005. Cytomegalovirus, p. 389-424. In J S Remington and J O Klein (ed), Infectious diseases of the fetus and newborn infant, 6htt edition. WB Saunders, Phliadelphia, Pa.
Britt W J, Vugler L, Butfiloski E J and Stephens E B. 1990. Cell surface expression of human cytomegalovirus (HCMV) gp55-116 (gB): use of HCMV-vaccinia recombinant virus infected cells in analysis of the human neutralizing antibody response. J. Virol. 64:1079-85.
Reap E A, Dryga S A, Morris J, Rivers B, Norberg P K, Olmsted R A and Chulay J D. 2007. Cellular and Humoral Immune Responses to Alphavirus Replicon Vaccines expressing Cytomegalovirus pp 65, IL1 and gB proteins. Clin. Vacc. Immunol. 14:748-55.
Balasuriya U B R, Heidner H W, Hedges J F, Williams J C, Davis N L, Johnston R E and MacLachlan N J. 2000. Expression of the two major envelope proteins of equine arteritis virus as a heterodimer is necessary for induction of neutralizing antibodies in mice immunized with recombinant Venezuelan equine encephalitis virus replicon particles. J. Virol. 74:10623-30.
Dunn W, Chou C, Li H, Hai R, Patterson D, Stoic V, Zhu H and Liu F. 2003. Functional profiling of a human cytomegalovirus genome. Proc. Natl. Acad. Sci USA 100:14223-28.
Hobom U, Brune W, Messerle M, Hahn G and Kosinowski U H. 2000. Fast screening procedures for random transposon libraries of cloned herpesvirus genomes: mutational analysis of human cytomegalovirus envelope glycoprotein genes. J. Virol. 74:7720-29.
Ryckman B J, Chase M C and Johnson D C. 2009. HCMV TR strain glycoprotein O acts as a chaperone promoting gH/gL incorporation into virions, but is not present in virions. J. Virol.
Wille P T, Knoche A J, Nelson J A, Jarvis M A and Johnson J C. 2009. An HCMV gO-null mutant fails to incorporate gH/gL into the virion envelope and is unable to enter fibroblasts, epithelial, and endothelial cells. J. Virol.
Shimamura M, Mach M and Britt W J. 2006. Human Cytomegalovirus infection elicits a glycoprotein M (gM)/gN-specific virus-neutralizing antibody response. J. Virol. 80:4591-4600.
Cha T A, Tom E, Kemble G W, Duke G M, Mocarski E S and Spaete R R. 1996. Human cytomegalovirus clinical isolates carry at least 19 genes not found in laboratory strains. J. Virol. 70:78-83.
Wang D and Shenk T. 2005. Human cytomegalovirus virion protein complex required for epithelial and endothelial cell tropism. Proc. Natl. Acad. Sci. USA 102:18153-58.
Adler B, Scrivano L, Ruzcics Z, Rupp B, Sinzger C and Kosinowski U. 2006. Role of human cytomegalovirus UL131A in cell type-specific virus entry and release. J. Gen. Virol. 87:2451-60.
Ryckman B J, Rainish B L, Chase M C, Borton J A, Nelson J A, Jarvis J A and Johnson D C. 2008. Characterization of the human cytomegalovirus gH/gL/UL128-UL131 complex that mediates entry into epithelial and endothelial cells. J. Virol. 82: 60-70.

1. A self-replicating RNA molecule comprising a polynucleotide which comprises:
a) a first nucleotide sequence encoding a first protein or fragment thereof that is operably linked to a first subgenomic promoter (SGP); and
b) a second nucleotide sequence encoding a second protein or fragment thereof that is operably linked to a second SGP;
c) a third nucleotide sequence encoding a third protein or fragment thereof that is operably linked to a third SGP; and
d) a fourth nucleotide sequence encoding a fourth protein or fragment thereof that is operably linked to a fourth SGP;
with the proviso that the first protein, the second protein, the third protein and the fourth protein are not the same protein or fragments of the same protein, the first protein is not a fragment of the second, third or fourth protein, the second protein is not a fragment of the first, third or fourth protein, the third protein is not a fragment of the first, second or fourth protein, and the fourth protein is not a fragment of the first, second or third protein;
wherein when the self-replicating RNA molecule is introduced into a suitable cell, the first, second, third and fourth proteins or fragments thereof are produced.

2. The self-replicating RNA molecule of claim 1, further comprising a fifth nucleotide sequence encoding a fifth protein or fragment thereof that is operably linked to a fifth SGP.

3. The self-replicating RNA molecule of claim 1, wherein the first protein or fragment thereof, the second protein or fragment thereof, the third protein or fragment thereof, and the fourth protein or fragment thereof, and when present, the fifth protein or fragment thereof, form a protein complex.

4. The self-replicating RNA molecule of claim 1, wherein the first protein or fragment thereof, the second protein or fragment thereof, the third protein or fragment thereof, the fourth protein or fragment thereof, and, when present, the fifth protein or fragment thereof are each from a herpes virus.

5. The self replicating RNA molecule of any claim 5, wherein the herpes virus is selected from the group consisting of HHV-1, HHV-2, HHV-3, HHV-4, HHV-5, HHV-6, HHV-7, HHV-8 and HHV-9.

6. The self replicating RNA molecule of claim 5 wherein the herpes virus is HHV-5 (CMV).

7. The self-replicating RNA molecule of claim 6 wherein the first protein or fragment, the second protein or fragment, the third protein or fragment, the fourth protein or fragment, and the fifth protein or fragment are independently selected from the group consisting of gB, gH, gL, gO, gM, gN, UL128, UL130, UL131, and a fragment of any one of the foregoing.

8. The self-replicating RNA molecule of claim 7, wherein the first protein or fragment is gH or a fragment thereof, and the second protein or fragment is gL or a fragment thereof, the third protein or fragment is UL128 or a fragment thereof, the fourth protein or fragment is UL130 or a fragment thereof, and the fifth protein or fragment is UL131 or a fragment thereof.

9. The self-replicating RNA molecule of claim 6, wherein the herpes virus is HHV-3 (VZV).

10. The self-replicating RNA molecule of claim 9, wherein the first protein or fragment, the second protein or fragment, the third protein or fragment, the fourth protein or fragment, and the fifth protein or fragment are independently selected from the group consisting of gB, gE, gH, gI, gL, and a fragment of any one of the foregoing.

11. The self-replicating RNA molecule of any one of claim 1, wherein the self-replicating RNA molecule is an alphavirus replicon.

12. An alphavirus replicon particle (VRP) comprising the alphavirus replicon of claim 11.

13. A composition comprising the self-replicating RNA of any one of claim 1 and a pharmaceutically acceptable vehicle.

14. The composition of claim 13, further comprising an RNA delivery system.

15. The composition of claim 14, wherein the RNA delivery system is a liposome, a polymeric nanoparticle, an oil-in-water cationic nanoemulsion or combinations thereof.

16. A method of forming a protein complex, comprising delivering the self-replicating RNA of claim 1 to a cell, and maintaining the cell under conditions suitable for expression of the alphavirus replicon, wherein a protein complex is formed.

17. The method of claim 16 wherein the cell is in vivo.

18. A method of inducing an immune response in an individual, comprising administering to the individual a self-replicating RNA of claim 1.

19. A recombinant DNA molecule that encodes the self-replicating RNA molecule of any one of claim 1.

20. The recombinant DNA molecule of claim 19, wherein the recombinant DNA molecule is a plasmid.
